(19) United States
(12) Patent Application Publication
(10) Pub. No.: US 2026/0176270 A1
(43) Pub. Date: Jun. 25, 2026

(54) Compounds that Inhibit PKMYT1

(71) Applicant: Exelixis, Inc., Alameda, CA (US)

(72) Inventors: Wei XU, Alameda, CA (US); Paige Erin MAHANEY, Alameda, CA (US); Benjamin R. BOSWELL, Alameda, CA (US); David J. COLE, Alameda, CA (US); Paul GILLESPIE, Alameda, CA (US); Faming JIANG, Alameda, CA (US); Ryan KELLEY, Alameda, CA (US); Rachael LANSDOWN, Alameda, CA (US); Xiaodong LIN, Alameda, CA (US); Sunghoon MA, Alameda, CA (US); Jack MAUNG, Alameda, CA (US); Danny NG, Alameda, CA (US); Jian Mehr-Dean PAYANDEH, Alameda, CA (US); Derek PIPER, Alameda, CA (US); Sandeep N. RAIKAR, Alameda, CA (US); Lynne Bannen, Alameda, CA (US); Alice C. RICO, Alameda, CA (US); Justin SALVANT, Alameda, CA (US); Andre H. ST. AMANT, Alameda, CA (US); Yong WANG, Alameda, CA (US); Zhensheng ZHAO, Alameda, CA (US); Guosheng WU, Louisville, KY (US); Beichen ZHANG, Beijing (CN)

(21) Appl. No.: 19/132,531

(22) PCT Filed: Nov. 22, 2023
(86) PCT No.: PCT/US2023/080855

§ 371 Date: May 23, 2025

(30) Foreign Application Priority Data
Nov. 24, 2022 (WO) PCT/CN2022/134096

(51) Int. Cl. C07D 471/04; A61K 31/4375; A61K 31/444; A61K 31/47; A61K 31/4709; A61K 31/472; A61K 31/498; A61K 31/4985; A61K 31/501; A61K 31/506; A61K 31/517; A61K 31/519; A61K 31/5377; A61P 35/00; C07D 215/48; C07D 215/50; C07D 217/02; C07D 217/24; C07D 241/42; C07D 241/44; C07D 401/04; C07D 403/04; C07D 413/04; C07D 417/04; C07D 487/04; C07D 491/107; C07D 519/00

(52) U.S. Cl. CPC C07D 471/04; A61K 31/4375; A61K 31/444; A61K 31/47; A61K 31/4709; A61K 31/472; A61K 31/498; A61K 31/4985; A61K 31/501; A61K 31/506; A61K 31/517; A61K 31/519; A61K 31/5377; A61P 35/00; C07D 215/48; C07D 215/50; C07D 217/02; C07D 217/24; C07D 241/42; C07D 241/44; C07D 401/04; C07D 403/04; C07D 413/04; C07D 417/04; C07D 487/04; C07D 491/107; C07D 519/00

(57) ABSTRACT

Disclosed herein are compounds according to Formula I. (I). Compounds according to Formula I inhibit PKMYT1 and are useful for the treatment of cancer and other disease. The present invention also provides methods for making compounds as mentioned above, and compositions which contain these compounds.

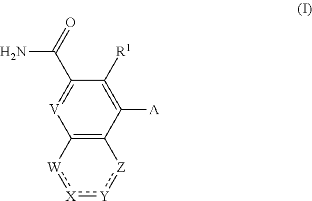

### CROSS-REFERENCES TO RELATED APPLICATIONS

The present application claims priority to International Pat. Appl. No. PCT/CN2022/134096, filed on Nov. 24, 2022, which application is incorporated herein by reference in its entirety.

### FIELD OF THE INVENTION

The present disclosure relates to compounds that inhibit protein kinase, membrane associated tyrosine/threonine 1 (PKMYT1). The disclosure also provides processes for preparing these compounds, pharmaceutical compositions comprising these compounds, and methods of using these compounds to treat diseases, disorders, or conditions responsive to inhibition of PKMYT1.

### BACKGROUND OF THE INVENTION

Protein kinase, membrane associated tyrosine/threonine 1 (PKMYT1) is a member of the WEE family of serine/threonine kinases. PKMYT1 phosphorylates threonine 14 (Thr14) of cyclin-dependent kinase 1 (CDK1), inhibiting its ability to trigger mitosis. Genetically vulnerable tumors that lack a functional PKMYT1 lose major checkpoint regulation, resulting in cell death due to hyperactive CDK1, unscheduled mitosis and catastrophic DNA damage. Abnormal expression of PKMYT1 is associated with certain types of cancers including CCNE1-amplified cancers.

The CCNE1 locus encodes the protein cyclin E1, which complexes with cyclin-dependent kinase 2 (CDK2) and drives cells from the G1 phase to the S phase. Amplification of the CCNE1 locus on chromosome 19q12 is prevalent in multiple tumor types. Disrupting checkpoint regulation via PKMYT1 inhibition may therefore be particularly advantageous in promoting the death of target cancer cells exhibiting irregular expression of CCNE1 and/or cyclin regulators such as F-box/WD repeat-containing protein 7 (FBXW7) and protein phosphatase 2 regulatory subunit A alpha (PPP2R1A). To date, small molecule PKMYT1 inhibitors have been lacking. Thus, a need exists for compounds that inhibit PKMYT1 for the treatment of cancers.

### SUMMARY OF THE INVENTION

In one aspect, the present disclosure provides novel compounds that are PKMYT1 inhibitors.

In one aspect, the present disclosure provides a compound according to Formula I:

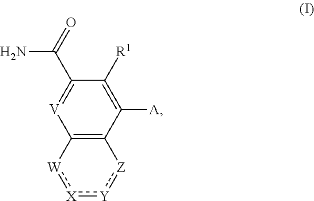

or a pharmaceutically acceptable salt thereof,
wherein:
each [symbol] represents a single bond, a double bond, or a delocalized πbond;
V is selected from the group consisting of CH and N;
W is selected from the group consisting of N, NR^{a}, CR², and C═O;
X is selected from the group consisting of CR³, C═O, N, and NR^{a};
Y is selected from the group consisting of CR⁴, C═O, N, and NR^{a};
Z is selected from the group consisting of N, NR^{a}, CR⁵, and C═O;
R¹ is selected from the group consisting of amino, hydroxy, C₁₋₆ alkyl, and hydrogen;
R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently substituted with 0, 1, 2, 3, or 4 R⁶;
each L is independently selected from the group consisting of a covalent bond, —O—, C₁₋₆ alkylene, and NR^{a};
any two adjacent R², R³, R⁴, and R⁵ are optionally taken together to form a 5- or 6-membered carbocyle or heterocycle;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂; and
each R^{a} is independently hydrogen or C₁₋₆ alkyl;
provided that when R¹ is hydroxy or hydrogen, ring A is selected from the group consisting of:
6- to 14-membered carbocyclyl substituted with (i) at least one R⁶ selected from the group consisting of hydroxy, C₁₋₆ hydroxyalkyl, C₁₋₆ alkoxy, and oxo, and (ii) at least one R⁶ selected from the group consisting of C₁₋₆ alkyl and halogen,
6- or 7-membered monocyclic heterocyclyl comprising at least one oxygen atom, nitrogen atom, or sulfur atom, which heterocycle is substituted with at least one R⁶ selected from the group consisting of hydroxy, C₁₋₆ hydroxyalkyl, oxo, C₁₋₆ alkoxy, C₁₋₆ alkyl, and halogen, and
9- to 14-membered bicyclic heterocyclyl comprising at least one oxygen atom, nitrogen atom, or sulfur atom.

Another aspect provides a compound having a structure according to Formula Ta:

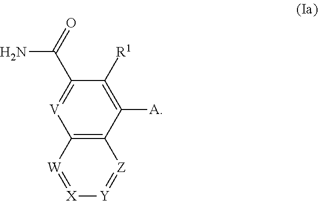

Another aspect of the present disclosure provides a method of treating or preventing conditions or diseases associated with enzymatic activity of PKMYT1.

Another aspect of the present disclosure provides a medical use for preventing conditions or diseases associated with enzymatic activity of PKMYT1.

Another aspect provides methods of using compounds according to Formula I, or a pharmaceutically acceptable salt thereof, for the treatment of cancer.

A further aspect provides a pharmaceutical composition comprising a compound of the disclosure and an excipient and/or pharmaceutically acceptable carrier.

A further aspect provides a pharmaceutical composition comprising a compound of the disclosure and an excipient and/or pharmaceutically acceptable carrier for treating or preventing conditions or diseases associated with enzymatic activity of PKMYT1.

A further aspect provides processes for making compounds of formula I.

These and other aspects and embodiments are described below.

### DETAILED DESCRIPTION OF THE INVENTION

### Abbreviations and Definitions

The following abbreviations and terms have the indicated meanings throughout:

| Abbreviation | Meaning |
|---|---|
| Ac₂O | Acetic anhydride |
| anhyd | Anhydrous |
| Aq | Aqueous |
| Ar | Argon |
| Boc | Tert-butoxycarbonyl |
| ° C. | Degrees Celsius |
| calcd | Calculated |
| chiral SFC | Chiral Supercritical fluid chromatography |
| d | Doublet |
| dd | Doublet of doublets |
| ddd | Doublet of doublets of doublets |
| dt | Doublet of triplets |
| DCM | Dichloromethane |
| DIEA | N,N-Diisopropylethylamine |
| DMB-NH₂ | (2,4-dimethoxyphenyl)methanamine |
| DME | Dimethoxyethane |
| DMF | N,N-Dimethylformamide |
| DMSO | Dimethyl sulfoxide |
| DPPA | Diphenylphosphoryl azide |
| dppp | 1,3-Bis(diphenylphosphino)propane |
| eq or equiv | Equivalent |
| EtOAc | Ethyl acetate |
| EtOH | Ethanol |
| FCC | Flash column chromatography |
| g | Gram(s) |
| h or hr | Hour(s) |
| HATU | 1-[Bis(dimethylamino)methylene]-1H-1,2,3- |
| | triazolo[4,5-b]pyridinium 3-oxide |
| | hexafluorophosphate; Hexafluorophosphate |
| | Azabenzotriazole Tetramethyl Uronium |
| HOAc | Acetic acid |
| HPLC | High pressure liquid chromatography |
| H₂ | Hydrogen |
| L | Liter(s) |
| M | Molar or molarity |
| m | Multiplet |
| MeCN | Acetonitrile |
| MeOH | Methanol |
| MHz | Megahertz (frequency) |
| Min | Minute(s) |
| mL | Milliliter(s) |
| m/z | Mass to charge ratio |
| μL | Microliter(s) |
| mol | Mole(s) |
| mmol | Millimole(s) |
| MS | Mass spectral analysis |
| N₂ | Nitrogen |
| N | Normal or normality |
| NBS | N-bromosuccinimide |
| nM | Nanomolar |
| NMR | Nuclear magnetic resonance spectroscopy |
| Ph₂O | Diphenyl ether |
| Pd/C | Palladium on carbon |
| Pd/L | [2-(2-aminophenyl)phenyl]-methylsulfonyloxy- |
| | palladium dicyclohexyl-[2-(2,6- |
| | dimethoxyphenyl)phenyl]phosphane |
| Pd₂(dba)₃ | Tris(dibenzylideneacetone)dipalladium(0) |
| Pd(dppf)Cl₂•DCM | [1,1′- |
| | Bis(diphenylphosphino)ferrocene]dichlo- |
| | ropalladium(II), |
| | complex with dichloromethane |
| Py | Pyridine |
| Q | Quartet |
| RT | Room temperature |
| s | Singlet |
| soln | Solution |
| SPhos | dicyclohexyl(2′,6′-dimethoxy[1,1′-biphenyl]-2- |
| | yl)phosphane |
| SPhosPdG3 | (2-dicyclohexylphosphino-2′,6′- |
| | dimethoxybiphenyl) [2-(2′-amino-1,1′- |
| | biphenyl)]palladium(II) methanesulfonate |
| t or tr | Triplet |
| TFA | Trifluoroacetic acid |
| t-BuOH | Tert-butyl alcohol |
| TMSCl | Chloro(trimethyl)silane |
| THF | Tetrahydrofuran |

The symbol “-” represents a single bond, and “=” represents a double bond. The symbol [symbol] represents a single bond, a double bond, or a delocalized r bond.

As used herein, the singular forms “a,” “an,” and “the” include plural references unless the context clearly dictates otherwise.

When a variable is defined generically, with a number of possible substituents, each individual radical can be defined with or without the bond. For example, if R¹ can be hydrogen, this can be indicated as “—H” or “H” in the definition of R¹.

When chemical structures are depicted or described, unless explicitly stated otherwise, all carbons are assumed to have hydrogen substitution to conform to a valence of four. For example, in the structure on the left-hand side of the schematic below, there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive expressions are common in the chemical arts to provide brevity and simplicity to description of otherwise complex structures.

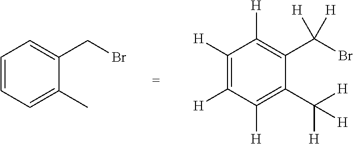

If a group “R” is depicted as “floating” on a ring system, as for example in the formula:

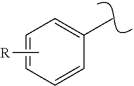

then, unless otherwise defined, a substituent “R” may reside on any atom of the ring system, assuming replacement of a depicted, implied, or expressly defined hydrogen from one of the ring atoms, so long as a stable structure is formed.

“Halogen” or “halo” refers to fluorine, chlorine, bromine, or iodine.

The term “Cₙ₋ₘ” or “Cₙ-Cₘ” indicates a range which includes the endpoints, wherein n and m are integers and indicate the number of carbons. Examples include C₁₋₄, C₁-C₄, C₁₋₆, C₁-C₆, and the like.

“Alkyl” refers to a branched or straight hydrocarbon chain having from 1 to 10 carbon atoms, 1 to 8 carbon atoms, 1 to 6 carbon atoms, 1 to 4 carbon atoms, or 1 to 2 carbon atoms, such as methyl, ethyl, n-propyl, isopropyl, n-butyl, isobutyl, sec-butyl, t-butyl, pentyl, hexyl, and heptyl. The term “C-m alkyl” or (Cₙ-Cₘ) alkyl, refers to an alkyl group having n to m carbon atoms. In some embodiments, alkyl refers to (C₁-C₆)alkyl.

“Alkylene” refers to an optionally substituted bivalent saturated aliphatic radical having from 1 to 10 carbon atoms, 1 to 8 carbon atoms, 1 to 6 carbon atoms, 1 to 4 carbon atoms, or 1 to 2 carbon atoms. In some embodiments, the alkylene group is unsubstituted or not optionally substituted. The term “Cₙ₋ₘ alkylene” refers to an alkylene group having n to m carbon atoms. Examples of alkylene groups include, but are not limited to, methylene, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

“Alkoxy” refers to a moiety of the formula —OR′, wherein R′ is an (C₁-C₆)alkyl moiety as defined herein. The term “Cₙ₋ₘ alkoxy” or (Cₙ-Cₘ) alkoxy refers to an alkoxy group, the alkyl group of which has n to m carbons. Examples of alkoxy moieties include, but are not limited to, methoxy, ethoxy, isopropoxy, and the like.

“Acyl” refers to a moiety of the formula —C(O)R′, wherein R′ is an (C₁-C₆)alkyl moiety as defined herein. The term “Cₙ₋ₘ acyl” or (Cₙ-Cₘ) acyl refers to an acyl group, having a total of n to m carbons. Examples of alkoxy moieties include, but are not limited to, acetyl, propionyl, butyryl, and the like.

The term “amino” refers to a group of formula —NH₂.

“Aryl” means a monovalent six- to fourteen-membered, mono-, bi, or tri-carbocyclic ring (e.g., having two or three fused rings), wherein the monocyclic ring is aromatic and at least one of the rings in the bi- or tri-cyclic ring is aromatic. The term “Cₙ₋ₘ aryl” or “(Cₙ-Cₘ) aryl” refers to an aryl group having from n to m ring carbon atoms. In some embodiments, aryl groups have from 6 to about 10 carbon atoms. In some embodiments, aryl groups have 6 carbon atoms. In some embodiments, aryl groups have 10 carbon atoms. Unless stated otherwise, the point of attachment of the group may be located on any atom of any ring within the radical, valency rules permitting. Representative examples include phenyl, naphthyl, indanyl, and the like.

“Carbocyclyl” refers to a saturated, partially unsaturated, or aromatic ring having 3 to 14 carbon atoms. The carbocyclyl can be a 3 to 7 membered monocycle, 6 to 12 membered bicycle or polycycle. The bicycle or polycycle can be a bridged-ring, fused-ring, or spiro-ring system.

“Cycloalkyl” refers to a non-aromatic hydrocarbon ring system (monocyclic, bicyclic, or polycyclic), including cyclized alkyl and alkenyl groups. The term “Cₙ₋ₘ cycloalkyl” or “(Cₙ-Cₘ) cycloalkyl” refers to a cycloalkyl that has n to m ring member carbon atoms. Cycloalkyl groups can include mono- or polycyclic (e.g., having 2, 3, or 4 fused rings) groups and spirocycles. Cycloalkyl groups can have 3, 4, 5, 6, 7, 8, 9, 10, 11, 12, 13, or 14 ring-forming carbons (C₃₋₁₄). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₁₃ cycloalkyl group. In some embodiments, the cycloalkyl group is a C₃₋₆ monocyclic cycloalkyl group. Ring-forming carbon atoms of a cycloalkyl group can be optionally oxidized to form an oxo group. Cycloalkyl groups also include cycloalkylidenes. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaranyl, bicyclo[1.1.1]pentanyl, bicyclo[2.1.1]hexanyl, and the like. In some embodiments, cycloalkyl includes a single saturated carbocyclic ring of three to eight ring carbons, such as cyclopropyl, cyclobutyl, cyclopentyl, cyclohexyl, cycloheptyl, or cyclooctyl. In some embodiments, the cycloalkyl group is cyclopropyl, cyclobutyl, cyclopentyl, or cyclohexyl.

A cycloalkyl group can be unsubstituted or optionally substituted. When optionally substituted, one or more hydrogen atoms of the cycloalkyl group (e.g., from 1 to 4, from 1 to 2, or 1) may be replaced with a moiety as described. In some embodiments, a substituted cycloalkyl group can incorporate an exo- or endocyclic alkene (e.g., cyclohex-2-en-1-yl).

The term “cyano” or “nitrile” refers to a group of formula —C—N, which also may be written as —CN or CN.

The term “heteroatom” used herein is meant to include boron, phosphorus, sulfur, oxygen, and nitrogen.

The term “haloalkyl” as used herein refers to an alkyl group in which one or more of the hydrogen atoms has been replaced by a halogen atom. The term “Cₙ₋ₘ haloalkyl” or (Cₙ-Cₘ) haloalkyl refers to a Cₙ₋ₘ alkyl group having n to m carbon atoms and from at least one up to {2(n to m)+1}halogen atoms, which may either be the same or different. In some embodiments, the haloalkyl group has 1 to 6 or 1 to 4 carbon atoms. In some embodiments, the halogen atoms include fluoro atoms. In some embodiments, the haloalkyl group is a fluoroalkyl group. Example haloalkyl groups include CF₃, C₂F₅, CHF₂, CCl₃, CHCl₂, C₂C₅, and the like.

The term “haloalkoxy” refers to a group of formula —O-haloalkyl, wherein the haloalkyl group is as defined above. The term “Cₙ₋ₘ haloalkoxy” or (Cₙ-Cₘ) haloalkoxy refers to a haloalkoxy group, the haloalkyl group of which has n to m carbons. In some embodiments, the haloalkoxy group has 1 to 6, 1 to 4, or 1 to 3 carbon atoms. Example haloalkoxy groups include trifluoromethoxy and the like.

“Heteroaryl” means a monocyclic, fused bicyclic, or fused tricyclic, monovalent radical of 5 to 14 ring atoms containing one or more (e.g., one, two, three, or four) ring members independently selected from —O—, —S(O)ₙ— (n is 0, 1, or 2), —N—, and —N(R′)—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R′ is hydrogen, alkyl, hydroxy, alkoxy, acyl, or alkylsulfonyl. Unless stated otherwise, the point of attachment may be located on any atom of any ring of the heteroaryl group, valency rules permitting. In particular, when the point of attachment is located on the nitrogen, an additional nitrogen substituent is not present. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, isoindolyl, indolinyl (including, for example, 2,3-dihydro-1H-indol-2-yl or 2,3-dihydro-1H-indol-5-yl, and the like), isoindolinyl, benzimidazolyl, benzodioxol-4-yl, benzofuranyl, cinnolinyl, indolizinyl, naphthyridinyl, naphthyridin-3-yl, phthalazin-3-yl, phthalazin-4-yl, pyridazopyrazinyl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), pyrazolopyridinyl, tetrahydropyrazolopyridinyl, benzopyranyl, thiazolyl, isothiazolyl, thiadiazolyl, benzothiazolyl, benzothienyl, and the derivatives thereof, and N-oxide or a protected derivative thereof.

As used herein, “heterocyclyl” refers to a saturated, partially unsaturated, or aromatic ring or ring system, which has at least one heteroatom ring member independently selected from boron, nitrogen, sulfur, oxygen, and phosphorus, and which has 4-14 ring members, 4-10 ring members, 4-7 ring members, or 4-6 ring members. Heterocyclyl groups can include mono- or bicyclic or polycyclic (e.g., having two or three fused or bridged rings) ring systems or spirocycles. In some embodiments, the heterocyclyl group is a monocyclic group having 1, 2, or 3 heteroatoms independently selected from nitrogen, sulfur, and oxygen. Ring-forming carbon atoms and heteroatoms of a heterocycloalkyl group can be optionally oxidized to form an oxo or sulfonyl group or other oxidized linkage (e.g., C(O), S(O), C(S), S(O)₂, N-oxide, and the like) or a nitrogen atom can be quaternized. In some embodiments, the heterocyclyl group is an aromatic monocyclic or bicyclic group having 1, 2, or 3 heteroatoms independently selected from nitrogen, sulfur, and oxygen. Example of heterocyclyl groups include pyridinyl, naphthyridinyl, pyridazopyrazinyl, pyridopyrazinyl, quinolinyl, quinoxalinyl, etc.

The term “hydroxyl” or “hydroxy” refers to an —OH moiety.

“Hydroxyalkyl” means an alkyl group, as defined herein, substituted with at least one, particularly, 1, 2, 3, or 4, hydroxy groups. The term “Cₙ₋ₘ hydroxyalkyl” or (Cₙ-Cₘ) hydroxyalkyl refers to a hydroxyalkyl group, the hydroxyalkyl group of which has n to m carbons. In some embodiments, the hydroxyalkyl group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

As used herein, a “leaving group” (Lv) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo), tosyl, mesyl, and besyl. In certain embodiments, the leaving group is a halogen.

“Subject” for the purposes of the present disclosure includes humans and any other animals, particularly mammals, and other organisms. Thus the methods are applicable to both human therapy and veterinary applications. In some embodiments, the subject is a human or other mammal. Examples of other mammals include mice, rats, other rodents, rabbits, dogs, cats, swine, cattle, sheep, horses, and non-human primates.

“Therapeutically effective amount” is an amount of a compound as described herein that, when administered to a patient, ameliorates a symptom of the disease. The amount of a compound which constitutes a “therapeutically effective amount” will vary depending on the compound, the disease state and its severity, the age of the patient to be treated, and the like.

“Cancer” refers to cellular-proliferative disease states, including carcinomas, sarcomas, leukemias, and lymphomas. The term “cancerous cell” as provided herein, includes a cell afflicted by any one of the above-identified conditions.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids as well as organic acids.

“Pharmaceutically acceptable base addition salts” include those derived from inorganic bases and organic bases.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

Compounds as described herein can also include all isotopes of atoms occurring in synthetic intermediates or final compounds. Isotopes include those atoms having the same atomic number but different mass numbers. For example, isotopes of hydrogen include tritium and deuterium.

Any one of the process steps or sequences disclosed and/or claimed herein can be performed under an inert gas atmosphere, more particularly under argon or nitrogen. In addition, the methods of the present disclosure may be carried out as semi-continuous or continuous processes.

In general, the nomenclature used in this Application is based on naming conventions adopted by the International Union of Pure and Applied Chemistry (IUPAC). Chemical structures shown herein were prepared using CHEMDRAW®. Any open valency appearing on a carbon, oxygen, or nitrogen atom in the structures herein indicates the presence of a hydrogen atom.

### Compounds of the Disclosure

One aspect of the present disclosure provides a compound according to Formula I:

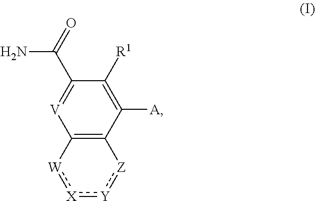

or a pharmaceutically acceptable salt thereof,
wherein:
each [symbol] represents a single bond, a double bond, or a delocalized π bond;
V is selected from the group consisting of CH and N;
W is selected from the group consisting of N, NR^{a}, CR², and C═O;
X is selected from the group consisting of CR³, C═O, N, and NR^{a};
Y is selected from the group consisting of CR⁴, C═O, N, and NR^{a};
Z is selected from the group consisting of N, NR^{a}, CR⁵, and C═O;
R¹ is selected from the group consisting of amino, hydroxy, C₁₋₆ alkyl, and hydrogen;
R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently substituted with 0, 1, 2, 3, or 4 R⁶;
each L is independently selected from the group consisting of a covalent bond, —O—, C₁₋₆ alkylene, and NR^{a};
any two adjacent R², R³, R⁴, and R⁵ are optionally taken together to form a 5- or 6-membered carbocyle or heterocycle,
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂; and
each R^{a} is independently hydrogen or C₁₋₆ alkyl;
provided that when R¹ is hydroxy or hydrogen, ring A is selected from the group consisting of:
6- to 14-membered carbocyclyl substituted with (i) at least one R⁶ selected from the group consisting of hydroxy, C₁₋₆ hydroxyalkyl, C₁₋₆ alkoxy, and oxo, and (ii) at least one R⁶ selected from the group consisting of C₁₋₆ alkyl and halogen,
6- or 7-membered monocyclic heterocyclyl comprising at least one oxygen atom, nitrogen atom, or sulfur atom, which heterocycle is substituted with at least one R⁶ selected from the group consisting of hydroxy, C₁₋₆ hydroxyalkyl, oxo, C₁₋₆ alkoxy, C₁₋₆ alkyl, or halogen, and
9- to 14-membered bicyclic heterocyclyl comprising at least one oxygen atom, nitrogen atom, or sulfur atom.

Some embodiments of the present disclosure provide compounds according to Formula I as set forth above, and pharmaceutically acceptable salts thereof, provided that when R¹ is C₁₋₆ alkyl (e.g., methyl), ring A is selected from the group consisting of:
6- to 14-membered carbocyclyl substituted with (i) at least one R⁶ selected from the group consisting of hydroxy, C₁₋₆ hydroxyalkyl, C₁₋₆ alkoxy, and oxo, and (ii) at least one R⁶ selected from the group consisting of C₁₋₆ alkyl and halogen,
6- or 7-membered monocyclic heterocyclyl comprising at least one oxygen atom, nitrogen atom, or sulfur atom, which heterocycle is substituted with at least one R⁶ selected from the group consisting of hydroxy, C₁₋₆ hydroxyalkyl, oxo, C₁₋₆ alkoxy, C₁₋₆ alkyl, or halogen, and
9- to 14-membered bicyclic heterocyclyl comprising at least one oxygen atom, nitrogen atom, or sulfur atom.

In some embodiments, the compound according to Formula I, or a pharmaceutically acceptable salt thereof, is a compound having a structure according to Formula Ia:

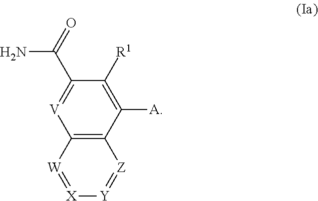

In some embodiments, at least one of V, W, X, Y, and Z is N.

In some embodiments, at least two of V, W, X, Y, and Z are N.

In some embodiments, at least three of V, W, X, Y, and Z are N.

In some embodiments, V is N.

In some embodiments, V is CH.

In some embodiments, W is CR².

In some embodiments, W is N.

In some embodiments, W is C═O.

In some embodiments, X is N.

In some embodiments, X is C═O.

In some embodiments, X is CR³.

In some embodiments, Y is N.

In some embodiments, Y is C═O.

In some embodiments, Y is CR⁴.

In some embodiments, Z is N.

In some embodiments, Z is C═O.

In some embodiments, Z is CR⁵.

In some embodiments, R¹ is amino.

In some embodiments, R¹ is hydrogen.

In some embodiments, R¹ is hydroxy.

In some embodiments, R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently substituted with 0, 1, 2, 3, or 4 R⁶.

In some embodiments, each L is independently selected from the group consisting of a covalent bond, —O—, C₁₋₆ alkylene, and NR^{a}. In some embodiments, L is a covalent bond.

In some embodiments, R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), and -L-(5- to 14-membered heteroaryl). In some embodiments, the 5- to 14-membered heteroaryl is substituted with 0, 1, or 2 R⁶. In some embodiments, the 5- to 14-membered heteroaryl is substituted with C₁₋₆ alkyl.

In some embodiments, two adjacent R², R³, R⁴, and R⁵ (e.g., R² and R³; R³ and R⁴; or R⁴ and R⁵) are optionally taken together to form a 5- or 6-membered carbocyle or heterocycle (e.g., a benzo ring or a pyrido ring). The carbocycle or heterocycle may optionally be substituted with one or more substituents independently selected from amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁₋₆ alkyl)

In some embodiments, R² is selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁₋₆ alkyl).

In some embodiments, R² is selected from the group consisting of hydrogen, halogen, cyano, C₁₋₆ alkyl, and C₁₋₆ haloalkyl.

In some embodiments, R² is selected from the group consisting of —H, —F, —Cl, —CH₃, and —CF₃. In some embodiments, R² is —H.

In some embodiments, R³ is selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁₋₆ alkyl).

In some embodiments, R³ is selected from the group consisting of hydrogen, hydroxy, halogen, C₁₋₆ alkyl, C₁₋₆ haloalkyl, and C₁₋₆ alkoxy. In some embodiments, R³ is selected from the group consisting of hydrogen, hydroxy, halogen, C₁₋₆ alkyl, C₁₋₆ haloalkyl, C₁₋₆ alkoxy, and -L-(5- to 14-membered heteroaryl), wherein the 5- to 14-membered heteroaryl is substituted with 0, 1, or 2 R⁶. In some embodiments, L is a covalent bond. In some embodiments, the 5- to 14-membered heteroaryl is substituted with 1 or 2 R⁶. In some embodiments, the 5- to 14-membered heteroaryl is unsubstituted or is substituted with C₁₋₆ alkyl.

In some embodiments, R³ is selected from the group consisting of —H, —F, —Cl, —CH₃, —OH, —CF₃, and —OCH₃. In some embodiments, R³ is —H. In some embodiments, R³ is

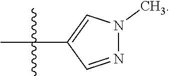 [SMILES: *c1cnn(C)c1]

In some embodiments, R⁴ is selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁₋₆ alkyl).

In some embodiments, R⁴ is selected from the group consisting of hydrogen, hydroxy, halogen, C₁₋₆ alkyl, C₁₋₆ haloalkyl, and C₁₋₆ alkoxy.

In some embodiments, R⁴ is selected from the group consisting of —H, —F, —Cl, —CH₃, —OH, —CF₃, —OCH₃, and —OCH₂CH₃. In some embodiments, R⁴ is —H.

In some embodiments, X is CR³ and Y is CR⁴; and R³ and R⁴ are selected from the group consisting of hydrogen, hydroxy, halogen, cyano, C₁₋₆ alkyl, C₁₋₆ haloalkyl, and C₁₋₆ alkoxy.

In some embodiments, R⁵ is selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁₋₆ alkyl).

In some embodiments, R⁵ is selected from the group consisting of hydrogen, hydroxy, halogen, cyano, and C₁₋₆ alkyl.

In some embodiments, R⁵ is selected from the group consisting of —H, —F, —Cl, —CN, —OH, and —CH₃. In some embodiments, R⁵ is —H.

In some embodiments, Z is CR⁵, and R⁵ is selected from the group consisting of hydrogen, hydroxy, halogen, cyano, and C₁₋₆ alkyl.

In some embodiments, each R⁶ of ring A is independently selected from the group consisting of hydroxy, cyano, amino, halogen, —CHO, C₁₋₆ alkyl, C₁₋₆ haloalkyl, C₁₋₆ hydroxyalkyl, C₁₋₆ alkoxy, and oxo.

In some embodiments, each R⁶ of ring A is independently selected from the group consisting of hydroxy, C₁₋₆ hydroxyalkyl, C₁₋₆ alkoxy, and oxo.

In some embodiments, each R⁶ of ring A is independently selected from the group consisting of —OH, —F, —Cl, —CN, —CH₃, —CF₃, —OCH₃, —CHO, and oxo.

In some embodiments, ring A is a 3- to 14-membered carbocyclyl substituted with 1, 2, or 3 R⁶, and each R⁶ is independently selected from the group consisting of hydroxy, cyano, amino, halogen, —CHO, C₁₋₆ alkyl, C₁₋₆ haloalkyl, C₁₋₆ hydroxyalkyl, C₁₋₆ alkoxy, and oxo.

In some embodiments, ring A is a 6- to 10-membered cycloalkyl substituted with 1, 2, or 3 R⁶. In some embodiments, ring A is a 6- to 10-membered cycloalkyl substituted with 1, 2, or 3 R⁶, and each R⁶ is independently selected from the group consisting of hydroxy, amino, halogen, C₁₋₆ alkyl, C₁₋₆ haloalkyl, C₁₋₆ hydroxyalkyl, C₁₋₆ alkoxy, and oxo.

In some embodiments, ring A is 6- to 10-membered aryl substituted with 1, 2, or 3 R⁶. In some embodiments, ring A is 6- to 10-membered aryl substituted with 1, 2, or 3 R⁶, and each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, and oxo.

In some embodiments, ring A is phenyl substituted with 1, 2, 3, or 4 R⁶. In some embodiments, ring A is phenyl substituted with 1, 2, or 3 R⁶, and each R⁶ is independently selected from the group consisting of hydroxy, amino, cyano, halogen, C₁₋₆ alkyl, C₁₋₆ haloalkyl, C₁₋₆ hydroxyalkyl, and C₁₋₆ alkoxy.

In some embodiments, ring A is 5- to 14-membered nitrogen-containing heterocyclyl substituted with 0, 1, 2, or 3 R⁶. In some embodiments, ring A is 5- to 14-membered nitrogen-containing heterocyclyl substituted with 0, 1, 2, or 3 R⁶, and each R⁶ is independently selected from the group consisting of hydroxy, amino, halogen, —CHO, C₁₋₆ alkyl, C₁₋₆ haloalkyl, C₁₋₆ hydroxyalkyl, C₁₋₆ alkoxy, and oxo.

In some embodiments, ring A is 5- to 7-membered monocyclic nitrogen-containing heterocyclyl substituted with 0, 1, 2, or 3 R⁶. In some embodiments, ring A is 5- to 7-membered monocyclic nitrogen-containing heterocyclyl substituted with 0, 1, 2, or 3 R⁶, and each R⁶ is independently selected from the group consisting of hydroxy, amino, halogen, C₁₋₆ alkyl, C₁₋₆ haloalkyl, C₁₋₆ hydroxyalkyl, C₁₋₆ alkoxy, and oxo. In some embodiments, ring A is 5- or 6-membered monocyclic nitrogen-containing heteroayl substituted with 0, 1, 2, or 3 R⁶, and each R⁶ is independently selected from the group consisting of hydroxy, amino, halogen, C₁₋₆ alkyl, C₁₋₆ haloalkyl, and C₁₋₆ alkoxy.

In some embodiments, ring A is 8- to 10-membered bicyclic nitrogen-containing heterocyclyl substituted with 0, 1, 2, or 3 R⁶. In some embodiments, ring A is 8- to 10-membered bicyclic nitrogen-containing heterocyclyl substituted with 0, 1, or 2 R⁶, and each R⁶ is independently selected from the group consisting of hydroxy, amino, halogen, —CHO, C₁₋₆ alkyl, C₁₋₆ haloalkyl, C₁₋₆ hydroxyalkyl, C₁₋₆ alkoxy, and oxo.

In some embodiments, R¹ is amino, and ring A is: 3- to 14-membered carbocyclyl substituted with 1, 2, 3, or 4 R⁶, or 5- to 14-membered nitrogen-containing heterocyclyl substituted with 0, 1, 2, 3, or 4 R⁶.

In some embodiments, R¹ is amino, and ring A is phenyl substituted with 1, 2, or 3 R⁶. In some embodiments, R¹ is amino, and ring A is phenyl substituted with 1, 2, or 3 R⁶, and each R⁶ is independently selected from the group consisting of hydroxy, amino, cyano, halogen, C₁. 6 alkyl, C₁₋₆ haloalkyl, C₁₋₆ hydroxyalkyl, C₁₋₆ alkoxy, and oxo.

In some embodiments, R¹ is amino, and ring A is 3- to 14-membered carbocyclyl substituted with 1, 2, or 3 R⁶, and each R⁶ is independently selected from the group consisting of hydroxy, amino, halogen, C₁₋₆ alkyl, C₁₋₆ haloalkyl, C₁₋₆ hydroxyalkyl, C₁₋₆ alkoxy, and oxo.

In some embodiments, R¹ is amino, and ring A is 5- to 14-membered nitrogen-containing heterocyclyl substituted with 0, 1, 2, or 3 R⁶, and each R⁶ is independently selected from the group consisting of hydroxy, amino, halogen, —CHO, C₁₋₆ alkyl, C₁₋₆ haloalkyl, C₁₋₆ hydroxyalkyl, C₁₋₆ alkoxy, and oxo.

In some embodiments, R¹ is amino, and ring A is 5- to 7-membered monocyclic nitrogen-containing heterocyclyl substituted with 0, 1, 2, or 3 R⁶. In some embodiments, R¹ is amino, and ring A is 5- to 7-membered monocyclic nitrogen-containing heterocyclyl substituted with 0, 1, 2, or 3 R⁶, and each R⁶ is independently selected from the group consisting of hydroxy, amino, halogen, C₁₋₆ alkyl, C₁₋₆ haloalkyl, C₁₋₆ hydroxyalkyl, C₁₋₆ alkoxy, and oxo.

In some embodiments, R¹ is amino, and ring A is 8- to 10-membered bicyclic nitrogen-containing heterocyclyl substituted with 0, 1, 2, or 3 R⁶. In some embodiments, R¹ is amino, and ring A is 8- to 10-membered monocyclic nitrogen-containing heterocyclyl substituted with 0, 1, 2, or 3 R⁶, and each R⁶ is independently selected from the group consisting of hydroxy, amino, halogen, —CHO, C₁₋₆ alkyl, C₁₋₆ haloalkyl, C₁₋₆ hydroxyalkyl, C₁₋₆ alkoxy, and oxo.

In some embodiments, R¹ is hydrogen or hydroxy, and ring A is selected from the group consisting of:
a 6- to 14-membered carbocyclyl substituted with (i) at least one of hydroxy, C₁₋₆ hydroxyalkyl, C₁₋₆ alkoxy, or oxo, and (ii) at least one C₁₋₆ alkyl or halogen,
a 6- or 7-membered monocyclic nitrogen containing heterocyclyl substituted with 1, 2, or 3 substituents independently selected from the group consisting of hydroxy, C₁₋₆ hydroxyalkyl, oxo, C₁₋₆ alkoxy, C₁₋₆ alkyl, or halogen, and
a 9- to 14-membered bicyclic nitrogen containing heterocyclyl substituted with 0, 1, 2, or 3 substituents independently selected from the group consisting of C₁₋₆ alkyl, C₁₋₆ alkoxy, C₁₋₆ haloalkyl, and halogen.

In some embodiments, R¹ is hydrogen, and ring A is a 6- to 10-membered aryl substituted with (i) at least one of hydroxy, hydroxy(C₁₋₆ alkyl), C₁₋₆ alkoxy, or oxo, and (ii) at least one of C₁₋₆ alkyl or halogen.

In some embodiments, R¹ is hydrogen, and ring A is a 6- or 7-membered monocyclic nitrogen-containing heteroaryl substituted with 1, 2, or 3 R⁶, and each R⁶ is independently selected from the group consisting of hydroxy, C₁₋₆ hydroxyalkyl, oxo, C₁₋₆ alkoxy, C₁₋₆ alkyl, and halogen.

In some embodiments, R¹ is hydrogen, and ring A is a 9- to 10-membered bicyclic nitrogen-containing heteroaryl substituted with 0, 1, 2, or 3 R⁶, and each R⁶ is independently selected from the group consisting of C₁₋₆ alkyl, C₁₋₆ alkoxy, C₁₋₆ haloalkyl, and halogen.

Any of the ring A moieties described above may be present, for example, in compounds according to Formula I, Ia, Ib, Ic, Ic-1, Id, Ie, If, If-1, Ig, Ih, IIa, IIb, and/or IIc as set forth herein.

In some embodiments, ring A is selected from the group consisting of:

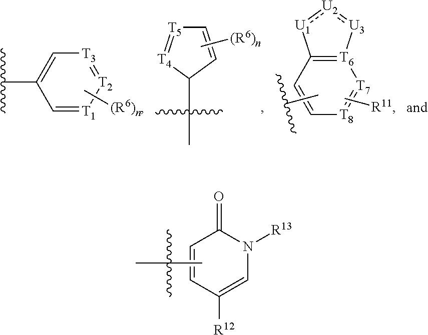

wherein
each [symbol] represents a single bond, a double bond, or a delocalized π bond;
T₁, T₂, T₃, T₄, T₅, T₆, T₇, and T₈ are independently selected from the group consisting of N, C, and CH;
U₁, U₂, and U₃ are independently selected from the group consisting of N, NR¹⁰, O, S, and CR¹⁴;
each R⁶ is independently selected from the group consisting of amino, oxo, cyano, hydroxy, C₁₋₆ alkyl, C₁₋₆ alkoxy, C₁₋₆ haloalkyl, C₁₋₆ hydroxyalkyl, and halogen;
R¹¹, R¹², and R¹⁴ are independently selected from the group consisting of hydrogen, amino, oxo, cyano, hydroxy, C₁₋₆ alkyl, C₁₋₆ alkoxy, C₁₋₆ haloalkyl, C₁₋₆ hydroxyalkyl, and halogen;
each R¹⁰ and R¹³ are independently rselected from the group consisting of hydrogen, C₁₋₆ alkyl, and CHO; and
n is 1, 2, 3, or 4.

In some embodiments, ring A in compounds according to Formula I, Ia, Ib, Ic, Ic-1, Id, Ie, If, If-1, Ig, Ih, Ila, IIb, and/or IIc is

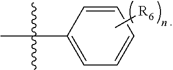

In some embodiments, ring A is selected from the group consisting of

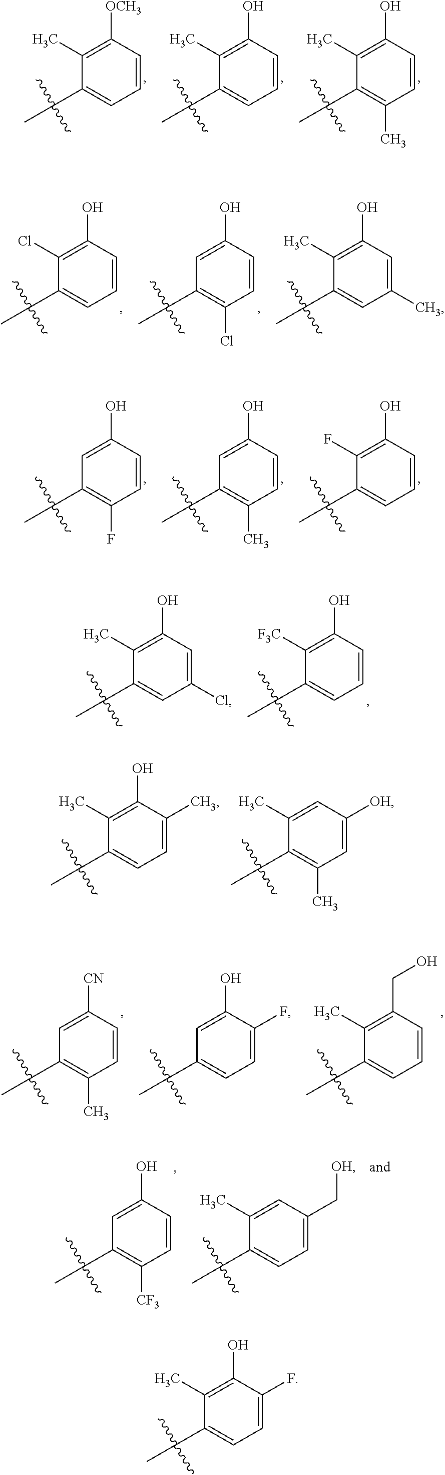

In some embodiments, ring A in compounds according to Formula I, Ia, Ib, Ic, Ic-1, Id, Ie, If, If-1, Ig, Ih, IIa, IIb, and/or IIc is

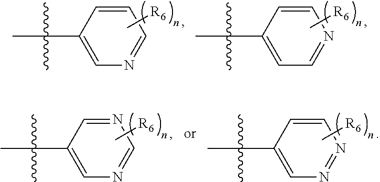

In some embodiments, ring A is selected from the group consisting of

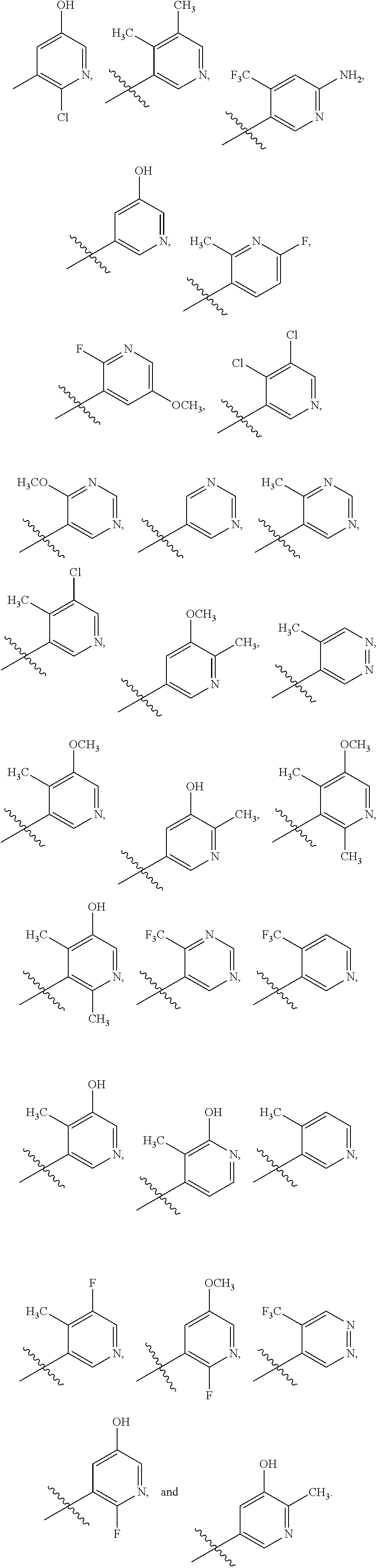

In some embodiments, ring A in compounds according to Formula I, Ta, Ib, Ic, Ic-1, Id, Ie, If, If-1, Ig, Ih, IIa, IIb, and/or IIc is

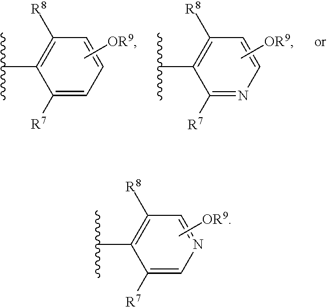

wherein R⁷ and R⁸ are independently selected from the group consisting of hydrogen, amino, cyano, C₁₋₆ alkyl, C₁₋₆ alkoxy, C₁₋₆ haloalkyl, C₁₋₆ hydroxyalkyl, and halogen; and R⁹ is hydrogen or C₁₋₆ alkyl.

In some embodiments, ring A in compounds according to Formula I, Ia, Ib, Ic, Ic-1, Id, Ie, If, If-1, Ig, Ih, IIa, IIb, and/or IIc is selected from the group consisting of

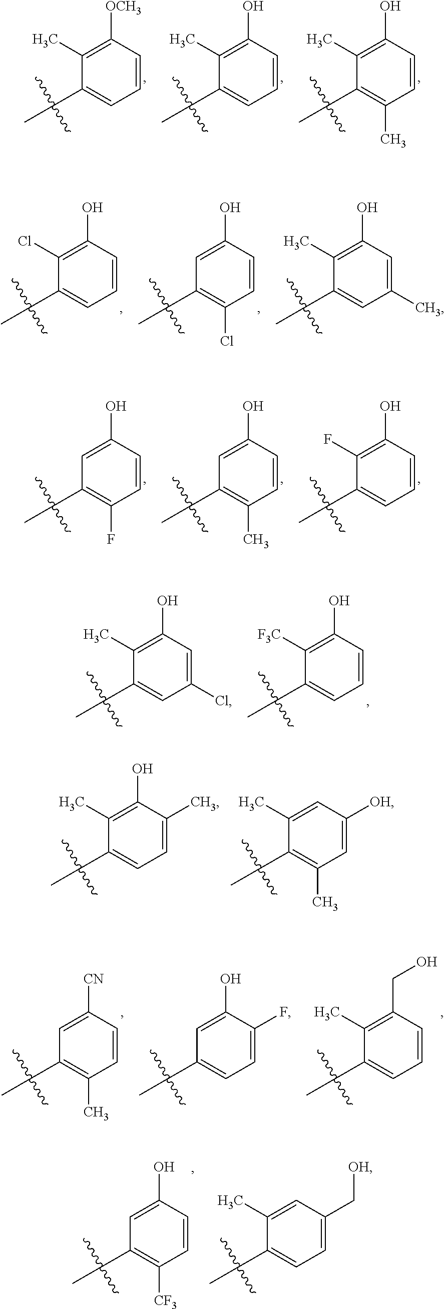

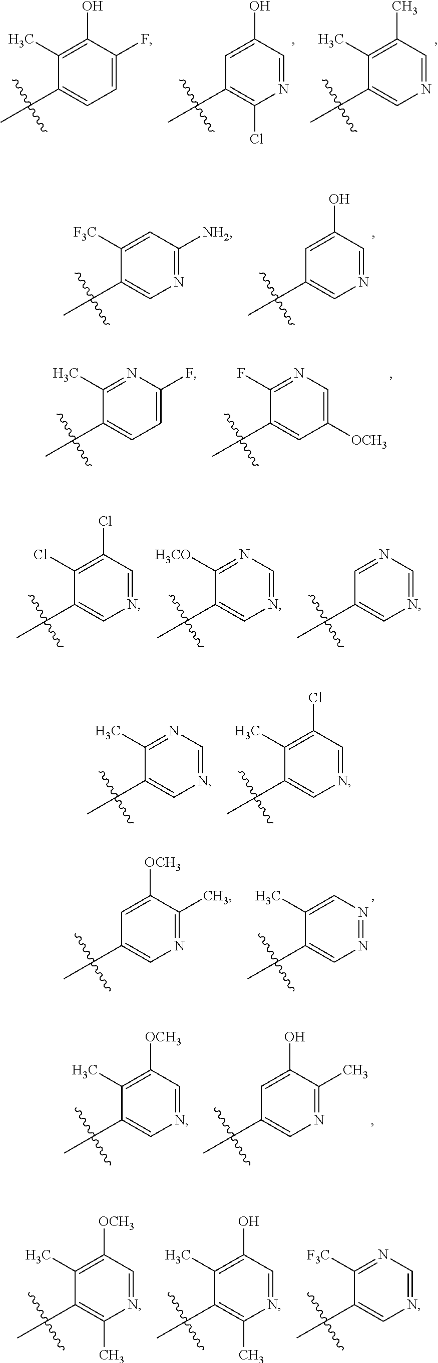

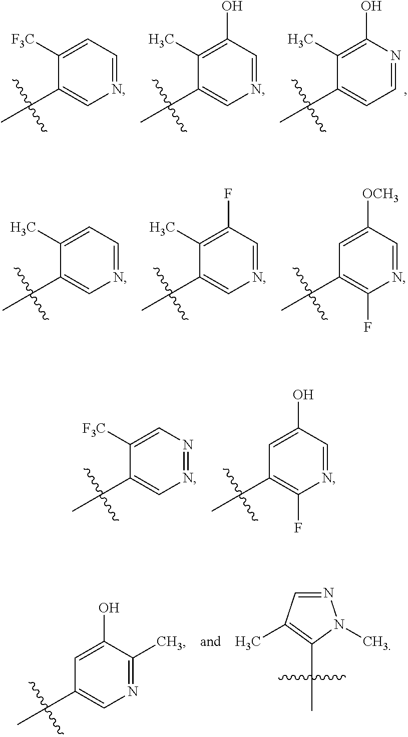

In some embodiments, ring A in compounds according to Formula I, la, Ib, Ic, Ic-1, Id, Ie, If, If-1, Ig, Ih, IIa, IIb, and/or IIc is selected from the group consisting of

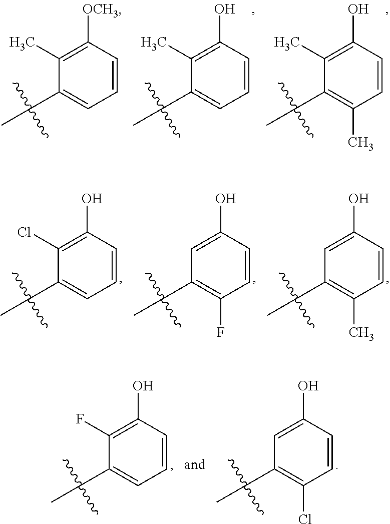

In some embodiments, ring A in compounds according to Formula I, Ia, Ib, Ic, Ic-1, Id, Ie, If, If-1, Ig, Ih, IIa, IIb, and/or IIc is:

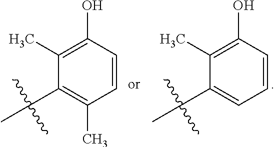

In some embodiments, ring A in compounds according to Formula I, Ia, Ib, Ic, Ic-1, Id, Ie, If, If-1, Ig, Ih, IIa, IIb, and/or IIc is:

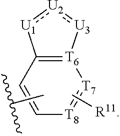

In some embodiments, ring A is selected from the group consisting of:

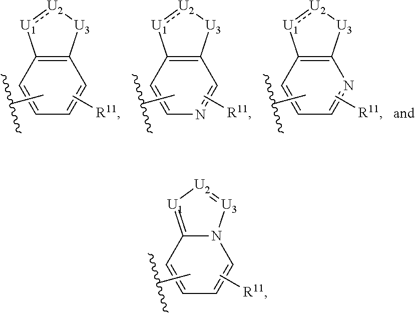

wherein [symbol] represents a single bond or a double bond.

In some embodiments, ring A is selected from the group consisting of:

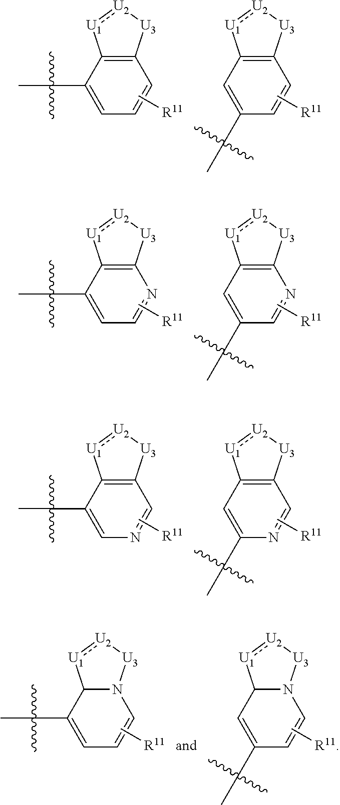

In some embodiments, each R¹¹ is independently selected from the group consisting of hydrogen, cyano, C₁₋₆ alkyl, C₁₋₆ alkoxy, C₁₋₆ haloalkyl, C₁₋₆ hydroxyalkyl, and halogen; and
R¹⁴ are independently selected from the group consisting of hydrogen, amino, oxo, cyano, hydroxy, C₁₋₆ alkyl, and halogen.

In some embodiments, ring A is:

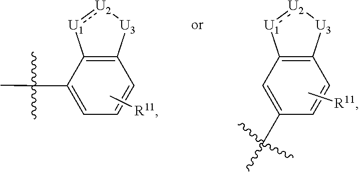

wherein R¹¹ is selected from the group consisting of hydrogen, C₁₋₆ alkyl and halogen; and
R¹⁴ is selected from the group consisting of hydrogen, amino, oxo, and C₁₋₆ alkyl.

In some embodiments, ring A is selected from the group consisting of:

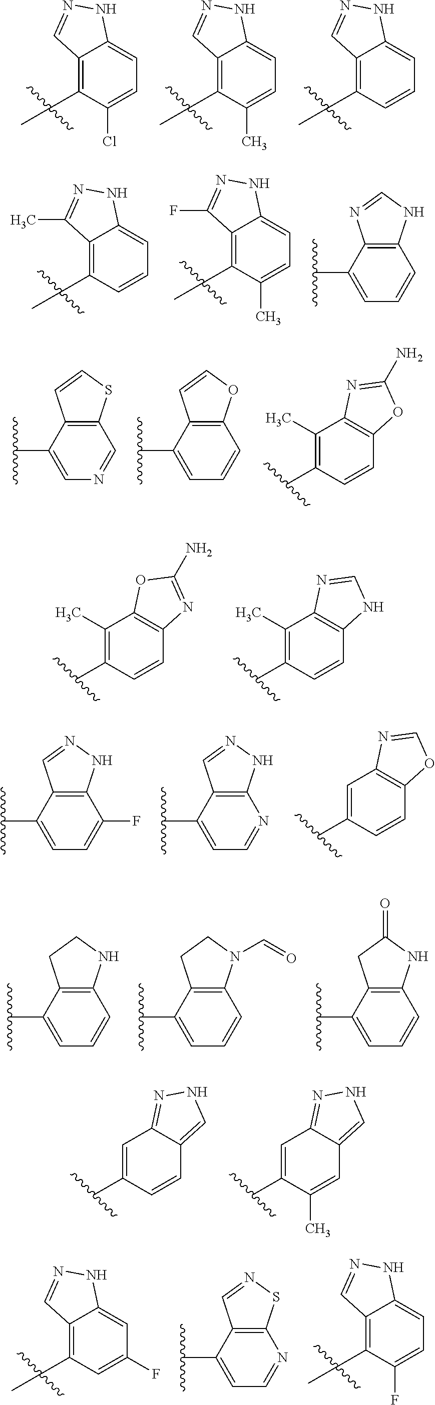

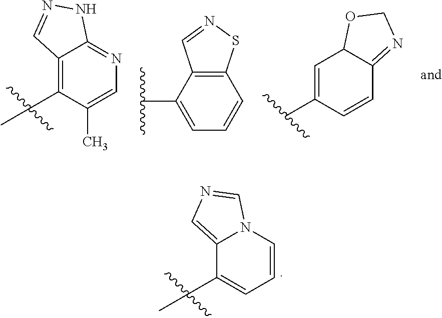

In some embodiments, ring A in compounds according to Formula I, Ia, Ib, Ic, Ic-1, Id, Ie, If, If-1, Ig, Ih, IIa, IIb, and/or IIc is:

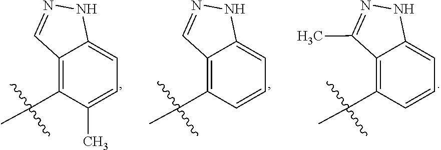 [SMILES: *c1c(C)ccc2[nH]ncc12 ; *c1cccc2[nH]ncc12 ; *c1cccc2[nH]nc(C)c12]

In some embodiments, ring A in compounds according to Formula I, Ta, Ib, Ic, Ic-1, Id, Ie, If, If-1, Ig, Ih, IIa, IIb, and/or IIc is:

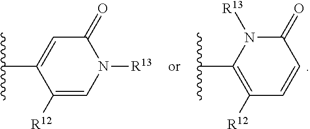

In some such embodiments, R¹² and R¹³ are each independently hydrogen or C₁₋₆ alkyl.

In some embodiments, ring A in compounds according to Formula I, Ia, Ib, Ic, Ic-1, Id, Ie, If, If-1, Ig, Ih, IIa, IIb, and/or IIc is:

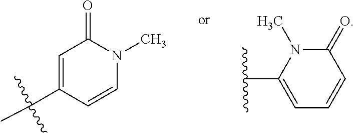

In some embodiments, ring A in compounds according to Formula I, Ia, Ib, Ic, Ic-1, Id, Ie, If, If-1, Ig, Ih, IIa, IIb, and/or IIc is:

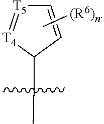

In some embodiments, ring A in compounds according to Formula I, Ia, Ib, Ic, Ic-1, Id, Ie, If, If-1, Ig, Ih, IIa, IIb, and/or IIc is:

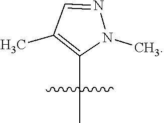 [SMILES: *c1c(C)cnn1C]

In some embodiments, the compound according to Formula I, or a pharmaceutically acceptable salt thereof, is a compound having a structure according to Formula Ib:

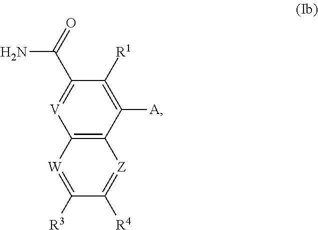

wherein ring A, V, W, Z, R¹, R³, and R⁴ are as defined herein.

In some embodiments, the compound according to Formula Ib, or a pharmaceutically acceptable salt thereof, is a compound having a structure according to Formula Ic:

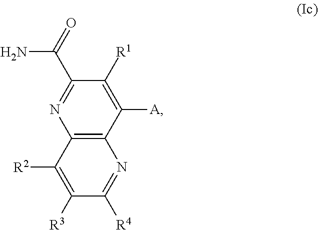

wherein ring A, R¹, R², R³, and R⁴ are as defined herein.

In some embodiments, the compound according to Formula Ic, or a pharmaceutically acceptable salt thereof, is a compound having a structure according to Formula Ic-1:

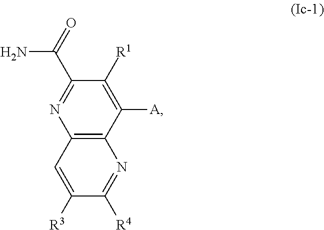

Wherein ring A, R¹, R³, and R⁴ are as defined herein.

In some embodiments, the compound according to Formula (Ic) is selected from any combination of compounds 1, 2, 3, 6, 153, 154, 155, 157, 158, 160, 161, 162, 181, 183, 184, 190, 191, 203, 204, 205, 206, 207, 208, 210, 220, 221, 228, 229, 230, 231, 232, 248, 249, 250, 253, 254, 255, 256, 257, 258, 259, 271, 272, 273, 278, 291, 292, 293, 303, 309, 310, 314, 315, 317, 320, 322, 323, and 332 as set forth in Table 1A, Table 1B, and/or Table 1C.

In some embodiments, the compound according to Formula (Ic) is selected from any combination of compounds 4, 5, 7, 8, 9, 10, 166, 167, 174, 175, 185, 186, 211, 212, 225, 226, 239, 240, 244, 245, 265, 266, 267, 268, 269, 270, 285, 286, 287, 288, 289, 290, 304, 305, and 306 as set forth in Table 1A, Table 1B, and/or Table 1C.

In some embodiments, the compound according to Formula Ib, or a pharmaceutically acceptable salt thereof, is a compound having a structure according to Formula Id:

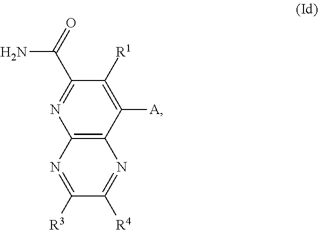

wherein ring A, R¹, R³, and R⁴ are as defined herein.

In some embodiments, the compound according to Formula (Id) is selected from compounds 11 and 126 as set forth in Table 1A.

In some embodiments, the compound according to Formula (Id) is selected from any combination of compounds 127, 128, 194, and 195 as set forth in Table 1A and/or Table 1B.

In some embodiments, the compound according to Formula Tb, or a pharmaceutically acceptable salt thereof, is a compound having a structure according to Formula Ie:

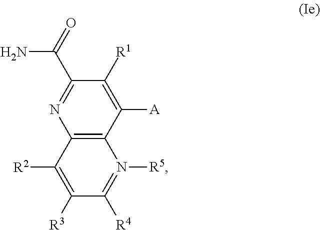

wherein ring A, R¹, R², R³, R⁴, and R⁵ are as defined herein.

In some embodiments, the compound according to Formula (Ie) is selected from any combination of compounds 12, 13, 14, 15, 17, 18, 19, 20, 22, 25, 27, 28, 34, 36, 37, 42, 43, 46, 47, 48, 51, 52, 55, 56, 59, 60, 129, 130, 134, 236, and 338 as set forth in Table 1A, Table 1B, and/or Table 1C.

In some embodiments, the compound according to Formula (Ie) is selected from any combination of compounds 23, 24, 30, 31, 32, 33, 38, 39, 40, 41, 44, 45, 49, 50, 53, 54, 57, 58, 147, 148, 149, 150, 242, and 243 as set forth in Table 1A and/or Table 1B.

In some embodiments, the compound according to Formula Ib, or a pharmaceutically acceptable salt thereof, is a compound having a structure according to Formula If:

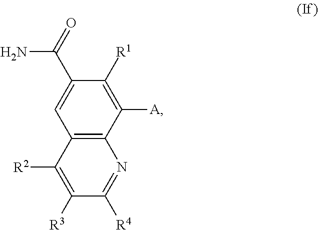

wherein ring A, R¹, R², R³, and R⁴ are as defined herein.

In some embodiments, the compound according to Formula (If) is selected from any combination of compounds 61, 64, 143, 146, 177, 196, 318, and 330 as set forth in Table 1A, Table 1B, and/or Table 1C.

In some embodiments, the compound according to Formula (If) is selected from any combination of compounds 62, 63, 65, 66, 179, 180, 213, 235, 325, and 327 as set forth in Table 1A, Table 1B, and/or Table 1C.

In some embodiments, the compound according to Formula If, or a pharmaceutically acceptable salt thereof, is a compound having a structure according to Formula If-1:

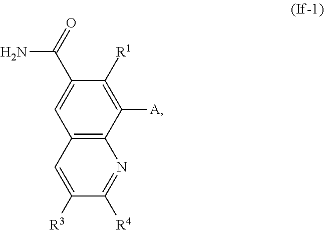

wherein ring A, R¹, R³, and R⁴ are as defined herein.

In some embodiments, the compound according to Formula Ib, or a pharmaceutically acceptable salt thereof, is a compound having a structure according to Formula Ig:

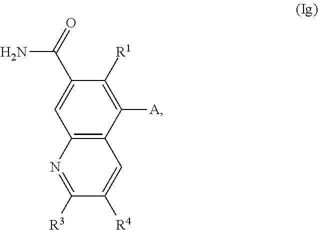

wherein ring A, R¹, R³, and R⁴ are as defined herein.

In some embodiments, the compound according to Formula (Ig) is selected from any combination of compounds 67, 144, 241, and 333 as set forth in Table 1A, Table 1B, and/or Table 1C.

In some embodiments, the compound according to Formula (Ig) is selected from compounds 68 and 69 as set forth in Table 1A.

In some embodiments, the compound according to Formula Ib, or a pharmaceutically acceptable salt thereof, is a compound having a structure according to Formula Ih:

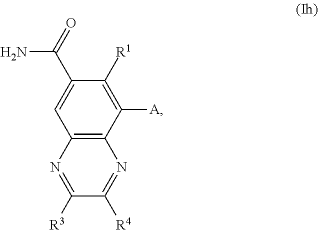

wherein ring A, R¹, R³, and R⁴ are as defined herein.

In some embodiments, the compound according to Formula (Ih) is selected from any combination of compounds 70, 72, 73, 74, 75, 76, 77, 80, 81, 82, 91, 94, 96, 97, 101, 102, 104, 107, 108, 109, 110, 113, 114, 115, 120, 121, 122, 123, 131, 132, 133, 137, 139, 142, 145, 156, 159, 172, 173, 176, 178, 182, 189, 197, 198, 199, 200, 214, 215, 216, 217, 227, 234, 251, 252, 279, 308, 311, 316, 321, 331, and 339 as set forth in Table 1A, Table 1B, and/or Table 1C.

In some embodiments, the compound according to Formula (Ih) is selected from any combination of compounds 78, 79, 83, 85, 87, 89, 92, 93, 98, 99, 100, 103, 105, 106, 111, 112, 116, 117, 118, 119, 124, 125, 135, 136, 140, 141, 151, 152, 163, 164, 165, 168, 169, 187, 188, 192, 193, 201, 209, 218, 222, 223, 224, 246, 247, 275, 280, 281, 299, 300, and 307 as set forth in Table 1A, Table 1B, and/or Table 1C.

In some embodiments, compounds of Formula Ib, Ic, Ic-1, Id, le, If, If-1, Ig, and/or Ih are provided, as well as pharmaceutically acceptable salts thereof, wherein R³ and R⁴ are independently hydrogen or C₁₋₆ alkyl. In some such embodiments, R³ is H and R⁴ is C₁₋₆ alkyl (e.g., methyl). In some embodiments, R³ is C₁₋₆ alkyl (e.g., methyl) and R⁴ is C₁₋₆ alkyl. In some embodiments, R³ and R⁴ are H. Compounds of Formula Ib, Ic, Ic-1, Id, Ie, If, If-1, Ig, and/or Ih in such embodiments may include any of the ring A groups set forth herein, containing any combination of R⁶ groups (including any combination of R⁷, R⁸, —OR⁹, R¹, R¹², R¹³, and R¹⁴). In some such embodiments, R¹ is amino.

In some embodiments,
R², R³, R⁴, and R⁵, when present (e.g., in compounds of Formula Ib, Ic, Ic-1, Id, le, If, If-1, Ig, and/or Ih), are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁. 6 haloalkoxy, C₃₋ₛ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently substituted with C₁₋₆ alkyl; and
ring A is selected from the group consisting of

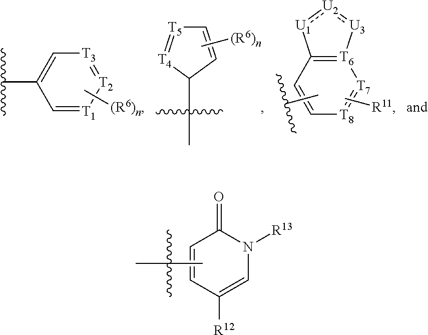

wherein:
each [symbol] represents a single bond, a double bond, or a delocalized n bond;
T₁, T₂, T₃, T₄, T₅, T₆, T₇, and T₈ are independently selected from the group consisting of N, C, and CH;
U₁, U₂, and U₃ are independently selected from the group consisting of N, NR¹⁰, O, S, and CR¹⁴;
each R⁶ is independently selected from the group consisting of amino, oxo, cyano, hydroxy, C₁₋₆ alkyl, C₁₋₆ alkoxy, C₁₋₆ haloalkyl, C₁₋₆ hydroxyalkyl, and halogen;
R¹¹, R¹², and R¹⁴ are independently selected from the group consisting of hydrogen, amino, oxo, cyano, C₁₋₆ alkyl, C₁₋₆ alkoxy, C₁₋₆ haloalkyl, C₁₋₆ hydroxyalkyl, and halogen;
each R¹⁰ and R¹³ are independently selected from the group consisting of hydrogen, C₁₋₆ alkyl, and CHO; and
n is 1, 2, 3, or 4.

In some embodiments,
R¹ is amino or hydrogen;
R², R³, R⁴, and R⁵ are independently selected from the group consisting of hydrogen, hydroxy, cyano, halogen, C₁₋₆ alkyl, C₁₋₆ haloalkyl, and C₁₋₆ alkoxy; and
ring A is selected from the group consisting of

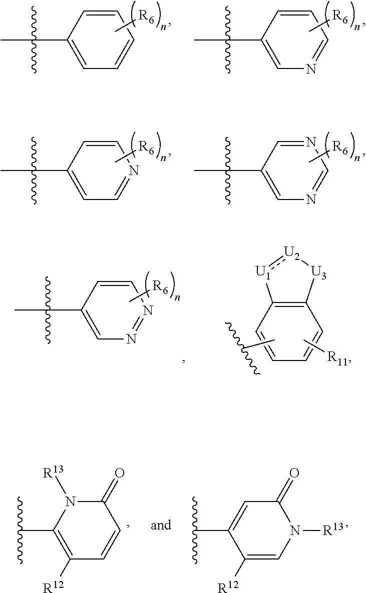

wherein [symbol] represents a single bond or a double bond;
each R¹¹ is independently selected from the group consisting of hydrogen, cyano, C₁₋₆ alkyl, C₁₋₆ alkoxy, C₁₋₆ haloalkyl, C₁₋₆ hydroxyalkyl, and halogen; and
each R¹⁴ is independently selected from the group consisting of hydrogen, amino, oxo, cyano, C₁₋₆ alkyl, and halogen.

In some embodiments,
R¹ is amino or hydrogen;
R², R³, R⁴, and R⁵ are independently selected from the group consisting of hydrogen, hydroxy, halogen, C₁₋₆ alkyl, C₁₋₆ haloalkyl, and C₁₋₆ alkoxy; and
ring A is selected from the group consisting of

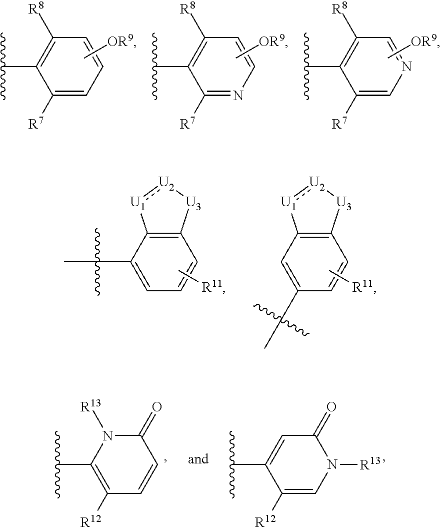

wherein R⁷ and R⁸ are independently selected from the group consisting of hydrogen, amino, cyano, C₁₋₆ alkyl, C₁₋₆ alkoxy, C₁₋₆ haloalkyl, C₁₋₆ hydroxyalkyl, and halogen;
R⁹ is hydrogen or C₁₋₆ alkyl;
R¹¹ is selected from the group consisting of hydrogen, C₁₋₆ alkyl, and halogen; and
R¹⁴ is selected from the group consisting of hydrogen, amino, oxo, C₁₋₆ alkyl, and halogen.

In some embodiments,
R¹ is amino;
R², R³, R⁴, and R⁵ are independently selected from the group consisting of hydrogen, hydroxy, halogen, C₁₋₆ alkyl, C₁₋₆ haloalkyl, and C₁₋₆ alkoxy; and
ring A is selected from the group consisting of:

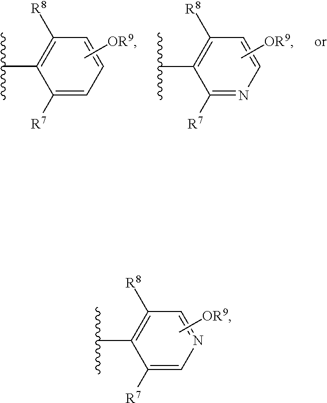

wherein R⁷ and R⁸ are independently selected from the group consisting of hydrogen, C₁₋₆ alkyl, and halogen.

In some embodiments, the compound according to Formula I, Ia, or Ib, or a pharmaceutically acceptable salt thereof, is a compound having a structure according to Formula Ik′:

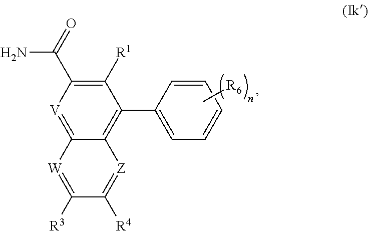

wherein
at least one of V, W, and Z is N;
each R⁶ is independently selected from the group consisting of amino, cyano, hydroxy, C₁₋₆ alkyl, C₁₋₆ alkoxy, C₁₋₆ haloalkyl, C₁₋₆ hydroxyalkyl, and halogen; and
n is 1, 2, or 3.

In some embodiments, the compound according to Formula I, Ia, or Ib, or a pharmaceutically acceptable salt thereof, is a compound having a structure according to Formula Ik:

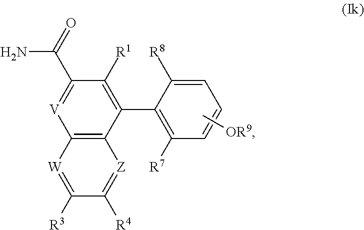

wherein
at least one of V, W, and Z is N;
R⁷ and R⁸ are independently selected from the group consisting of hydrogen, C₁₋₆ alkyl, C₁₋₆ alkoxy, C₁₋₆ haloalkyl, and halogen; and
R⁹ is hydrogen or C₁₋₆ alkyl.

In some embodiments, the compound according to Formula Ik, or a pharmaceutically acceptable salt thereof, is a compound having a structure according to Formula Ik-1 or Ik-2:

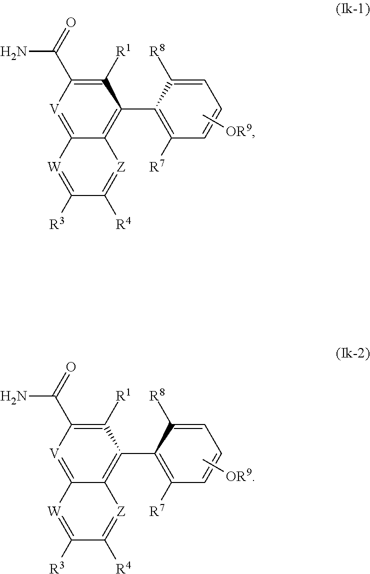

In some embodiments, the compound according to Formula Ik, or a pharmaceutically acceptable salt thereof, is a compound having a structure according to Formula Ik-3 or Ik-4:

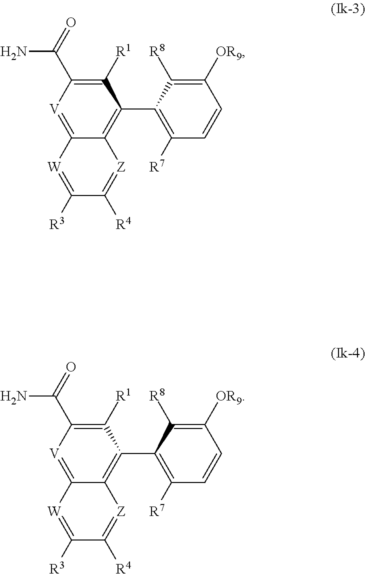

In some embodiments, the compound according to Formula Ik, or a pharmaceutically acceptable salt thereof, is a compound having a structure according to Formula Ik-5 or Ik-6:

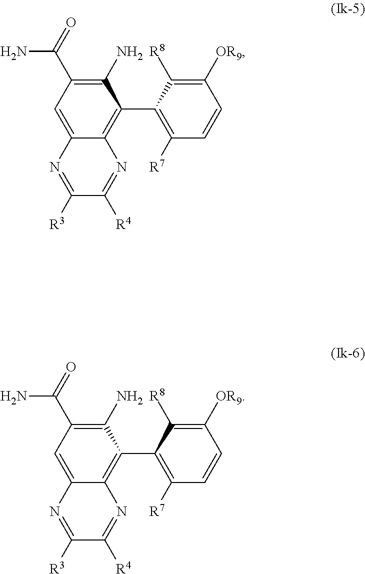

In some embodiments,
R³ and R⁴ are each independently selected from the group consisting of hydrogen, hydroxy, halogen, C₁₋₆ alkyl, C₁₋₆ haloalkyl, and C₁₋₆ alkoxy; and
R⁷ and R⁸ are each independently selected from the group consisting of hydrogen, C₁₋₆ alkyl, C₁₋₆ haloalkyl, and halogen.

In some embodiments,
R³ and R⁴ are each independently selected from the group consisting of hydrogen, hydroxy, C₁₋₆ alkyl, C₁₋₆ haloalkyl, and C₁₋₆ alkoxy;
R⁷ and R⁸ are each independently selected from the group consisting of hydrogen, C₁₋₆ alkyl, and halogen; and
R⁹ is hydrogen or C₁₋₆ alkyl.

In some embodiments, the compound according to Formula I, Ia, or Ib, or a pharmaceutically acceptable salt thereof, is a compound having a structure according to Formula Im:

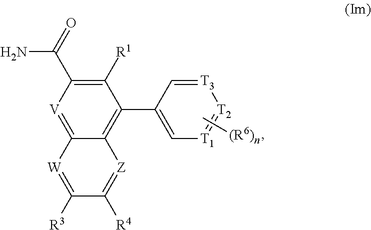

wherein
at least one of V, W, and Z is N;
at least one of T₁, T₂, and T₃ is N;
each R⁶ is independently selected from the group consisting of amino, cyano, C₁₋₆ alkyl, C₁₋₆ alkoxy, C₁₋₆ haloalkyl, C₁₋₆ hydroxyalkyl, and halogen; and
n is 1, 2, or 3.

In some embodiments, the compound according to Formula I, Ia, or Ib, or a pharmaceutically acceptable salt thereof, is a compound having a structure according to Formula Im-1 or Im-2:

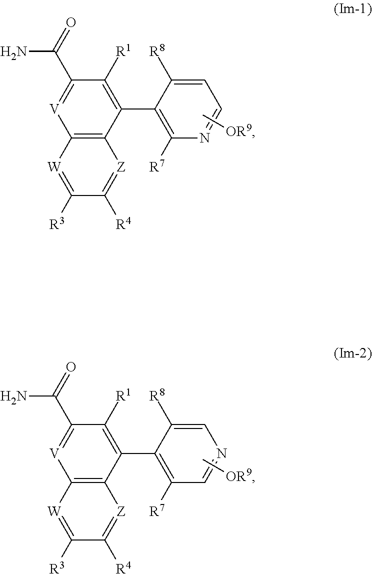

wherein
at least one of V, W, and Z is N;
R⁷ and R⁸ are each independently selected from the group consisting of hydrogen, C₁₋₆ alkyl, C₁₋₆ alkoxy, C₁₋₆ haloalkyl, and halogen; and
R⁹ is hydrogen or C₁₋₆ alkyl.

In some embodiments, the compound according to Formula Im-1 or Im-2, or a pharmaceutically acceptable salt thereof, is a compound having a structure according to Formula Im-3, Im-4, Im-5, or Im-6:

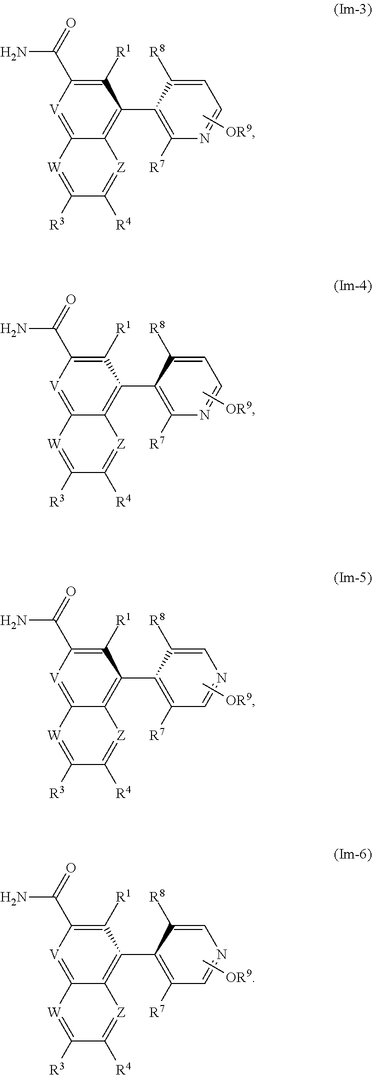

In some embodiments, compounds of Formula Ik, Ik-1, Ik-2, Ik-3, Ik-4, Ik-5, Ik-6, Im-1, Im-2, Im-3, Im-4, Im-5, and Im-6 are provided, as well as pharmaceutically acceptable salts thereof, wherein R³ and R⁴ are independently hydrogen or C₁₋₆ alkyl. In some such embodiments, R³ is H and R⁴ is C₁₋₆ alkyl (e.g., methyl). In some embodiments, R³ is C₁₋₆ alkyl (e.g., methyl) and R⁴ is C₁₋₆ alkyl (e.g., methyl). In some embodiments, R³ and R⁴ are H. Compounds of Formula Ik, Ik-1, Ik-2, Ik-3, Ik-4, Ik-5, Ik-6, Im-1, Im-2, Im-3, Im-4, Im-5, and Im-6 in such embodiments may include any combination of R⁷, R⁸, and —OR⁹. For example, R⁷ and R⁸ may be C₁₋₆ alkyl (e.g., methyl), and —OR⁹ may be —OH. Alternatively, R⁷ may be C₁₋₆ alkyl (e.g., methyl), R⁸ may be hydrogen, and —OR⁹ may be —OH. In some such embodiments, R¹ is amino.

In some embodiments, the compound according to Formula I, Ia, or Ib, or a pharmaceutically acceptable salt thereof, is a compound having a structure according to Formula In:

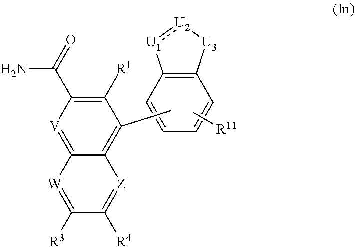

wherein at least one of V, W, and Z is N.

In some embodiments, the compound according to Formula In, or a pharmaceutically acceptable salt thereof, is a compound having a structure according to Formula In-1 or In-2:

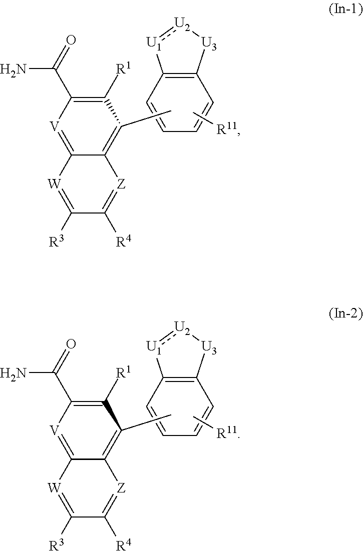

In some embodiments,
R¹ is amino;
R², R³, R⁴, and R⁵ are independently selected from the group consisting of hydrogen, hydroxy, cyano, halogen, C₁₋₆ alkyl, C₁₋₆ haloalkyl, and C₁₋₆ alkoxy;
R⁷ and R⁸ are independently selected from the group consisting of hydrogen, C₁₋₆ alkyl, C₁₋₆ alkoxy, C₁₋₆ haloalkyl, and halogen;
R⁹ is hydrogen or C₁₋₆ alkyl; and

R¹¹ and R¹⁴ are independently selected from the group consisting of hydrogen, amino, oxo, cyano, C₁₋₆ alkyl, C₁₋₆ alkoxy, C₁₋₆ haloalkyl, C₁₋₆ hydroxyalkyl, and halogen. In some embodiments, compounds of Formula In, In-1, and/or In-2 are provided, as well as pharmaceutically acceptable salts thereof, wherein R³ and R⁴ are independently hydrogen or C₁₋₆ alkyl. In some such embodiments, R³ is H and R⁴ is C₁₋₆ alkyl (e.g., methyl). In some embodiments, R³ is C₁₋₆ alkyl (e.g., methyl) and R⁴ is C₁₋₆ alkyl. In some embodiments, R³ and R⁴ are H. Compounds of Formula In, In-1, and/or In-2 in such embodiments may include any combination of R¹⁰, R⁸, and —R¹¹. In some such embodiments, R¹ is amino.

In some embodiments, the compound according to Formula I, or a pharmaceutically acceptable salt thereof, is a compound having a structure according to Formula IIa, IIb, or IIc:

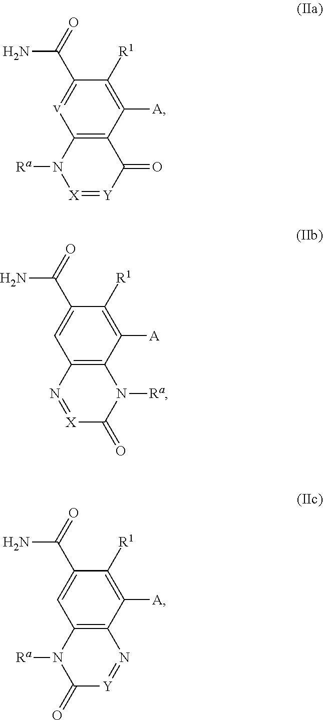

wherein the variables are as defined herein.

In some embodiments, the present disclosure provides a compound according to Formula I which is selected from any combination of the compounds in provided in Table 1A, Table 1B, and/or Table 1C below, or a pharmaceutically acceptable salt thereof.

TABLE 1A
Compounds of the present disclosure.

| Compound | |
|---|---|
| No. | Structure |
| 1 | 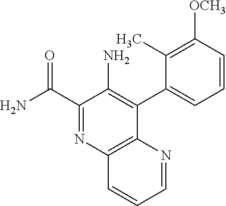 [SMILES: COc1cccc(-c2c(N)c(C(N)=O)nc3cccnc23)c1C] |
| 2 | 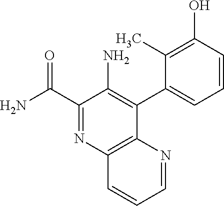 [SMILES: Cc1c(O)cccc1-c1c(N)c(C(N)=O)nc2cccnc12] |
| 3 | 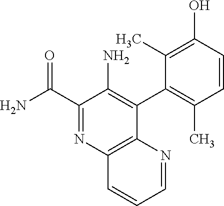 [SMILES: Cc1ccc(O)c(C)c1-c1c(N)c(C(N)=O)nc2cccnc12] |
| 4 | 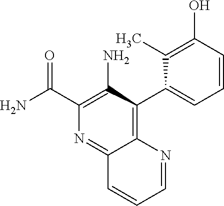 |
| 5 | 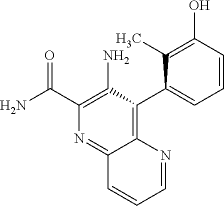 |
| 6 | 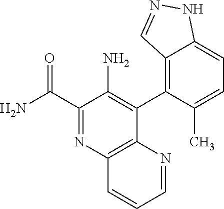 [SMILES: Cc1ccc2[nH]ncc2c1-c1c(N)c(C(N)=O)nc2cccnc12] |
| 7 | 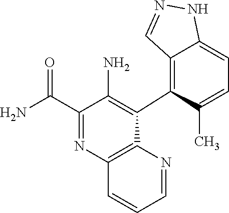 |
| 8 | 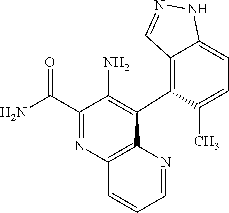 |
| 9 | 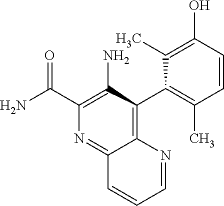 |
| 10 | 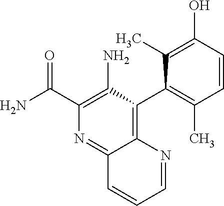 |
| 11 | 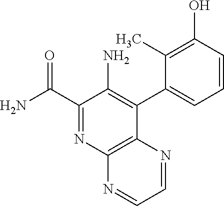 [SMILES: Cc1c(O)cccc1-c1c(N)c(C(N)=O)nc2nccnc12] |
| 12 | 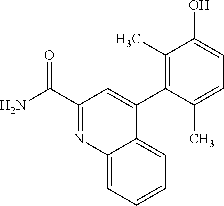 [SMILES: Cc1ccc(O)c(C)c1-c1cc(C(N)=O)nc2ccccc12] |
| 13 | 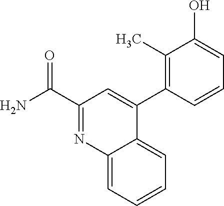 [SMILES: Cc1c(O)cccc1-c1cc(C(N)=O)nc2ccccc12] |
| 14 | 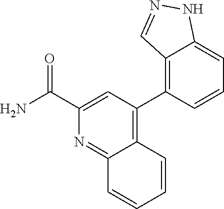 [SMILES: NC(=O)c1cc(-c2cccc3[nH]ncc23)c2ccccc2n1] |
| 15 | 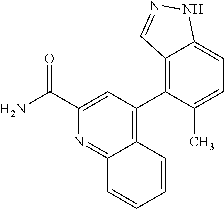 [SMILES: Cc1ccc2[nH]ncc2c1-c1cc(C(N)=O)nc2ccccc12] |
| 17 | 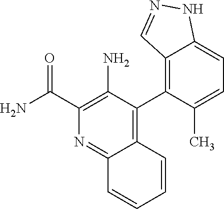 [SMILES: Cc1ccc2[nH]ncc2c1-c1c(N)c(C(N)=O)nc2ccccc12] |
| 18 | 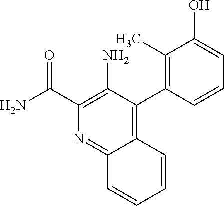 [SMILES: Cc1c(O)cccc1-c1c(N)c(C(N)=O)nc2ccccc12] |
| 19 | 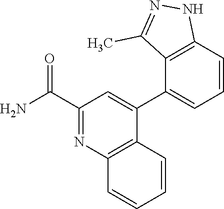 [SMILES: Cc1n[nH]c2cccc(-c3cc(C(N)=O)nc4ccccc34)c12] |
| 20 | 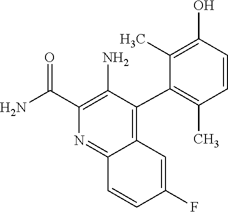 [SMILES: Cc1ccc(O)c(C)c1-c1c(N)c(C(N)=O)nc2ccc(F)cc12] |
| 22 | 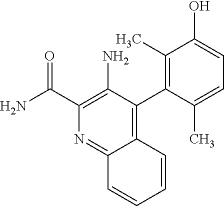 [SMILES: Cc1ccc(O)c(C)c1-c1c(N)c(C(N)=O)nc2ccccc12] |
| 23 | 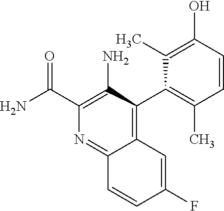 |
| 24 | 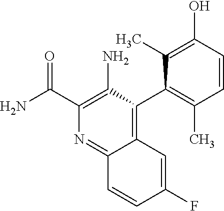 |
| 25 | 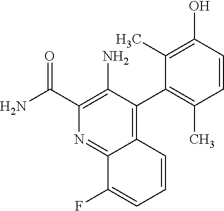 [SMILES: Cc1ccc(O)c(C)c1-c1c(N)c(C(N)=O)nc2c(F)cccc12] |
| 27 | 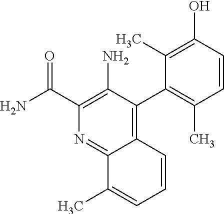 [SMILES: Cc1ccc(O)c(C)c1-c1c(N)c(C(N)=O)nc2c(C)cccc12] |
| 28 | 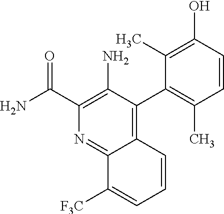 [SMILES: Cc1ccc(O)c(C)c1-c1c(N)c(C(N)=O)nc2c(C(F)(F)F)cccc12] |
| 30 | 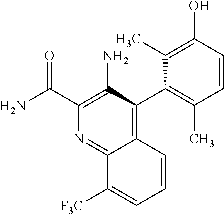 |
| 31 | 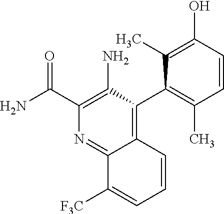 |
| 32 | 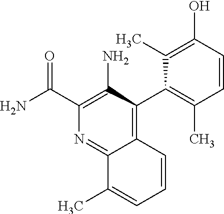 |
| 33 | 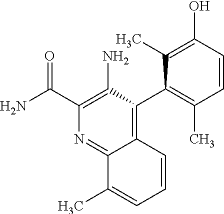 |
| 34 | 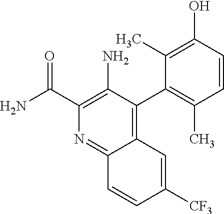 [SMILES: Cc1ccc(O)c(C)c1-c1c(N)c(C(N)=O)nc2ccc(C(F)(F)F)cc12] |
| 36 | 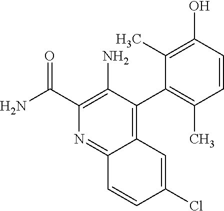 [SMILES: Cc1ccc(O)c(C)c1-c1c(N)c(C(N)=O)nc2ccc(Cl)cc12] |
| 37 | 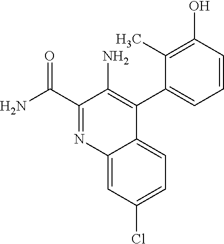 [SMILES: Cc1c(O)cccc1-c1c(N)c(C(N)=O)nc2cc(Cl)ccc12] |
| 38 | 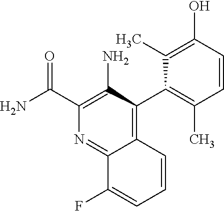 |
| 39 | 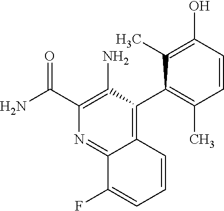 |
| 40 | 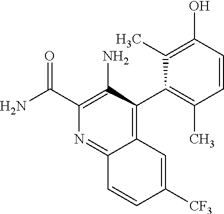 |
| 41 | 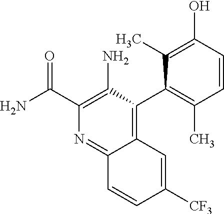 |
| 42 | 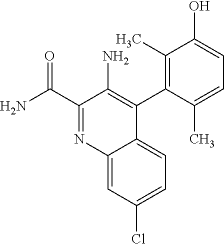 [SMILES: Cc1ccc(O)c(C)c1-c1c(N)c(C(N)=O)nc2cc(Cl)ccc12] |
| 43 | 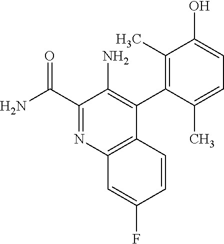 [SMILES: Cc1ccc(O)c(C)c1-c1c(N)c(C(N)=O)nc2cc(F)ccc12] |
| 44 | 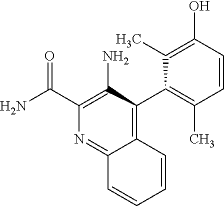 |
| 45 | 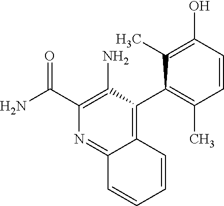 |
| 46 | 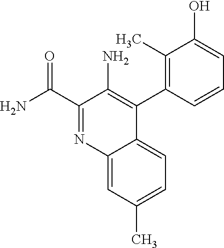 [SMILES: Cc1ccc2c(-c3cccc(O)c3C)c(N)c(C(N)=O)nc2c1] |
| 47 | 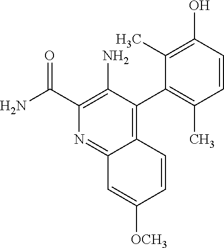 [SMILES: COc1ccc2c(-c3c(C)ccc(O)c3C)c(N)c(C(N)=O)nc2c1] |
| 48 | 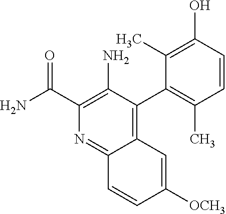 [SMILES: COc1ccc2nc(C(N)=O)c(N)c(-c3c(C)ccc(O)c3C)c2c1] |
| 49 | 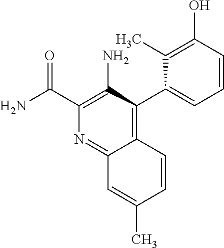 |
| 50 | 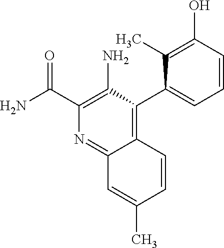 |
| 51 | 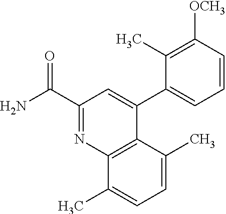 [SMILES: COc1cccc(-c2cc(C(N)=O)nc3c(C)ccc(C)c23)c1C] |
| 52 | 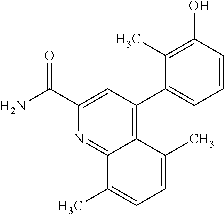 [SMILES: Cc1c(O)cccc1-c1cc(C(N)=O)nc2c(C)ccc(C)c12] |
| 53 | 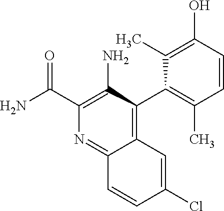 |
| 54 | 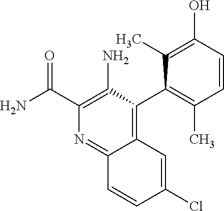 |
| 55 | 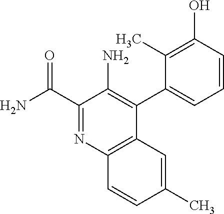 [SMILES: Cc1ccc2nc(C(N)=O)c(N)c(-c3cccc(O)c3C)c2c1] |
| 56 | 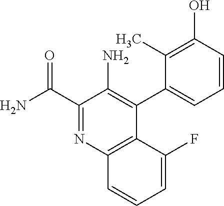 [SMILES: Cc1c(O)cccc1-c1c(N)c(C(N)=O)nc2cccc(F)c12] |
| 57 | 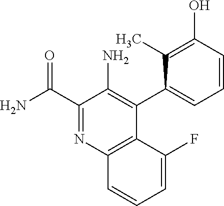 |
| 58 | 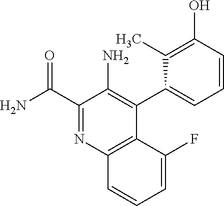 |
| 59 | 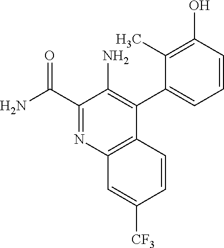 [SMILES: Cc1c(O)cccc1-c1c(N)c(C(N)=O)nc2cc(C(F)(F)F)ccc12] |
| 60 | 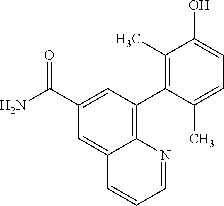 [SMILES: Cc1ccc(O)c(C)c1-c1cc(C(N)=O)cc2cccnc12] |
| 61 | 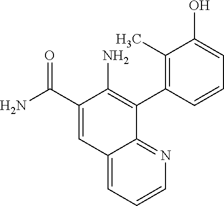 [SMILES: Cc1c(O)cccc1-c1c(N)c(C(N)=O)cc2cccnc12] |
| 62 | 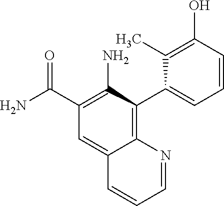 |
| 63 | 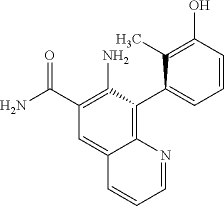 |
| 64 | 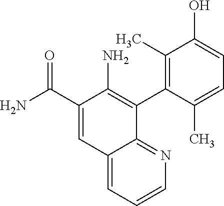 [SMILES: Cc1ccc(O)c(C)c1-c1c(N)c(C(N)=O)cc2cccnc12] |
| 65 | 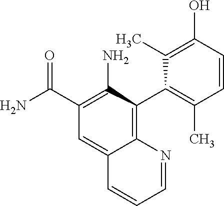 |
| 66 | 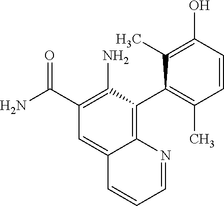 |
| 67 | 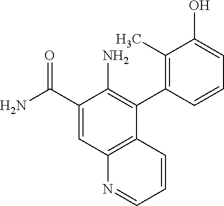 [SMILES: Cc1c(O)cccc1-c1c(N)c(C(N)=O)cc2ncccc12] |
| 68 | 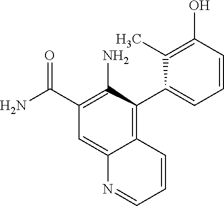 |
| 69 | 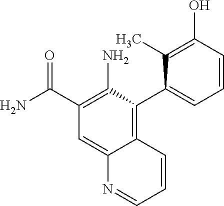 |
| 70 | 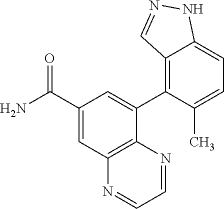 [SMILES: Cc1ccc2[nH]ncc2c1-c1cc(C(N)=O)cc2nccnc12] |
| 72 | 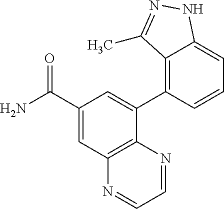 [SMILES: Cc1n[nH]c2cccc(-c3cc(C(N)=O)cc4nccnc34)c12] |
| 73 | 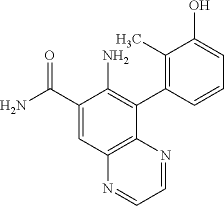 [SMILES: Cc1c(O)cccc1-c1c(N)c(C(N)=O)cc2nccnc12] |
| 74 | 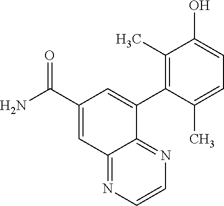 [SMILES: Cc1ccc(O)c(C)c1-c1cc(C(N)=O)cc2nccnc12] |
| 75 | 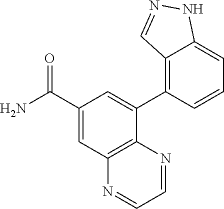 [SMILES: NC(=O)c1cc(-c2cccc3[nH]ncc23)c2nccnc2c1] |
| 76 | 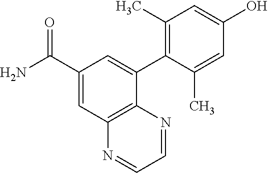 [SMILES: Cc1cc(O)cc(C)c1-c1cc(C(N)=O)cc2nccnc12] |
| 77 | 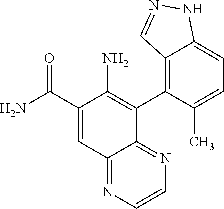 [SMILES: Cc1ccc2[nH]ncc2c1-c1c(N)c(C(N)=O)cc2nccnc12] |
| 78 | 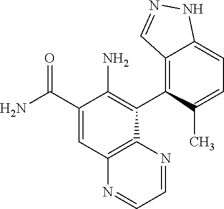 |
| 79 | 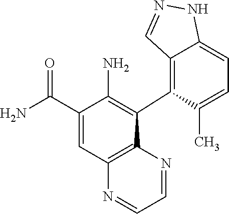 |
| 80 | 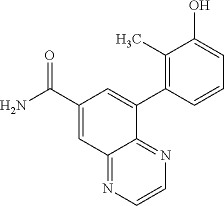 [SMILES: Cc1c(O)cccc1-c1cc(C(N)=O)cc2nccnc12] |
| 81 | 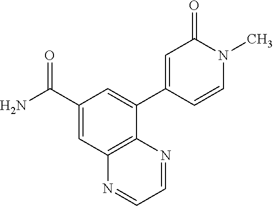 [SMILES: Cn1ccc(-c2cc(C(N)=O)cc3nccnc23)cc1=O] |
| 82 | 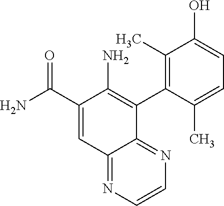 [SMILES: Cc1ccc(O)c(C)c1-c1c(N)c(C(N)=O)cc2nccnc12] |
| 83 | 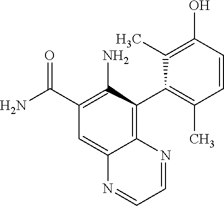 |
| 85 | 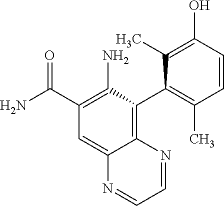 |
| 87 | 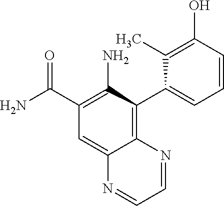 |
| 89 | 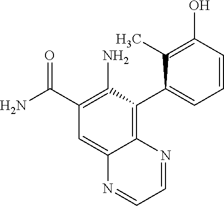 |
| 91 | 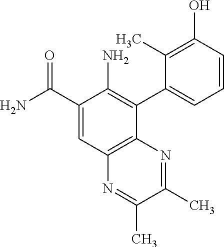 [SMILES: Cc1nc2cc(C(N)=O)c(N)c(-c3cccc(O)c3C)c2nc1C] |
| 92 | 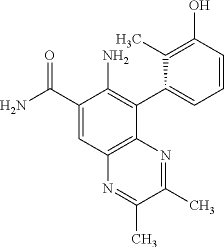 |
| 93 | 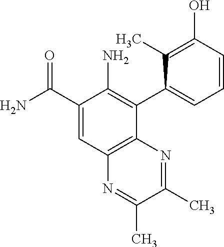 |
| 94 | 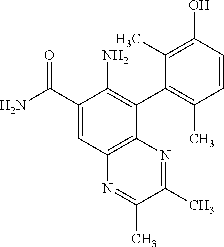 [SMILES: Cc1ccc(O)c(C)c1-c1c(N)c(C(N)=O)cc2nc(C)c(C)nc12] |
| 96 | 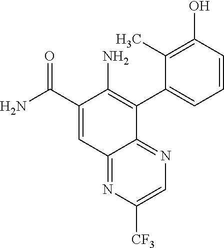 [SMILES: Cc1c(O)cccc1-c1c(N)c(C(N)=O)cc2nc(C(F)(F)F)cnc12] |
| 97 | 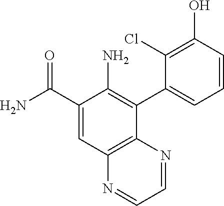 [SMILES: NC(=O)c1cc2nccnc2c(-c2cccc(O)c2Cl)c1N] |
| 98 | 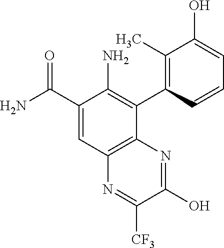 |
| 99 | 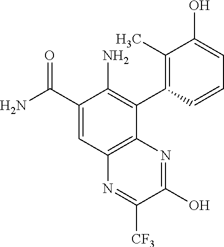 |
| 100 | 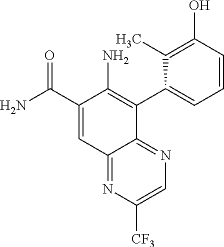 |
| 101 | 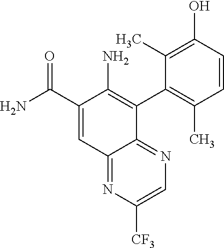 [SMILES: Cc1ccc(O)c(C)c1-c1c(N)c(C(N)=O)cc2nc(C(F)(F)F)cnc12] |
| 102 | 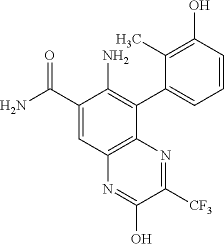 [SMILES: Cc1c(O)cccc1-c1c(N)c(C(N)=O)cc2nc(O)c(C(F)(F)F)nc12] |
| 103 | 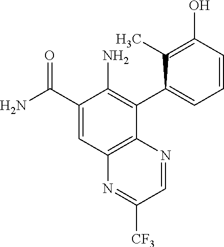 |
| 104 | 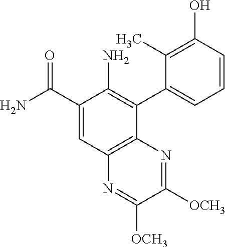 [SMILES: COc1nc2cc(C(N)=O)c(N)c(-c3cccc(O)c3C)c2nc1OC] |
| 105 | 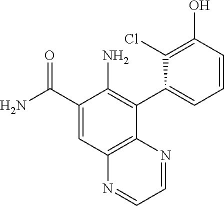 |
| 106 | 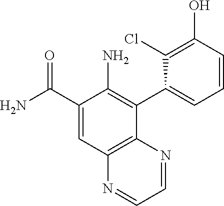 |
| 107 | 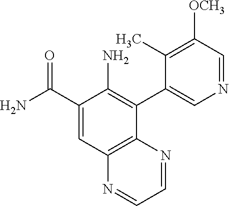 [SMILES: COc1cncc(-c2c(N)c(C(N)=O)cc3nccnc23)c1C] |
| 108 | 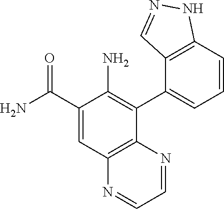 [SMILES: NC(=O)c1cc2nccnc2c(-c2cccc3[nH]ncc23)c1N] |
| 109 | 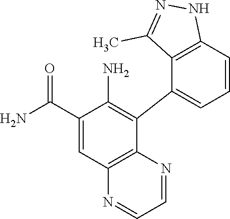 [SMILES: Cc1n[nH]c2cccc(-c3c(N)c(C(N)=O)cc4nccnc34)c12] |
| 110 | 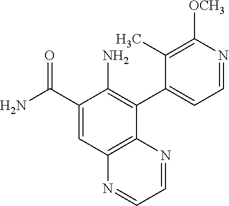 [SMILES: COc1nccc(-c2c(N)c(C(N)=O)cc3nccnc23)c1C] |
| 111 | 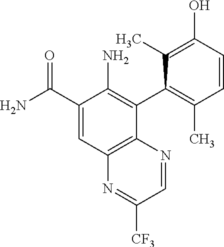 |
| 112 | 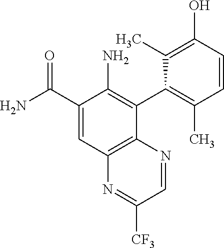 |
| 113 | 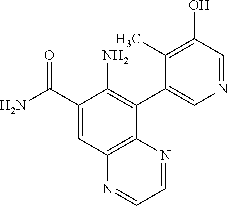 [SMILES: Cc1c(O)cncc1-c1c(N)c(C(N)=O)cc2nccnc12] |
| 114 | 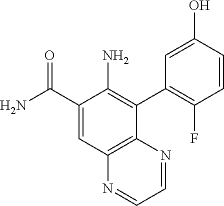 [SMILES: NC(=O)c1cc2nccnc2c(-c2cc(O)ccc2F)c1N] |
| 115 | 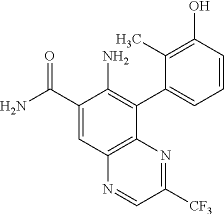 [SMILES: Cc1c(O)cccc1-c1c(N)c(C(N)=O)cc2ncc(C(F)(F)F)nc12] |
| 116 | 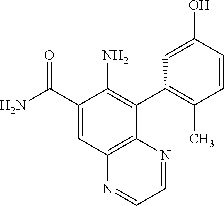 |
| 117 | 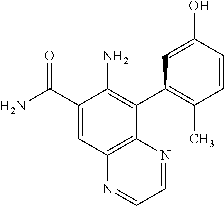 |
| 118 | 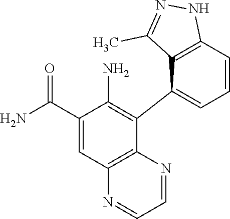 |
| 119 | 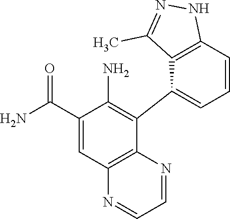 |
| 120 | 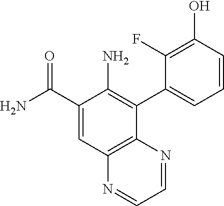 [SMILES: NC(=O)c1cc2nccnc2c(-c2cccc(O)c2F)c1N] |
| 121 | 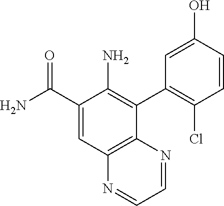 [SMILES: NC(=O)c1cc2nccnc2c(-c2cc(O)ccc2Cl)c1N] |
| 122 | 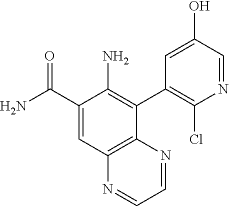 [SMILES: NC(=O)c1cc2nccnc2c(-c2cc(O)cnc2Cl)c1N] |
| 123 | 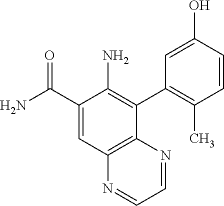 [SMILES: Cc1ccc(O)cc1-c1c(N)c(C(N)=O)cc2nccnc12] |
| 124 | 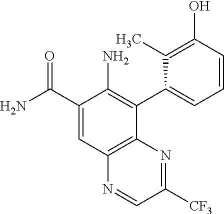 |
| 125 | 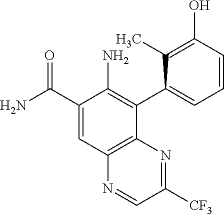 |
| 126 | 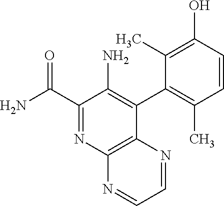 [SMILES: Cc1ccc(O)c(C)c1-c1c(N)c(C(N)=O)nc2nccnc12] |
| 127 | 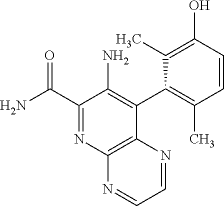 |
| 128 | 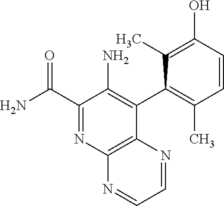 |
| 129 | 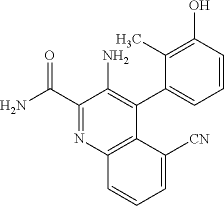 [SMILES: Cc1c(O)cccc1-c1c(N)c(C(N)=O)nc2cccc(C#N)c12] |

TABLE 1B
Compounds of the present disclosure.

| Compound | |
|---|---|
| No. | Structure |
| 130 | 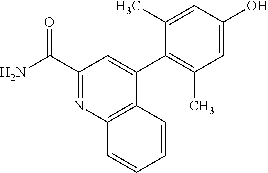 [SMILES: Cc1cc(O)cc(C)c1-c1cc(C(N)=O)nc2ccccc12] |
| 131 | 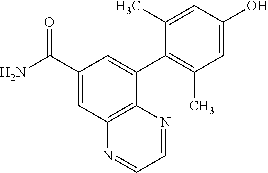 [SMILES: Cc1cc(O)cc(C)c1-c1cc(C(N)=O)cc2nccnc12] |
| 132 | 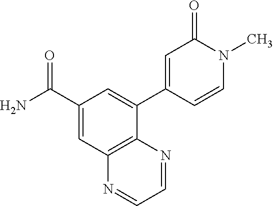 [SMILES: Cn1ccc(-c2cc(C(N)=O)cc3nccnc23)cc1=O] |
| 133 | 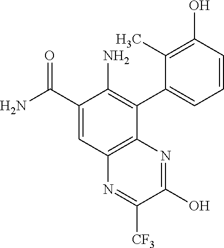 [SMILES: Cc1c(O)cccc1-c1c(N)c(C(N)=O)cc2nc(C(F)(F)F)c(O)nc12] |
| 134 | 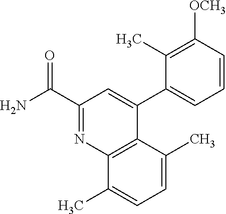 [SMILES: COc1cccc(-c2cc(C(N)=O)nc3c(C)ccc(C)c23)c1C] |
| 135 | 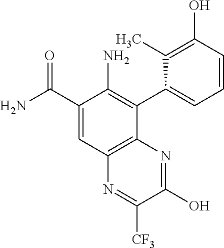 |
| 136 | 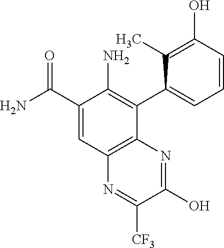 |
| 137 | 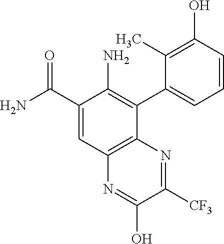 [SMILES: Cc1c(O)cccc1-c1c(N)c(C(N)=O)cc2nc(O)c(C(F)(F)F)nc12] |
| 138 | 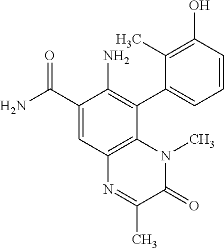 [SMILES: Cc1c(O)cccc1-c1c(N)c(C(N)=O)cc2nc(C)c(=O)n(C)c12] |
| 139 | 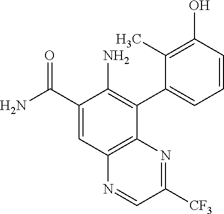 [SMILES: Cc1c(O)cccc1-c1c(N)c(C(N)=O)cc2ncc(C(F)(F)F)nc12] |
| 140 | 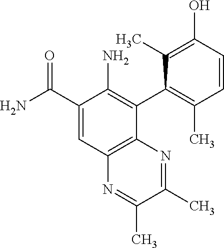 |
| 141 | 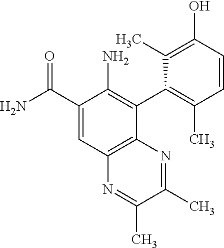 |
| 142 | 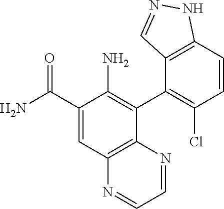 [SMILES: NC(=O)c1cc2nccnc2c(-c2c(Cl)ccc3[nH]ncc23)c1N] |
| 143 | 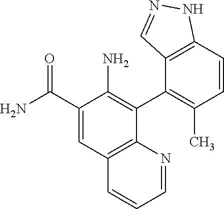 [SMILES: Cc1ccc2[nH]ncc2c1-c1c(N)c(C(N)=O)cc2cccnc12] |
| 144 | 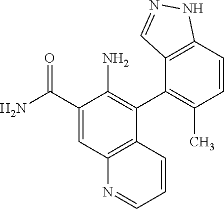 [SMILES: Cc1ccc2[nH]ncc2c1-c1c(N)c(C(N)=O)cc2ncccc12] |
| 145 | 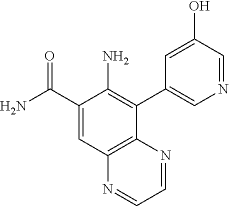 [SMILES: NC(=O)c1cc2nccnc2c(-c2cncc(O)c2)c1N] |
| 146 | 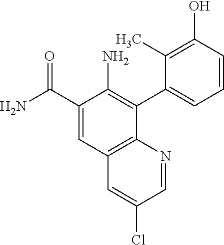 [SMILES: Cc1c(O)cccc1-c1c(N)c(C(N)=O)cc2cc(Cl)cnc12] |
| 147 | 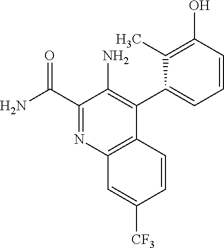 |
| 148 | 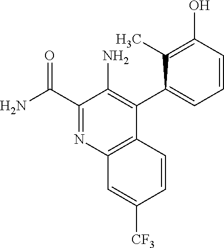 |
| 149 | 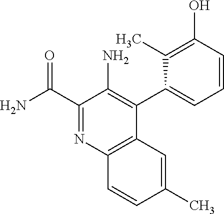 |
| 150 | 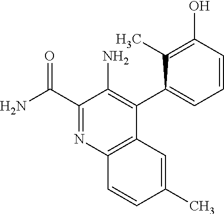 |
| 151 | 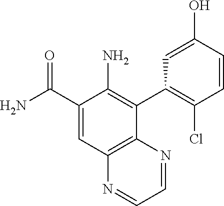 |
| 152 | 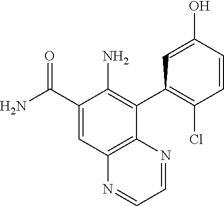 |
| 153 | 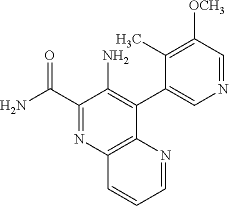 [SMILES: COc1cncc(-c2c(N)c(C(N)=O)nc3cccnc23)c1C] |
| 154 | 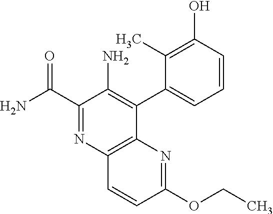 [SMILES: CCOc1ccc2nc(C(N)=O)c(N)c(-c3cccc(O)c3C)c2n1] |
| 155 | 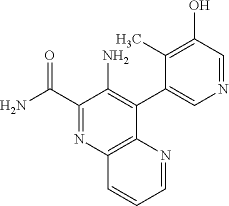 [SMILES: Cc1c(O)cncc1-c1c(N)c(C(N)=O)nc2cccnc12] |
| 156 | 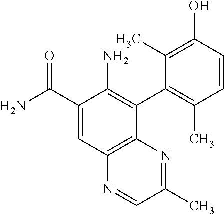 [SMILES: Cc1cnc2cc(C(N)=O)c(N)c(-c3c(C)ccc(O)c3C)c2n1] |
| 157 | 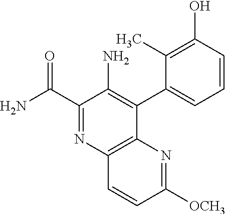 [SMILES: COc1ccc2nc(C(N)=O)c(N)c(-c3cccc(O)c3C)c2n1] |
| 158 | 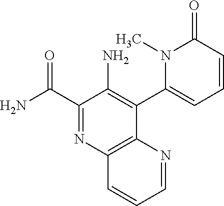 [SMILES: Cn1c(-c2c(N)c(C(N)=O)nc3cccnc23)cccc1=O] |
| 159 | 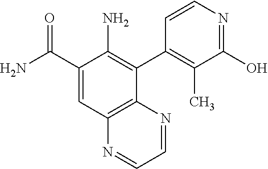 [SMILES: Cc1c(-c2c(N)c(C(N)=O)cc3nccnc23)ccnc1O] |
| 160 | 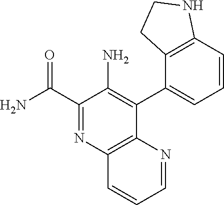 [SMILES: NC(=O)c1nc2cccnc2c(-c2cccc3c2CCN3)c1N] |
| 161 | 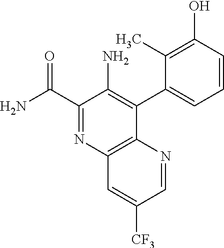 [SMILES: Cc1c(O)cccc1-c1c(N)c(C(N)=O)nc2cc(C(F)(F)F)cnc12] |
| 162 | 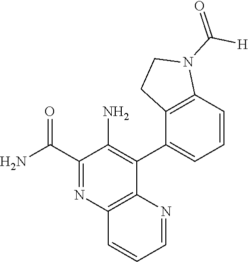 [SMILES: NC(=O)c1nc2cccnc2c(-c2cccc3c2CCN3C=O)c1N] |
| 163 | 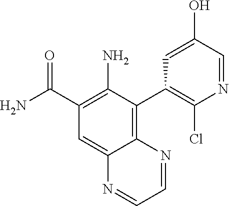 |
| 164 | 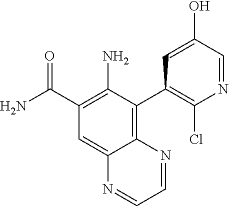 |
| 165 | 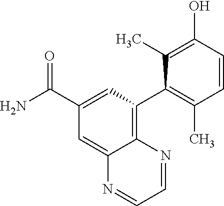 |
| 166 | 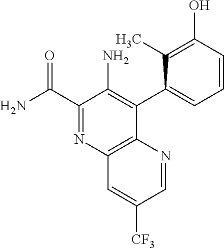 |
| 167 | 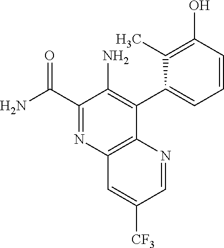 |
| 168 | 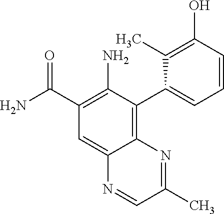 |
| 169 | 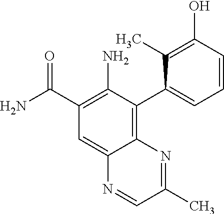 |
| 170 | 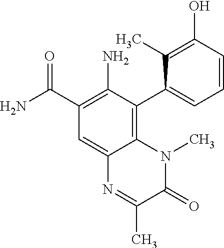 |
| 171 | 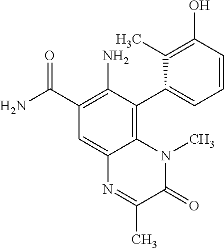 |
| 172 | 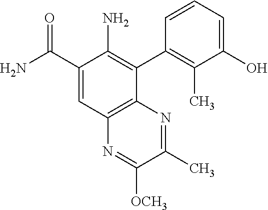 [SMILES: COc1nc2cc(C(N)=O)c(N)c(-c3cccc(O)c3C)c2nc1C] |
| 173 | 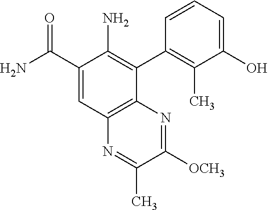 [SMILES: COc1nc2c(-c3cccc(O)c3C)c(N)c(C(N)=O)cc2nc1C] |
| 174 | 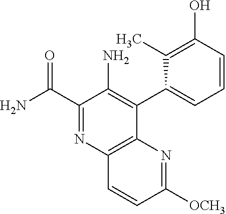 |
| 175 | 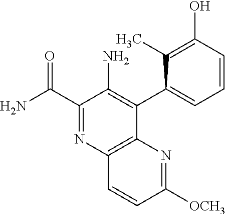 |
| 176 | 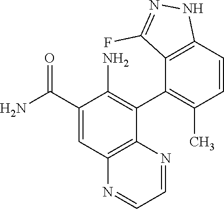 [SMILES: Cc1ccc2[nH]nc(F)c2c1-c1c(N)c(C(N)=O)cc2nccnc12] |
| 177 | 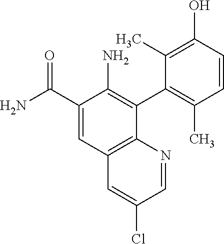 [SMILES: Cc1ccc(O)c(C)c1-c1c(N)c(C(N)=O)cc2cc(Cl)cnc12] |
| 178 | 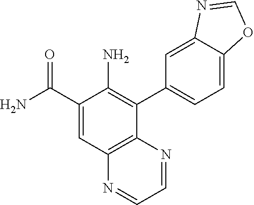 [SMILES: NC(=O)c1cc2nccnc2c(-c2ccc3ocnc3c2)c1N] |
| 179 | 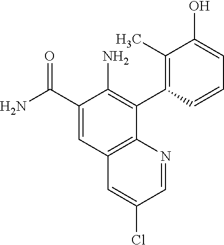 |
| 180 | 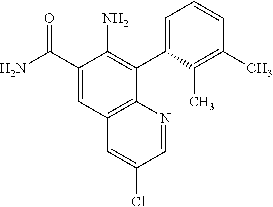 |
| 181 | 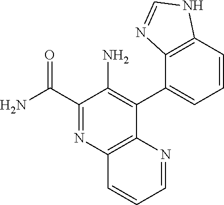 [SMILES: NC(=O)c1nc2cccnc2c(-c2cccc3[nH]cnc23)c1N] |
| 182 | 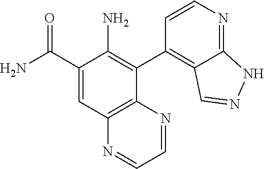 [SMILES: NC(=O)c1cc2nccnc2c(-c2ccnc3[nH]ncc23)c1N] |
| 183 | 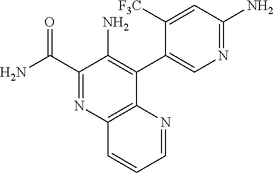 [SMILES: NC(=O)c1nc2cccnc2c(-c2cnc(N)cc2C(F)(F)F)c1N] |
| 184 | 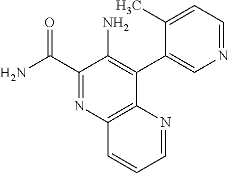 [SMILES: Cc1ccncc1-c1c(N)c(C(N)=O)nc2cccnc12] |
| 185 | 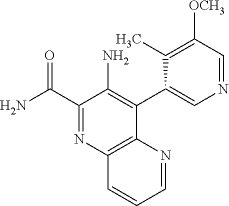 |
| 186 | 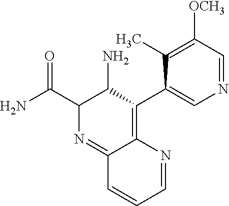 |
| 187 | 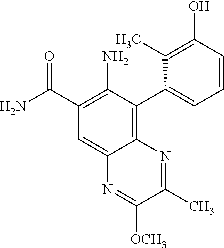 |
| 188 | 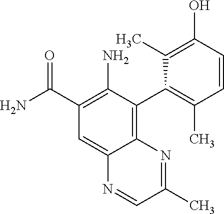 |
| 189 | 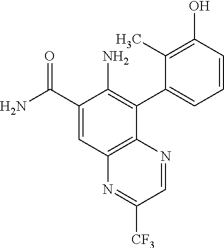 [SMILES: Cc1c(O)cccc1-c1c(N)c(C(N)=O)cc2nc(C(F)(F)F)cnc12] |
| 190 | 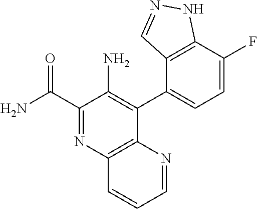 [SMILES: NC(=O)c1nc2cccnc2c(-c2ccc(F)c3[nH]ncc23)c1N] |
| 191 | 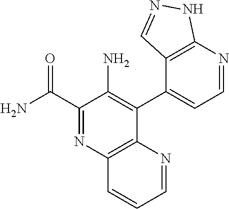 [SMILES: NC(=O)c1nc2cccnc2c(-c2ccnc3[nH]ncc23)c1N] |
| 192 | 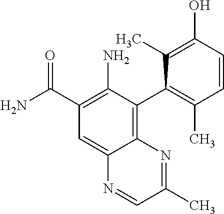 |
| 193 | 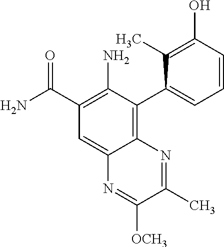 |
| 194 | 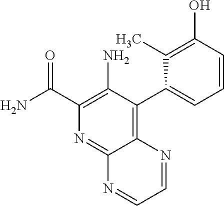 |
| 195 | 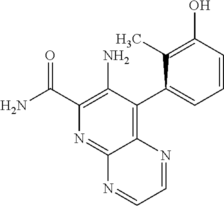 |
| 196 | 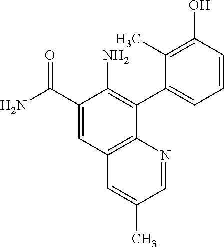 [SMILES: Cc1cnc2c(-c3cccc(O)c3C)c(N)c(C(N)=O)cc2c1] |
| 197 | 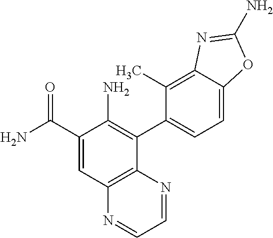 [SMILES: Cc1c(-c2c(N)c(C(N)=O)cc3nccnc23)ccc2oc(N)nc12] |
| 198 | 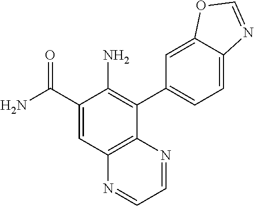 [SMILES: NC(=O)c1cc2nccnc2c(-c2ccc3ncoc3c2)c1N] |
| 199 | 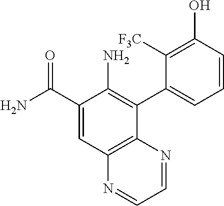 [SMILES: NC(=O)c1cc2nccnc2c(-c2cccc(O)c2C(F)(F)F)c1N] |
| 200 | 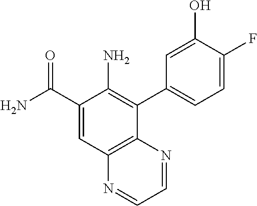 [SMILES: NC(=O)c1cc2nccnc2c(-c2ccc(F)c(O)c2)c1N] |
| 201 | 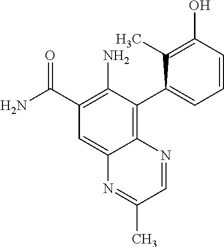 |
| 202 | 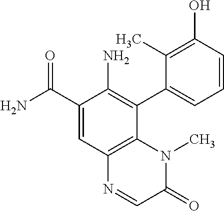 [SMILES: Cc1c(O)cccc1-c1c(N)c(C(N)=O)cc2ncc(=O)n(C)c12] |
| 203 | 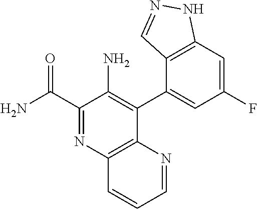 [SMILES: NC(=O)c1nc2cccnc2c(-c2cc(F)cc3[nH]ncc23)c1N] |
| 204 | 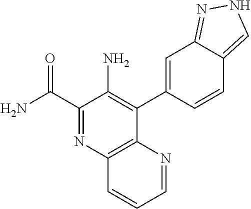 [SMILES: NC(=O)c1nc2cccnc2c(-c2ccc3c[nH]nc3c2)c1N] |
| 205 | 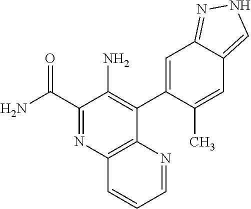 [SMILES: Cc1cc2c[nH]nc2cc1-c1c(N)c(C(N)=O)nc2cccnc12] |
| 206 | 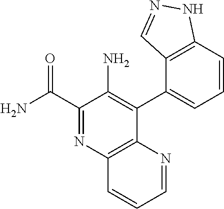 [SMILES: NC(=O)c1nc2cccnc2c(-c2cccc3[nH]ncc23)c1N] |
| 207 | 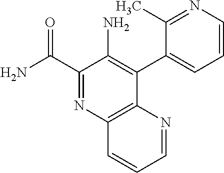 [SMILES: Cc1ncccc1-c1c(N)c(C(N)=O)nc2cccnc12] |
| 208 | 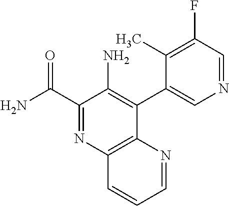 [SMILES: Cc1c(F)cncc1-c1c(N)c(C(N)=O)nc2cccnc12] |
| 209 | 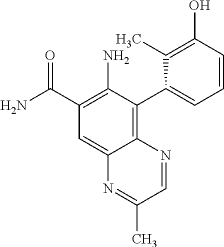 |
| 210 | 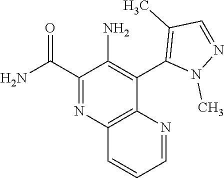 [SMILES: Cc1cnn(C)c1-c1c(N)c(C(N)=O)nc2cccnc12] |
| 211 | 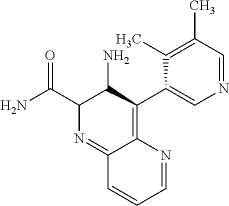 |
| 212 | 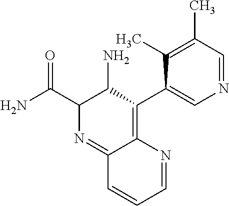 |
| 213 | 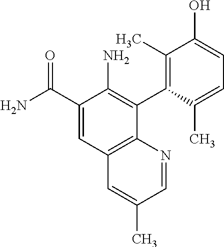 |
| 214 | 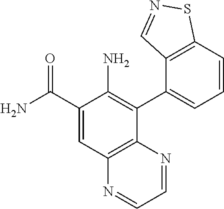 [SMILES: NC(=O)c1cc2nccnc2c(-c2cccc3sncc23)c1N] |
| 215 | 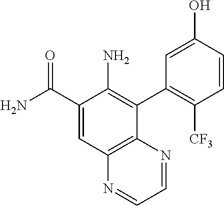 [SMILES: NC(=O)c1cc2nccnc2c(-c2cc(O)ccc2C(F)(F)F)c1N] |
| 216 | 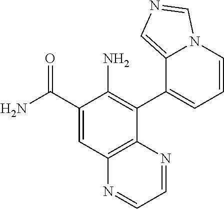 [SMILES: NC(=O)c1cc2nccnc2c(-c2cccn3cncc23)c1N] |
| 217 | 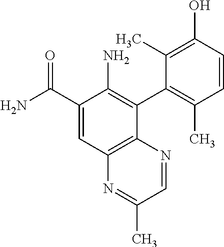 [SMILES: Cc1cnc2c(-c3c(C)ccc(O)c3C)c(N)c(C(N)=O)cc2n1] |
| 218 | 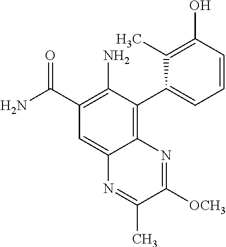 |
| 219 | 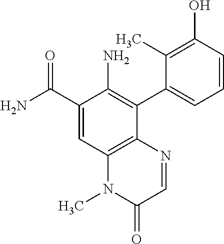 [SMILES: Cc1c(O)cccc1-c1c(N)c(C(N)=O)cc2c1ncc(=O)n2C] |
| 220 | 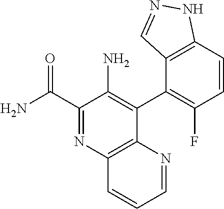 [SMILES: NC(=O)c1nc2cccnc2c(-c2c(F)ccc3[nH]ncc23)c1N] |
| 221 | 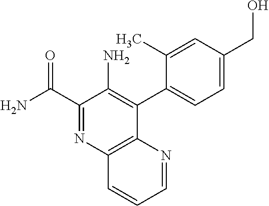 [SMILES: Cc1cc(CO)ccc1-c1c(N)c(C(N)=O)nc2cccnc12] |
| 222 | 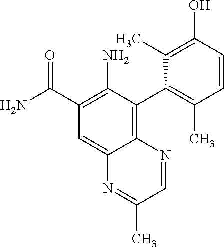 |
| 223 | 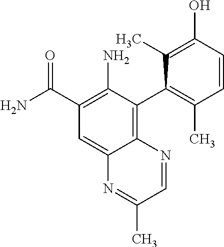 |
| 224 | 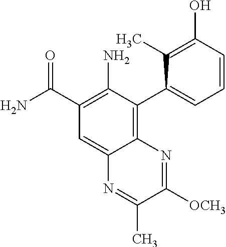 |
| 225 | 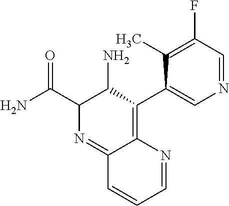 |
| 226 | 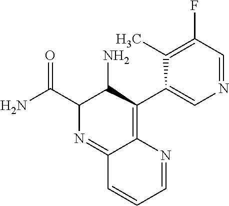 |
| 227 | 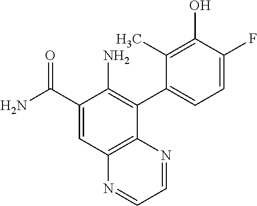 [SMILES: Cc1c(-c2c(N)c(C(N)=O)cc3nccnc23)ccc(F)c1O] |
| 228 | 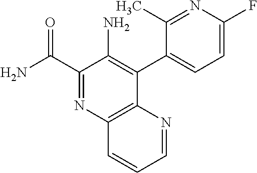 [SMILES: Cc1nc(F)ccc1-c1c(N)c(C(N)=O)nc2cccnc12] |
| 229 | 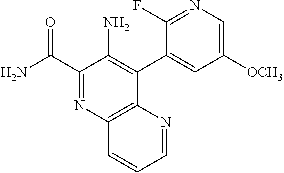 [SMILES: COc1cnc(F)c(-c2c(N)c(C(N)=O)nc3cccnc23)c1] |
| 230 | 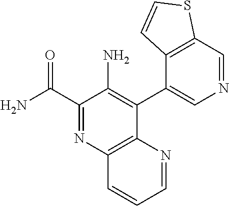 [SMILES: NC(=O)c1nc2cccnc2c(-c2cncc3sccc23)c1N] |
| 231 | 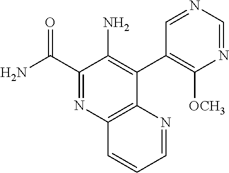 [SMILES: COc1ncncc1-c1c(N)c(C(N)=O)nc2cccnc12] |
| 232 | 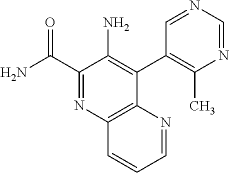 [SMILES: Cc1ncncc1-c1c(N)c(C(N)=O)nc2cccnc12] |
| 233 | 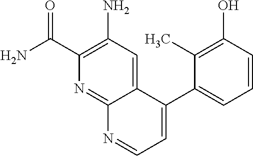 [SMILES: Cc1c(O)cccc1-c1ccnc2nc(C(N)=O)c(N)cc12] |
| 234 | 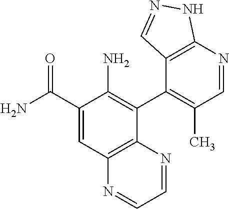 [SMILES: Cc1cnc2[nH]ncc2c1-c1c(N)c(C(N)=O)cc2nccnc12] |
| 235 | 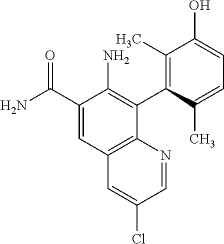 |
| 236 | 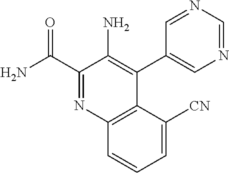 [SMILES: N#Cc1cccc2nc(C(N)=O)c(N)c(-c3cncnc3)c12] |
| 237 | 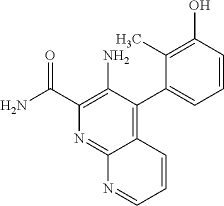 [SMILES: Cc1c(O)cccc1-c1c(N)c(C(N)=O)nc2ncccc12] |
| 238 | 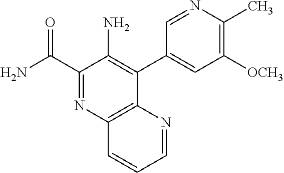 [SMILES: COc1cc(-c2c(N)c(C(N)=O)nc3cccnc23)cnc1C] |
| 239 | 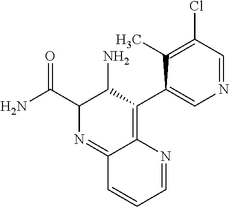 |
| 240 | 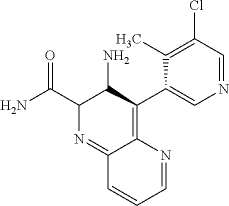 |
| 241 | 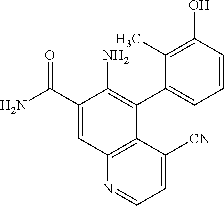 [SMILES: Cc1c(O)cccc1-c1c(N)c(C(N)=O)cc2nccc(C#N)c12] |
| 242 | 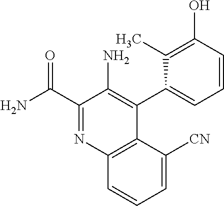 |
| 243 | 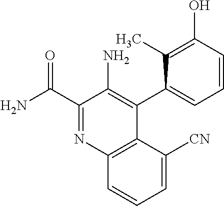 |
| 244 | 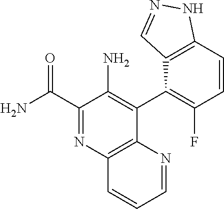 |
| 245 | 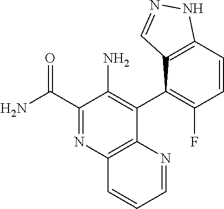 |
| 246 | 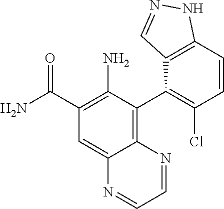 |
| 247 | 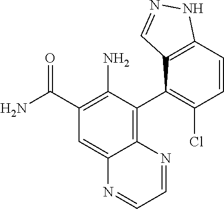 |
| 248 | 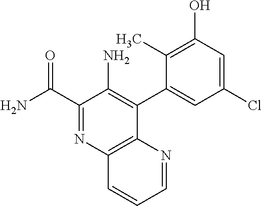 [SMILES: Cc1c(O)cc(Cl)cc1-c1c(N)c(C(N)=O)nc2cccnc12] |
| 249 | 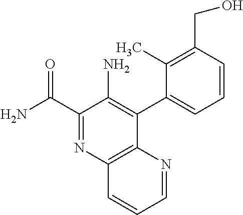 [SMILES: Cc1c(CO)cccc1-c1c(N)c(C(N)=O)nc2cccnc12] |
| 250 | 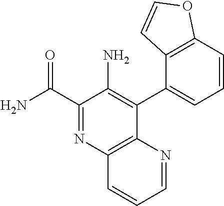 [SMILES: NC(=O)c1nc2cccnc2c(-c2cccc3occc23)c1N] |
| 251 | 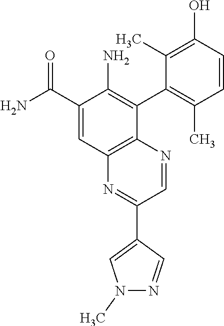 [SMILES: Cc1ccc(O)c(C)c1-c1c(N)c(C(N)=O)cc2nc(-c3cnn(C)c3)cnc12] |
| 252 | 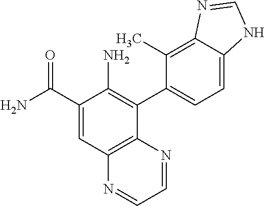 [SMILES: Cc1c(-c2c(N)c(C(N)=O)cc3nccnc23)ccc2[nH]cnc12] |
| 253 | 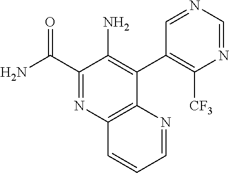 [SMILES: NC(=O)c1nc2cccnc2c(-c2cncnc2C(F)(F)F)c1N] |
| 254 | 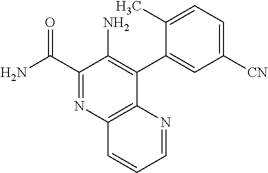 [SMILES: Cc1ccc(C#N)cc1-c1c(N)c(C(N)=O)nc2cccnc12] |
| 255 | 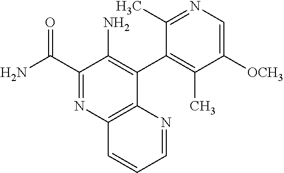 [SMILES: COc1cnc(C)c(-c2c(N)c(C(N)=O)nc3cccnc23)c1C] |
| 256 | 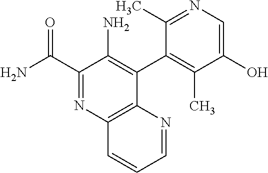 [SMILES: Cc1ncc(O)c(C)c1-c1c(N)c(C(N)=O)nc2cccnc12] |
| 257 | 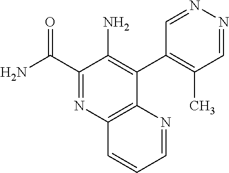 [SMILES: Cc1cnncc1-c1c(N)c(C(N)=O)nc2cccnc12] |
| 258 | 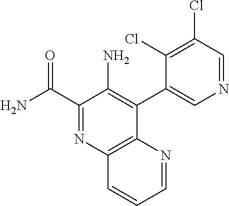 [SMILES: NC(=O)c1nc2cccnc2c(-c2cncc(Cl)c2Cl)c1N] |
| 259 | 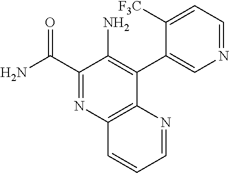 [SMILES: NC(=O)c1nc2cccnc2c(-c2cnccc2C(F)(F)F)c1N] |
| 260 | 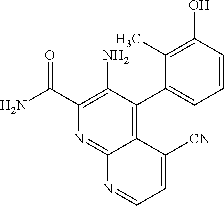 [SMILES: Cc1c(O)cccc1-c1c(N)c(C(N)=O)nc2nccc(C#N)c12] |
| 261 | 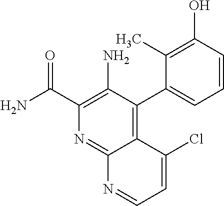 [SMILES: Cc1c(O)cccc1-c1c(N)c(C(N)=O)nc2nccc(Cl)c12] |
| 262 | 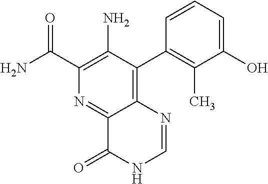 [SMILES: Cc1c(O)cccc1-c1c(N)c(C(N)=O)nc2c(=O)[nH]cnc12] |
| 263 | 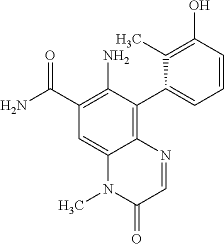 |
| 264 | 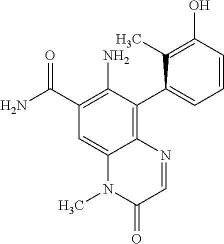 |
| 265 | 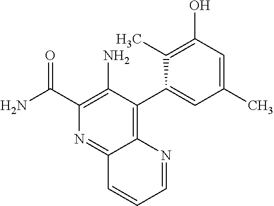 |
| 266 | 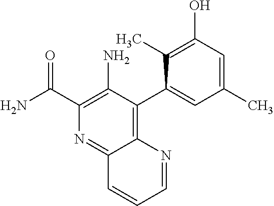 |
| 267 | 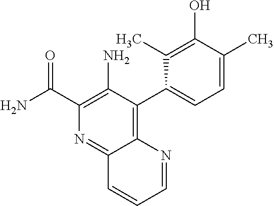 |
| 268 | 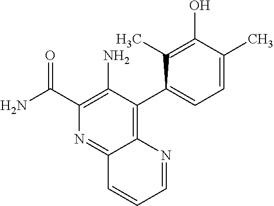 |
| 269 | 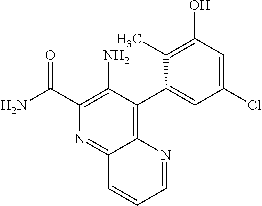 |
| 270 | 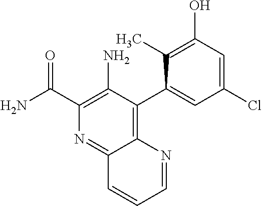 |
| 271 | 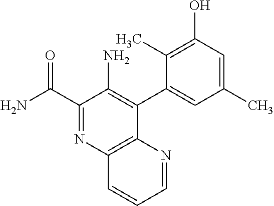 [SMILES: Cc1cc(O)c(C)c(-c2c(N)c(C(N)=O)nc3cccnc23)c1] |
| 272 | 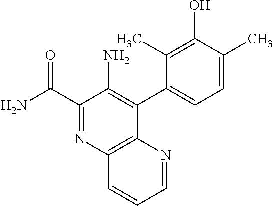 [SMILES: Cc1ccc(-c2c(N)c(C(N)=O)nc3cccnc23)c(C)c1O] |
| 273 | 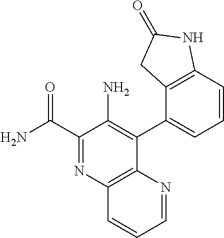 [SMILES: NC(=O)c1nc2cccnc2c(-c2cccc3c2CC(=O)N3)c1N] |
| 274 | 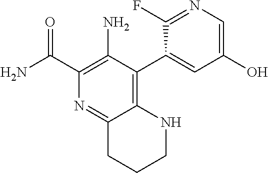 |
| 275 | 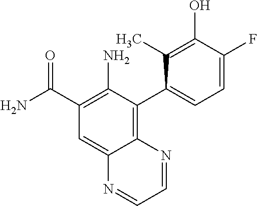 |
| 276 | 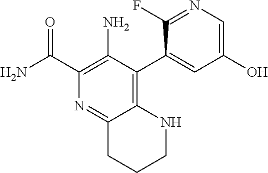 |
| 277 | 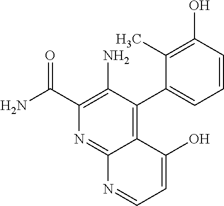 [SMILES: Cc1c(O)cccc1-c1c(N)c(C(N)=O)nc2nccc(O)c12] |
| 278 | 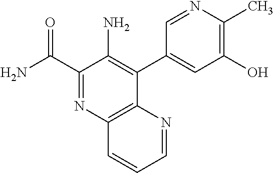 [SMILES: Cc1ncc(-c2c(N)c(C(N)=O)nc3cccnc23)cc1O] |
| 279 | 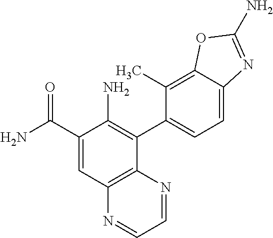 [SMILES: Cc1c(-c2c(N)c(C(N)=O)cc3nccnc23)ccc2nc(N)oc12] |
| 280 | 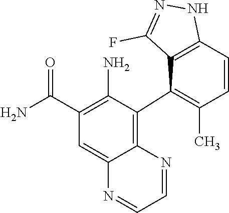 |
| 281 | 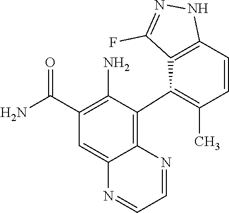 |

TABLE 1C
Compounds of the present disclosure

| Compound | |
|---|---|
| No. | Structure |
| 282 | 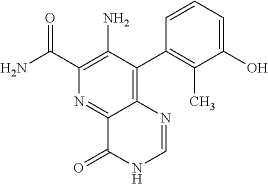 [SMILES: Cc1c(O)cccc1-c1c(N)c(C(N)=O)nc2c(=O)[nH]cnc12] |
| 283 | 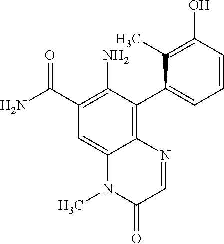 |
| 284 | 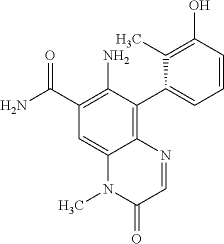 |
| 285 | 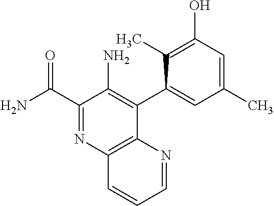 |
| 286 | 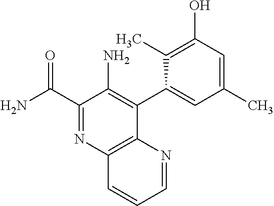 |
| 287 | 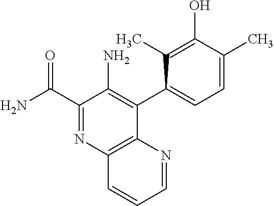 |
| 288 | 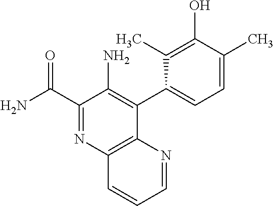 |
| 289 | 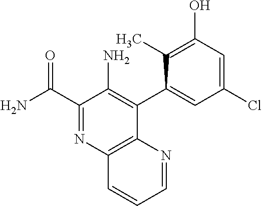 |
| 290 | 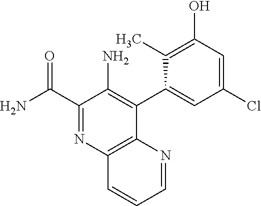 |
| 291 | 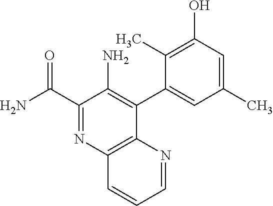 [SMILES: Cc1cc(O)c(C)c(-c2c(N)c(C(N)=O)nc3cccnc23)c1] |
| 292 | 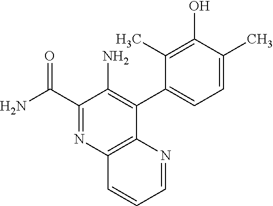 [SMILES: Cc1ccc(-c2c(N)c(C(N)=O)nc3cccnc23)c(C)c1O] |
| 293 | 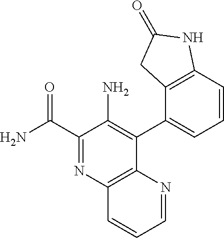 [SMILES: NC(=O)c1nc2cccnc2c(-c2cccc3c2CC(=O)N3)c1N] |
| 294 | 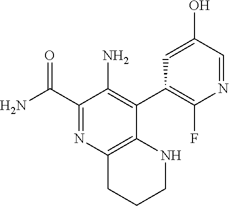 |
| 295 | 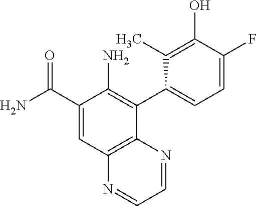 |
| 296 | 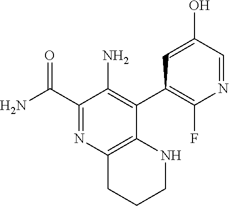 |
| 297 | 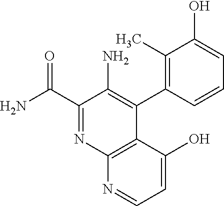 [SMILES: Cc1c(O)cccc1-c1c(N)c(C(N)=O)nc2nccc(O)c12] |
| 298 | 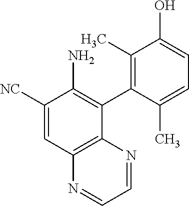 [SMILES: Cc1ccc(O)c(C)c1-c1c(N)c(C#N)cc2nccnc12] |
| 299 | 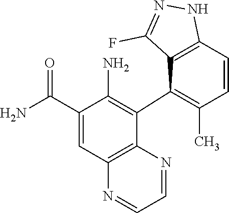 |
| 300 | 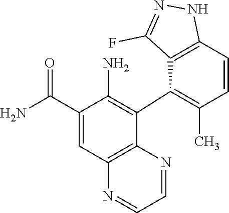 |
| 301 | 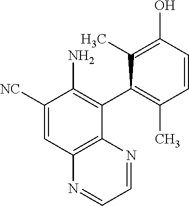 |
| 302 | 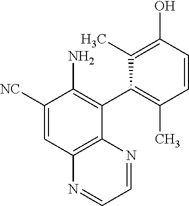 |
| 303 | 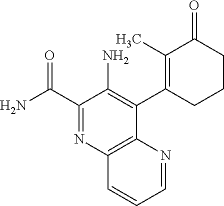 [SMILES: CC1=C(c2c(N)c(C(N)=O)nc3cccnc23)CCCC1=O] |
| 304 | 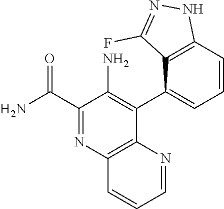 |
| 305 | 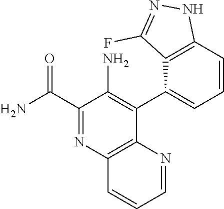 |
| 306 | 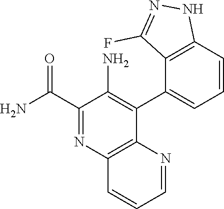 [SMILES: NC(=O)c1nc2cccnc2c(-c2cccc3[nH]nc(F)c23)c1N] |
| 307 | 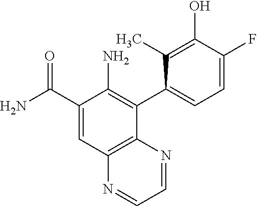 |
| 308 | 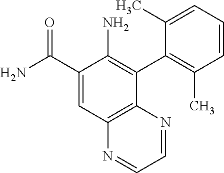 [SMILES: Cc1cccc(C)c1-c1c(N)c(C(N)=O)cc2nccnc12] |
| 309 | 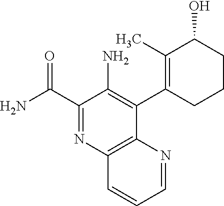 [SMILES: CC1=C(c2c(N)c(C(N)=O)nc3cccnc23)CCC[C@H]1O] |
| 310 | 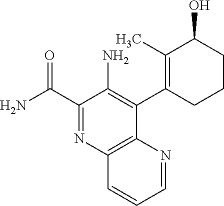 [SMILES: CC1=C(c2c(N)c(C(N)=O)nc3cccnc23)CCC[C@@H]1O] |
| 311 | 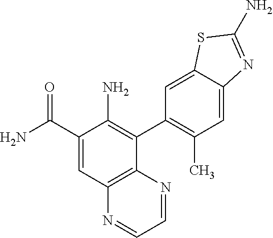 [SMILES: Cc1cc2nc(N)sc2cc1-c1c(N)c(C(N)=O)cc2nccnc12] |
| 312 | 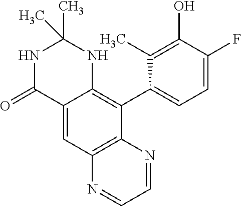 |
| 313 | 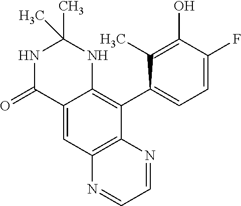 |
| 314 | 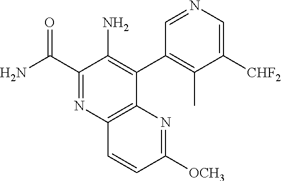 [SMILES: COc1ccc2nc(C(N)=O)c(N)c(-c3cncc(C(F)F)c3C)c2n1] |
| 315 | 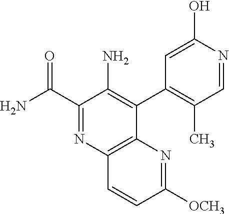 [SMILES: COc1ccc2nc(C(N)=O)c(N)c(-c3cc(O)ncc3C)c2n1] |
| 316 | 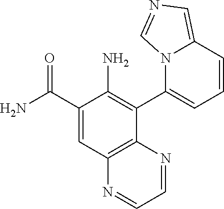 [SMILES: NC(=O)c1cc2nccnc2c(-c2cccc3cncn23)c1N] |
| 317 | 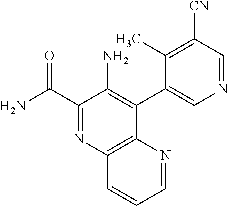 [SMILES: Cc1c(C#N)cncc1-c1c(N)c(C(N)=O)nc2cccnc12] |
| 318 | 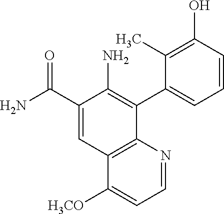 [SMILES: COc1ccnc2c(-c3cccc(O)c3C)c(N)c(C(N)=O)cc12] |
| 319 | 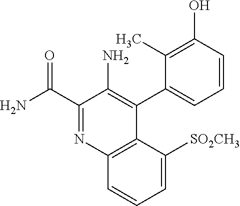 [SMILES: Cc1c(O)cccc1-c1c(N)c(C(N)=O)nc2cccc(S(C)(=O)=O)c12] |
| 320 | 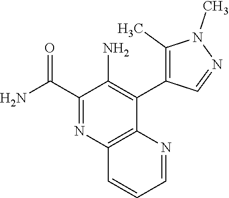 [SMILES: Cc1c(-c2c(N)c(C(N)=O)nc3cccnc23)cnn1C] |
| 321 | 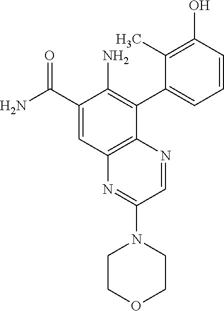 [SMILES: Cc1c(O)cccc1-c1c(N)c(C(N)=O)cc2nc(N3CCOCC3)cnc12] |
| 322 | 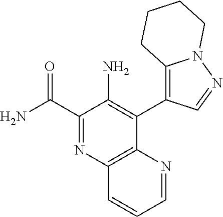 [SMILES: NC(=O)c1nc2cccnc2c(-c2cnn3c2CCCC3)c1N] |
| 323 | 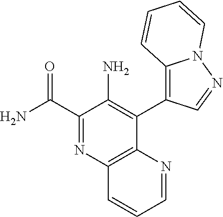 [SMILES: NC(=O)c1nc2cccnc2c(-c2cnn3ccccc23)c1N] |
| 324 | 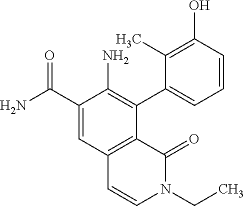 [SMILES: CCn1ccc2cc(C(N)=O)c(N)c(-c3cccc(O)c3C)c2c1=O] |
| 325 | 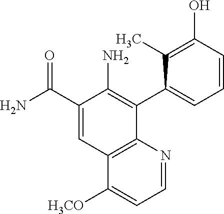 |
| 326 | 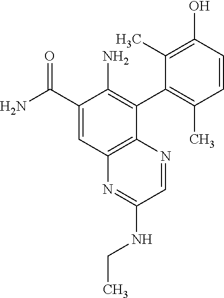 [SMILES: CCNc1cnc2c(-c3c(C)ccc(O)c3C)c(N)c(C(N)=O)cc2n1] |
| 327 | 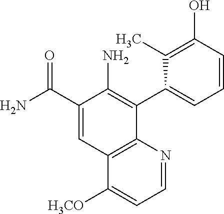 |
| 328 | 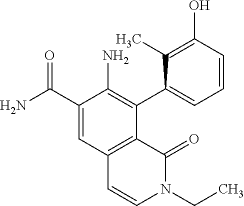 |
| 329 | 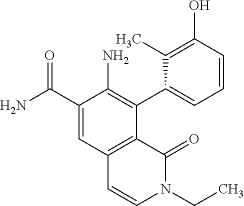 |
| 330 | 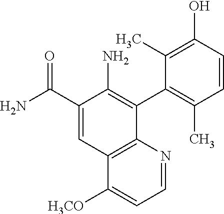 [SMILES: COc1ccnc2c(-c3c(C)ccc(O)c3C)c(N)c(C(N)=O)cc12] |
| 331 | 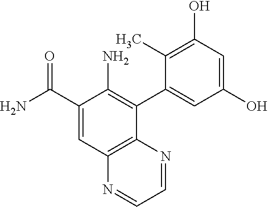 [SMILES: Cc1c(O)cc(O)cc1-c1c(N)c(C(N)=O)cc2nccnc12] |
| 332 | 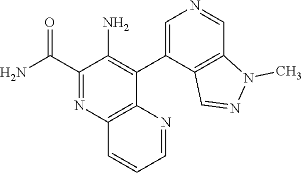 [SMILES: Cn1ncc2c(-c3c(N)c(C(N)=O)nc4cccnc34)cncc21] |
| 333 | 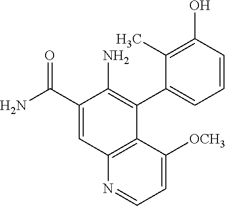 [SMILES: COc1ccnc2cc(C(N)=O)c(N)c(-c3cccc(O)c3C)c12] |
| 334 | 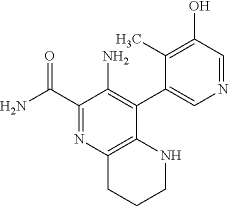 [SMILES: Cc1c(O)cncc1-c1c(N)c(C(N)=O)nc2c1NCCC2] |
| 335 | 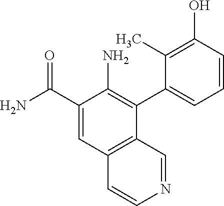 [SMILES: Cc1c(O)cccc1-c1c(N)c(C(N)=O)cc2ccncc12] |
| 336 | 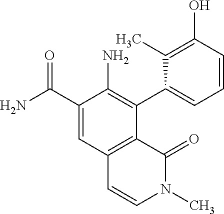 |
| 337 | 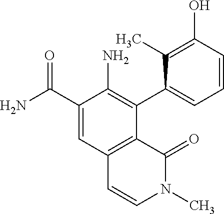 |
| 338 | 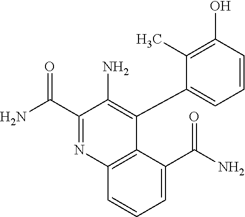 [SMILES: Cc1c(O)cccc1-c1c(N)c(C(N)=O)nc2cccc(C(N)=O)c12] |
| 339 | 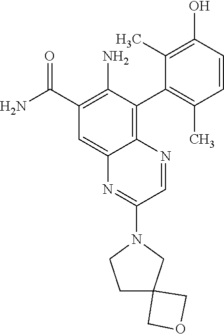 [SMILES: Cc1ccc(O)c(C)c1-c1c(N)c(C(N)=O)cc2nc(N3CCC4(COC4)C3)cnc12] |

### Pharmaceutical Composition and Medical Treatment or Uses

Another aspect provides a pharmaceutical composition comprising any of the compounds of Formula I disclosed herein, or a pharmaceutically acceptable salt thereof, and one or more pharmaceutically acceptable excipients.

Compounds of the present disclosure inhibit PKMYT1 and are thus useful in the treatment or prevention of a variety of diseases and conditions. In particular, compounds of the present disclosure are useful in methods of treating or preventing a disease or condition wherein inhibition of PKMYT1 provides a benefit. It has further been discovered that particular compounds exhibit advantageously low rates of hepatic metabolic clearance as assessed, for example, by stability assays employing liver microsomes or hepatocytes obtained from humans or other species. Certain compounds of the present disclosure have also exhibited an advantageously low propensity for induction of liver enzyme expression (e.g., induction of CYP3A4 expression) and high selectivity for inhibition of PKMYT1 over other kinases, such as Src-family kinases Lck and Lyn that regulate signal transduction and the WEE-family kinase Weel that regulates cycle progression. Accordingly, another aspect provides methods for treating cancer comprising administering a therapeutically effective amount of a compound of Formula I disclosed herein, or a pharmaceutically acceptable salt thereof, or a pharmaceutical composition comprising any of the compounds of Formula I disclosed herein, or a pharmaceutically acceptable salt thereof, to a subject in need thereof. Examples of cancers for treatment according to the methods include, but are not limited to, cancers of the adrenal glands, bladder, brain and spine, breast, cervix, colorectal, endometrium, esophagus, head & neck, blood and lymph, kidneys, liver, lung, ovaries, pancreas, skin, soft tissue (sarcoma), stomach, and uterus. In some embodiments, the cancer is a cancer overexpressing CCNE1. In some embodiments, the cancer is a cancer expressing one or more mutations in F-box/WD repeat-containing protein 7 (FBXW7) and/or protein phosphatase 2 regulatory subunit A alpha (PPP2R1A). A related aspect provides for the use of any of the compounds of Formula I disclosed herein, or a pharmaceutically acceptable salt thereof, or use of a pharmaceutical composition comprising any of the compounds of Formula I disclosed herein, or a pharmaceutically acceptable salt thereof in methods for treating cancer, or in the manufacture of medicaments for treating cancer. In some embodiments, the cancer in such uses is a cancer overexpressing CCNE1 or expressing one or more mutations in FBXW7 and/or PPP2R1A.

Administration of the compounds provided herein, or their pharmaceutically acceptable salts, in pure form or in an appropriate pharmaceutical composition, can be carried out via any of the accepted modes of administration or agents for serving similar utilities. Thus, administration can be, for example, oral, nasal, parenteral (intravenous, intramuscular, or subcutaneous), topical, transdermal, intravaginal, intravesical, intracisternal, or rectal, in the form of solid, semi-solid, lyophilized powder, or liquid dosage forms, such as, for example, tablets, suppositories, pills, soft elastic and hard gelatin capsules, powders, solutions, suspensions, aerosols, and the like, optionally in unit dosage forms suitable for simple administration of precise dosages.

The compositions will typically include a conventional pharmaceutical carrier or excipient and a compound of Formula I as the/an active agent, and, in addition, carriers, adjuvants, and the like. Excipients may include, for example, preserving, wetting, suspending, sweetening, flavoring, perfuming, emulsifying, and dispensing agents. Prevention of the action of microorganisms can be ensured by various antibacterial and antifungal agents. If desired, a pharmaceutical composition may also contain substances such as wetting or emulsifying agents, pH buffering agents, isotonic agents, and antioxidants.

Compositions suitable for parenteral injection may comprise physiologically acceptable sterile aqueous or nonaqueous solutions, dispersions, suspensions or emulsions, and sterile powders for reconstitution into sterile injectable solutions or dispersions.

Solid dosage forms for oral administration include capsules, tablets, pills, powders, and granules.

Liquid dosage forms for oral administration include pharmaceutically acceptable emulsions, solutions, suspensions, syrups, and elixirs. Such dosage forms are prepared, for example, by dissolving, dispersing, or otherwise combining, a compound(s) of Formula I, or a pharmaceutically acceptable salt thereof, and optional pharmaceutical adjuvants in an aqueous or non-aqueous carrier to thereby form a solution or suspension. Suspensions, in addition to the active compounds, may contain suspending agents.

Compositions for rectal administration include, for example, suppositories that can be prepared by mixing the compounds of the present invention with suitable non-irritating excipients or carriers which are solid at ordinary temperatures but liquid at body temperature and therefore melt while in a suitable body cavity and release the active component therein.

Dosage forms for topical administration include ointments, powders, sprays, and inhalants. The active component is typically admixed under sterile conditions with a physiologically acceptable carrier and any preservatives, buffers, or propellants as may be required. Ophthalmic formulations, eye ointments, powders, and solutions are also contemplated for use in topical administration.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients. Methods of preparing such dosage forms are known, or will be apparent, to those skilled in this art; for example, see Remington The Science and Practice of Pharmacy, 23rd Ed., (Academic Press 2020). The composition to be administered will, in any event, contain a therapeutically effective amount of a compound as described herein, or a pharmaceutically acceptable salt thereof, for treatment of a disease-state as described herein.

The compounds described herein (e.g., compounds of Formula I), or their pharmaceutically acceptable salts, are generally administered in a therapeutically effective amount which will vary depending upon a variety of factors including the activity of the specific compound employed, the metabolic stability and length of action of the compound, the age, body weight, general health, sex, diet, mode, and time of administration, rate of excretion, drug combination, the severity of the particular disease-states, and the host undergoing therapy. The compounds of the present invention can be administered, for example, to a patient at dosage levels in the range of about 0.1 to about 1,000 mg per day.

### General Synthetic Methods

Compounds of this invention can be made by the synthetic procedures described below. These schemes are merely illustrative of some methods by which the compounds of this invention can be synthesized, and various modifications to these schemes can be made. The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

The compounds disclosed and claimed herein have asymmetric carbon atoms or quaternized nitrogen atoms in their structure and may be prepared through the syntheses described herein as single stereoisomers, racemates, or mixtures of enantiomers and diastereomers. The compounds may also exist as geometric isomers. All such single stereoisomers, racemates, and geometric isomers, and mixtures thereof are intended to be within the scope of this invention.

The compounds may also exist as atropisomers, which are conformational stereoisomers that result from hindered rotation about a single bond where the steric strain barrier to rotation can be high enough to allow for the isolation of each conformer. The compounds provided herein include all atropisomers, both as pure individual atropisomer preparations, enriched preparations of each, or a non-specific mixture of each.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

Various methods for the preparation and/or separation and isolation of single stereoisomers from racemic mixtures or non-racemic mixtures of stereoisomers. For example, supercritical fluid chromatography (SFC) and/or HPLC can be used for separation of chiral molecules. Further, optically active and isomers may be prepared using chiral synthons or chiral reagents, or resolved using conventional techniques. Enantiomers (R- and S-isomers) and atropisomers (M- and P-isomers) may be resolved, for example, by: formation of diastereomeric salts or complexes which may be separated, for example, by crystallization; via formation of diastereomeric derivatives which may be separated, for example, by crystallization; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent.

In addition, the compounds can exist in unsolvated as well as solvated forms with pharmaceutically acceptable solvents such as water, ethanol, and the like. In general, the solvated forms are considered equivalent to the unsolvated forms for the purposes of the present invention.

Processes for preparation of compounds according to the present disclosure may be carried out as semi-continuous or continuous processes, more preferably as continuous processes. Reactions in the processes may be carried out in the presence of a solvent or a mixture of two or more solvents, or the reactions may be carried out in the absence of additional solvents. In particular, the solvent may be an aqueous solvent or an organic solvent such as an ether (e.g. tetrahydrofuran, methyltetrahydrofuran, diisopropyl ether, t-butyl methyl ether, or dibutyl ether), aliphatic hydrocarbon solvent (e.g. hexane, heptane, or pentane), saturated alicyclic hydrocarbon solvent (e.g. cyclohexane or cyclopentane), or aromatic solvent (e.g. toluene, o-, m-, or p-xylene, or t-butyl-benzene), or mixture thereof. The starting materials and reagents, which do not have their synthetic route explicitly disclosed herein, are generally available from commercial sources or are readily prepared using known methods.

The compounds disclosed and claimed herein can be prepared according to the general Scheme 1 shown below.

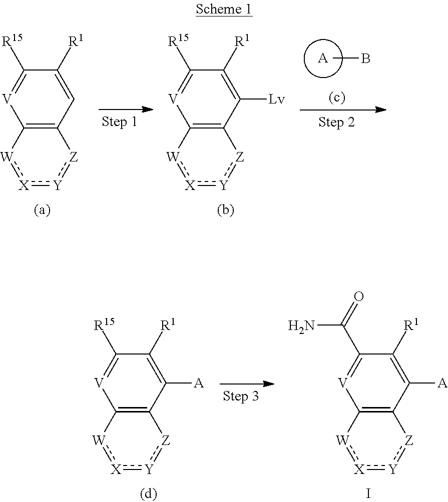

In the general scheme, variables such as ring A, V, W, X, Y, Z, R¹, and R^{a} are as defined herein. R¹⁵ is —COOR^{a}, —CONH₂, or CN. “Lv” refers to a leaving group including, but not limited to halogen, mesylates, tosylates and the like.

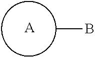

is a boronic acid or boronic ester of ring A.

One aspect of the present disclosure provides a process for making a compound of Formula I,

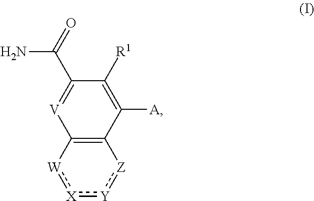

or a pharmaceutically acceptable salt thereof, comprising
(a) combining a compound of Formula (b) and a compound of Formula (c) to form a compound of Formula (d)

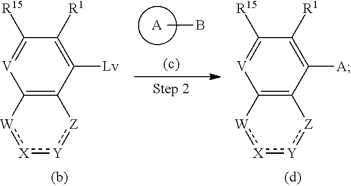

(b) converting the compound of Formula (d) to yield the compound of Formula I,

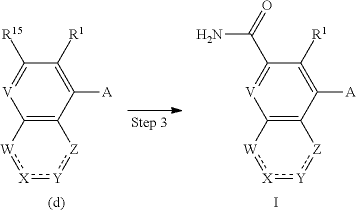

wherein
Lv is a leaving group;

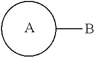

is a boronic acid or boronic ester of ring A;
each [symbol] represents a single bond, a double bond, or a delocalized π bond;
R¹⁵ is COOR^{a}, —COONH₂, or CN;
ring A, V, W, X, Y, Z, R¹, and R^{a} are as defined herein.

In some embodiments, the process further comprises converting a compound of Formula (a) to the compound of Formula (b):

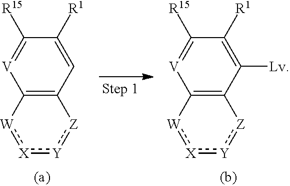

In some embodiments, the compound of formula I is a compound of formula Ia, Ib, Ic, Id, le, If, Ig, Ih, Ik, Im, or In, as described and defined herein.

In some embodiments, the compound of Formula (c) is:

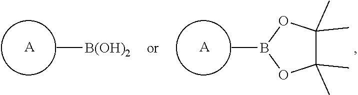

wherein ring A is as defined herein.

In some embodiments, Lv is halogen. In some embodiments, Lv is —Br. In some embodiments, Lv is —Cl.

In step 1 of the process, when Lv is halogen, the compound of formula (b) can be prepared by halogenation of the compound of formula (a). The reaction can be carried out by reacting the compound of formula (a) with chlorine, bromine, NBS, or the like in the presence of a suitable solvent. Examples of suitable solvents include, but are not limited to, DMF, THF, DMSO, acetonitrile, ether, ketone, and 1,4-dioxane.

Step 2 of the reaction can be carried out in the presence of a suitable base, catalyst, and solvent. The base may be an inorganic base or an organic base. Non-limiting examples of inorganic bases include bicarbonates, carbonates, phosphates, and acetates. Examples of organic bases include, but are not limited to, acyclic amines, e.g., tertiary amines, pyridine, and piperidine. The catalyst may be any catalyst suitable for Suzuki-type couplings, and includes but is not limited to a nickel, palladium, or platinum catalyst. Non-limiting examples of such catalysts include NiCl₂(dppf), SPhosPdG₃, Pd(PPh₃)₄, PdCl₂(PPh₃)₂, Pd(dppf)Cl₂-DCM, and Pd₂(dba)₃. Suitable solvents include, but are not limited to, protic and aprotic solvents such as water, methanol, ethanol, butanol, toluene, DMF, DME, DCM, THF, DMSO, ether, ketone, 1,4-dioxane, and the like. Combinations of two, three, or more solvents may also be employed. The reaction can be carried out in a temperature ranging from room temperature (about 20° C.) to about 200° C. In some embodiments, the reaction temperature is about 80° C. to about 150° C. In some embodiments, the reaction temperature is about 100° C. to about 120° C.

In step 3 of the process, the compound of formula (d) can be converted to the compound of formula (I) by amidation or partial hydrolysis. When R¹⁵ is —COOR^{a}, the compound of formula (d) can react with NH₃ in the presence of an alcohol such as methanol, ethanol, butanol, or the like. When R¹⁵ is —CN, the nitrile can be hydrolyzed to a carboxamide by acid or base hydrolysis. The acid hydrolysis can be carried out in the presence of a strong mineral acid, such as sulfuric acid, and water. The base hydrolysis can be carried out in the presence of a strong base such as NaOH, LiOH, or KOH. Hydrogen peroxide can also be used for the base hydrolysis.

In some embodiments, the compound of formula (I) exists as atropisomers. The atropisomers can be separated by chiral SFC.

The following examples are provided for the purpose of further illustration and are not intended to limit the scope of the claimed invention.

### Synthetic Examples

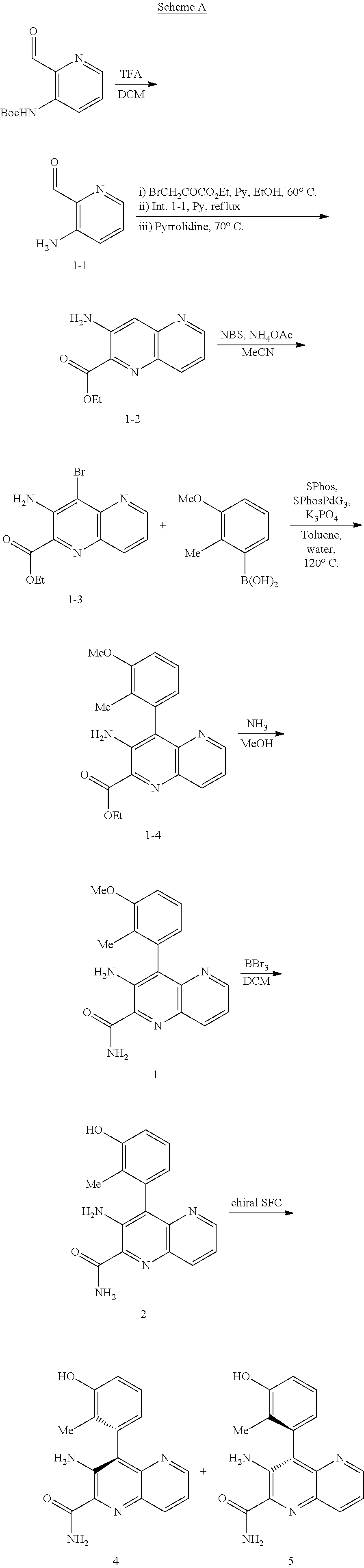

### Example 1. 3-Amino-4-(3-methoxy-2-methylphenyl)-1,5-naphthyridine-2-carboxamide (1)

3-Aminopicolinaldehyde (Intermediate 1-1): tert-Butyl (2-formylpyridin-3-yl)carbamate (2.50 g, 11.2 mmol, 1.00 equiv.) was added to a solution of DCM (25 mL) and trifluoroacetic acid (6 mL, 100 mass %) and stirred at room temperature for 2 hr. The solution was quenched with aqueous NaHCO₃ (75 mL) and extracted with DCM (2×75 mL). The organic layers were combined, dried over MgSO₄, and filtered to afford Intermediate 1-1 (1.10 g, 80% yield). LC-MS m/z 123 (M+H⁺).

Ethyl 3-amino-1,5-naphthyridine-2-carboxylate (Intermediate 1-2): Pyridine (791 μL, 9.82 mmol, 1.10 equiv.) was added to EtOH (24 mL) under an atmosphere of nitrogen gas. A solution of ethyl 3-bromo-2-oxopropanoate (1.23 mL, 9.82 mmol, 1.10 equiv.) in EtOH (16 mL) was added over 5 min then the solution was heated to 60° C. for 1.5 hr. The reaction mixture was cooled to room temperature then a solution of 3-aminopicolinaldehyde (1.09 g, 8.93 mmol, 1.00 equiv.) in pyridine (2 mL) and EtOH (5 mL) was added. The reaction mixture was heated to reflux for 6 hr. The mixture was then cooled to 70° C., pyrrolidine (1.79 mL, 21.4 mmol, 2.40 equiv.) was added, and the reaction mixture was stirred at 70° C. for 2 hr. The solvent was removed, the residue was adsorbed to Celite®, then subjected to flash column chromatography (FCC) (Hexanes:EtOAc, 50:50 to 0:100) to afford Intermediate 1-2 (598 mg, 31% yield). LC-MS m/z 218 (M+H⁺).

Ethyl 3-amino-4-bromo-1,5-naphthyridine-2-carboxylate (Intermediate 1-3): Ethyl 3-amino-1,5-naphthyridine-2-carboxylate (500 mg, 2.30 mmol, 1.00 equiv.) and ammonium acetate (17.7 mg, 0.230 mmol, 0.100 equiv.) were suspended in MeCN (10 mL). A solution of NBS (430 mg, 2.42 mmol, 1.05 equiv.) in MeCN (10 mL) was added dropwise over 2 min and the reaction mixture was stirred at room temperature for 30 min. The solvent was removed, the residue was adsorbed to Celite®, then subjected to FCC (Hexanes:EtOAc, 70:30 to 0:100). The solid obtained was added to water (50 mL) and extracted with DCM (2×50 mL). The organic layers were combined, dried over MgSO₄, filtered, and the solvent was removed to afford Intermediate 1-3 (595 mg, 87% yield). LC-MS m/z 296 (M+H⁺).

Ethyl 3-amino-4-(3-methoxy-2-methylphenyl)-1,5-naphthyridine-2-carboxylate (Intermediate 1-4): Ethyl 3-amino-4-bromo-1,5-naphthyridine-2-carboxylate (200 mg, 0.675 mmol, 1 equiv.), (3-methoxy-2-methylphenyl)boronic acid (224 mg, 1.35 mmol, 2.00 equiv), K₃PO₄ (287 mg, 1.35 mmol, 2.00 equiv.), and dicyclohexyl(2′,6′-dimethoxy[1,1′-biphenyl]-2-yl)phosphane (SPhos, 27.7 mg, 0.0675 mmol, 0.100 equiv.) were added to a microwave vial followed by toluene (3.6 mL) and water (0.4 mL). The mixture was sparged with nitrogen gas for 2 min, (2-dicyclohexylphosphino-2′,6′-dimethoxybiphenyl) [2-(2′-amino-1,1′-biphenyl)]palladium(II) methanesulfonate (SPhos Pd G3, 52.7 mg, 0.0675 mmol, 0.100 equiv.) was added, the mixture was sparged with nitrogen gas for 2 min, and then the vial was sealed. The vial was heated to 120° C. for 1 hr. The reaction mixture adsorbed to Celite®, then subjected to FCC (Hexanes:EtOAc, 70:30 to 0:100) to afford Intermediate 1-4 (242 mg, quantitative yield) of sufficient purity for the next step. LC-MS m/z 338 (M+H⁺).

3-amino-4-(3-methoxy-2-methylphenyl)-1,5-naphthyridine-2-carboxamide (1): Ethyl 3-amino-4-(3-methoxy-2-methyl-phenyl)-1,5-naphthyridine-2-carboxylate (229 mg, 0.679 mmol, 1.00 equiv.) was dissolved in a solution of ammonia in MeOH (7 M, 9 mL, 63 mmol, 93 equiv.) and ethanol (6 mL) in a microwave vial. The mixture was heated to 100° C. for 2.5 hr. The solvent was removed. The residue was resuspended in DCM and the solvent was removed to afford Compound 1 (208 mg, 99% yield). ¹H NMR (400 MHz, DMSO) δ 8.71 (s, 1H), 8.49 (s, 1H), 8.25 (d, 1H), 7.91 (s, 1H), 7.48-7.40 (m, 1H), 7.31 (t, 1H), 7.05 (d, 1H), 6.71 (d, 1H), 6.35 (s, 2H), 5.76 (DCM, s, 1H), 3.87 (s, 3H), 1.74 (s, 3H). LC-MS m/z 309 (M+H⁺).

### Example 2. 3-Amino-4-(3-hydroxy-2-methylphenyl)-1,5-naphthyridine-2-carboxamide (2)

3-amino-4-(3-methoxy-2-methyl-phenyl)-1,5-naphthyridine-2-carboxamide (186 mg, 0.604 mmol, 1.00 equiv.) was dissolved in DCM (5 mL) under an atmosphere of nitrogen gas. A solution of BBr₃ in DCM (1 M, 1.81 mL, 3.00 equiv.) was added dropwise over 1 min and the mixture was stirred at room temperature for 15 min. Another portion of BBr₃ in DCM (1 M, 905 μL, 1.50 equiv.) was added and the mixture was stirred for 10 min. The solvent was removed in vacuo, and the residue was resuspended in DCM. This mixture was poured into a saturated solution of NaHCO₃ (50 mL) and DCM (50 mL). The mixture was filtered and the precipitate was dissolved in DMF, filtered, and subjected to RP-HPLC (0.1% formic acid (FA), Water:MeCN, 90:10 to 0:100) to afford Compound 2 (60 mg, 34% yield). ¹H NMR (400 MHz, d6-DMSO) δ 9.45 (s, 1H), 8.71 (s, 1H), 8.48 (s, 1H), 8.25 (d, 1H), 7.89 (s, 1H), 7.47-7.42 (m, 1H), 7.13 (t, 1H), 6.90 (d, 1H), 6.55 (d, 1H), 6.31 (s, 2H), 1.70 (s, 3H). LC-MS m/z 295 (M+H⁺).

### Example 3. (P)-3-Amino-4-(3-hydroxy-2-methylphenyl)-1,5-naphthyridine-2-carboxamide (4) and (M)-3-amino-4-(3-hydroxy-2-methylphenyl)-1,5-naphthyridine-2-carboxamide (5)

Chiral SFC separation of Compound 2 (50 mg, 0.17 mmol) (Instrument: Waters SFC Prep 150 Mgm; Column: Daicel Chiralpak® OJ, 30×150 mm, 5 um; Conditions: Isocratic at 20% methanol with 80% CO2; Flow Rate: 100 mL/min) provided Compound 4 (18 mg) and Compound 5 (19 mg).

Peak 1 retention time 2.29 min. ¹H NMR (400 MHz, d6-DMSO) δ 9.45 (s, 1H), 8.75-8.68 (m, 1H), 8.48 (s, 1H), 8.24 (d, 1H), 7.90 (s, 1H), 7.48-7.40 (m, 1H), 7.13 (t, 1H), 6.90 (d, 1H), 6.55 (d, 1H), 6.31 (s, 2H), 1.70 (s, 3H). LC-MS m/z 295 (M+H).

Peak 2 retention time 2.57 min. ¹H NMR (400 MHz, d6-DMSO) δ 9.45 (s, 1H), 8.74-8.68 (m, 1H), 8.48 (s, 1H), 8.25 (d, 1H), 7.90 (s, 1H), 7.48-7.40 (m, 1H), 7.13 (t, 1H), 6.90 (d, 1H), 6.55 (d, 1H), 6.31 (s, 2H), 1.70 (s, 3H). LC-MS m/z 295 (M+H⁺).

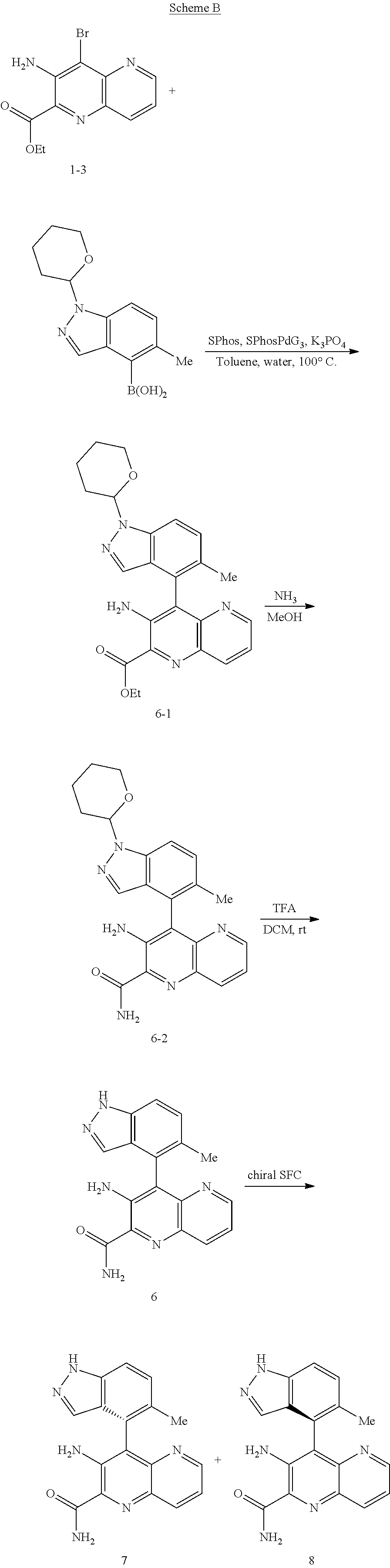

### Example 4. 3-Amino-4-(5-methyl-1H-indazol-4-yl)-1,5-naphthyridine-2-carboxamide (6)

Ethyl 3-amino-4-(5-methyl-1-(tetrahydro-2H-pyran-2-yl)-1H-indazol-4-yl)-1,5-naphthyridine-2-carboxylate (Intermediate 6-1): Ethyl 3-amino-4-bromo-1,5-naphthyridine-2-carboxylate (210 mg, 0.709 mmol, 1 equiv.), (5-methyl-1-tetrahydropyran-2-yl-indazol-4-yl)boronic acid (370 mg, 1.42 mmol, 2 equiv), K₃PO₄ (450 mg, 2.11 mmol, 3 equiv.), and dicyclohexyl(2′,6′-dimethoxy[1,1′-biphenyl]-2-yl)phosphane (SPhos, 60 mg, 0.146 mmol, 0.2 equiv.) were added to a vial followed by toluene (5 mL) and water (0.5 mL). The mixture was sparged with nitrogen gas for 2 min, (2-dicyclohexylphosphino-2′,6′-dimethoxybiphenyl) [2-(2′-amino-1,1′-biphenyl)]palladium(II) methanesulfonate (SPhos Pd G3, 55 mg, 0.07 mmol, 0.1 equiv.) was added; the mixture was sparged with nitrogen gas for 2 min and then sealed. The vial was heated to 100° C. overnight. The cooled reaction mixture was diluted with water (5 ml) and extracted with ethyl acetate (4×20 mL). The organic layers were combined, washed with brine, dried over MgSO₄, filtered, and evaporated to dryness to afford Intermediate 6-1 (250 mg, 82% yield) of sufficient purity for the next step. LC-MS m/z 432.3 (M+H⁺).

3-amino-4-(5-methyl-1-(tetrahydro-2H-pyran-2-yl)-1H-indazol-4-yl)-1,5-naphthyridine-2-carboxamide (Intermediate 6-2): Ethyl 3-amino-4-(5-methyl-1-tetrahydropyran-2-yl-indazol-4-yl)-1,5-naphthyridine-2-carboxylate (130 mg, 0.210 mmol, 1 equiv) was suspended in a solution of ammonia in MeOH (7 M, 2.3 mL, 16 mmol, 75 equiv.) in a sealed tube and heated to 75° C. for 1 h. The reaction mixture was adsorbed to Celite®, then subjected to FCC (Hexanes:EtOAc, 70:30 to 0:100) to obtain Intermediate 6-2 (65 mg, 76% yield). LC-MS m/z 403.2 (M+H⁺).

3-amino-4-(5-methyl-1H-indazol-4-yl)-1,5-naphthyridine-2-carboxamide (6): A suspension of 3-amino-4-(5-methyl-1-(tetrahydro-2H-pyran-2-yl)-1H-indazol-4-yl)-1,5-naphthyridine-2-carboxamide (60 mg, 0.149 mmol, 1 equiv) in dichloromethane (1 ml) at RT was treated with 2,2,2-trifluoroacetic acid (0.17 mL, 2.2 mmol, 15 equiv) and stirred overnight. The mixture was evaporated to dryness, redissolved in DCM, and basified with saturated aq. NaHCO₃ to pH 7-8. The organic layer was separated. The aqueous layer was extracted with DCM (3×10 ml). The combined organic layers were evaporated. The crude was dissolved in DMSO (3 ml) and subjected to RP-HPLC (0.1% FA, Water:MeCN, 90:10 to 0:100) to afford Compound 6 (40 mg, 84% yield). LC-MS m/z 319.2 (M+H⁺). ¹H NMR (400 MHz, d6-DMSO) δ 13.03 (s, 1H), 8.63 (s, 1H), 8.54 (s, 1H), 8.29 (d, 1H), 7.93 (s, 1H), 7.54 (d, 1H), 7.39 (d, 2H), 7.27 (s, 1H), 6.36 (s, 2H), 2.01 (s, 3H).

### Example 5. (P)-3-amino-4-(5-methyl-1H-indazol-4-yl)-1,5-naphthyridine-2-carboxamide (7) and (M)-3-amino-4-(5-methyl-1H-indazol-4-yl)-1,5-naphthyridine-2-carboxamide (8)

Chiral SFC separation of Compound 6 (40 mg, 0.12 mmol) (Instrument: Waters SFC Prep 150 Mgm; Column: Daicel Chiralpak® OJ, 30×150 mm, 5 um; Conditions: Isocratic at 20% methanol with 80% CO2; Flow Rate: 100 mL/min) provided Compound 7 (15 mg) and Compound 8 (14 mg).

Peak 1: Retention time 2.47 min. ¹H NMR (400 MHz, d6-DMSO) δ 13.03 (s, 1H), 8.63 (s, 1H), 8.54 (s, 1H), 8.29 (d, 1H), 7.93 (s, 1H), 7.54 (d, 1H), 7.39 (d, 2H), 7.27 (s, 1H), 6.36 (s, 2H), 2.01 (s, 3H). LC-MS m/z 319.20 (M+H).

Peak 2: Retention time 3.92 min. ¹H NMR (400 MHz, d6-DMSO) δ 13.03 (s, 1H), 8.63 (s, 1H), 8.54 (s, 1H), 8.29 (d, 1H), 7.93 (s, 1H), 7.54 (d, 1H), 7.39 (d, 2H), 7.27 (s, 1H), 6.36 (s, 2H), 2.01 (s, 3H). LC-MS m/z 319.20 (M+H).

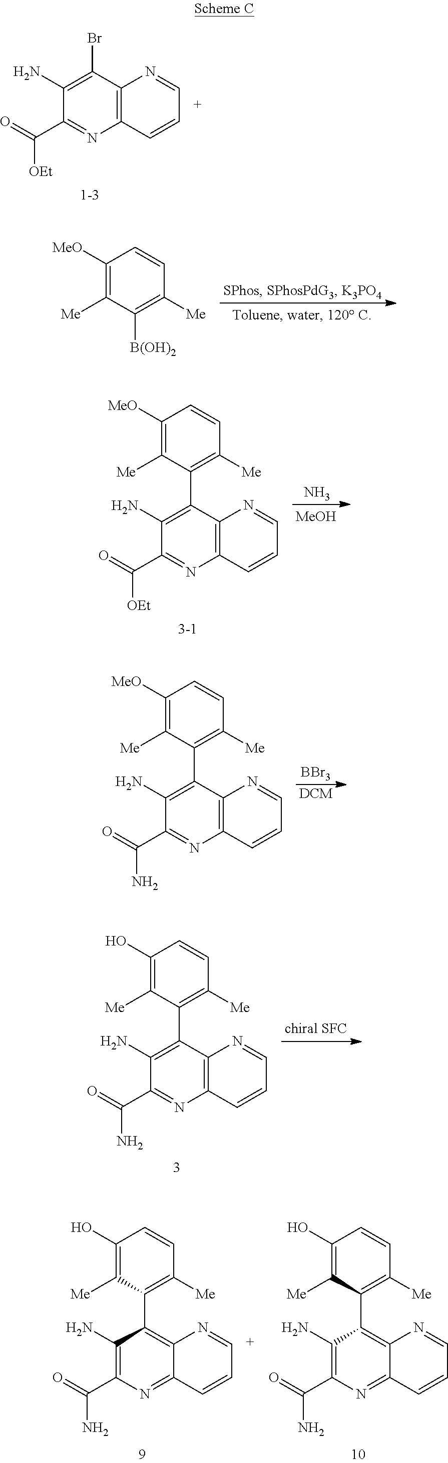

### Example 6. 3-Amino-4-(3-hydroxy-2,6-dimethylphenyl)-1,5-naphthyridine-2-carboxamide (3)

Ethyl 3-amino-4-(3-methoxy-2,6-dimethylphenyl)-1,5-naphthyridine-2-carboxylate (Intermediate 3-1): Ethyl 3-amino-4-bromo-1,5-naphthyridine-2-carboxylate (99.1 mg, 0.335 mmol, 1.00 equiv.), (3-methoxy-2,6-dimethylphenyl)boronic acid (120 mg, 0.669 mmol, 2.00 equiv.), K₃PO₄ (142 equiv., 0.669 mmol, 100 mass %), and dicyclohexyl(2′,6′-dimethoxy[1,1′-biphenyl]-2-yl)phosphane (SPhos, 27.5 mg, 0.0669 mmol, 0.200 equiv.) were added to a microwave vial followed by toluene (1.8 mL) and water (0.2 mL). The mixture was sparged with nitrogen gas for 2 min. (2-Dicyclohexylphosphino-2′,6′-dimethoxybiphenyl) [2-(2′-amino-1,1′-biphenyl)]palladium(II) methanesulfonate (SPhos Pd G3, 52.2 mg, 0.0669 mmol, 0.200 equiv.) was added. The mixture was sparged with nitrogen gas for 2 min and then sealed. The reaction mixture was heated to 90° C. for 4 hr then 100° C. for 6 hr. The reaction mixture was poured into water and extracted with DCM (3×). The combined organics were dried over MgSO₄, filtered, adsorbed to Celite®, then subjected to FCC (DCM:MeOH, 100:0 to 96:4) to afford Intermediate 3-1 (73.7 mg, 63% yield). LC-MS m/z 352 (M+H⁻).

3-amino-4-(3-hydroxy-2,6-dimethylphenyl)-1,5-naphthyridine-2-carboxamide (3): Prepared according to Example 2 using ethyl 3-amino-4-(3-methoxy-2,6-dimethylphenyl)-1,5-naphthyridine-2-carboxylate to give Compound 3 (29.1 mg, 52% yield over 2 steps). ¹H NMR (400 MHz, DMSO) δ 9.20 (s, 1H), 8.71 (s, 1H), 8.49 (s, 1H), 8.26 (d, 1H), 7.91 (s, 1H), 7.48-7.41 (m, 1H), 7.00 (d, 1H), 6.81 (d, 1H), 6.25 (s, 2H), 1.68 (s, 3H), 1.63 (s, 3H). LC-MS m/z 309 (M+H⁺).

### Example 7. (P)-3-Amino-4-(3-hydroxy-2,6-dimethylphenyl)-1,5-naphthyridine-2-carboxamide (9) and (M)-3-amino-4-(3-hydroxy-2,6-dimethylphenyl)-1,5-naphthyridine-2-carboxamide (10)

Chiral SFC separation of Compound 3 (29 mg) (Instrument: Instrument: Waters SFC Prep 150 Mgm; Column: Daicel Chiralpak® OJ, 30×150 mm, 5 um; Conditions: Isocratic at 20% methanol with 80% CO₂; Flow Rate: 100 mL/min) provided Compound 9 (14 mg) and Compound 10 (14 mg).

Peak 1 retention time 1.60 min. ¹H NMR (400 MHz, d6-DMSO) δ 9.19 (s, 1H), 8.71 (s, 1H), 8.49 (s, 1H), 8.26 (d, 1H), 7.91 (s, 1H), 7.49-7.41 (m, 1H), 7.00 (d, 1H), 6.81 (d, 1H), 6.25 (s, 2H), 1.68 (s, 3H), 1.62 (s, 3H). LC-MS m/z 309 (M+H⁺).

Peak 2 retention time 1.86 min. ¹H NMR (400 MHz, d6-DMSO) δ 9.19 (s, 1H), 8.70 (s, 1H), 8.49 (s, 1H), 8.26 (d, 1H), 7.91 (s, 1H), 7.49-7.40 (m, 1H), 7.00 (d, 1H), 6.81 (d, 1H), 6.25 (s, 2H), 1.68 (s, 3H), 1.62 (s, 3H). LC-MS m/z 309 (M+H⁺).

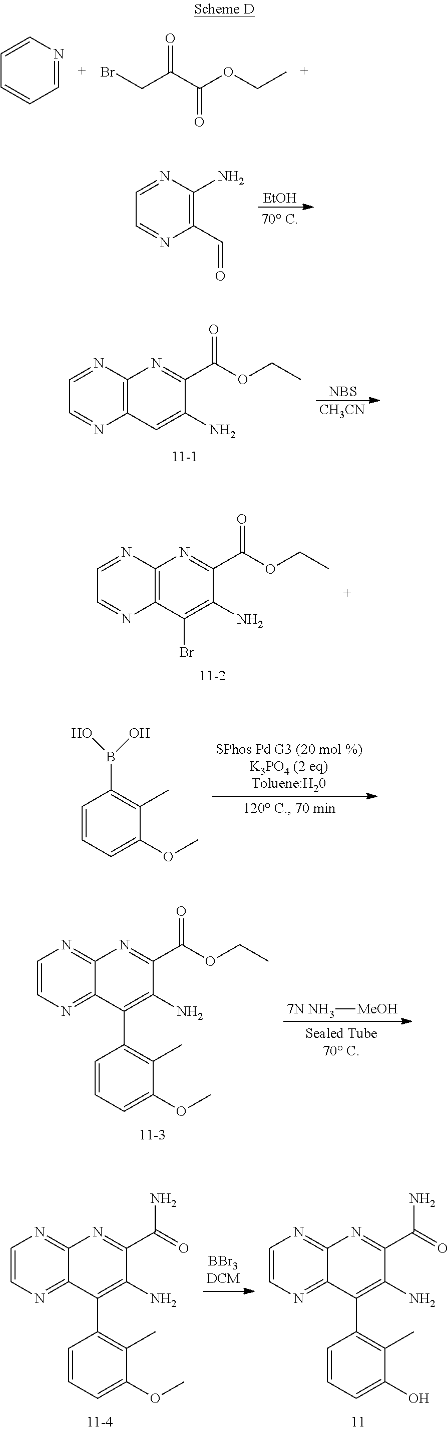

### Example 8. 7-Amino-8-(3-hydroxy-2-methylphenyl)pyrido[2,3-b]pyrazine-6-carboxamide (11)

Ethyl 7-aminopyrido[2,3-b]pyrazine-6-carboxylate (Intermediate 11-1): Under a nitrogen atmosphere, pyridine (1.05 equiv., 8.53 mmol) was dissolved in 24 mL EtOH. A solution of ethyl 3-bromo-2-oxo-propanoate (1.05 equiv., 8.53 mmol, 90 mass %) in 16 mL EtOH was added in portions over 1 h. The reaction was heated to 70° C. and refluxed for 1.3 h. The reaction was removed from heat and cooled for 2 minutes. Then 3-aminopyrazine-2-carbaldehyde (1000 mg, 8.12 mmol) was added along with pyridine (1.75 mL). The solution was again heated to 70° C. and refluxed overnight. The red-brown solution was removed from heat while pyrrolidine (2.00 equiv., 16.25 mmol) was added, and the reaction was refluxed for an additional overnight period. The reaction was cooled to room temperature and concentrated. The crude product was absorbed onto silica gel and purified by ISCO chromatography (80 g SiO₂, 0-100% EtOAc in Hexanes over 40 min then up to 10% MeOH in ethyl acetate) to give Intermediate 11-1 (410 mg). ¹H NMR (400 MHz, CDCl₃) δ 8.87 (d, 1H), 8.81 (s, 1H), 7.65 (t, 1H), 7.28 (d, 2H), 4.56 (q, 2H), 1.52 (t, 3H). LC-MS m/z 219.2 (M+H⁺).

Ethyl 7-amino-8-bromopyrido[2,3-b]pyrazine-6-carboxylate (Intermediate 11-2): Ethyl 7-aminopyrido[2,3-b]pyrazine-6-carboxylate (410 mg, 1.88 mmol) and ammonium acetate (0.1 equiv., 0.188 mmol) were suspended in MeCN (20 mL). NBS (1.05 equiv., 1.97 mmol) dissolved in an additional 5 mL of acetonitrile was added dropwise. The reaction was stirred at room temperature for 3 h then quenched with sat sodium thiosulfate. The solution was concentrated to dryness then partitioned between DCM and water. The aqueous layer was washed with DCM (4×) and the organics were dried over sodium sulfate, filtered and concentrated. 720 mg crude product was concentrated, absorbed onto Celite®, and purified by column. This product was purified by normal phase chromatography (40 g SiO2, Hex/EtOAc 0-60% EtOAc over 20 min.) to give Intermediate 11-2 (453.1 mg, 81% yield). ¹H NMR (400 MHz, CDCl3) δ 8.94 (s, 1H), 8.89 (s, 1H), 6.70 (s, 2H), 4.63-4.53 (m, 2H), 1.53 (dt, 3H). LC-MS in z 297.2/299.2 (M+H⁺).

Ethyl 7-amino-8-(3-methoxy-2-methylphenyl)pyrido[2,3-b]pyrazine-6-carboxylate (Intermediate 11-3): Ethyl 7-amino-8-bromo-pyrido[2,3-b]pyrazine-6-carboxylate (87.5 mg, 0.295 mmol), water (0.2 mL), toluene (1.8 mL), (3-methoxy-2-methyl-phenyl)boronic acid (99.8 mg, 0.601 mmol), potassium phosphate (128.9 mg, 0.6073 mmol), and dicyclohexyl-[2-(2,6-dimethoxyphenyl)phenyl]phosphane (25.6 mg, 0.0624 mmol) were added to a 2-5 mL microwave vial. The flask was purged by bubbling nitrogen through solution for 2 min then [2-(2-aminophenyl)phenyl]-methylsulfonyloxy-palladium; dicyclohexyl-[2-(2,6-dimethoxyphenyl)phenyl]phosphane (46.0 mg, 0.0589 mmol) was added, and the flask was sparged with N₂ again and sealed. The vial was heated to 120° C. for 70 min. The mixture was diluted with EtOAc and filtered through Celite®, washed with EtOAc and some EtOAc/MeOH (approximately 95:5) mixture. The filtrate was concentrated. The crude product (139.3 mg) was adsorbed to Celite®, then subjected to FCC (Hexanes:EtOAc 100 Hex to 50/50 Hexanes/Ethyl Acetate) to give Intermediate 11-3 (35 mg, 35% yield). ¹H NMR (400 MHz, CDCl3) δ 8.76 (d, 2H), 7.35 (dt, 1H), 6.98 (d, 1H), 6.80-6.73 (m, 1H), 5.85 (s, 2H), 4.53 (tt, 2H), 3.89 (d, 3H), 1.85 (d, 3H), 1.50 (td, 3H). LC-MS m/z 339.2 (M+H⁺).

7-amino-8-(3-methoxy-2-methylphenyl)pyrido[2,3-b]pyrazine-6-carboxamide (Intermediate 11-4): In a 15 mL sealed tube, ethyl 7-amino-8-(3-methoxy-2-methyl-phenyl)pyrido[2,3-b]pyrazine-6-carboxylate (23.5 mg, 0.0695 mmol) was dissolved in ammonia in methanol (3 mL, 21 mmol, 7 mol/L) in portions. The sealed tube was heated in an oil bath at 70° C. for 1.75 h and then at room temperature overnight. The reaction was concentrated in vacuo to give the crude product (15.1 mg) which was triturated with methanol to give Intermediate 11-4 (12.3 mg, 57% yield). ¹H NMR (400 MHz, CDCl3) δ 8.69 (d, 2H), 8.34-8.29 (m, 1H), 7.35-7.26 (m, 1H), 6.93 (d, 1H), 6.72 (d, 1H), 6.19 (s, 2H), 5.63 (s, 1H), 3.84 (d, 3H), 1.82 (d, 3H). LC-MS m/z 310.2 (M+H)

7-amino-8-(3-hydroxy-2-methylphenyl)pyrido[2,3-b]pyrazine-6-carboxamide (11): To a suspension of 7-amino-8-(3-methoxy-2-methyl-phenyl)pyrido[2,3-b]pyrazine-6-carboxamide (12.3 mg, 0.0398 mmol) in dichloromethane (0.020 M) in an ice bath was added boron tribromide (3 equiv., 0.119 mmol, 1.0 mol/L in DCM) slowly dropwise. The ice bath was removed after 20 min. The reaction was allowed to stir at room temperature overnight. The reaction was cooled to 0° C., 100 μL of diethanolamine was dissolved in 2 mL of DCM and was added to quench the reaction. The solution was allowed to stand overnight. The product was triturated with MeOH and filtered to give Compound 11 (7.6 mg, 65% yield). ¹H NMR (400 MHz, DMSO) δ 9.50 (s, 1H), 8.81-8.73 (m, 2H), 8.50 (s, 1H), 7.99 (s, 1H), 7.14 (t, 1H), 6.92 (d, 1H), 6.57 (d, 2H), 1.72 (s, 3H). LC-MS m/z 296.1 (M+H⁺).

### Example 9. Synthesis of 7-amino-8-(3-hydroxy-2,6-dimethylphenyl)pyrido[2,3-b]pyrazine-6-carboxamide (126)

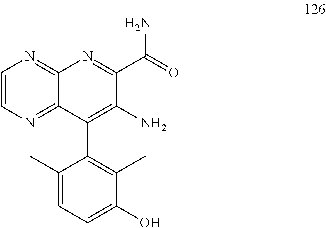 [SMILES: Cc1ccc(O)c(C)c1-c1c(N)c(C(N)=O)nc2nccnc12]

Prepared according to Example 8, steps 3 through 5, using (3-methoxy-2,6-dimethyl-phenyl)boronic acid to give Compound 126 (27 mg). ¹H NMR (400 MHz, DMSO-d₆) δ 9.19 (s, 1H), 8.73 (s, 1H), 8.68 (s, 1H), 8.46 (s, 1H), 7.94 (s, 1H), 6.94 (d, 1H), 6.76 (d, 1H), 6.47-6.41 (m, 2H), 1.62 (s, 3H), 1.57 (s, 3H). LC-MS m/z 310.2 [M+H⁺].

### Example 10. (P)-7-Amino-8-(3-hydroxy-2,6-dimethylphenyl)pyrido[2,3-b]pyrazine-6-carboxamide (127) and (M)-7-Amino-8-(3-hydroxy-2,6-dimethylphenyl)pyrido[2,3-b]pyrazine-6-carboxamide (128)

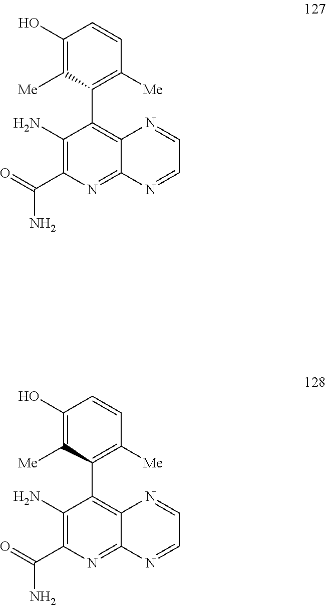

Chiral SFC separation of Compound 126 (20 mg) (Instrument: Instrument: Waters SFC Prep 150 Mgm; Column: Daicel Chiralpak® AD, 30×150 mm, 5 um; Conditions: Isocratic at 30% methanol with 70% CO₂, Flow Rate: 100 mL/min) provided Compound 127 (7.4 mg) and Compound 128 (7.3 mg).

Peak 1 retention time 1.92 min. ¹H NMR (400 MHz, d6-DMSO) δ 9.28 (s, 1H), 8.79 (s, 1H), 8.74 (s, 1H), 8.52 (s, 1H), 8.00 (s, 1H), 7.01 (d, 1H), 6.83 (d, 1H), 6.49 (s, 2H), 1.69 (s, 3H), 1.63 (s, 3H). LC-MS m/z 310.2 (M+H⁻).

Peak 2 retention time 2.92 min. ¹H NMR (400 MHz, d6-DMSO) δ 9.19 (d, 1H), 8.75-8.65 (m, 2H), 8.45 (s, 1H), 7.94 (s, 1H), 6.94 (d, 1H), 6.79-6.72 (m, 1H), 6.43 (s, 2H), 1.62 (d, 3H), 1.56 (d, 3H). LC-MS m/z 310.2 (M+H⁺).

### Example 11. 4-(3-Hydroxy-2,6-dimethylphenyl)quinoline-2-carboxamide (12)

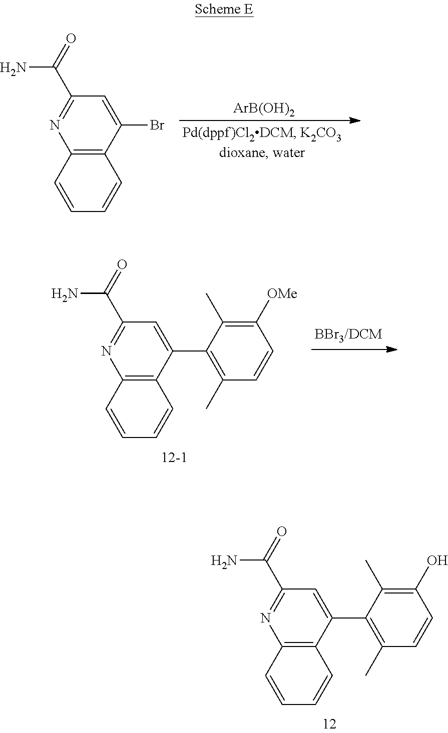 [SMILES: NC(=O)c1cc(Br)c2ccccc2n1 ; "ArB(OH)2" ; COc1ccc(C)c(-c2cc(C(N)=O)nc3ccccc23)c1C ; Cc1ccc(O)c(C)c1-c1cc(C(N)=O)nc2ccccc12]

4-(3-methoxy-2,6-dimethylphenyl)quinoline-2-carboxamide (Intermediate 12-1): To a solution of 4-bromoquinoline-2-carboxamide (100 mg, 0.40 mmol, 1.0 equiv.) and (3-methoxy-2,6-dimethylphenyl)boronic acid (108 mg, 0.60 mmol, 1.5 equiv.) in DME (2 mL) and H₂O (0.8 mL) was added Pd(dppf)Cl₂·DCM (32 mg, 0.040 mmol, 0.10 equiv.), Na₂CO₃ (422 mg, 3.98 mmol, 10 equiv.). The mixture was stirred at 120° C. for 3 h under microwave heating. The reaction mixture was diluted with water (50 mL) and extracted with DCM (50 mL×3). The combined organic layers were washed with brine (50 mL×3), dried over anhydrous Na₂SO₄, filtered and concentrated to give a residue. The residue was purified by flash silica gel chromatography to give 4-(3-methoxy-2,6-dimethylphenyl)quinoline-2-carboxamide (Intermediate 12-1) (70 mg, 0.23 mmol, 57% yield); LC-MS m/z 307.2 [M+H⁺].

4-(3-hydroxy-2,6-dimethyl-phenyl)quinoline-2-carboxamide (12): To a solution of 4-(3-methoxy-2,6-dimethyl-phenyl)quinoline-2-carboxamide (60 mg, 0.20 mmol, 1 equiv.) in DCM (4 mL) was added BBr₃ (245 mg, 0.98 mmol, 5 equiv.) at 0° C. The mixture was stirred at 20° C. for 1 hour. The reaction mixture was added dropwise into water (50 mL) at 20° C. The reaction mixture was adjusted to pH 7 by addition NaHCO₃ at 0° C. The mixture was extracted with DCM (50 mL×3). The combined organic layers were washed with brine (50 mL×3), dried over anhydrous Na₂SO₄, filtered and concentrated to give a residue. The residue was purified by prep-HPLC to give 4-(3-hydroxy-2,6-dimethylphenyl)quinoline-2-carboxamide (Compound 12) (25.5 mg, 45% yield). LC-MS m/z 293.1 [M+H⁺]; ¹H NMR: (400 MHz, DMSO-d₆) δ=9.38 (s, 1H), 8.33 (s, 1H), 8.18 (d, 1H), 7.86-7.80 (m, 3H), 7.63-7.58 (t, 1H), 7.32 (d, 1H), 7.01 (d, 1H), 6.85 (d, 1H), 1.66 (s, 3H), 1.59 (s, 3H).

### Example 12. 4-(3-Hydroxy-2,6-dimethylphenyl)quinoline-2-carboxamide (13)

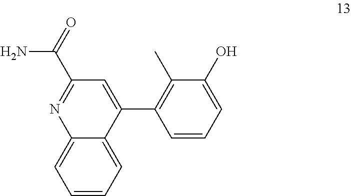 [SMILES: Cc1c(O)cccc1-c1cc(C(N)=O)nc2ccccc12]

Prepared according to Example 11, steps 1 and 2, using (3-methoxy-2-methylphenyl)boronic acid to give 4-(3-hydroxy-2,6-dimethylphenyl)quinoline-2-carboxamide (Compound 13, 21 mg); LC-MS m/z 279.1 [M+H⁺]; ¹H NMR (400 MHz, DMSO-d6) δ=8.35 (s, 1H), 8.19 (d, 1H), 7.93-7.80 (m, 3H), 7.69-7.60 (m, 1H), 7.50 (d, 1H), 7.21-7.11 (m, 1H), 6.98 (d, 1H), 6.69 (d, 1H), 1.75 (s, 3H).

### Example 13. 4-(1H-Indazol-4-yl)quinoline-2-carboxamide (14)

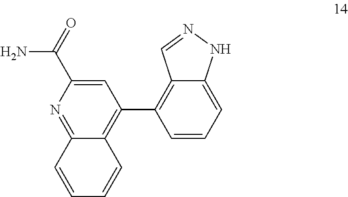 [SMILES: NC(=O)c1cc(-c2cccc3[nH]ncc23)c2ccccc2n1]

Prepared according to Example 11, step 1, using 1H-indazol-4-ylboronic acid to give 4-(1H-indazol-4-yl)quinoline-2-carboxamide (Compound 14, 73.6 mg, 31% yield); LC-MS m/z 289.1 [M+H⁺]; ¹H NMR (400 MHz, DMSO-d6) δ=13.55-13.31 (m, 1H), 8.46-8.36 (m, 1H), 8.27-8.20 (m, 1H), 8.17-8.12 (m, 1H), 7.94-7.85 (m, 2H), 7.83-7.72 (m, 2H), 7.71-7.62 (m, 2H), 7.59-7.53 (m, 1H), 7.33-7.25 (m, 1H).

### Example 14. 4-(5-Methyl-1H-indazol-4-yl)quinoline-2-carboxamide (15)

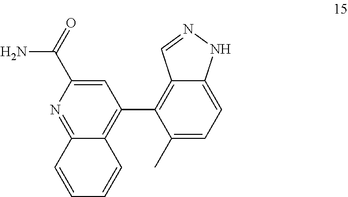 [SMILES: Cc1ccc2[nH]ncc2c1-c1cc(C(N)=O)nc2ccccc12]

Prepared according to Example 11, step 1, using (5-methyl-1H-indazol-4-yl)boronic acid to give 4-(5-methyl-1H-indazol-4-yl)quinoline-2-carboxamide (Compound 15, 39.0 mg, 16.2% yield); LC-MS m/z 303.3 [M+H⁺]; ¹H NMR (400 MHz, DMSO-d6) δ=13.23 (s, 1H), 8.41 (s, 1H), 8.25 (d, 1H), 8.02 (s, 1H), 7.93-7.82 (m, 2H), 7.68-7.56 (m, 2H), 7.47-7.31 (m, 3H), 2.04 (s, 3H).

### Example 15. 4-(4-Hydroxy-2,6-dimethylphenyl)quinoline-2-carboxamide (16)

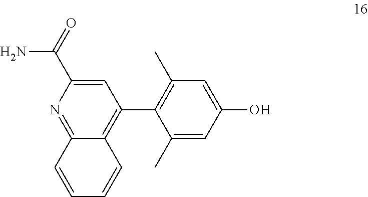 [SMILES: Cc1cc(O)cc(C)c1-c1cc(C(N)=O)nc2ccccc12]

Prepared according to Example 11, steps 1 and 2, using (4-methoxy-2,6-dimethyl-phenyl) boronic acid to give 4-(4-hydroxy-2,6-dimethylphenyl)quinoline-2-carboxamide (Compound 16, 4.8 mg, 2.5% yield); LC-MS m/z 293.0 [M+H⁺]; ¹H NMR (400 MHz, DMSO-d6) δ=9.49 (s, 1H), 8.36 (d, 1H), 8.19 (d, 1H), 7.84 (s, 3H), 7.63 (1H), 7.38 (d, 1H), 6.65 (s, 2H), 1.73 (s, 6H).

### Example 16. 4-(3-Methyl-2H-indazol-4-yl)quinoline-2-carboxamide (19)

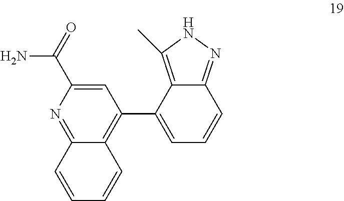 [SMILES: Cc1[nH]nc2cccc(-c3cc(C(N)=O)nc4ccccc34)c12]

Prepared according to Example 11, step 1, using (3-methyl-1H-indazol-4-yl)boronic acid to give 4-(3-methyl-2H-indazol-4-yl)quinoline-2-carboxamide (Compound 19, 98.7 mg, 41% yield); LC-MS m/z 303.4 [M+H⁺]; ¹H NMR (400 MHz, DMSO-d6) δ=12.97 (s, 1H), 8.39 (s, 1H), 8.24 (d, 1H), 8.04 (s, 1H), 7.95-7.81 (m, 2H), 7.71-7.59 (m, 2H), 7.54-7.42 (m, 2H), 7.09 (d, 1H), 1.56 (s, 3H).

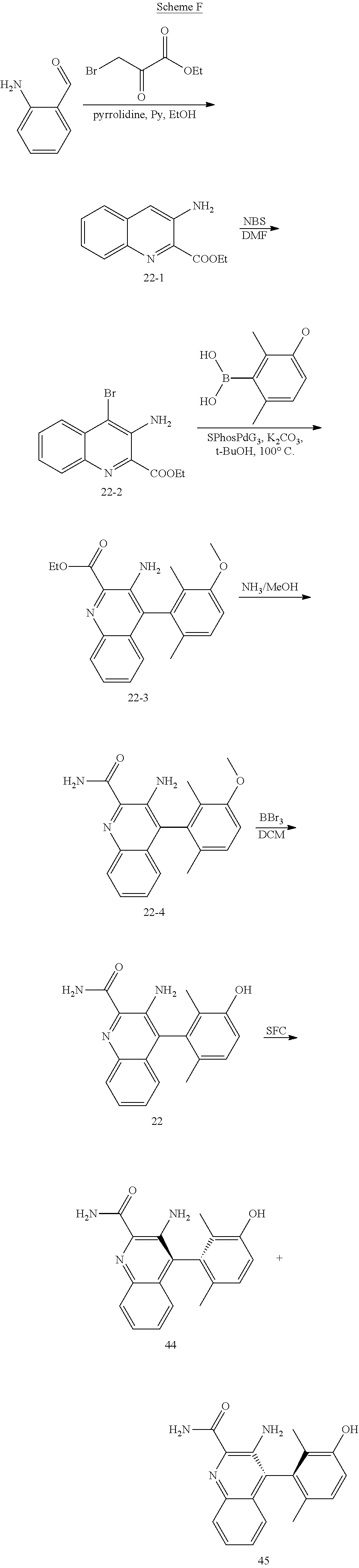

### Example 17. 3-Amino-4-(3-hydroxy-2,6-dimethylphenyl)quinoline-2-carboxamide (22)

Ethyl 3-aminoquinoline-2-carboxylate (Intermediate 22-1): To a mixture of pyridine (686 mg, 8.67 mmol, 0.7 mL, 1.05 equiv.) in EtOH (15 mL) was added ethyl 3-bromo-2-oxo-propanoate (1.7 g, 8.67 mmol, 1.08 mL, 1.05 equiv.) in EtOH (15 mL) dropwise over 20 min. The resulting mixture was heated at 60° C. for 1 hour and cooled to room temperature. Then 2-aminobenzaldehyde (1 g, 8.26 mmol, 1 equiv.) and pyridine (686 mg, 8.67 mmol, 0.7 mL, 1.05 equiv.) were added. After heating at 70° C. for 5 h, pyrrolidine (1.35 g, 18.99 mmol, 1.58 mL, 2.3 equiv.) was added. The resulting mixture was heated for another 2 h. The reaction mixture was concentrated, then dissolved in water 20 mL, followed by extraction with EtOAc 50 mL×3. The organic phase was washed with brine (50 mL×2) then dried over anhydrous Na₂SO₄, filtered and concentrated under vacuum. The crude product was purified by flash silica gel chromatography to give ethyl 3-aminoquinoline-2-carboxylate (Intermediate 22-1) (1.2 g, 5.46 mmol, 66.1% yield). LC-MS m/z 217.0 [M+H⁺]

Ethyl 3-amino-4-bromoquinoline-2-carboxylate (Intermediate 22-2): To a solution of ethyl 3-aminoquinoline-2-carboxylate (500 mg, 2.31 mmol, 1 equiv.) in DMF (5 mL) was added NBS (452 mg, 2.54 mmol, 1.1 equiv.). The mixture was stirred at 20° C. for 1 hour. The reaction mixture was concentrated, then dissolved in water (40 mL) and extracted with EtOAc (50 mL×3). The organic phase was washed with brine (50 mL×2) then dried over anhydrous Na₂SO₄, filtered, and concentrated under vacuum. The crude product was purified by flash silica gel chromatography to give ethyl 3-amino-4-bromo-quinoline-2-carboxylate (Intermediate 22-2) (503 mg, 69.8% yield); LC-MS m/z 296.8 [M+2+H⁺].

Ethyl 3-amino-4-(3-methoxy-2,6-dimethylphenyl)quinoline-2-carboxylate (Intermediate 22-3): To a solution of ethyl 3-amino-4-bromo-quinoline-2-carboxylate (400 mg, 1.36 mmol, 1 equiv.) and (3-methoxy-2,6-dimethylphenyl)boronic acid (368 mg, 2.04 mmol, 1.5 equiv.) in t-BuOH (4 mL) was added SPhos Pd G₃ (158 mg, 0.2 mmol, 0.15 equiv.) and K₂CO₃ (562 mg, 4.07 mmol, 3 equiv.). The mixture was stirred at 100° C. under N₂ for 20 h. The reaction mixture was dissolved in water (10 mL), then was extraction with EtOAc (30 mL×3). The organic phase was dried over anhydrous Na₂SO₄, filtered and concentrated under vacuum. The crude product was purified by flash silica gel chromatography to give ethyl 3-amino-4-(3-methoxy-2,6-dimethylphenyl)quinoline-2-carboxylate (Intermediate 22-3) (140 mg, 0.37 mmol, 27.5% yield, 93.3% purity). LC-MS m/z 351.0 [M+H]⁺

Ethyl 3-amino-4-(3-methoxy-2,6-dimethylphenyl)quinoline-2-carboxylate (Intermediate 22-4): A solution of ethyl 3-amino-4-(3-methoxy-2,6-dimethyl-phenyl)quinoline-2-carboxylate (22-4, 200 mg, 0.57 mmol, 1 equiv.) in NH₃/MeOH (7 M, 5 mL, 61 equiv.) was stirred at 60° C. for 4 h. The solution was concentrated under vacuum to give a crude product of 3-amino-4-(3-methoxy-2,6-dimethylphenyl)quinoline-2-carboxamide (Intermediate 22-4) (150 mg, crude). LC-MS m/z 322.1 [M+H]⁺

Ethyl 3-amino-4-(3-methoxy-2,6-dimethylphenyl)quinoline-2-carboxylate (22): To a solution of 3-amino-4-(3-methoxy-2,6-dimethylphenyl)quinoline-2-carboxamide (130 mg, 0.4 mmol, 1 equiv.) in dichloromethane (3 mL) was added BBr₃ (2.51 g, 10 mmol, 1 mL, 24.7 equiv.) at 0° C. under N₂. The mixture was stirred at 0° C. for 1 hour. The reaction mixture was quenched by adding H₂O (20 mL) at 20° C., then extracted with dichloromethane (10 mL×3). The organic phase was washed with brine, dried over anhydrous Na₂SO₄, filtered and concentrated under vacuum. The crude product was purified by prep-HPLC to give 3-amino-4-(3-hydroxy-2,6-dimethylphenyl)quinoline-2-carboxamide (Compound 22) (5.1 mg, 16.57 μmol, 4.10% yield). LC-MS m/z 308.0 [M+H]; ¹H NMR: (400 MHz, DMSO-d₆) δ=9.29-9.36 (s, 1H), 8.40-8.52 (m, 1H), 7.86-7.99 (m, 1H), 7.74 (d, 1H), 7.33-7.47 (m, 2H), 7.04-7.12 (d, 1H), 6.87 (d, 2H), 5.89-6.17 (m, 2H), 1.67-1.73 (s, 3H), 1.64 (s, 3H).

### Example 18. (M)-3-Amino-4-(3-hydroxy-2,6-dimethylphenyl)quinoline-2-carboxamide (44) and (P)-3-amino-4-(3-hydroxy-2,6-dimethylphenyl)quinoline-2-carboxamide (45)

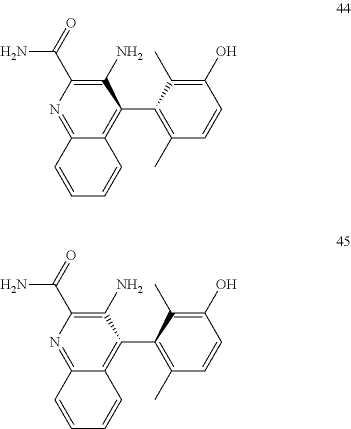

Chiral SFC separation of Compound 22 (Instrument: Waters SFC Prep 150 Mgm; Column: Daicel Chiralpak® OX-3, 30×150 mm, 5 um; Conditions: 10-40% gradient ethanol with 0.05% DEA and CO2; Flow Rate: 100 mL/min) provided Compound 44 (17.8 mg, 29.1% yield) and Compound 45 (12.9 mg, 21.0% yield).

Peak 1: SFC t_{R}=2.409 min. LC-MS m/z 308.1 [M+H]⁺; ¹H NMR: (400 MHz, DMSO-d₆) δ=9.33 (s, 1H), 8.47 (m, 1H), 7.92 (d, 1H), 7.79 (m, 1H), 7.30-7.49 (m, 2H), 7.02-7.13 (d, 1H), 6.81-6.92 (d, 2H), 5.82-6.22 (s, 2H), 1.69 (s, 3H), 1.64 (s, 3H).

Peak 2: SFC t_{R}=2.553 min. LC-MS m/z 308.1 [M+H]⁺; ¹H NMR: (400 MHz, DMSO-d₆) δ=9.33 (s, 1H), 8.47 (d, 1H), 7.92 (d, 1H), 7.79 (d, 1H), 7.40 (m, 2H), 7.07 (d, 1H), 6.87 (d, 2H), 6.03 (s, 2H), 1.69 (s, 3H), 1.64 (s, 3H).

### Example 19. 3-Amino-4-(5-methyl-1H-indazol-4-yl)quinoline-2-carboxamide (17)

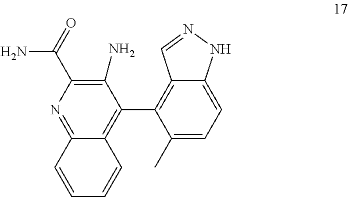 [SMILES: Cc1ccc2[nH]ncc2c1-c1c(N)c(C(N)=O)nc2ccccc12]

Prepared according to Example 17, steps 3 and 4, using (5-methyl-1H-indazol-4-yl)boronic acid to give 3-amino-4-(5-methyl-1H-indazol-4-yl)quinoline-2-carboxamide (Compound 17) (16.1 mg, 8.9% yield). LC-MS m/z 318.4 [M+H]⁺; ¹H NMR (400 MHz, DMSO-d6) δ=13.15 (s, 1H), 8.51 (s, 1H), 7.96 (d, 1H), 7.81 (s, 1H), 7.61 (d, 1H), 7.51-7.38 (m, 2H), 7.35-7.21 (m, 2H), 6.78 (d, 1H), 6.12 (s, 2H), 2.02 (s, 3H).

### Example 20. 3-Amino-4-(3-hydroxy-2-methylphenyl)quinoline-2-carboxamide (18)

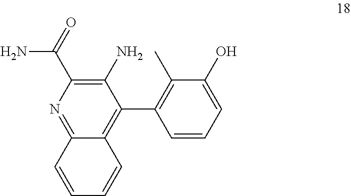 [SMILES: Cc1c(O)cccc1-c1c(N)c(C(N)=O)nc2ccccc12]

Prepared according to Example 17, steps 3-5, using (3-methoxy-2-methylphenyl)boronic acid to give 3-amino-4-(3-hydroxy-2-methylphenyl)quinoline-2-carboxamide (Compound 18) (21.5 mg). LC-MS m/z 294.1 [M+H]+; ¹H NMR (400 MHz, DMSO-d6) δ=9.61 (s, 1H), 8.45 (d, 1H), 7.95-7.87 (t, 1H), 7.77 (d, 1H), 7.43-7.34 (m, 2H), 7.20 (t, 1H), 6.98-6.94 (m, 2H), 6.56 (d, 1H), 6.06 (s, 2H), 1.72 (s, 3H).

### Example 21. 3-Amino-6-fluoro-4-(3-hydroxy-2,6-dimethylphenyl)quinoline-2-carboxamide (20)

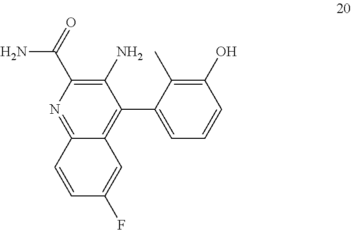 [SMILES: Cc1c(O)cccc1-c1c(N)c(C(N)=O)nc2ccc(F)cc12]

Prepared according to Example 17, steps 1-5, using 2-amino-5-fluorobenzaldehyde and 3-methoxy-2,6-dimethylphenylboronic acid to give 3-amino-6-fluoro-4-(3-hydroxy-2,6-dimethylphenyl)quinoline-2-carboxamide (Compound 20) (30 mg, 26% yield). LC-MS m/z 326.0 [M+H]+; ¹H NMR (400 MHz, DMSO-d6) δ=9.38 (s, 1H), 8.48 (d, 1H), 7.99 (dd, 1H), 7.83 (d, 1H), 7.38-7.29 (m, 1H), 7.09 (d, 1H), 6.89 (d, 1H), 6.41 (dd, 1H), 6.21 (s, 2H), 1.71 (s, 3H), 1.66 (s, 3H).

### Example 22. (M)-3-Amino-6-fluoro-4-(3-hydroxy-2,6-dimethylphenyl) quinoline-2-carboxamide (23) and (P)-3-amino-6-fluoro-4-(3-hydroxy-2,6-dimethylphenyl) quinoline-2-carboxamide (24)

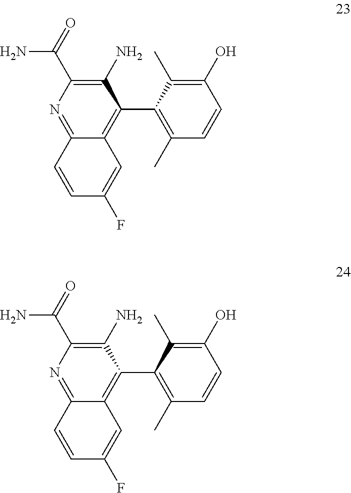

Chiral HPLC separation of Compound 20 (Column, Lux 5 um Cellulose-4, 2.12×25 cm, 5 um; mobile phase, Hex (0.5% 2 M NH₃ in MeOH) and IPA (hold 10% IPA in 37 min)) provided Compound 23 (12.5 mg, 35%) and Compound 24 (11.6 mg, 33%).

Peak 1: retention time 4.953 LC-MS m/z [M+H]=326.0; ¹H NMR (400 MHz, Methanol-d₄) δ 8.03 (dd, 1H), 7.24 (td, 1H), 7.13 (d, 1H), 6.90 (d, 1H), 6.52 (dd, 1H), 1.79 (d, 6H).

Peak 2: retention time 5.891 LC-MS m/z [M+H]=326.0; ¹H NMR (400 MHz, Methanol-d₄) δ 8.03 (dd, 1H), 7.24 (td, 1H), 7.13 (d, 1H), 6.90 (d, 1H), 6.52 (dd, 1H), 1.79 (d, 6H).

### Example 23. 3-Amino-6-fluoro-4-(3-hydroxy-2,6-dimethylphenyl)quinoline-2-carboxamide (25)

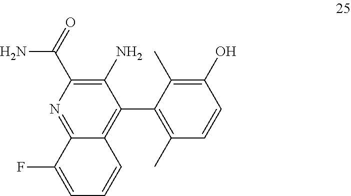 [SMILES: Cc1ccc(O)c(C)c1-c1c(N)c(C(N)=O)nc2c(F)cccc12]

Prepared according to Example 17, steps 1 through 5, using 2-amino-3-fluorobenzaldehyde and 3-methoxy-2,6-dimethylphenylboronic acid to give 3-amino-6-fluoro-4-(3-hydroxy-2,6-dimethylphenyl)quinoline-2-carboxamide (25, 100 mg, 86.1% yield). LC-MS m/z 326.05 [M+H⁺]; ¹H NMR (300 MHz, DMSO-d6) δ 9.34 (s, 1H), 8.26 (s, 1H), 7.89 (s, 1H), 7.35 (m, 1H), 7.21 (m, 1H), 7.08 (m, 1H), 6.88 (m, 1H), 6.66 (m, 1H), 6.18 (s, 2H), 1.67 (m, 6H).

### Example 24. (M)-3-Amino-8-fluoro-4-(3-hydroxy-2,6-dimethylphenyl) quinoline-2-carboxamide (38) and (P)-3-amino-8-fluoro-4-(3-hydroxy-2,6-dimethylphenyl)quinoline-2-carboxamide (39)

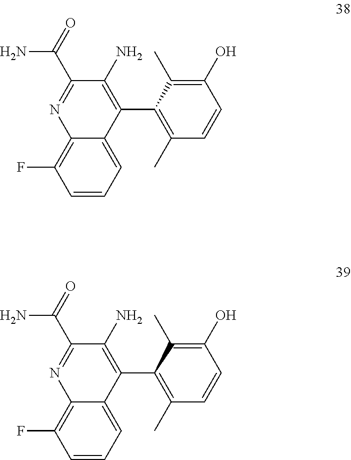

Chiral HPLC of Compound 25 (Column, Lux 5 um Cellulose-4, 2.12×25 cm, 5 um; mobile phase, Hex (0.5% 2M NH₃-MeOH) and EtOH (hold 10% EtOH in 26 min)) provided the Compound 38 (36.1 mg, 36.1%) and Compound 39 (39.4 mg, 39.4%).

Peak 1: retention time 2.972 min, LC-MS m/z [M+H⁺]=326.0; ¹H NMR (300 MHz, Methanol-d4) δ 7.30 (m, 1H), 7.11 (m, 2H), 6.86 (m, 1H), 6.78 (m, 1H), 1.76 (m, 6H).

Peak2: retention time 3.865 min, LC-MS m/z [M+H⁺]=326.0; ¹H NMR (300 MHz, Methanol-d4) δ 7.30 (m, 1H), 7.11 (m, 2H), 6.86 (m, 1H), 6.78 (m, 1H), 1.76 (m, 6H).

### Example 25. 3-Amino-4-(3-hydroxy-2,6-dimethylphenyl)-8-methylquinoline-2-carboxamide (27)

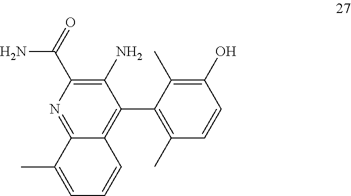 [SMILES: Cc1ccc(O)c(C)c1-c1c(N)c(C(N)=O)nc2c(C)cccc12]

Prepared according to Example 17, steps 1 through 5, using 2-amino-3-methylbenzaldehyde and (3-methoxy-2,6-dimethylphenyl)boronic acid to give Compound 27 (28 mg, 97.4% yield). LC-MS m/z 322.1 [M+H⁺]; ¹H NMR: (400 MHz, DMSO-d₆) δ: 9.34 (br s, 1H), 8.36 (br d, 1H), 7.81 (br d, 1H), 7.21-7.29 (m, 2H), 7.17 (br s, 2H), 7.06 (d, 1H), 6.87 (d, 1H), 6.67-6.73 (m, 1H), 5.99 (br s, 1H), 2.73 (s, 3H), 1.68 (s, 3H), 1.62 (s, 3H).

### Example 26. (P)-3-Amino-4-(3-hydroxy-2,6-dimethyl-phenyl)-8-(trifluoromethyl) quinoline-2-carboxamide (32) and (M)-3-amino-4-(3-hydroxy-2,6-dimethyl-phenyl)-8-(trifluoromethyl) quinoline-2-carboxamide (33)

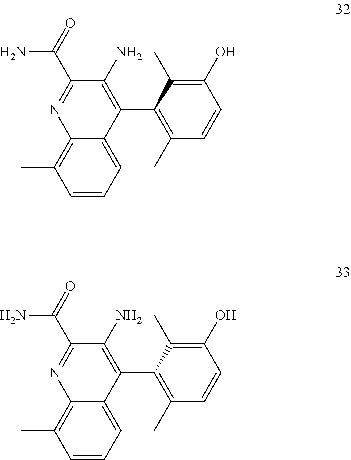

Chiral SFC separation of Compound 27 (Instrument: Waters, SFC-80Q; column: Daicel Chiralpak® IG (250×30 mm, 10 um); mobile phase: [0.1% NH₃H₂O IPA]; B %: 30%) provided Compound 33 (19.7 mg, 46.9% yield) and Compound 32 (25.3 mg, 60.2% yield).

Peak 1: retention time 5.686 min; LC-MS m/z [M+H⁺]=322.1; ¹H NMR (400 MHz, DMSO-d₆) δ: 9.33 (s, 1H), 8.36 (br s, 1H), 7.82 (br s, 1H), 7.22-7.28 (m, 2H), 7.06 (d, 1H), 6.87 (d, 1H), 6.70 (d, 1H), 5.99 (br s, 2H), 2.74 (s, 3H), 1.68 (s, 3H), 1.62 (s, 3H).

Peak 2: retention time 5.942 min; LC-MS m/z [M+H⁺]=322.1; ¹H NMR: (400 MHz, DMSO-d₆) δ: 9.33 (s, 1H), 8.36 (br s, 1H), 7.82 (br s, 1H), 7.21-7.30 (m, 2H), 7.06 (d, 1H), 6.87 (d, 1H), 6.70 (br d, 1H), 5.99 (br s, 2H), 2.73 (s, 3H), 1.68 (s, 3H), 1.62 (s, 3H).

### Example 27. 3-Amino-4-(3-hydroxy-2,6-dimethylphenyl)-8-(trifluoromethyl)quinoline-2-carboxamide (28)

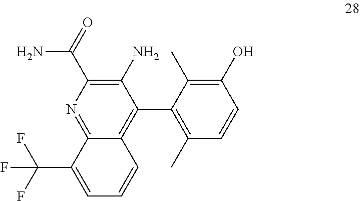 [SMILES: Cc1ccc(O)c(C)c1-c1c(N)c(C(N)=O)nc2c(C(F)(F)F)cccc12]

Prepared according to Example 17, steps 1-5, using 2-amino-3-(trifluoromethyl)benzaldehyde and (3-methoxy-2,6-dimethylphenyl)boronic acid to give Compound 28 (135 mg, 68% yield). LC-MS m/z 376.0 [M+H⁺]; ¹H NMR: (400 MHz, DMSO-d₆) δ: 9.41 (s, 1H), 8.09 (d, 1H), 7.96 (d, 1H), 7.86 (d, 1H), 7.50 (q, 1H), 7.20-7.07 (m, 2H), 6.90 (d, 1H), 6.31 (s, 2H), 1.70 (s, 3H), 1.65 (s, 3H).

### Example 28. (M)-3-Amino-4-(3-hydroxy-2,6-dimethylphenyl)-8-(trifluoromethyl) quinoline-2-carboxamide (30) and (P)-3-amino-4-(3-hydroxy-2,6-dimethylphenyl)-8-(trifluoromethyl) quinoline-2-carboxamide (31)

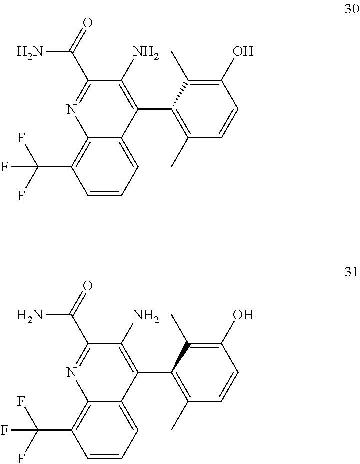

Chiral HPLC separation of Compound 28 (Column: Lux 5 um Cellulose-4, 2.12×25 cm, 5 um; mobile phase: Hex (0.5% 2 M NH₃ in MeOH) and 10% EtOH provided Compound 30 (50.7 mg, 39% yield) and Compound 31 (52.3 mg, 40% yield).

Peak 1: retention time 2.896 min, LC-MS m/z [M+H⁺]=376.0; ¹H NMR (400 MHz, Methanol-d₄) δ 7.82 (d, 1H), 7.46 (t, 1H), 7.25 (dd, 1H), 7.13 (d, 1H), 6.90 (d, 1H), 1.78 (d, 6H).

Peak 2: retention time 3.611 min, LC-MS m/z [M+H⁺]=376.0; ¹H NMR: (400 MHz, DMSO-d₆) δ: 7.82 (d, 1H), 7.46 (t, 1H), 7.25 (d, 1H), 7.13 (d, 1H), 6.90 (d, 1H), 1.78 (d, 6H).

### Example 29. 3-Amino-4-(3-hydroxy-2,6-dimethylphenyl)-8-(trifluoromethyl) quinoline-2-carboxamide (34)

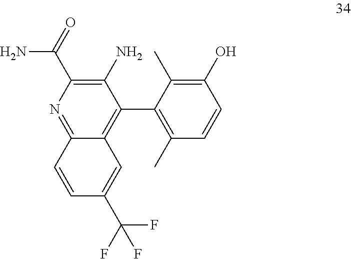 [SMILES: Cc1ccc(O)c(C)c1-c1c(N)c(C(N)=O)nc2ccc(C(F)(F)F)cc12]

Prepared according to Example 17, steps 1-5, using 2-amino-5-(trifluoromethyl)benzaldehyde and (3-methoxy-2,6-dimethylphenyl)boronic acid to give the Compound 34 (5.8 mg, 20.1% yield). LC-MS m/z 376.05 [M+H⁺]; ¹H NMR: (400 MHz, DMSO-d₆) δ: 9.45 (s, 1H), 8.59 (d, 1H), 8.14 (d, 1H), 7.98-7.93 (m, 1H), 7.66 (dd, 1H), 7.16-7.08 (m, 2H), 6.91 (d, 1H), 6.31 (s, 2H), 1.69 (s, 3H), 1.64 (s, 3H).

### Example 30. (M)-3-Amino-4-(3-hydroxy-2,6-dimethylphenyl)-6-(trifluoromethyl)quinoline-2-carboxamide (40) and (P)-3-amino-4-(3-hydroxy-2,6-dimethylphenyl)-6-(trifluoromethyl)quinoline-2-carboxamide (41)

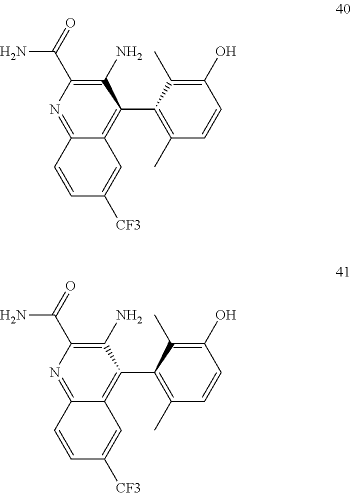

Chiral HPLC separation of Compound 34 (Column: Daicel Chiralpak® IF, 2×25 cm, 5 μm; Mobile Phase A: Hex (0.5% 2M NH₃-MeOH), Mobile Phase B: IPA; Gradient: 5% B to 5% B) provided Compound 40 (26.7 mg, 17.55%) and Compound 41 (28.2 mg, 18.67%).

Peak 1: retention time 2.902 min, LC-MS m/z [M+H⁺]=376.0; ¹H NMR (400 MHz, DMSO-d₆) δ 9.46 (s, 1H), 8.60 (d, 1H), 8.14 (d, 1H), 7.96 (d, 1H), 7.67 (dd, 1H), 7.16-7.09 (m, 2H), 6.91 (d, 1H), 6.31 (s, 2H), 1.70 (s, 3H), 1.65 (s, 3H).

Peak 2: retention time 3.486 min, LC-MS m/z [M+H⁺]=376.0; ¹H NMR (400 MHz, DMSO-d₆) δ 9.43 (s, 1H), 8.59 (s, 1H), 8.14 (d, 1H), 7.95 (s, 1H), 7.66 (dd, 1H), 7.12 (d, 2H), 6.91 (d, 1H), 6.31 (s, 2H), 1.69 (s, 3H), 1.64 (s, 3H).

### Example 31. 3-Amino-6-chloro-4-(3-hydroxy-2,6-dimethyl-phenyl) quinoline-2-carboxamide (36)

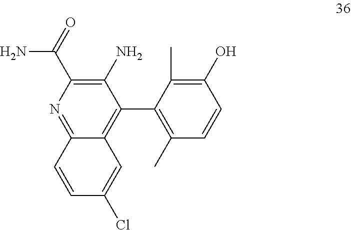 [SMILES: Cc1ccc(O)c(C)c1-c1c(N)c(C(N)=O)nc2ccc(Cl)cc12]

Prepared according to Example 17, steps 1-5, using 2-amino-5-chlorobenzaldehyde and (3-methoxy-2,6-dimethylphenyl)boronic acid to give 3-amino-6-chloro-4-(3-hydroxy-2,6-dimethyl-phenyl) quinoline-2-carboxamide (Compound 36; 14.1 mg). LC-MS m/z 342.3 [M+H⁺]; ¹H NMR: (400 MHz, DMSO-d₆) δ: 9.39 (s, 1H), 8.49 (s, 1H), 7.95-7.93 (d, 1H), 7.84 (s, 1H), 7.42 (d, 1H), 7.09 (d, 1H), 6.89 (d, 1H), 6.77 (s, 1H), 6.22 (s, 2H), 1.70-1.65 (m, 6H).

### Example 32. (M)-3-Amino-6-chloro-4-(3-hydroxy-2,6-dimethylphenyl)quinoline-2-carboxamide (53) and (P)-3-amino-6-chloro-4-(3-hydroxy-2,6-dimethylphenyl)quinoline-2-carboxamide (54)

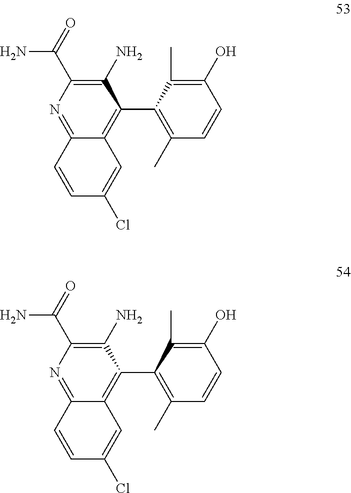

Chiral SFC purification of Compound 36 (Instrument: Waters, SFC-80Q; Column: Daicel Chiralpak® AD (250 mm×30 mm, 10 um); mobile phase: [0.1% NH₃H₂O IPA]; B %: 35%-35%) provided Compound 53 (40.9 mg) and Compound 54 (28.6 mg).

Peak 1: retention time 1.536 min, LC-MS m/z [M+H⁺]=342.3; ¹H NMR (400 MHz, DMSO-d₆) δ 9.39 (s, 1H), 8.49 (s, 1H), 7.93 (d, 1H), 7.84 (s, 1H), 7.43-7.40 (m, 1H), 7.09 (d, 1H), 6.89 (d, 1H), 6.76 (d, 1H), 6.21 (s, 2H), 1.70 (s, 3H), 1.64 (s, 3H).

Peak 2: retention time 1.735 min, LC-MS m/z [M+H⁺]=342.3; ¹H NMR (400 MHz, DMSO-d₆) δ 9.39 (s, 1H), 8.48 (d, 1H), 7.93 (d, 1H), 7.84 (s, 1H), 7.43-7.40 (m, 1H), 7.09 (d, 1H), 6.89 (d, 1H), 6.76 (d, 1H), 6.21 (s, 2H), 1.70 (s, 3H), 1.64 (s, 3H).

### Example 33. 3-Amino-4-(3-hydroxy-2-methylphenyl)-7-methylquinoline-2-carboxamide (46)

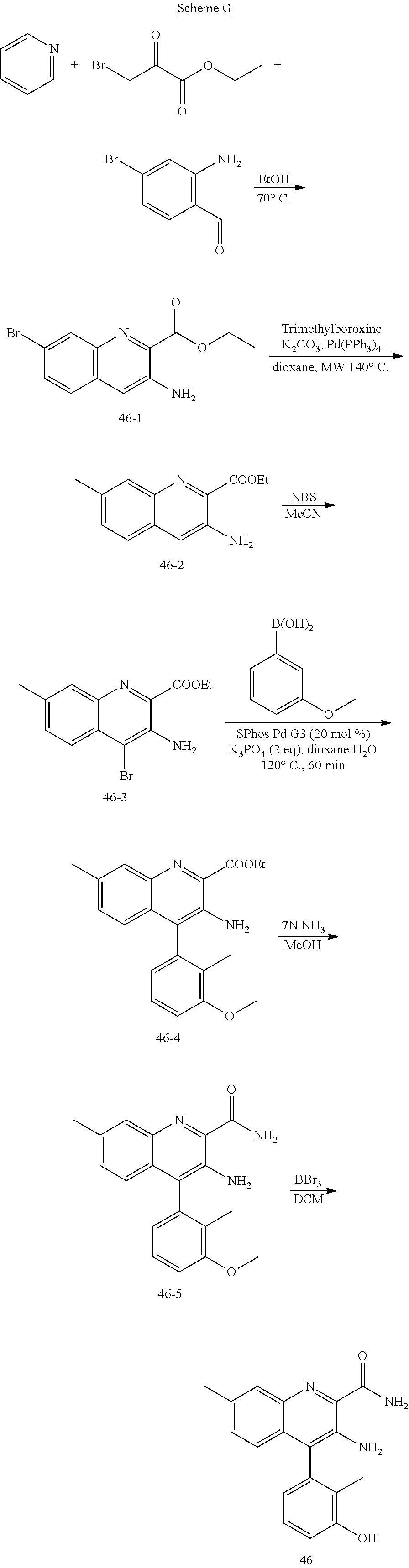

Ethyl 3-amino-7-bromoquinoline-2-carboxylate (Intermediate 46-1): Pyridine (1.05 equiv. 7.87 mmol) was dissolved in 36 mL EtOH, and a solution of ethyl 3-bromo-2-oxo-propanoate (1.05 equiv. 7.87 mmol, 90 mass %) in 24 mL EtOH was added dropwise over 20 min. The reaction was heated to 67° C. and refluxed for 50 min. Then 2-amino-4-bromo-benzaldehyde (1500 mg, 7.49 mmol) and pyridine (2.4 mL) were added. The solution was refluxed overnight. Then pyrrolidine (2 equiv. 14.998 mmol) was added, and the reaction was refluxed for an additional 4 h. The reaction mixture was concentrated and adsorbed onto silica gel and purified by ISCO chromatography to afford a 1.22 g (55% yield) of Intermediate 46-1; LC-MS mi z 295.2 [M+H⁺].

Ethyl 3-amino-7-methylquinoline-2-carboxylate (Intermediate 46-2): In a microwave reactor vial, ethyl 3-amino-7-methyl-quinoline-2-carboxylate (44.9 mg, 0.195 mmol) was dissolved in dioxane. Potassium carbonate (1.50 equiv. 0.292 mmol) and trimethylboroxine (5.00 equiv. 0.974 mmol) were added. The vial was flushed with nitrogen then tetrakis(triphenylphosphine) palladium(0) (0.1 equiv. 0.0195 mmol) was added. The reaction was heated to 140° C. for 20 min in the microwave. The mixture was filtered through Celite® rinsing with EtOAc and concentrated. The residue was purified by chromatography to provide a 32.4 mg of Intermediate 46-2; LC-MS m/z 231.2 [M+H⁺].

Ethyl 3-amino-4-bromo-7-methylquinoline-2-carboxylate (Intermediate 46-3): To a solution of acetonitrile (10 mL) ethyl 3-amino-7-methyl-quinoline-2-carboxylate (178 mg, 0.77 mmol) and ammonium acetate (0.1 equiv. 0.077 mmol), NBS (1.05 equiv. 0.809 mmol) in MeCN (2 mL) was added dropwise over 2 minutes. The reaction mixture was stirred at room temperature for 85 min and treated with additional 14.2 mg NBS. After 2 h, the reaction mixture was concentrated, dissolved in DCM and quenched with saturated aqueous sodium thiosulfate. The aqueous layer was washed with DCM then the combined organic layer was washed with water. The organics were dried over sodium sulfate, filtered, and concentrated. The residue was adsorbed to silica then purified by column chromatography to afford 159 mg (66% yield) of Intermediate 46-3; LC-MS m/z 309.2/311.2 [M++/M+2+H⁺]. ¹H NMR (400 MHz, CDCl3) δ 7.87 (s, 1H), 7.83 (dd, 1H), 7.47-7.39 (m, 1H), 6.20 (s, 2H), 4.56 (qd, 2H), 2.51 (d, 3H), 1.52 (td, 3H).

Ethyl 3-amino-4-(3-methoxy-2-methylphenyl)-7-methylquinoline-2-carboxylate (Intermediate 46-4): To a solution of toluene (2.4 mL) and water (0.27 mL) in microwave reactor vial were added ethyl 3-amino-4-bromo-7-methyl-quinoline-2-carboxylate (138.2 mg, 0.447 mmol), (3-methoxy-2-methyl-phenyl)boronic acid (168 mg, 1.012 mmol), potassium phosphate (2 equiv. 0.894 mmol), and dicyclohexyl-[2-(2,6-dimethoxyphenyl)phenyl]phosphane (0.2 equiv. 0.089 mmol). The reaction vial was purged by bubbling nitrogen for 2 min and then [2-(2-aminophenyl)phenyl]-methylsulfonyloxy-palladium dicyclohexyl-[2-(2,6-dimethoxyphenyl)phenyl]phosphane (0.2 equiv. 0.089 mmol) was added. The reaction vial was purged again with nitrogen for a minute and sealed. The vial was heated to 120° C. for an hour. After completion, the reaction mixture was poured into water, extracted with DCM (3×50 mL), dried over sodium sulfate, filtered, and adsorbed to Celite®, and was purified by column chromatography to yield 135 mg, (86% yield) of Intermediate 46-4. LC-MS m/z 351.2 [M+H⁺]; ¹H NMR (400 MHz, CDCl3) δ 7.39 (t, 1H), 7.28 (m, 2H), 7.04 (t, 2H), 6.77 (d, 1H), 5.48 (Br, s, 2H), 4.62 (d, 2H), 3.95 (d, 3H), 2.51 (s, 3H), 1.88 (d, 3H).

3-Amino-4-(3-methoxy-2-methylphenyl)-7-methylquinoline-2-carboxamide (Intermediate 46-5): Ethyl 3-amino-4-(3-methoxy-2-methyl-phenyl)-7-methyl-quinoline-2-carboxylate (133.6 mg, 0.381 mmol) was dissolved in ammonia in methanol (20 mL, 140 mmol, 7 M). The sealed reaction vessel was heated in an oil bath at 70° C. for 1.75 h. The reaction was stirred at room temperature overnight. The reaction mixture was concentrated in vacuo and purified by column chromatography to provide Intermediate 46-5 (113 mg, 92% yield). LC-MS m/z 322.2 [M+H⁺].

3-Amino-4-(3-hydroxy-2-methylphenyl)-7-methylquinoline-2-carboxamide (46): To a suspension of 3-amino-4-(3-methoxy-2-methyl-phenyl)-7-methyl-quinoline-2-carboxamide (113.4 mg, 0.352 mmol) in dichloromethane (0.1 M) was added boron tribromide (3.00 equiv. 1.059 mmol, 1 M) slowly dropwise; and the reaction was stirred at room temperature for an hour. The reaction mixture was poured into a solution of DCM and 2-(2-hydroxyethylamino)ethanol in an ice bath. The DCM/MeOH and triethylamine were added to quench the reaction. The solution was partially concentrated in vacuo, then was taken up for injection with MeOH/acetonitrile and was separated on RP-HPLC (0-100% MeCN in water with 0.1% FA to give Compound 46 (94 mg, 87% yield). LC-MS m/z 308.2 [M+H⁺]; ¹H NMR (400 MHz, DMSO) δ 9.61 (d, 1H), 8.42 (s, 1H), 7.77 (s, 1H), 7.69 (s, 1H), 7.21 (q, 2H), 6.95 (d, 1H), 6.92-6.85 (m, 1H), 6.55 (d, 1H), 5.99 (s, 2H), 2.42 (s, 3H), 1.71 (s, 3H).

### Example 34. (M)-3-Amino-4-(3-hydroxy-2-methylphenyl)-7-methylquinoline-2-carboxamide (49) and (P)-3-amino-4-(3-hydroxy-2-methylphenyl)-7-methylquinoline-2-carboxamide (50)

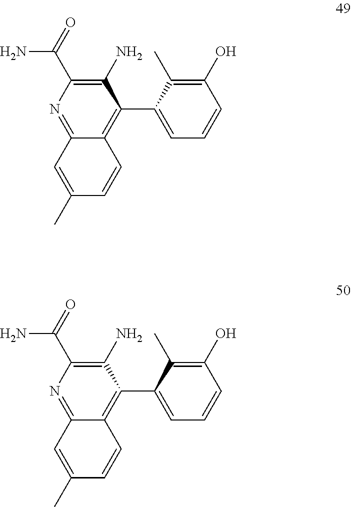

Chiral SFC separation of Compound 46 (Instrument: Waters Prep100 SFC-MS; Column: Phenomenex Lux Cellulose-2, 30×250 mm, 5 um; Conditions: Isocratic at 30% methanol with 70% CO₂; Flow Rate: 40 mL/min) provided Compound 49 (34.7 mg, 48% yield) and Compound 50 (34.6 mg, 48% yield);

Peak 1: SFC t_{R}=1.81 min; LC-MS m/z 308.2 [M+H⁺]; ¹H NMR: (400 MHz, DMSO-d₆) δ=9.62 (s, 1H), 8.42 (s, 1H), 7.77 (s, 1H), 7.69 (s, 1H), 7.21 (q, 2H), 6.95 (d, 1H), 6.88 (d, 1H), 6.55 (d, 1H), 5.99 (s, 2H), 2.42 (s, 3H), 1.71 (s, 3H).

Peak 2: SFC t_{R}=2.22 min.; LC-MS m/z 308.2 [M+H]; ¹H NMR: (400 MHz, DMSO-d₆) δ=9.55 (s, 1H), 8.35 (s, 1H), 7.71 (s, 1H), 7.62 (s, 1H), 7.14 (q, 2H), 6.88 (d, 1H), 6.82 (d, 1H), 6.48 (d, 1H), 5.92 (s, 2H), 2.35 (s, 3H), 1.65 (s, 3H).

### Example 35. 3-Amino-5-fluoro-4-(3-hydroxy-2-methyl-phenyl)quinoline-2-carboxamide (56)

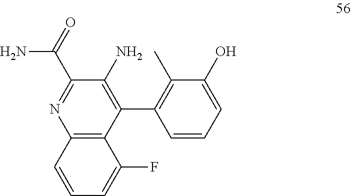 [SMILES: Cc1c(O)cccc1-c1c(N)c(C(N)=O)nc2cccc(F)c12]

Prepared according to Example 17, steps 1-5, using 2-amino-6-fluorobenzaldehyde and (3-methoxy-2-methylphenyl)boronic acid to give 3-amino-5-fluoro-4-(3-hydroxy-2-methyl-phenyl)quinoline-2-carboxamide (Compound 56). LC-MS m/z 312.0 [M+H⁺]; ¹H NMR: (400 MHz, DMSO-d₆) δ: 9.44 (s, 1H), 8.54-8.43 (m, 1H), 7.85 (d, 1H), 7.77 (dd, 1H), 7.37 (ddd, 1H), 7.18-7.06 (m, 2H), 6.88 (dd, 1H), 6.56 (dd, 1H), 6.09 (s, 2H), 1.77 (s, 3H).

### Example 36. (P)-3-Amino-5-fluoro-4-(3-hydroxy-2-methylphenyl)quinoline-2-carboxamide (57) and (M)-3-amino-5-fluoro-4-(3-hydroxy-2-methylphenyl)quinoline-2-carboxamide (58)

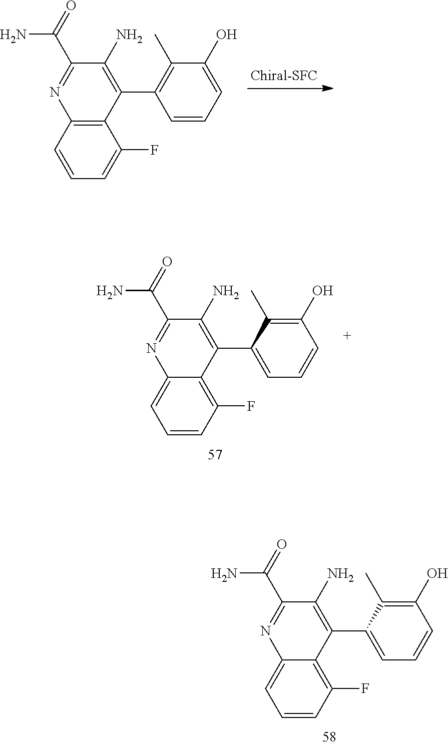

Chiral SFC purification (Instrument: Waters SFC Prep 150 Mgm; Column: Daicel Chiralpak® OJ, 30×150 mm, 5 um; Conditions: Isocratic at 25% methanol with 75% CO2; Flow Rate: 100 mL/min) of Compound 56 (40 mg) provided Compound 57 (18 mg) and Compound 58 (21 mg).

Peak 1: retention time 2.34 min, LC-MS m/z [M+H]=312.0; ¹H NMR (400 MHz, DMSO-d₆) δ 9.45 (s, 1H), 8.49 (d, 1H), 7.85 (d, 1H), 7.77 (dd, 1H), 7.37 (ddd, 1H), 7.18-7.07 (m, 2H), 6.91-6.85 (m, 1H), 6.56 (dd, 1H), 6.09 (s, 2H), 1.77 (s, 3H).

Peak 2: retention time 2.64 min, LC-MS m/z [M+H]=312.0; ¹H NMR (400 MHz, DMSO-d₆) δ 9.45 (s, 1H), 8.48 (d, 1H), 7.85 (d, 1H), 7.77 (dd, 1H), 7.37 (ddd, 1H), 7.18-7.07 (m, 2H), 6.88 (dd, 1H), 6.56 (dd, 1H), 6.09 (s, 2H), 1.77 (s, 3H).

### Example 37. 3-Amino-7-chloro-4-(3-hydroxy-2-methylphenyl)quinoline-2-carboxamide (37)

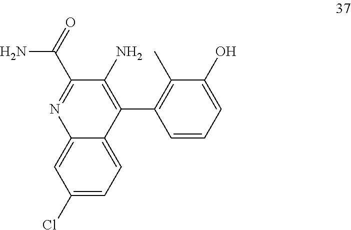 [SMILES: Cc1c(O)cccc1-c1c(N)c(C(N)=O)nc2cc(Cl)ccc12]

Prepared according to Example 17, steps 1-5, using 2-amino-4-chlorobenzaldehyde and 3-methoxy-2-methylphenylboronic acid to give 3-amino-7-chloro-4-(3-hydroxy-2-methylphenyl)quinoline-2-carboxamide (Compound 37); LC-MS m/z 328.2 [M+H]; ¹H NMR: (400 MHz, DMSO-d₆) δ 9.60 (s, 1H), 8.43 (s, 1H), 7.87 (s, 1H), 7.80 (s, 1H), 7.34 (d, 1H), 7.14 (t, 1H), 6.89 (s, 2H), 6.50 (d, 1H), 6.13 (s, 2H), 1.65 (s, 3H).

### Example 38. 3-Amino-7-chloro-4-(3-hydroxy-2,6-dimethylphenyl)quinoline-2-carboxamide (42)

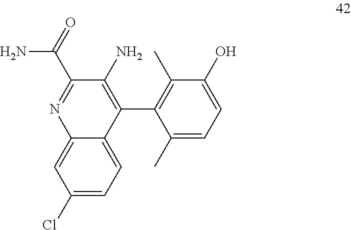 [SMILES: Cc1ccc(O)c(C)c1-c1c(N)c(C(N)=O)nc2cc(Cl)ccc12]

Prepared according to Example 17, steps 1-5, using 2-amino-4-chlorobenzaldehyde and 3-methoxy-2,6-dimethylphenylboronic acid to give 3-amino-7-chloro-4-(3-hydroxy-2,6-dimethylphenyl)quinoline-2-carboxamide (Compound 42); LC-MS m/z 342.2 [M+H⁺]; ¹H NMR: (400 MHz, DMSO-d₆) δ 9.40 (s, 1H), 8.51 (s, 1H), 7.96 (d, 1H), 7.88 (s, 1H), 7.45-7.37 (m, 1H), 7.08 (d, 1H), 6.88 (dd, 2H), 6.16 (s, 2H), 1.69 (d, 3H), 1.63 (d, 3H).

### Example 39. 3-Amino-7-fluoro-4-(3-hydroxy-2,6-dimethylphenyl)quinoline-2-carboxamide (43)

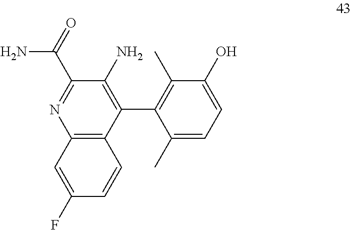 [SMILES: Cc1ccc(O)c(C)c1-c1c(N)c(C(N)=O)nc2cc(F)ccc12]

Prepared according to Example 17, steps 1-5, using 2-amino-4-fluorobenzaldehyde and 3-methoxy-2,6-dimethylphenylboronic acid to give 3-amino-7-fluoro-4-(3-hydroxy-2,6-dimethylphenyl)quinoline-2-carboxamide (Compound 43); LC-MS m/z 326.0 [M+H⁺]; ¹H NMR: (400 MHz, DMSO-d₆) δ 9.35 (s, 1H), 8.46 (d, 1H), 7.83 (d, 1H), 7.63 (dd, 1H), 7.34 (ddd, 1H), 7.08 (d, 1H), 6.95-6.84 (m, 2H), 6.01 (s, 2H), 1.69 (s, 3H), 1.63 (s, 3H).

### Example 40. 3-Amino-4-(3-hydroxy-2,6-dimethylphenyl)-7-methoxyquinoline-2-carboxamide (47)

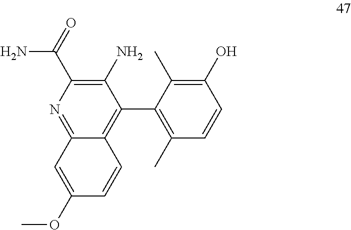 [SMILES: COc1ccc2c(-c3c(C)ccc(O)c3C)c(N)c(C(N)=O)nc2c1]

Prepared according to Example 17, steps 1-5 using 2-amino-4-methoxybenzaldehyde and 2-[3-(ethoxymethoxy)-2,6-dimethylphenyl]-4,4,5,5-tetramethyl-1,3,2-dioxaborolane and using TFA in place of BBr₃ in Step 5 to give 3-amino-4-(3-hydroxy-2,6-dimethylphenyl)-7-methoxyquinoline-2-carboxamide (Compound 47); LC-MS m/z 338.1 [M+H⁺]; ¹H NMR (300 MHz, Methanol-d₄) δ 7.34 (d, 1H), 7.12-6.99 (m, 2H), 6.87 (dd, 2H), 3.92 (s, 3H), 1.75 (d, 6H).

### Example 41. 3-Amino-4-(3-hydroxy-2,6-dimethylphenyl)-6-methoxyquinoline-2-carboxamide (48)

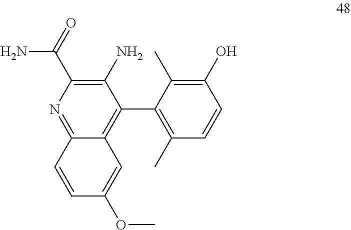 [SMILES: COc1ccc2nc(C(N)=O)c(N)c(-c3c(C)ccc(O)c3C)c2c1]

Prepared according to Example 17, steps 1-5, using 2-amino-5-methoxybenzaldehyde and 2-[3-(ethoxymethoxy)-2,6-dimethylphenyl]-4,4,5,5-tetramethyl-1,3,2-dioxaborolane and using TFA in place of BBr₃ in Step 5 to give 3-amino-4-(3-hydroxy-2,6-dimethylphenyl)-6-methoxyquinoline-2-carboxamide (Compound 48); LC-MS m/z 338.05 [M+H]; ¹H NMR (400 MHz, Methanol-d4) δ 7.87 (m, 1H), 7.15-7.06 (m, 2H), 6.88 (m, 1H), 6.27 (m, 1H), 3.66 (s, 3H), 1.81 (m, 6H).

### Example 42. 3-Amino-4-(3-hydroxy-2-methylphenyl)-6-methylquinoline-2-carboxamide (55)

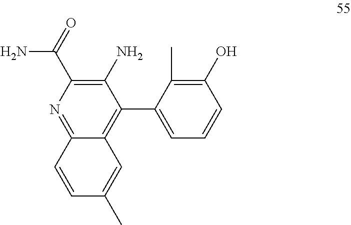 [SMILES: Cc1ccc2nc(C(N)=O)c(N)c(-c3cccc(O)c3C)c2c1]

Prepared according to Example 17, steps 1-5, using 2-amino-5-methylbenzaldehyde and 3-methoxy-2-methylphenylboronic acid to give 3-amino-4-(3-hydroxy-2-methylphenyl)-6-methylquinoline-2-carboxamide (Compound 55); LC-MS m/z 308.1 [M+H⁺]; ¹H NMR: (400 MHz, DMSO-d₆) δ 9.60 (s, 1H), 8.40 (s, 1H), 7.87 (s, 1H), 7.75 (dd, 1H), 7.70 (s, 1H), 7.26-7.20 (m, 2H), 7.18 (d, 1H), 6.95 (s, 1H), 6.55 (d, 1H), 6.09 (s, 2H), 2.39 (s, 3H), 1.77 (s, 3H).

### Example 43. 3-Amino-4-(3-hydroxy-2-methylphenyl)-7-(trifluoromethyl) quinoline-2-carboxamide (59)

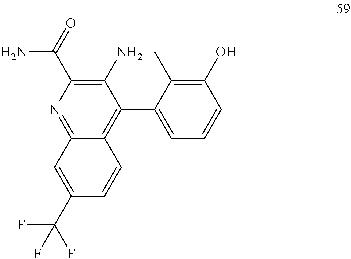 [SMILES: Cc1c(O)cccc1-c1c(N)c(C(N)=O)nc2cc(C(F)(F)F)ccc12]

Prepared according to Example 17, steps 1-5, using 2-amino-4-(trifluoromethyl)benzaldehyde and 3-methoxy-2-methyl-phenylboronic acid to give 3-amino-4-(3-hydroxy-2-methylphenyl)-7-(trifluoromethyl) quinoline-2-carboxamide (Compound 59); LC-MS m/z 362.0 [M+H⁺]; ¹H NMR: (400 MHz, DMSO-d₆) δ 9.63 (s, 1H), 8.57 (s, 1H), 8.25 (s, 1H), 7.89 (dd, 1H), 7.88-7.60 (m, 1H), 7.24-7.20 (m, 1H), 7.15-7.13 (m, 1H), 6.99-6.96 (m, 1H), 6.58-6.57 (m, 1H), 6.41 (bs, 2H), 1.73 (s, 3H).

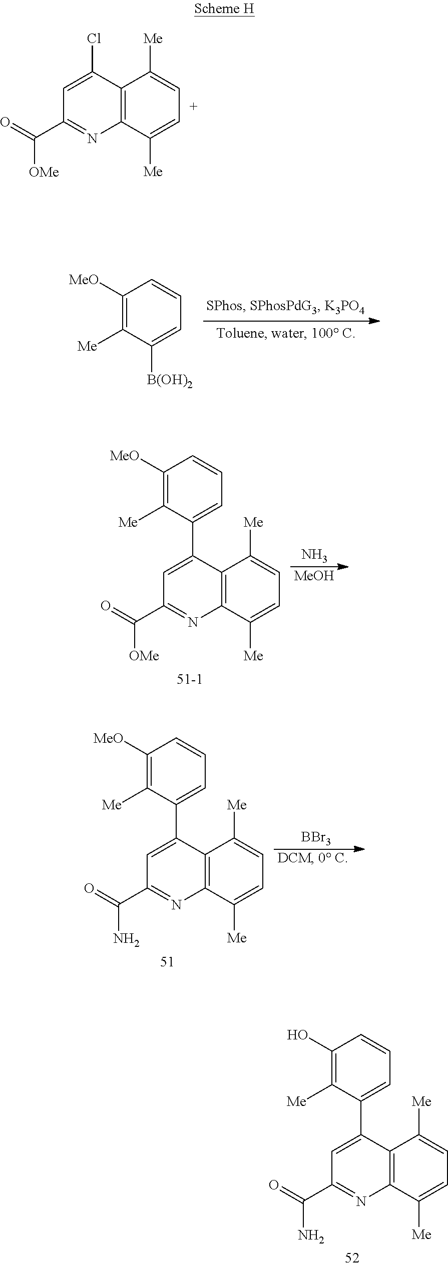

### Example 44. 4-(3-Methoxy-2-methylphenyl)-5,8-dimethylquinoline-2-carboxamide (51)

Prepared according to Example 17, Steps 3 and 4, using methyl 4-chloro-5,8-dimethylquinoline-2-carboxylate and 3-methoxy-2-methylphenylboronic acid to give Compound 51 (206 mg, 88% yield over 2 steps). ¹H NMR (400 MHz, DMSO) δ 8.28 (s, 1H), 7.86 (s, 1H), 7.73 (s, 1H), 7.60 (d, 1H), 7.35-7.22 (m, 2H), 7.08 (d, 1H), 6.80 (d, 1H), 3.87 (s, 3H), 2.82 (s, 3H), 1.88 (s, 3H), 1.74 (s, 3H). LC-MS m/z 321 (M+H⁺).

### Example 45. 4-(3-Hydroxy-2-methylphenyl)-5,8-dimethylquinoline-2-carboxamide (52)

4-(3-methoxy-2-methylphenyl)-5,8-dimethyl-quinoline-2-carboxamide (194 mg, 0.606 mmol, 1 equiv.) was suspended in DCM (10 mL) under an atmosphere of nitrogen gas and cooled in an ice bath. A solution of BBr₃ in DCM (1 M, 3.03 mL, 3.03 mmol, 5 equiv.) was added dropwise over 1 min then the mixture was stirred at 0° C. for 45 min. The solvent was removed. The residue was suspended in DCM (20 mL) and quenched with a saturated solution of NaHCO₃ (20 mL). The mixture was filtered, the precipitate and organic layer were combined, and the solvent was removed. The solid was dissolved in DMF, filtered, and subjected to RP-HPLC (0.1% FA, Water:MeCN 90:10 to 0:100) to afford Compound 52 (124 mg, 67% yield). ¹H NMR (400 MHz, DMSO) δ 9.62 (s, 1H), 8.27 (s, 1H), 7.86 (s, 1H), 7.73 (s, 1H), 7.59 (d, 1H), 7.32 (d, 1H), 7.10 (t, 1H), 6.91 (d, 1H), 6.63 (d, 1H), 2.81 (s, 3H), 1.91 (s, 3H), 1.70 (s, 3H). LC-MS m/z 307 (M+H⁺).

### Example 46. 8-(3-Hydroxy-2,6-dimethyl-phenyl)quinoline-6-carboxamide (60)

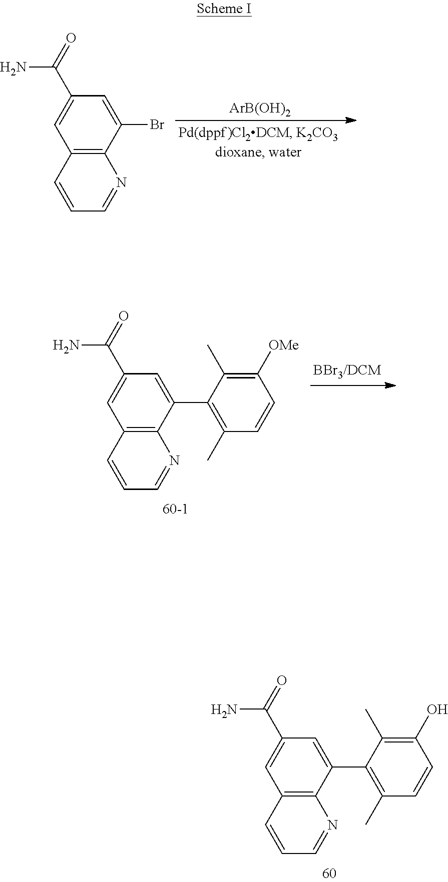 [SMILES: NC(=O)c1cc(Br)c2ncccc2c1 ; "ArB(OH)2" ; COc1ccc(C)c(-c2cc(C(N)=O)cc3cccnc23)c1C ; Cc1ccc(O)c(C)c1-c1cc(C(N)=O)cc2cccnc12]

8-(3-methoxy-2,6-dimethylphenyl)quinoline-6-carboxamide (Intermediate 60-1): To a solution of 8-bromoquinoline-6-carboxamide (20 mg, 0.40 mmol, 1.0 equiv.) and (3-methoxy-2,6-dimethylphenyl) boronic acid (108 mg, 0.60 mmol, 1.5 equiv.) in 1,4-dioxane (0.5 mL) and 2M aqueous potassium carbonate (0.2 mL) was added (1,3-bis(2,6-diisopropylphenyl)imidazolidene)(3-chloropyridyl)palladium(II) dichloride (2.7 mg, 0.0040 mmol, 0.050 equiv.). The mixture was stirred at 70° C. for 2 h. The reaction mixture was concentrated and diluted with water (2 mL). The aqueous phase was decanted. The residue was purified by flash silica gel chromatography to give Intermediate 60-1 (23 mg, 94% yield). LC-MS m/z 307.2 [M+H⁺].

8-(3-hydroxy-2,6-dimethyl-phenyl)quinoline-6-carboxamide (60): To a mixture of 8-(3-methoxy-2,6-dimethylphenyl)quinoline-6-carboxamide (23 mg, 0.075 mmol, 1.0 equiv.) in DCM (0.3 mL) was added BBr₃ (LOM in DCM, 0.23 mmol, 3.0 equiv.) at 0° C. The mixture was stirred, warming to ambient temperature, for 0.5 hour. The reaction mixture was concentrated to dryness. The residue was suspended in DCM and treated with methanol. The mixture was concentrated to dryness. The crude product was triturated with saturated aqueous NaHCO₃ and stirred for 5 min. Solids were collected by vacuum filtration, washed with water and air-dried. The material was purified by RP-HPLC (water+0.1% FA, ACN gradient) and lyophilized to give Compound 60 (8.0 mg, 36% yield). LC-MS m/z 293.2 [M+H⁺]. ¹H NMR (400 MHz, DMSO) δ 9.10 (s, 1H), 8.87-8.82 (m, 1H), 8.58-8.47 (m, 3H), 8.22 (s, 1H), 7.93 (d, 1H), 7.63-7.55 (m, 2H), 6.92 (d, 1H), 6.77 (d, 1H), 1.67 (d, 3H), 1.60 (d, 3H).

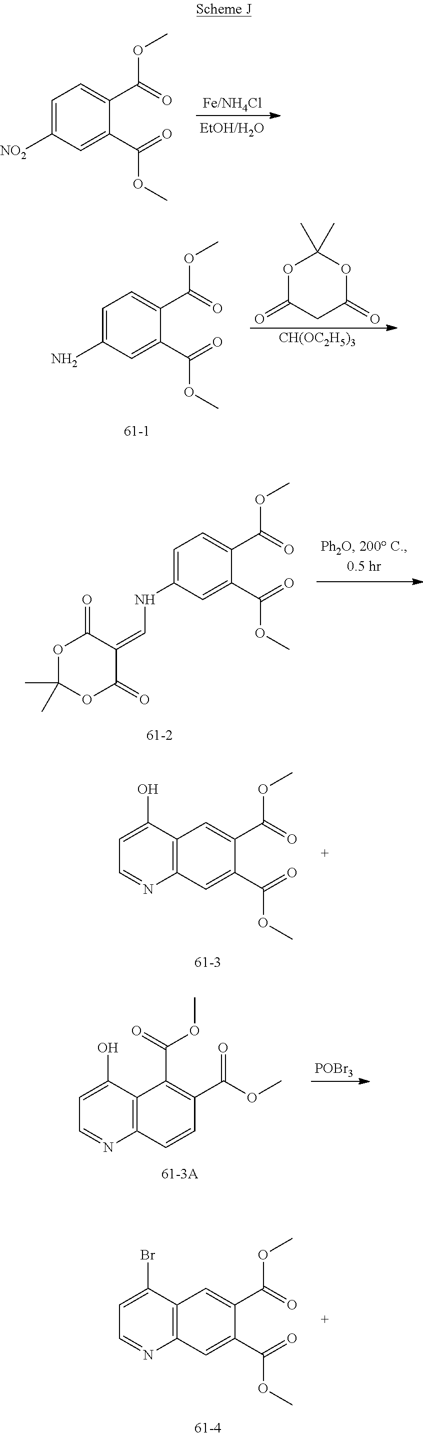

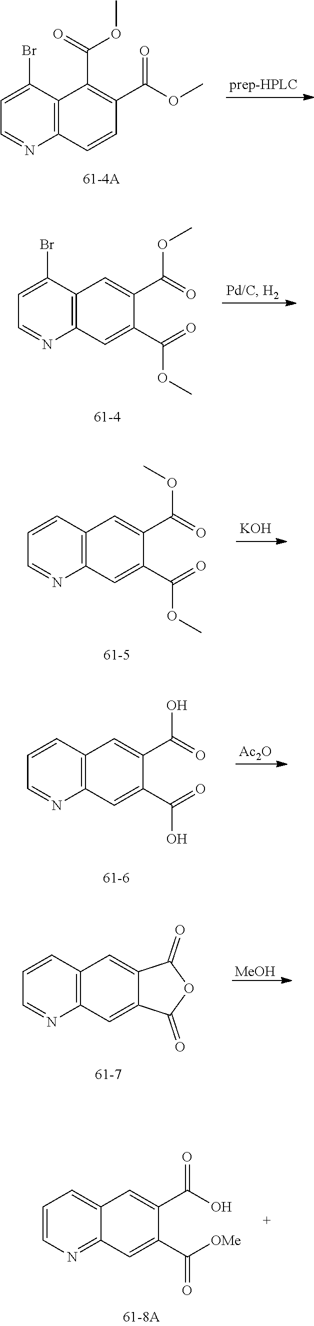

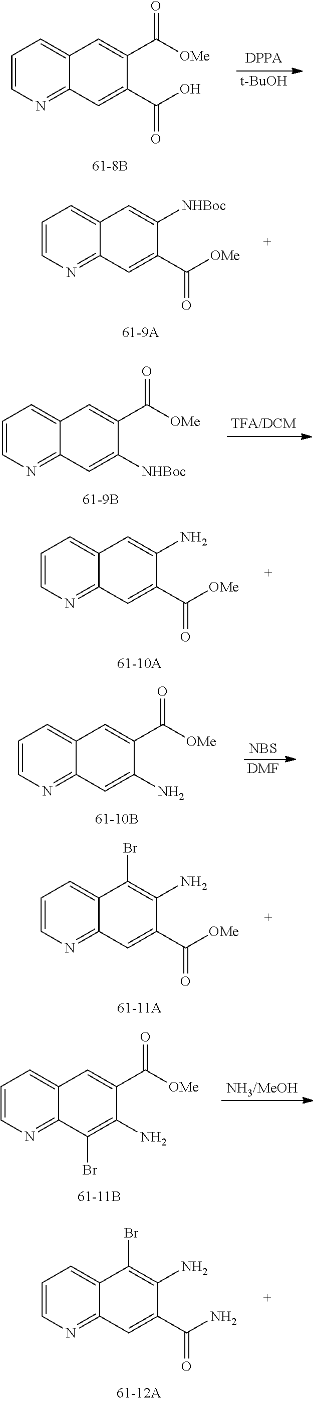

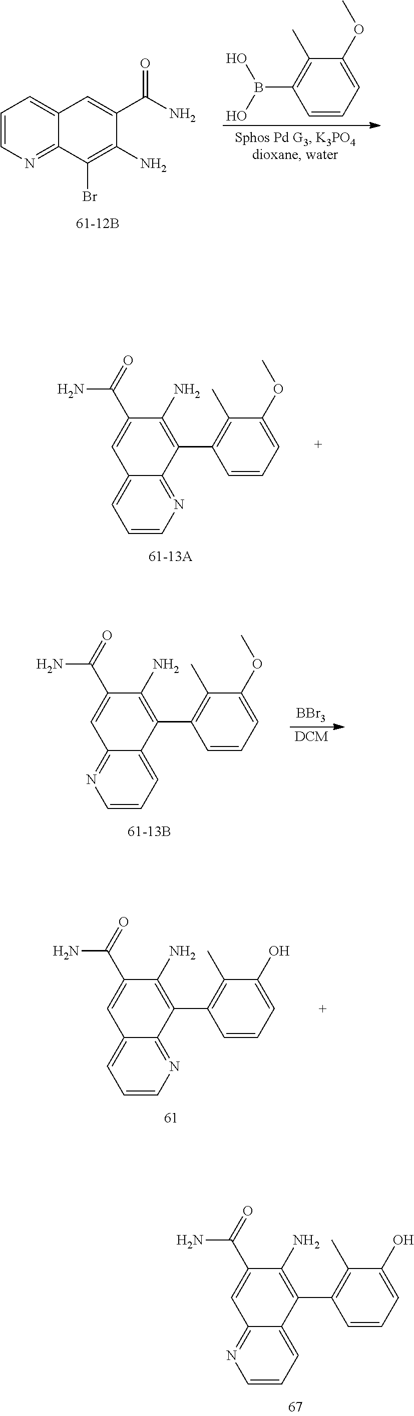

### Example 47. 7-Amino-8-(3-hydroxy-2-methylphenyl)pyrido[2,3-b]pyrazine-6-carboxamide (61) and 6-Amino-5-(3-hydroxy-2-methylphenyl)quinoline-7-carboxamide (67)

Dimethyl 4-aminophthalate (Intermediate 61-1): To a solution of dimethyl 4-nitrobenzene-1,2-dicarboxylate (10 g, 41.81 mmol, 1.0 equiv.) in EtOH (90 mL) and H₂O (30 mL) was added Fe (12.0 g, 209.05 mmol, 5.00 equiv.) and NH₄Cl (11 g, 209.05 mmol, 5.0 equiv.). The mixture was stirred at 85° C. for 1 hour. The mixture was filtered. The filtrate was diluted with water (500 mL) and extracted with EtOAc (200 mL×3). The combined organic phases were washed with brine (200 mL×2), dried over anhydrous Na₂SO₄, filtered and concentrated to give dimethyl 4-aminobenzene-1, 2-dicarboxylate (Intermediate 61-1; 25 g, crude); LC-MS m/z 209.9 [M+H⁺].

Dimethyl 4-(((2,2-dimethyl-4,6-dioxo-1,3-dioxan-5-ylidene)methyl)amino)phthalate (Intermediate 61-2): A solution of 2,2-dimethyl-1,3-dioxane-4,6-dione (17 g, 119.50 mmol, 1.0 equiv.) in CH(OC₂H₅)₃ (100 mL) was stirred at 105° C. for 2 hr. To this solution at the same temperature was added dimethyl 4-aminobenzene-1,2-dicarboxylate (25 g, 119.50 mmol, 1.0 equiv.). The mixture was stirred at 105° C. for another 1 hour. After cooling to room temperature, the mixture was diluted with petroleum ether (200 mL). Solids were collected by vacuum filtration. The filter cake was further washed with petroleum ether (500 mL) to give dimethyl 4-[(2,2-dimethyl-4,6-dioxo-1,3-dioxan-5-ylidene)methylamino]benzene-1,2-dicarboxylate (Intermediate 61-2; 35 g, 96.33 mmol, 80.61% yield); ¹H NMR (400 MHz, DMSO-d₆) δ 11.32 (d, 1H), 8.70-8.56 (m, 1H), 7.93 (d, 1H), 7.82 (d, 2H), 3.82 (m, 6H), 1.68 (d, 6H).

Dimethyl 4-hydroxyquinoline-6,7-dicarboxylate (Intermediate 61-3): A solution of dimethyl 4-[(2, 2-dimethyl-4,6-dioxo-1,3-dioxan-5-ylidene)methylamino]benzene-1,2-dicarboxylate (5.0 g, 13.76 mmol, 1.0 equiv.) in Ph₂O (30 mL) was stirred at 200° C. for 30 min. The mixture was cooled to room temperature and diluted with petroleum ether (100 mL). The filtrate was concentrated and purified by flash silica gel chromatography to give dimethyl 4-hydroxyquinoline-6,7-dicarboxylate (800 mg, 61-3) along with a minor regioisomer (61-3A). LC-MS m/z 261.9 [M+H⁺].

Dimethyl 4-bromoquinoline-6,7-dicarboxylate (Intermediate 61-4): To a solution of dimethyl 4-hydroxyquinoline-6, 7-dicarboxylate (18.6 g, 71.20 mmol, 1 equiv.) in DMF (200 mL) was added POBr₃ (31.4 g, 109.53 mmol, 11.1 mL, 1.54 equiv.). The mixture was stirred at 70° C. for 2 hr. The reaction mixture was slowly poured into ice water (50 mL) and adjusted to pH 8-9 with aq. NaOH (2 M). After stirring for 20 minutes, the mixture was extracted with EtOAc (250 mL×2). The combined organic layers were washed with brine (200 mL×3), dried over anhydrous Na₂SO₄, filtered, and concentrated. The filtrate was concentrated and purified by flash silica gel chromatography to give dimethyl 4-bromoquinoline-6,7-dicarboxylate (61-4; 17.12 g) along with a minor regioisomer (61-4A). The resulting product was further purified by prep-HPLC (column: YMC-Triart Prep C18 250×50 mm×10 um; mobile phase: [water(NH₃H₂O+NH₄HCO₃)-ACN]; B %: 25%-65%, 20 min) to give dimethyl 4-bromoquinoline-6,7-dicarboxylate (Intermediate 61-4; 5.5 g, 16.97 mmol, 57.90% yield); LC-MS m/z 324.0 [M+H⁺]; ¹H NMR (400 MHz, DMSO-d₆) δ=8.91 (d, 1H), 8.52 (s, 1H), 8.38 (s, 1H), 8.15 (d, 1H), 3.91 (d, 6H).

Quinoline-6,7-dicarboxylic acid (Intermediate 61-5): To a solution of dimethyl 4-bromoquinoline-6, 7-dicarboxylate (2 g, 6.17 mmol, 1 equiv.) in EtOAc (20 mL) was added Pd/C (1 g, 6.17 mmol, 10% purity, 1 equiv.). The mixture was degassed with H₂ and stirred at 25° C. for 5 hr under 25° C., 15 psi. The mixture was filtered, and the filtrate was concentrated directly to give dimethyl quinoline-6, 7-dicarboxylate (Intermediate 61-5; 1.4 g, 5.71 mmol, 92% yield); LC-MS m/z 245.9 [M+H⁺].

Quinoline-6,7-dicarboxylic acid (Intermediate 61-6): To a solution of dimethyl quinoline-6, 7-dicarboxylate (200 mg, 0.81 mmol, 1 equiv.) in THE (2 mL) and H₂O (2 mL) was added KOH (183 mg, 3.26 mmol, 4 equiv.). The mixture was stirred at 40° C. for 2 hr. The reaction mixture was concentrated to remove THF and was adjusted to a pH of 5 by addition of HCl (1M) at 0° C., filtered to give quinoline-6,7-dicarboxylic acid (Intermediate 61-6; 170 mg, 95.9% yield); LC-MS m/z 217.9 [M+H].

Furo[3,4-g]quinoline-6,8-dione (Intermediate 61-7): A solution of quinoline-6,7-dicarboxylic acid (170 mg, 0.78 mmol, 1 equiv.) in Ac₂O (3 mL) was stirred at 70° C. for 2 hr. The reaction mixture was concentrated to remove Ac₂O to give furo[3,4-g]quinoline-6,8-dione (Intermediate 61-7; 150 mg, crude); LC-MS m/z 199.9 [M+H⁺].

7-(Methoxycarbonyl)quinoline-6-carboxylic acid and 6-(methoxycarbonyl) quinoline-7-carboxylic acid (Intermediates 61-8A and 61-8B): A solution of furo[3,4-g]quinoline-6,8-dione (1.5 g, 7.53 mmol, 1 equiv.) in MeOH (3 mL) was stirred at 70° C. for 2 hr. LCMS showed the desired product was detected. The reaction mixture was concentrated under reduced pressure to remove MeOH. The residue was purified by reversed phase HPLC (mobile phase:[H₂O-MeOH]; B %:0%-14%) to give a mixture of 7-(methoxycarbonyl)quinoline-6-carboxylic acid (61-8A) and 6-(methoxycarbonyl) quinoline-7-carboxylic acid (61-8B) (584 mg); LC-MS m/z 231.9 [M+H⁺].

Methyl 6-((tert-butoxycarbonyl)amino)quinoline-7-carboxylate and methyl 7-((tert-butoxycarbonyl)amino)quinoline-6-carboxylate (Intermediates 61-9A and 61-9B): To a mixture of 7-(methoxycarbonyl)quinoline-6-carboxylic acid and 6-(methoxycarbonyl) quinoline-7-carboxylic acid (584 mg, 2.53 mmol, 1 equiv.) in t-BuOH (5 mL) was added DPPA (1.04 g, 3.79 mmol, 1.5 equiv.) and triethyl amine (TEA) (511 mg, 5.05 mmol, 2 equiv.). The mixture was stirred at 85° C. for 16 hr. The mixture was concentrated to give a residue. The residue was purified by flash silica gel chromatography to give a mixture of methyl 6-(tert-butoxycarbonylamino)quinoline-7-carboxylate (61-9A) and methyl 7-(tert-butoxycarbonyl)amino)quinoline-6-carboxylate (61-9B) (397 mg); LC-MS m/z 303.1 [M+H⁺].

Methyl 6-aminoquinoline-7-carboxylate and methyl 7-aminoquinoline-6-carboxylate (Intermediates 61-10A and 61-10B): To a mixture of methyl 6-(tert-butoxycarbonylamino)quinoline-7-carboxylate and methyl 7-((tert-butoxycarbonyl)amino)quinoline-6-carboxylate (350 mg, 1.16 mmol, 1 equiv.) in DCM (3 mL) was added TFA (4.62 g, 3.00 mL). The mixture was stirred at 25° C. for 1 hour. The reaction mixture was adjusted to pH=8 with saturated aqueous NaHCO₃. The residue was diluted with water (100 mL) and extracted with DCM (100 mL×2). The combined organic layers were washed with brine (100 mL), dried over anhydrous Na₂SO₄, filtered and concentrated under reduced pressure to give a mixture of methyl 6-aminoquinoline-7-carboxylate (61-10A) and methyl 7-aminoquinoline-6-carboxylate (61-10B) (182 mg); LC-MS m/z 203.1 [M+H⁺].

Methyl 6-amino-5-bromoquinoline-7-carboxylate and methyl 7-amino-8-bromo quinoline-6-carboxylate (Intermediates 61-11A and 61-11B): To a mixture of methyl 6-aminoquinoline-7-carboxylate and methyl 7-aminoquinoline-6-carboxylate (132 mg, 0.65 mmol, 1 equiv.) in DMF (2 mL) was added NBS (116 mg, 0.65 mmol, 1 equiv). The mixture was stirred at 25° C. for 16 hr. The reaction mixture was poured into water (50 mL), then extracted with EtOAc (50 mL×2). The combined organic layers were washed with brine 100 mL (50 mL×2), dried over anhydrous Na₂SO₄, filtered, and concentrated to give a residue. The residue was purified by flash silica gel chromatography to give a mixture of methyl 6-amino-5-bromoquinoline-7-carboxylate (61-11A) and methyl 7-amino-8-bromo quinoline-6-carboxylate (61-11B) (350 mg); LC-MS m/z 280.8 [M+H].

6-Amino-5-bromoquinoline-7-carboxamide and 7-amino-8-bromoquinoline-6-carboxamide (Intermediates 61-12A and 61-12B): A solution of methyl 6-amino-5-bromoquinoline-7-carboxylate and methyl 7-amino-8-bromo quinoline-6-carboxylate (200 mg, 0.71 mmol, 1 equiv.) in NH₃/MeOH (3 mL) was stirred at 60° C. for 6 hr. The reaction mixture was concentrated under reduced pressure. The crude product was triturated with MTBE (5 mL) at 25° C. for 5 min and filtered to give a mixture of 6-amino-5-bromoquinoline-7-carboxamide (61-12A) and 7-amino-8-bromoquinoline-6-carboxamide (61-12B) (92 mg); LC-MS m/z 265.8 [M+H⁺].

7-Amino-8-(3-methoxy-2-methylphenyl)quinoline-6-carboxamide and 6-amino-5-(3-methoxy-2-methylphenyl)quinoline-7-carboxamide (Intermediates 61-13A and 61-13B): To a solution of 6-amino-5-bromoquinoline-7-carboxamide and 7-amino-8-bromoquinoline-6-carboxamide (80 mg, 0.30 mmol, 1 equiv.) and (3-methoxy-2-methyl-phenyl)boronic acid (100 mg, 0.60 mmol, 2 equiv.) in dioxane (2 mL) and H₂O (0.4 mL) was added SPhos Pd G₃ (23 mg, 0.03 mmol, 0.1 equiv.) and K₃PO₄ (511 mg, 2.41 mmol, 8 equiv.). The mixture was stirred at 100° C. for 16 hr. The reaction mixture was diluted with H₂O (50 mL) and extracted with DCM (50 mL×3). The organic layer was separated, washed with brine (50 mL×2), dried over anhydrous Na2SO4, filtered and concentrated give to 7-amino-8-(3-methoxy-2-methylphenyl)quinoline-6-carboxamide (61-13A) and 6-amino-5-(3-methoxy-2-methylphenyl)quinoline-7-carboxamide (61-13B) (150 mg); LC-MS m/z 308.0 [M+H⁺].

7-Amino-8-(3-hydroxy-2-methylphenyl)quinoline-6-carboxamide (61) and 6-amino-5-(3-hydroxy-2-methylphenyl)quinoline-7-carboxamide (67): To a solution of 7-amino-8-(3-methoxy-2-methylphenyl)quinoline-6-carboxamide and 6-amino-5-(3-methoxy-2-methylphenyl)quinoline-7-carboxamide (150 mg, 0.49 mmol, 1 equiv.) in DCM (2 mL) was added BBr₃ (122 mg, 0.49 mmol, 1 equiv.). The mixture was stirred at 0° C. for 1 hour. The reaction mixture was quenched by addition water 10 mL at 25° C., and then extracted with DCM (25 mL×2). The combined organic layers were washed with brine (50 mL×2), dried over anhydrous Na₂SO₄, filtered and concentrated to give a mixture of 7-amino-8-(3-hydroxy-2-methylphenyl)quinoline-6-carboxamide and 6-amino-5-(3-hydroxy-2-methylphenyl)quinoline-7-carboxamide. The crude material was purified by prep-HPLC (column: Welch Xtimate® C18 150×30 mm×5 um; mobile phase: [water(NH₃H₂O+NH₄HCO₃)-ACN]; B %: 4%-44%) to give Compound 61 (21.2 mg, 14.8% yield) and Compound 67 (28.4 mg, 19.8% yield).

7-Amino-8-(3-hydroxy-2-methylphenyl)quinoline-6-carboxamide (61): LC-MS m/z 294.1 [M+H⁺]; ¹H NMR (400 MHz, DMSO-d₆) δ=8.54 (dd, 1H), 8.34-8.09 (m, 2H), 7.25-7.16 (m, 2H), 6.89 (d, 1H), 6.69-6.58 (m, 1H), 1.82 (s, 3H).

6-Amino-5-(3-hydroxy-2-methylphenyl)quinoline-7-carboxamide (67), LC-MS m/z 294.2 [M+H⁺]; ¹H NMR (400 MHz, DMSO-d₆) δ=8.56 (dd, 1H), 8.24 (s, 1H), 7.47 (d, 1H), 7.30 (dd, 1H), 7.21 (t, 1H), 6.93 (d, 1H), 6.64 (d, 1H), 1.82 (s, 3H).

### Example 48. (P)-3-Amino-4-(3-hydroxy-2-methylphenyl)-7-methylquinoline-2-carboxamide (62) and (M)-3-amino-4-(3-hydroxy-2-methylphenyl)-7-methylquinoline-2-carboxamide (63)

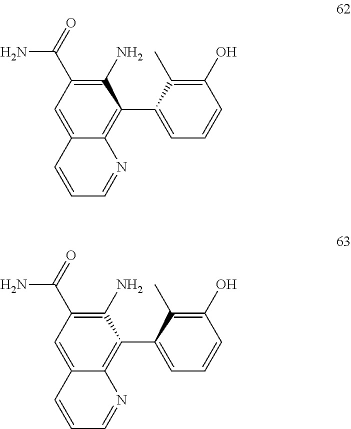

Chiral SFC purification (Instrument: Waters SFC Prep 150 Mgm; Column: Daicel Chiralpak® AD, 30×150 mm, 5 um; Conditions: Isocratic at 25% methanol+0.1% TEA with 75% CO2; Flow Rate: 100 mL/min) of Compound 61 gave Compound 62 (14.7 mg) and Compound 63 (8.5 mg).

Peak 1: retention time 3.18 min, LC-MS m/z 294.2 [M+H⁺]; ¹H NMR (400 MHz, MeOD) δ 8.44 (d, 1H), 8.17 (d, 1H), 8.13 (s, 1H), 7.11 (dt, 2H), 6.80 (d, 1H), 6.55 (d, 1H), 1.72 (s, 3H);

Peak 2: retention time 3.67 min, LC-MS m/z 294.2 [M+H⁺]; ¹H NMR (400 MHz, MeOD) δ 8.44 (d, 1H), 8.16 (d, 1H), 8.12 (d, 1H), 7.17-7.05 (m, 2H), 6.80 (d, 1H), 6.55 (d, 1H), 1.72 (d, 3H).

### Example 49. (M)-6-Amino-5-(3-hydroxy-2-methylphenyl)quinoline-7-carboxamide (68) and (P)-6-amino-5-(3-hydroxy-2-methylphenyl)quinoline-7-carboxamide (69)

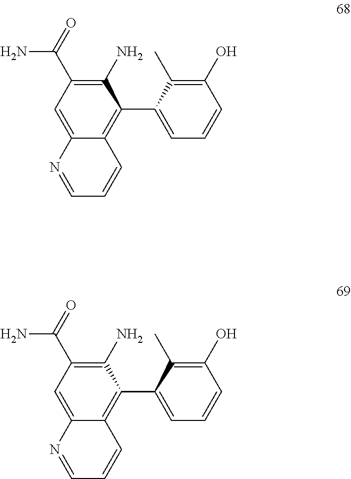

Chiral SFC purification (Instrument: Waters SFC Prep 150 Mgm; Column: Daicel Chiralpak® IG, 30×150 mm, 5 um; Conditions: Isocratic at 30% methanol+0.1% TEA with 70% CO2; Flow Rate: 100 mL/min) of Compound 67 gave Compound 68 (10.7 mg) and Compound 69 (12.5 mg).

Peak 1: retention time 2.60 min, LC-MS m/z 294.15 [M+H⁺]; ¹H NMR (400 MHz, MeOD) δ 8.47 (d, 1H), 8.14 (d, 1H), 7.38 (d, 1H), 7.21 (dt, 1H), 7.12 (t, 1H), 6.83 (d, 1H), 6.55 (d, 1H), 1.72 (d, 3H).

Peak 2: retention time 2.93 min, LC-MS m/z 294.15 [M+H⁺]; ¹H NMR (400 MHz, MeOD) δ 8.46 (d, 1H), 8.13 (s, 1H), 7.37 (d, 1H), 7.20 (dd, 1H), 7.11 (t, 1H), 6.82 (d, 1H), 6.54 (d, 1H), 1.72 (s, 3H).

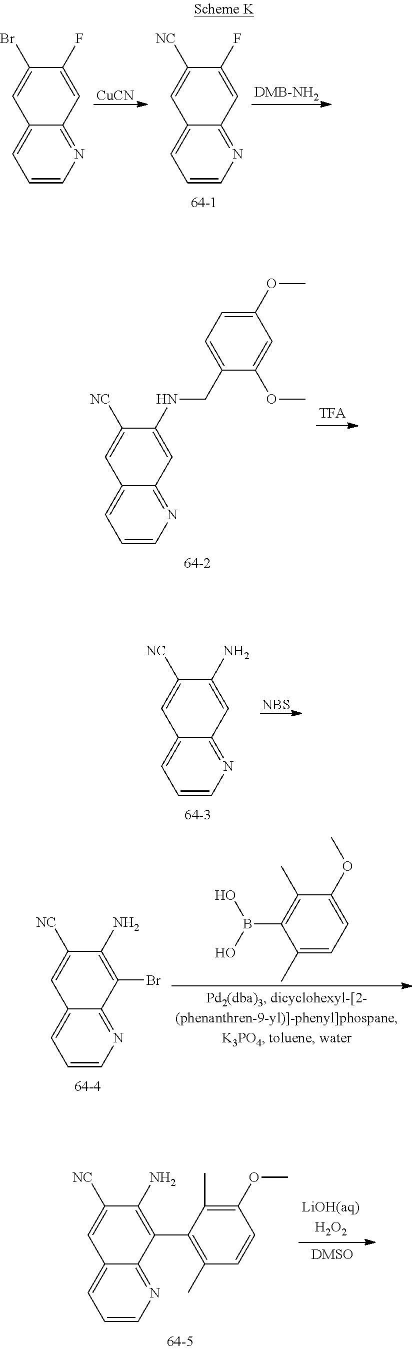

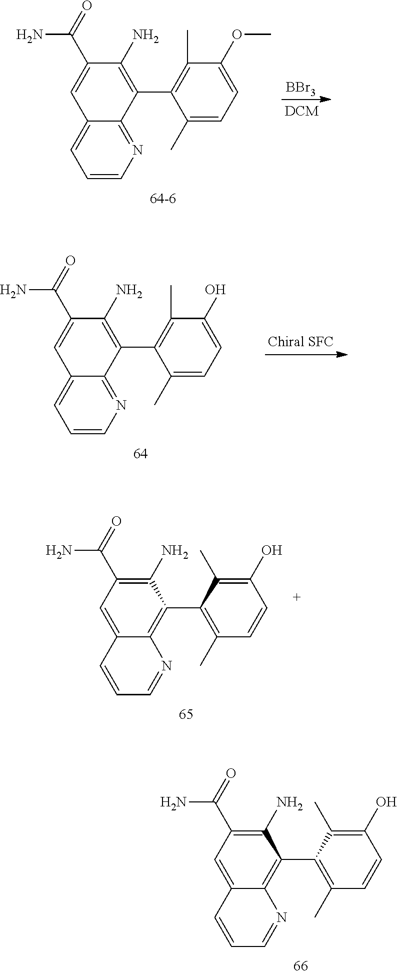

### Example 50. 7-Amino-8-(3-hydroxy-2,6-dimethylphenyl)quinoline-6-carboxamide (64)

7-fluoroquinoline-6-carbonitrile (Intermediate 64-1): To a solution of N, N-dimethylformamide (15 mL) was added 6-bromo-7-fluoroquinoline (450 mg, 1.990 mmol, 1.0 equiv.) and copper(I) cyanide (220 mg, 2.456 mmol, 1.24 equiv). The reaction mixture was stirred at 140° C. overnight. Additional copper(I) cyanide (0.1 eq) was added, and the reaction was stirred at 140° C. for another 4 hr. The reaction mixture was cooled to room temperature and quenched with water. The solid precipitate was collected by vacuum filtration, dried and purified further by column chromatography to give 7-((2,4-dimethoxybenzyl)amino)quinoline-6-carbonitrile (Intermediate 64-1) (250 mg, 72.9% yield); LC-MS m/z 173.20 [M+H⁺].

7-((2,4-Dimethoxybenzyl)amino)quinoline-6-carbonitrile (Intermediate 64-2): To a solution of 7-fluoroquinoline-6-carbonitrile (60 mg, 0.348 mmol, 1.0 equiv) in DMSO (1 mL) was added (2,4-dimethoxyphenyl)methanamine (0.27 mL, 1.8 mmol, 5.1 equiv.). The mixture was stirred at 90° C. for 4 hr. The mixture was cooled and quenched with water. The resulting mixture was extracted with EtOAc (50 mL×3) was washed with brine. The combined organic layers were adsorbed onto silica gel and purified by column chromatography to give 7-[(2,4-dimethoxyphenyl)methylamino]quinoline-6-carbonitrile (Intermediate 64-2) (110 mg, 98.8% yield); LC-MS m/z 320.25; [M+H]; ¹H NMR (400 MHz, DMSO-d₆) δ 8.72 (s, 1H), 8.39 (s, 1H), 8.15 (d, 1H), 7.27-7.08 (m, 2H), 6.81 (d, 2H), 6.61 (s, 1H), 6.45 (d, 1H), 4.39 (d, 2H), 3.89 (s, 3H), 3.72 (s, 3H).

7-Aminoquinoline-6-carbonitrile (Intermediate 64-3): A mixture of 7-[(2,4-dimethoxyphenyl)methylamino]quinoline-6-carbonitrile (430 mg, 1.34 mmol, 1.0 equiv.) in 2,2,2-trifluoroacetic acid (1 mL, 13.06 mmol, 10 equiv.) was stirred at 50° C. for 30 min. The reaction mixture was evaporated to dryness then dissolved in EtOAc. The organic layer was washed sequentially with saturated aq. NaHCO₃ then brine, dried over MgSO₄, filtered, and concentrated to obtain 7-aminoquinoline-6-carbonitrile (Intermediate 64-3); LC-MS m/z 170.1 [M+H⁺].

7-Amino-8-bromoquinoline-6-carbonitrile (Intermediate 64-4): To a suspension of 7-aminoquinoline-6-carbonitrile (230 mg, 1.359 mmol, 1.0 equiv.) in N,N-dimethylformamide (7 mL) was added NBS (277 mg, 1.556 mmol, 1.14 equiv.). The mixture was stirred at room temperature for 1.5 h. The mixture was quenched with water. The resulting precipitated solid was filtered, washed with water, and air-dried. The aqueous layer was extracted with EtOAc, and the combined organic layers were adsorbed onto silica and purified by column chromatography along with the solid portion to provide 7-amino-8-bromo-quinoline-6-carbonitrile (Intermediate 64-4) (275 mg, 81.5% yield); LC-MS m/z 248.1/250.1 [M+/M+2+H⁺].

7-Amino-8-(3-methoxy-2,6-dimethylphenyl)quinoline-6-carbonitrile (Intermediate 64-5): To a 2-5 mL microwave vial were added 7-amino-8-bromo-quinoline-6-carbonitrile (95 mg, 0.383 mmol, 1.0 equiv.), toluene (3 mL), (3-methoxy-2,6-dimethyl-phenyl)boronic acid (99.8 mg, 0.601 mmol, 2.0 equiv.), potassium phosphate (245 mg, 1.154 mmol, 3.0 equiv.), Tris(dibenzylideneacetone)dipalladium(0) (35 mg, 0.038 mmol, 0.1 equiv.), and dicyclohexyl[2-(phenanthren-9-yl)phenyl]phosphane and (35 mg, 1.154 mmol, 0.2 equiv). The flask was purged by bubbling nitrogen through the solution for 2 min and then heated to 105° C. for 3 hr. The mixture was filtered through Celite®, washed with EtOAc, and the filtrate was concentrated. The crude product was adsorbed on Celite®, then subjected to flash column chromatography to afford Intermediate 64-5 (180 mg, 71% yield); LC-MS m/z 304.2 [M+H⁺].

7-Amino-8-(3-methoxy-2,6-dimethylphenyl)quinoline-6-carboxamide (Intermediate 64-6): To a solution of 7-amino-8-(3-methoxy-2,6-dimethyl-phenyl)quinoline-6-carbonitrile (260 mg, 0.857 mmol, 1.0 equiv) in DMSO (1 mL, 0.2 M,) was added LiOH (1 mL, 2 mmol, 2 M) and added dropwise 30% hydrogen peroxide (0.2 mL, 2 mmol, 2.33 equiv). After 20 min, the mixture was quenched with water and extracted with EtOAc (25 mL×5). The combined organic layers were adsorbed onto silica gel and purified by column chromatography to afford 7-amino-8-(3-methoxy-2,6-dimethyl-phenyl)quinoline-6-carboxamide (Intermediate 64-6) (255 mg, 92.6% yield); LC-MS m/z 322.2 [M+H⁺].

7-Amino-8-(3-hydroxy-2,6-dimethylphenyl)quinoline-6-carboxamide (64): To a solution of 7-amino-8-(3-methoxy-2,6-dimethyl-phenyl)quinoline-6-carboxamide (22 mg, 0.068 mmol) in DCM (0.35 mL) was added boron tribromide (0.2 mL, 0.2 mmol, 1 M in DCM) at 0° C. The reaction mixture was allowed to warm to room temperature and stirred for 30 min. The reaction was diluted with DCM (5 ml) and quenched with water (1 mL) slowly. The resulting mixture was basified with aq. saturated NaHCO₃ to pH=8-9 and extracted with EtOAc (5 mL×5). The combined organic layers were evaporated and further purified by RP-Prep HPLC to yield 7-amino-8-(3-hydroxy-2,6-dimethyl-phenyl)quinoline-6-carboxamide (64) (9.5 mg, 0.031 mmol, 45% yield); LC-MS m/z 308.2 [M+H⁺]; ¹H NMR (400 MHz, DMSO) δ 8.99 (s, 1H), 8.50 (s, 1H), 8.14 (s, 2H), 8.06 (d, 1H), 7.54 (s, 1H), 7.10 (s, 1H), 6.89 (d, 1H), 6.69 (d, 1H), 5.55 (s, 2H), 1.61 (s, 3H), 1.55 (s, 3H).

### Example 51. (M)-7-Amino-8-(3-hydroxy-2,6-dimethyl-phenyl)quinoline-6-carboxamide (65) and (P)-7-amino-8-(3-hydroxy-2,6-dimethyl-phenyl)quinoline-6-carboxamide (66)

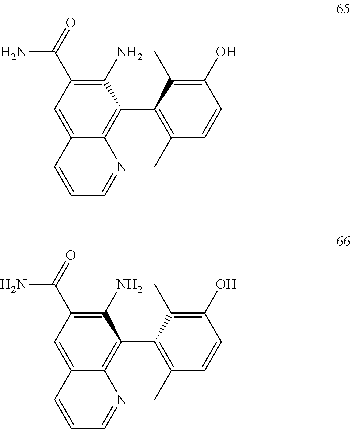

Chiral SFC separation (Instrument: Waters SFC Prep 150 Mgm; Column: Daicel Chiralpak® IG, 30×150 mm, 5 um; Conditions: Isocratic at 35% methanol+0.1% TEA with 65% CO2; Flow Rate: 100 mL/min) of Compound 64 gave Compound 65 (37.6 mg) and Compound 66 (46.5 mg).

Peak 1: retention time 1.53 min, LC-MS m/z 308.2 [M+H⁺]; ¹H NMR (400 MHz, DMSO) δ 9.06 (d, 1H), 8.60-8.55 (m, 1H), 8.25-8.18 (m, 2H), 8.13 (d, 1H), 7.61 (s, 1H), 7.17 (dt, 1H), 6.96 (d, 1H), 6.80-6.73 (m, 1H), 5.63 (s, 2H), 1.67 (d, 3H), 1.62 (d, 3H).

Peak 2: retention time 1.83 min, LC-MS m/z 308.2 [M+H⁺]; ¹H NMR (400 MHz, DMSO) δ 8.99 (d, 1H), 8.51 (dd, 1H), 8.19-8.12 (m, 2H), 8.07 (dd, 1H), 7.55 (s, 1H), 7.10 (dt, 1H), 6.90 (d, 1H), 6.70 (dd, 1H), 5.56 (s, 2H), 1.61 (d, 3H), 1.55 (d, 3H).

### Example 52. 8-(5-Methyl-1H-indazol-4-yl)quinoxaline-6-carboxamide (70)

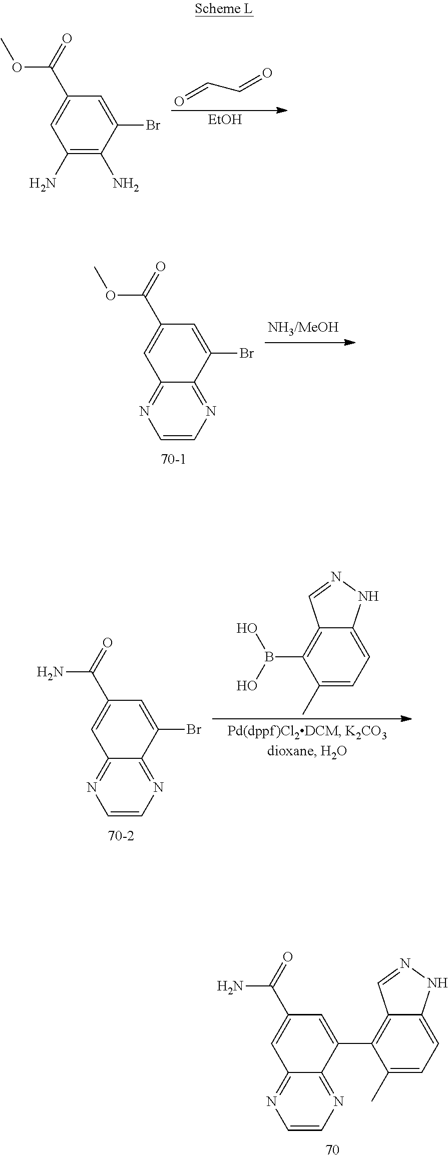

Methyl 8-bromoquinoxaline-6-carboxylate (Intermediate 70-1): A solution of methyl 3,4-diamino-5-bromobenzoate (1.5 g, 6.1 mmol, 1 equiv.) and glyoxal (1.1 g, 18 mmol, 0.96 mL, 3 equiv.) in EtOH (15 mL) was stirred at 80° C. for 17 h. Water (10 mL) was added, and the mixture was filtered. The solid was washed with water (10 mL). The solid was purified by recrystallization from EtOH/water to give Intermediate 70-1 (1.47 g, 4.61 mmol, 75% yield) which was used without further purification in the next step. LC-MS n/z 269 (M+H⁺+2).

8-bromoquinoxaline-6-carboxamide (Intermediate 70-2): A solution of methyl 8-bromoquinoxaline-6-carboxylate (400 mg, 1.50 mmol, 1 equiv.) in NH₃/MeOH (7 M, 8 mL) was stirred at 60° C. for 6 h. The reaction mixture was concentrated and triturated with methanol (5 mL) at 20° C. for 30 min, then filtered to give Intermediate 70-2 (298 mg, 1.13 mmol, 76% yield) which was used without further purification in the next step. LC-MS m/z 254 (M+H⁺+2).

8-(5-methyl-1H-indazol-4-yl)quinoxaline-6-carboxamide (70): To a solution of 8-bromoquinoxaline-6-carboxamide (200 mg, 0.79 mmol, 1 equiv.) and (5-methyl-1H-indazol-4-yl)boronic acid (209 mg, 1.19 mmol, 1.5 equiv.) in dioxane (3 mL) and H₂O (1 mL) was added Pd(dppf)Cl₂·DCM (65 mg, 0.08 mmol, 0.1 equiv.) and K₂CO₃ (329 mg, 2.38 mmol, 3 equiv.). The mixture was stirred at 90° C. for 16 h. The mixture was diluted with water (30 mL) and extracted with DCM (3×20 mL). The combined organic layers were washed with brine (2×20 mL), dried over Na₂SO₄, filtered, and concentrated. The residue was purified by RP-HPLC to give Compound 70 (50 mg, 0.16 mmol, 21% yield). ¹H NMR (400 MHz, DMSO-d₆) δ=13.06 (s, 1H), 9.04 (d, 1H), 8.88 (d, 1H), 8.75 (d, 1H), 8.43 (s, 1H), 8.24 (d, 1H), 7.74 (s, 1H), 7.54 (d, 1H), 7.38-7.31 (m, 2H), 2.02 (s, 3H). LC-MS m/z 304 (M+H⁺).

### Example 53. 8-(3-Methyl-1H-indazol-4-yl)quinoxaline-6-carboxamide (72)

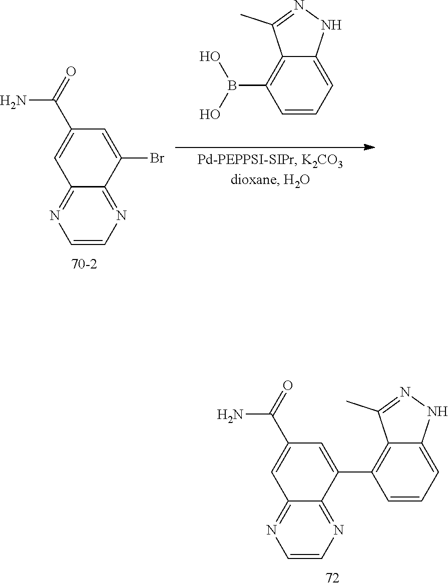 [SMILES: NC(=O)c1cc(Br)c2nccnc2c1 ; Cc1n[nH]c2cccc(B(O)O)c12 ; Cc1n[nH]c2cccc(-c3cc(C(N)=O)cc4nccnc34)c12]

To a solution of 8-bromoquinoxaline-6-carboxamide 70-2 (38 mg, 0.15 mmol, 1.0 equiv.) and (3-methyl-1H-indazol-4-yl)boronic acid (55 mg, 0.31 mmol, 2.0 equiv.) in dioxane (1 mL) and H₂O (0.1 mL) was added (1,3-bis(2,6-diisopropylphenyl)imidazolidene)(3-chloropyridyl) palladium(II) dichloride (2.5 mg, 0.0037 mmol, 0.024 equiv.) and K₂CO₃ (68 mg, 0.49 mmol, 3.3 equiv.). The mixture was stirred at 100° C. for 16 h. The cooled reaction mixture was diluted with water (1 mL) and extracted with EtOAc (3×5 mL). The combined organic layers were concentrated. The residue was purified by RP-HPLC to give Compound 72 (26 mg, 0.086 mmol, 57% yield). ¹H NMR (400 MHz, DMSO-d₆) δ=12.74 (s, 1H), 8.98 (s, 1H), 8.83 (s, 1H), 8.67 (s, 1H), 8.38 (s, 1H), 8.20 (s, 1H), 7.70 (s, 1H), 7.49 (d, 1H), 7.40-7.31 (m, 1H), 6.97 (d, 1H), 1.43 (s, 3H). LC-MS m/z 304.2 (M+H⁺).

### Example 54. 8-(3-Hydroxy-2,6-dimethyl-phenyl)quinoxaline-6-carboxamide (74)

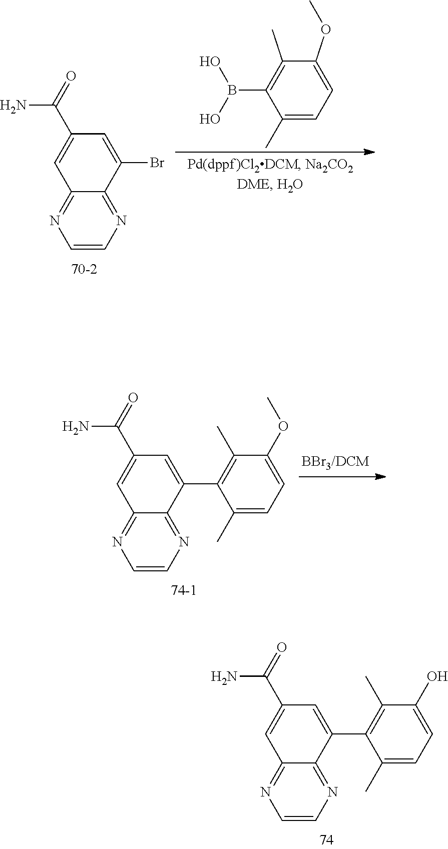 [SMILES: NC(=O)c1cc(Br)c2nccnc2c1 ; COc1ccc(C)c(B(O)O)c1C ; COc1ccc(C)c(-c2cc(C(N)=O)cc3nccnc23)c1C ; Cc1ccc(O)c(C)c1-c1cc(C(N)=O)cc2nccnc12]

8-(3-methoxy-2,6-dimethyl-phenyl)quinoxaline-6-carboxamide (Intermediate 74-1): To a solution of 8-bromoquinoxaline-6-carboxamide 70-2 (200 mg, 0.79 mmol, 1 equiv.) and (3-methoxy-2,6-dimethyl-phenyl)boronic acid (214 mg, 1.19 mmol, 1.5 equiv.) in DME (9 mL) and H₂O (3 mL) was added Na₂CO₃ (841 mg, 7.93 mmol, 10 equiv.) and Pd(dppf)Cl₂·DCM (65 mg, 0.08 mmol, 0.1 equiv.). The mixture was stirred at 120° C. for 3 h. The mixture was diluted with water (30 mL) and extracted with DCM (3×20 mL). The combined organic layers were washed with brine (2×20 mL), dried over Na₂SO₄, filtered and concentrated. The residue was purified by FCC (DCM:MeOH, 0:100 to 97:3) to give Intermediate 74-1 (65 mg, 0.19 mmol, 24% yield). LC-MS m/z 308 (M+H⁺).

8-(3-hydroxy-2,6-dimethyl-phenyl)quinoxaline-6-carboxamide (74): To a solution of 8-(3-methoxy-2,6-dimethyl-phenyl)quinoxaline-6-carboxamide (60 mg, 0.20 mmol, 1 equiv.) in DCM (1 mL) was added BBr₃ (147 mg, 0.59 mmol, 3 equiv.). The mixture was stirred at 20° C. for 2 hr. The mixture was quenched with H₂O (10 mL) at 0° C. and adjusted to pH=7 with sat. NaHCO₃. The mixture was diluted with water (30 mL) and extracted with DCM (3×20 mL). The combined organic layers were washed with brine (2×20 mL), dried over Na₂SO₄, filtered and concentrated. The residue was purified by RP-HPLC to give Compound 74 (28 mg, 0.096 mmol, 49% yield). ¹H NMR (400 MHz, DMSO-d₆) δ=9.18 (s, 1H), 9.02 (d, 1H), 8.90 (d, 1H), 8.66 (d, 1H), 8.38 (s, 1H), 8.02 (d, 1H), 7.71 (s, 1H), 6.94 (d, 1H), 6.79 (d, 1H), 1.68 (s, 3H), 1.60 (s, 3H). LC-MS m/z 294 (M+H⁺).

### Example 55. 8-(1H-indazol-4-yl)quinoxaline-6-carboxamide (75)

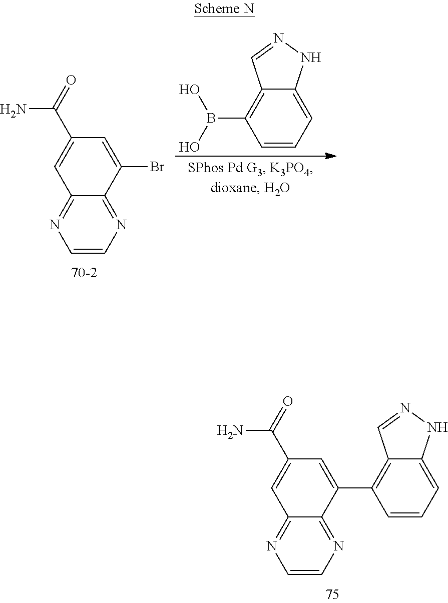

Prepared according to Example 54 using 8-bromoquinoxaline-6-carboxamide 70-2 and 1H-indazol-4-ylboronic acid to give Compound 75 (17.1 mg, 7% yield). ¹H NMR (400 MHz, DMSO) δ 13.18 (s, 1H), 9.06 (s, 1H), 8.93 (s, 1H), 8.71 (s, 1H), 8.47-8.37 (m, 2H), 7.74 (s, 1H), 7.67-7.61 (m, 2H), 7.50 (s, 1H), 7.30 (d, 1H). LC-MS m/z 290 (M+H⁺).

### Example 56. 8-(4-Hydroxy-2,6-dimethylphenyl)quinoxaline-6-carboxamide (76)

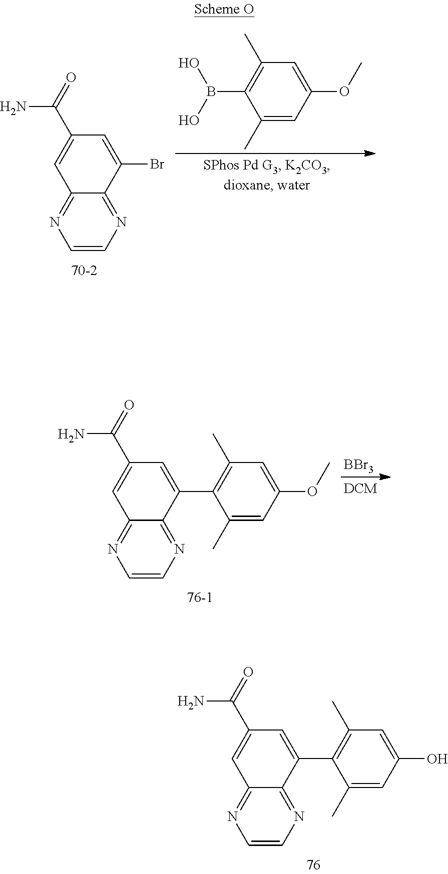

Prepared according to Example 54 using 8-bromoquinoxaline-6-carboxamide 70-2 and (4-methoxy-2,6-dimethylphenyl)boronic acid to give Compound 76 (6.1 mg, 2% yield over 2 steps). ¹H NMR (400 MHz, DMSO) δ 9.27 (s, 1H), 9.01 (d, 1H), 8.90 (d, 1H), 8.63 (d, 1H), 8.37 (s, 1H), 8.03 (d, 1H), 7.69 (s, 1H), 6.57 (s, 2H), 1.72 (s, 6H). LC-MS m/z 294 (M+H⁺).

### Example 57. 8-(3-Hydroxy-2-methylphenyl)quinoxaline-6-carboxamide (80)

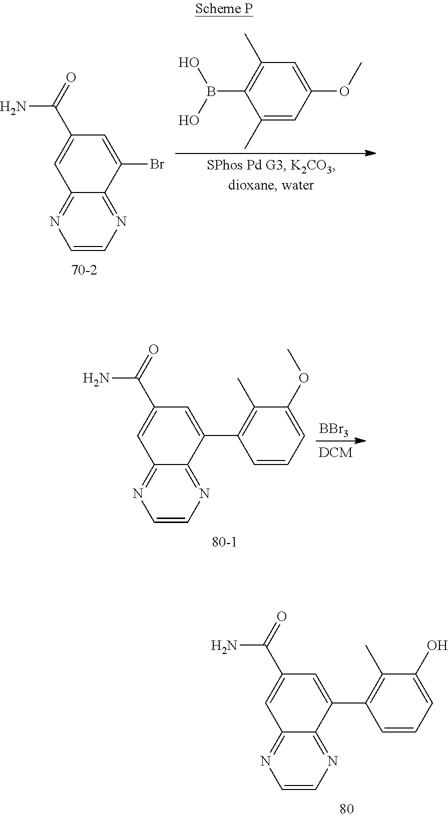

Prepared according to Example 54 using 8-bromoquinoxaline-6-carboxamide 70-2 and (3-methoxy-2-methylphenyl)boronic acid to give Compound 80 (17 mg, 9% yield over 2 steps). ¹H NMR (400 MHz, DMSO) δ 9.42 (s, 1H), 8.98-9.07 (d, 1H), 8.87-8.97 (d, 1H), 8.59-8.69 (m, 1H), 8.31-8.46 (m, 1H), 8.04-8.20 (m, 1H), 7.62-7.80 (m, 1H), 7.03-7.13 (t, 1H), 6.86-6.94 (d, 1H), 6.60-6.76 (d, 1H), 1.71 (s, 3H). LC-MS m/z 280 (M+H⁺).

### Example 58. 8-(1-Methyl-2-oxo-1,2-dihydropyridin-4-yl)quinoxaline-6-carboxamide (81)

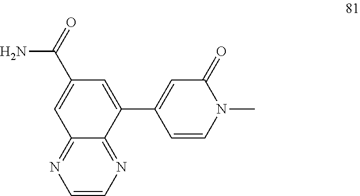 [SMILES: Cn1ccc(-c2cc(C(N)=O)cc3nccnc23)cc1=O]

Prepared according to Example 52 using 8-bromoquinoxaline-6-carboxamide and (1-methyl-2-oxo-4-pyridyl)boronic acid (136.5 mg, 0.881 mmol, 4.8 equiv.) to give Compound 81 (1.0 mg, 1.9% yield). ¹H NMR (400 MHz, DMSO) δ 9.02 (s, 1H), 8.97 (s, 1H), 8.61 (s, 1H), 8.40 (s, 1H), 8.26 (s, 1H), 7.73 (s, 2H), 6.64 (s, 1H), 6.56 (s, 1H), 6.48 (d, 1H), 3.44 (s, 3H). LC-MS m/z 281.2 (M+H⁺).

### Example 59. 7-Amino-8-(3-hydroxy-2-methyl-phenyl)quinoxaline-6-carboxamide (73)

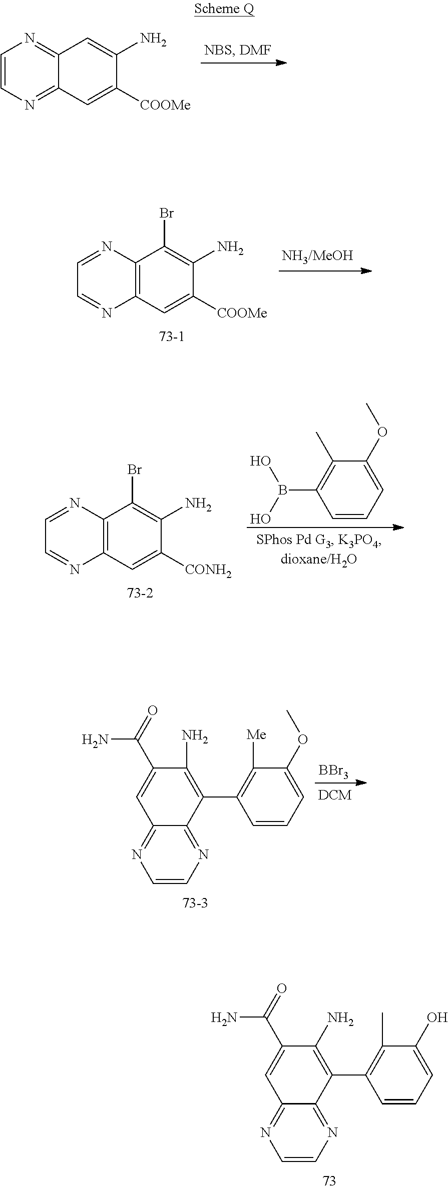

Methyl 7-amino-8-bromo-quinoxaline-6-carboxylate (Intermediate 73-1): To a solution of methyl 7-aminoquinoxaline-6-carboxylate (2.80 g, 13.6 mmol, 1 equiv.; A. Jeanguenat et al.; Bioorg. Med. Chem. 24 (2016) 403-427.) in DMF (20 mL) was added NBS (2.4 g, 13.58 mmol, 1 equiv.). The reaction was stirred at 25° C. for 2 h under an atmosphere of nitrogen gas. The reaction mixture was poured into water (150 mL). Solids were collected by vacuum filtration, washed with water (3×10 mL), and dried to give Intermediate 73-1 (2.60 g, 8.41 mmol, 62% yield). LC-MS m/z 284 (M+H⁺).

7-amino-8-bromo-quinoxaline-6-carboxamide (Intermediate 73-2): A solution of methyl 7-amino-8-bromo-quinoxaline-6-carboxylate (520 mg, 1.8 mmol, 1 equiv.) in NH₃/MeOH (7 M, 10 mL) was stirred at 60° C. for 9 h in a microwave reactor. The reaction mixture was concentrated under vacuum, triturated with methyl tert-butyl ether (MTBE) (5 mL), then filtered to give Intermediate 73-2 (500 mg, 1.7 mmol, 92% yield). LC-MS m/z 267 (M+H⁺).

7-amino-8-(3-methoxy-2-methylphenyl)quinoxaline-6-carboxamide (Intermediate 73-3): To a solution of 7-amino-8-bromo-quinoxaline-6-carboxamide (200 mg, 0.75 mmol, 1 equiv.) and (3-methoxy-2-methylphenyl)boronic acid (149 mg, 0.90 mmol, 1.2 equiv.) in dioxane (4 mL) and H₂O (2 mL) was added SPhos Pd G₃ (58 mg, 0.07 mmol, 0.1 equiv.) and K₃PO₄ (1.27 g, 5.99 mmol, 8 equiv.). The mixture was stirred at 100° C. for 3 hr. The residue was diluted with water (100 mL) and extracted with DCM (3×60 mL). The combined organic layers were washed with brine (2×60 mL), dried over Na₂SO₄, filtered and concentrated. The residue was purified by FCC (DCM:MeOH, 100:0 to 90:10) to give Intermediate 73-3 (190 mg, 0.61 mmol, 81.47% yield, 99% purity). LC-MS m/z 309 (M+H⁺).

7-amino-8-(3-hydroxy-2-methylphenyl)quinoxaline-6-carboxamide (73): To a solution of 7-amino-8-(3-methoxy-2-methylphenyl)quinoxaline-6-carboxamide (180 mg, 0.58 mmol, 1 equiv.) in DCM (4 mL) was added BBr₃ (731 mg, 2.92 mmol, 0.28 mL, 5 equiv.) at 0° C. under N₂. The mixture was stirred at 0° C. for 1 hour, then at 25° C. for another 1 hour. The reaction was adjusted to a pH of approximately 7 with aq. saturated NaHCO₃. The reaction mixture was diluted with water (50 mL) and extracted with DCM (3×50 mL). The combined organic layers were dried over Na₂SO₄, filtered, and concentrated under reduced pressure. The residue was purified by RP-HPLC to give Compound 73 (25 mg, 0.08 mmol, 15% yield). ¹H NMR (400 MHz, DMSO-d₆) δ=9.40 (s, 1H), 8.60 (d, 1H), 8.56 (d, 1H), 8.40 (s, 1H), 8.31 (s, 1H), 7.71 (s, 1H), 7.12-7.08 (m, 1H), 6.87 (d, 1H), 6.54 (d, 1H), 5.91 (s, 2H), 1.69 (s, 3H). LC-MS mA z 295 (M+H⁺).

### Example 60. (P)-7-Amino-8-(3-hydroxy-2-methylphenyl)quinoxaline-6-carboxamide (87) and (M)-7-amino-8-(3-hydroxy-2-methylphenyl)quinoxaline-6-carboxamide (89)

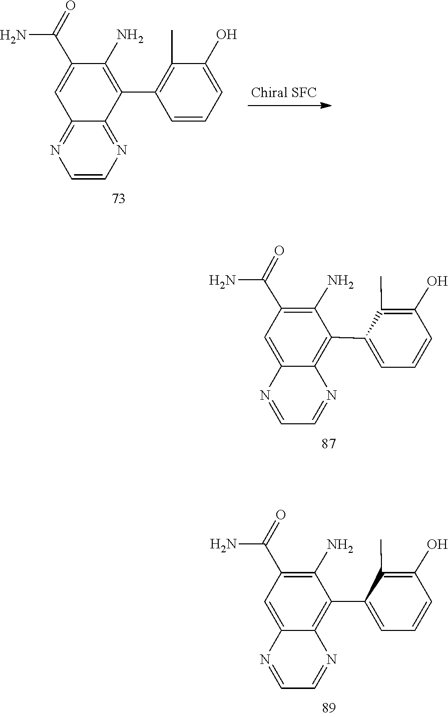

Chiral SFC separation of Compound 73 (12 mg, 0.041 mmol) (Instrument: Waters SFC Prep 150 Mgm; Column: Daicel Chiralpak® AD, 30×150 mm, 5 um; Conditions: Isocratic at 40% methanol with 60% CO₂; Flow Rate: 100 mL/min) provided Compound 87 (5 mg) and Compound 89 (5 mg).

Peak 1: retention time 1.87 min. ¹H NMR (400 MHz, d6-DMSO) δ 9.39 (s, 1H), 8.58 (d, J=15.7 Hz, 2H), 8.41 (s, 1H), 8.32 (s, 1H), 7.74 (s, 1H), 7.10 (t, J=8.0 Hz, 1H), 6.87 (d, J=8.1 Hz, 1H), 6.54 (d, J=7.5 Hz, 1H), 5.93 (s, 2H), 1.69 (s, 3H). LC-MS m/z 295.2 (M+H⁺)

Peak 2: retention time 2.92 min. ¹H NMR (400 MHz, d6-DMSO) δ 9.38 (s, 1H), 8.58 (d, J=15.6 Hz, 2H), 8.41 (s, 1H), 8.31 (s, 1H), 7.74 (s, 1H), 7.10 (t, J=7.8 Hz, 1H), 6.86 (d, J=8.0 Hz, 1H), 6.53 (d, J=7.5 Hz, 1H), 5.93 (s, 2H), 1.68 (s, 2H). LC-MS m/z 295.2 (M+H⁺).

### Example 61. 7-Amino-8-(5-hydroxy-2-methylphenyl)quinoxaline-6-carboxamide (123)

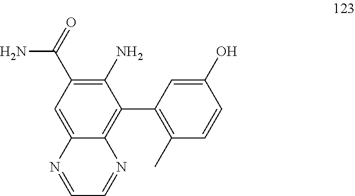 [SMILES: Cc1ccc(O)cc1-c1c(N)c(C(N)=O)cc2nccnc12]

Prepared according to Example 59 using (5-methoxy-2-methylphenyl)boronic acid to give Compound 123 (186 mg). ¹H NMR (400 MHz, DMSO-d₆) δ 9.20 (s, 1H), 8.62 (d, 1H), 8.57 (d, 1H), 8.40 (s, 1H), 8.32 (s, 1H), 7.72 (s, 1H), 7.16 (d, 1H), 6.74 (m, 1H), 6.50 (d, 1H), 5.95 (s, 2H), 1.76 (s, 3H). LC-MS m/z 295.1 [M+H⁻].

### Example 62. (M)-7-Amino-8-(5-hydroxy-2-methylphenyl)quinoxaline-6-carboxamide (116) and (P)-7-amino-8-(5-hydroxy-2-methylphenyl)quinoxaline-6-carboxamide (117)

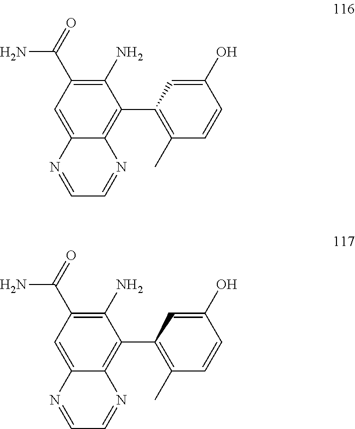

Chiral SFC separation of Compound 123 (Instrument: Waters, SFC-80Q; Daicel Chiralpak® IG (250 mm×30 mm, 10 um); mobile phase: [0.1% NH₃H₂O EtOH]; B %: 35%-35%) provided Compound 116 (30.4 mg) and Compound 117 (25.4 mg).

Peak 1: retention time 1.41 min. ¹H NMR (400 MHz, d6-DMSO) δ 9.19 (s, 1H), 8.62 (d, 1H), 8.57 (d, 1H), 8.39 (s, 1H), 8.32 (s, 1H), 7.72 (s, 1H), 7.16 (d, 1H), 6.74 (m, 1H), 6.49 (d, 1H), 5.95 (s, 2H), 1.76 (s, 3H). LC-MS m/z 295.2 (M+H⁺).

Peak 2: retention time 1.47 min. ¹H NMR (400 MHz, d6-DMSO) δ 9.19 (s, 1H), 8.59 (m, 2H), 8.39 (s, 1H), 8.32 (s, 1H), 7.72 (s, 1H), 7.16 (d, 1H), 6.74 (m, 1H), 6.49 (d, 1H), 5.95 (s, 2H), 1.76 (s, 3H). LC-MS m/z 295.2 (M+H).

### Example 63. 7-Amino-8-(2-chloro-3-hydroxyphenyl)quinoxaline-6-carboxamide (97)

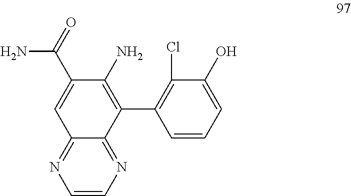 [SMILES: NC(=O)c1cc2nccnc2c(-c2cccc(O)c2Cl)c1N]

Prepared according to Example 59, steps 3 and 4, using (2-chloro-3-methoxyphenyl)boronic acid to give Compound 97 (83.6 mg, 37.8% yield). LC-MS m/z 314.9 [M+H⁺]; ¹H NMR (400 MHz, DMSO-d6) δ 10.12 (s, 1H), 8.60-8.56 (m, 2H), 8.41 (s, 1H), 8.34 (s, 1H), 7.73 (s, 1H), 7.23 (t, 1H), 7.03 (d, 1H), 6.71 (d, 1H), 6.07 (s, 2H).

### Example 64. (P)-7-Amino-8-(2-chloro-3-hydroxyphenyl)quinoxaline-6-carboxamide (105) and (M)-7-amino-8-(2-chloro-3-hydroxyphenyl)quinoxaline-6-carboxamide (106)

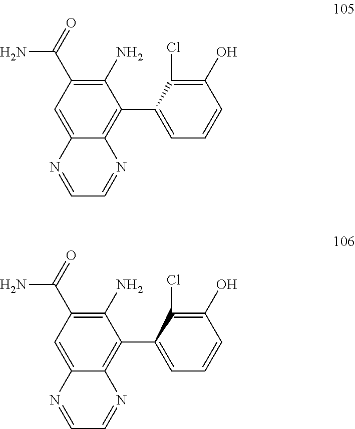

Chiral SFC separation of Compound 97 (73 mg, 0.041 mmol) (Instrument: Waters, SFC-80Q; Column: Daicel Chiralpak® IBN, 30×250 mm, 10 um; Conditions: Isocratic at 40% ethanol+0.1% NH₄OH with 60% CO2) provided Compound 105 (20.2 mg) and Compound 106 (29.2 mg).

Peak 1: retention time 2.02 min. ¹H NMR (400 MHz, d6-DMSO) δ 10.10 (s, 1H), 8.53 (d, 2H), 8.42 (s, 1H), 8.34 (s, 1H), 7.66 (s, 1H), 7.24 (t, 1H), 7.03 (d, 1H), 6.71 (d, 1H), 6.08 (s, 2H). LC-MS m/z 315.0 [M+H]⁺

Peak 2: retention time 2.36 min. ¹H NMR (400 MHz, d6-DMSO) δ 10.04 (s, 1H), 8.53 (d, 2H), 8.41 (s, 1H), 8.34 (s, 1H), 7.73 (s, 1H), 7.18 (t, 1H), 6.98 (d, 1H), 6.65 (d, 1H), 6.01 (s, 2H). LC-MS m/z 315.0 [M+H]⁺

### Example 65. 7-Amino-8-(2-fluoro-3-hydroxyphenyl)quinoxaline-6-carboxamide (120)

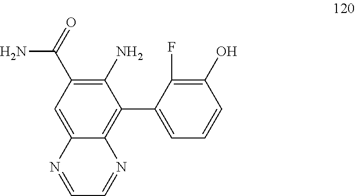 [SMILES: NC(=O)c1cc2nccnc2c(-c2cccc(O)c2F)c1N]

Prepared according to Example 59, steps 3 and 4, using (2-fluoro-3-methoxyphenyl)boronic acid to give Compound 120 (90 mg). ¹H NMR (400 MHz, DMSO-d₆) δ 9.82 (s, 1H), 8.63 (d, 1H), 8.58 (d, 1H), 8.42 (s, 1H), 8.33 (s, 1H), 7.74 (s, 1H), 7.09 (d, 1H), 7.02 (d, 1H), 6.68 (s, 1H), 6.19 (s, 2H). LC-MS m/z 299.3 [M+H⁺].

### Example 66. 7-Amino-8-(2-chloro-5-hydroxyphenyl)quinoxaline-6-carboxamide (121)

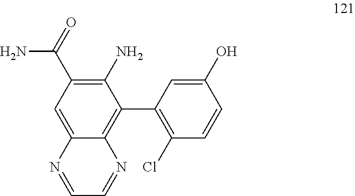 [SMILES: NC(=O)c1cc2nccnc2c(-c2cc(O)ccc2Cl)c1N]

Prepared according to Example 59, using (2-chloro-5-methoxyphenyl)boronic acid to give Compound 121 (18.7 mg). ¹H NMR (400 MHz, DMSO-d₆) δ 9.73 (m, 1H), 8.63 (d, 1H), 8.57 (d, 1H), 8.42 (s, 1H), 8.34 (s, 1H), 7.75 (s, 1H), 7.38 (d, 1H), 6.85 (m, 1H), 6.65 (d, 1H), 6.14 (s, 2H). LC-MS m/z 315.3 [M+H⁺].

### Example 67. 7-Amino-8-(2-chloro-5-hydroxy-3-pyridyl)quinoxaline-6-carboxamide (122)

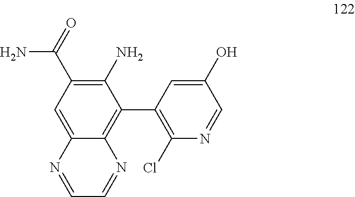 [SMILES: NC(=O)c1cc2nccnc2c(-c2cc(O)cnc2Cl)c1N]

Prepared according to Example 59 using (2-chloro-5-methoxy-3-pyridyl)boronic acid to give Compound 122 (41.2 mg). ¹H NMR (400 MHz, DMSO-d₆) δ 10.25 (s, 1H), 8.63 (d, 1H), 8.58 (d, 1H), 8.44 (s, 1H), 8.36 (s, 1H), 8.00 (d, 1H), 7.77 (br s, 1H), 7.12 (d, 1H), 6.45 (s, 2H). LC-MS m/z 316.3 [M+H⁺].

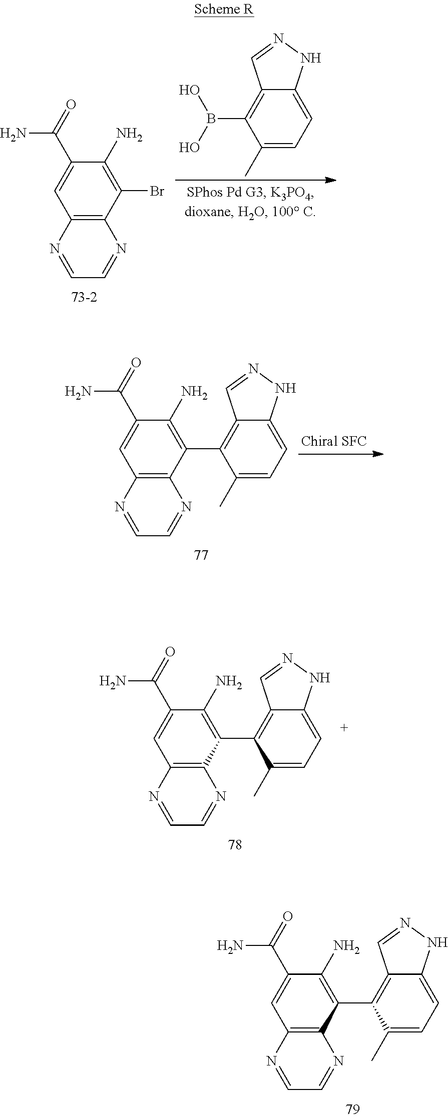

### Example 68. 7-Amino-8-(5-methyl-1H-indazol-4-yl)quinoxaline-6-carboxamide (77)

7-Amino-8-(5-methyl-1H-indazol-4-yl)quinoxaline-6-carboxamide (77): To a solution of 7-amino-8-bromo-quinoxaline-6-carboxamide (200 mg, 0.749 mmol, 1 equiv.) and (5-methyl-1H-indazol-4-yl)boronic acid (264 mg, 1.50 mmol, 2 equiv.) in dioxane (2 mL) and H₂O (0.4 mL) was added SPhos Pd G₃ (58 mg, 0.075 mmol, 0.1 equiv.) and K₃PO₄ (1.27 g, 5.99 mmol, 8 equiv.). The mixture was stirred at 100° C. for 3 hours. The reaction mixture was quenched by the addition of water (40 mL) then extracted with ethyl acetate (3×30 mL). The combined organic layers were washed with brine (20 mL×3), dried over Na₂SO₄, filtered and concentrated. The residue was purified by FCC (EtOAc:pet ether, 30:70 to 100:0). The crude product was purified by RP-HPLC to afford Compound 77 (48 mg, 0.15 mmol, 20% yield). ¹H NMR (400 MHz, DMSO-d₆) δ=12.98 (s, 1H), 8.72-8.33 (m, 4H), 7.76 (s, 1H), 7.51 (d, 1H), 7.37 (d, 1H), 7.22 (s, 1H), 5.98 (s, 2H), 2.00 (s, 3H). LC-MS m/z 319 (M+H⁺).

### Example 69. (P)-7-Amino-8-(5-methyl-1H-indazol-4-yl)quinoxaline-6-carboxamide (78) and (M)-7-amino-8-(5-methyl-1H-indazol-4-yl)quinoxaline-6-carboxamide (79)

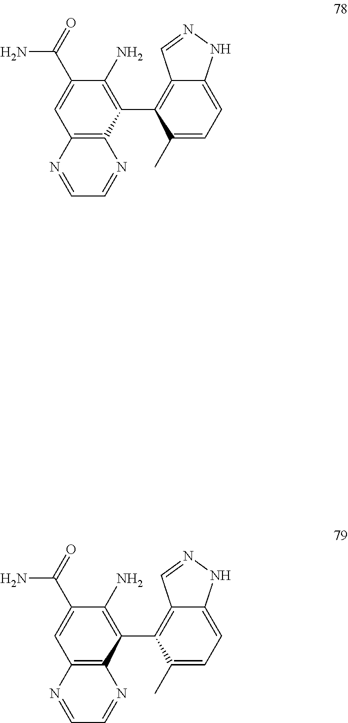

Chiral SFC separation of Compound 77 (40 mg, 0.126 mmol) (Instrument: Waters, SFC-80Q; Daicel Chiralpak® AD (250 mm×30 mm, 10 um); mobile phase: [0.1% NH₃H₂O EtOH]; B %: 50%-50%) provided Compound 78 (11 mg, 0.034 mmol, 27% yield) and Compound 79 (10 mg, 0.033 mmol, 26% yield).

Peak 1 (78): retention time 1.398 min. ¹H NMR (400 MHz, d6-DMSO) δ 13.14-12.78 (m, 1H), 8.65-8.27 (m, 4H), 7.85-7.67 (m, 1H), 7.58-7.44 (m, 1H), 7.42-7.31 (m, 1H), 7.30-7.13 (m, 1H), 6.16-5.86 (m, 2H), 2.00 (s, 3H). LC-MS m/z 319 (M+H⁺).

Peak 2 (79): retention time 1.987 min. ¹H NMR (400 MHz, d6-DMSO) δ 12.98 (s, 1H), 8.59-8.37 (m, 4H), 7.76 (s, 1H), 7.51 (d, 1H), 7.37 (d, 1H), 7.22 (s, 1H), 5.99 (s, 2H), 2.00 (s, 3H). LC-MS m/z 319 (M+H⁺).

### Example 70. 7-Amino-8-(1H-indazol-4-yl)quinoxaline-6-carboxamide (108)

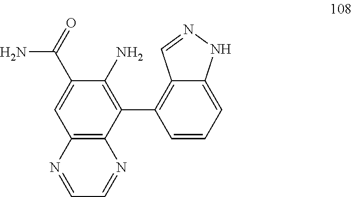 [SMILES: NC(=O)c1cc2nccnc2c(-c2cccc3[nH]ncc23)c1N]

Prepared according to Example 68 using 1H-indazol-4-ylboronic acid to give Compound 108 (186 mg, 57.1% yield). ¹H NMR (400 MHz, DMSO-d₆) δ 13.11 (s, 1H), 8.57 (d, 11H), 8.54 (d, 1H), 8.45 (s, 1H), 8.39 (s, 1H), 7.76 (s, 1H), 7.60 (d, 1H), 7.48 (dd, 1H), 7.40 (s, 1H), 7.03 (d, 1H), 6.09 (s, 2H). LC-MS m/z 305.3 [M+H⁻].

### Example 71. 7-Amino-8-(3-methyl-1H-indazol-4-yl)quinoxaline-6-carboxamide (109)

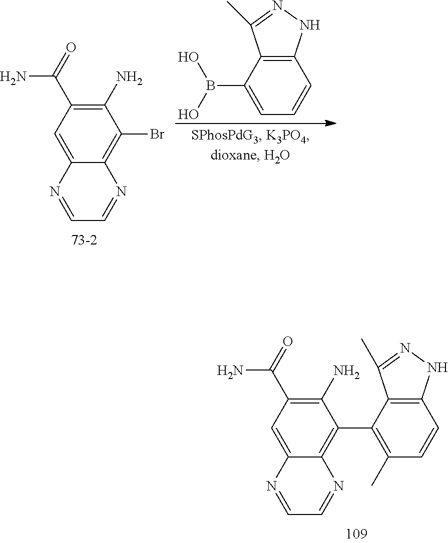

Prepared according to Example 68 using (3-methyl-2H-indazol-4-yl)boronic acid to give Compound 109 (72.3 mg, 48.0% yield). ¹H NMR (400 MHz, DMSO-d₆) δ 12.69 (s, 1H), 8.56 (m, 2H), 8.46 (s, 1H), 8.40 (s, 1H), 7.76 (s, 1H), 7.49-7.58 (m, 1H), 7.36-7.48 (m, 1H), 6.84-6.98 (m, 1H), 5.95-6.13 (m, 2H), 1.61 (s, 3H). LC-MS m/z 319.1 [M+H⁺].

### Example 72. (M)-7-Amino-8-(3-methyl-1H-indazol-4-yl)quinoxaline-6-carboxamide (118) and (P)-7-amino-8-(3-methyl-1H-indazol-4-yl)quinoxaline-6-carboxamide (119)

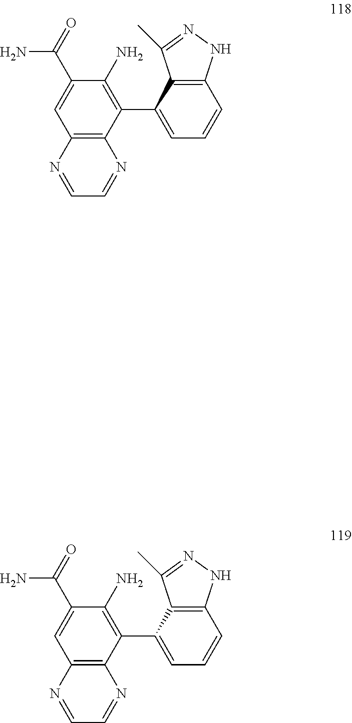

Chiral SFC separation of Compound 109 (Instrument: Waters, SFC-80Q; Daicel Chiralpak® AD (250 mm×30 mm, 10 um); mobile phase: [0.05% DEA IPA]; B %: 5%-40%) provided Compound 118 (23.8 mg) and Compound 119 (21.1 mg).

Peak 1: retention time 2.39 min. ¹H NMR (400 MHz, d6-DMSO) δ 12.60-12.79 (m, 1H), 8.57 (m, 2H), 8.46 (s, 1H), 8.41 (s, 1H), 7.69-7.85 (m, 1H), 7.50-7.60 (m, 1H), 7.40-7.50 (m, 1H), 6.84-6.99 (m, 1H), 5.93-6.15 (m, 2H), 1.62 (s, 3H). LC-MS m/z 319.3 (M+H⁺).

Peak 2: retention time 2.72 min. ¹H NMR (400 MHz, d6-DMSO) δ 12.59-12.81 (m, 1H), 8.56 (m, 2H), 8.45 (s, 1H), 8.40 (s, 1H), 7.75 (s, 1H), 7.47-7.56 (m, 1H), 7.39-7.47 (m, 1H), 6.88 (d, 1H), 5.89-6.16 (m, 2H), 1.50-1.75 (m, 3H). LC-MS m/z 319.3 (M+H⁺).

### Example 73. 7-Amino-8-(2-fluoro-5-hydroxyphenyl)quinoxaline-6-carboxamide (114)

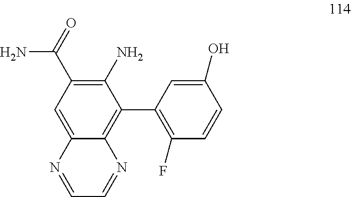 [SMILES: NC(=O)c1cc2nccnc2c(-c2cc(O)ccc2F)c1N]

Prepared according to Example 68 using (2-fluoro-5-hydroxyphenyl)boronic acid to give Compound 114 (2.8 mg). ¹H NMR (400 MHz, DMSO-d₆) δ 9.43 (s, 1H), 8.64 (s, 1H), 8.58 (s, 1H), 8.42 (br s, 1H), 8.33 (s, 1H), 7.75 (br s, 1H), 7.12 (m, 1H), 6.76-6.89 (m, 1H), 6.64 (m, 1H), 6.23 (br s, 2H). LC-MS m/z 298.9 [M+H].

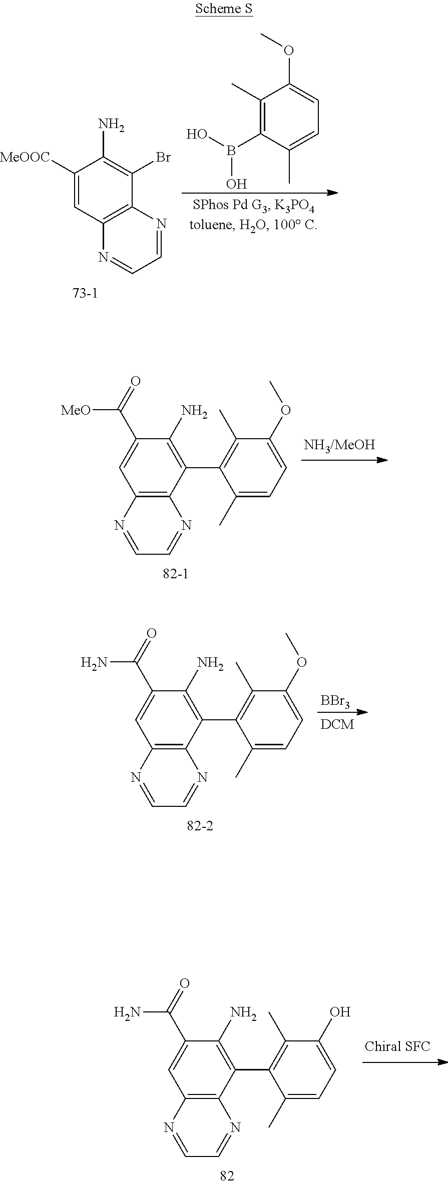

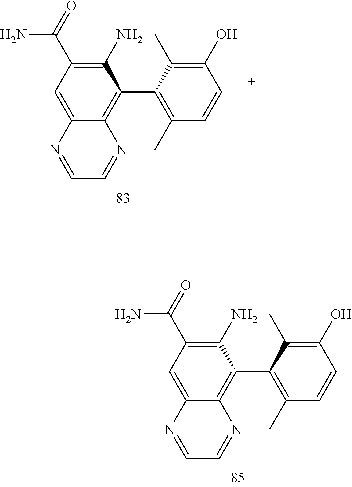

### Example 74. 7-Amino-8-(3-hydroxy-2,6-dimethylphenyl)quinoxaline-6-carboxamide (82)

Methyl 7-amino-8-(3-methoxy-2, 6-dimethylphenyl)quinoxaline-6-carboxylate (Intermediate 82-1): To a solution of methyl 7-amino-8-bromo-quinoxaline-6-carboxylate (300 mg, 1.06 mmol, 1 equiv.) and (3-methoxy-2,6-dimethylphenyl)boronic acid (383 mg, 2.13 mmol, 2 equiv.) in toluene (9 mL) and H₂O (1 mL) was added SPhos Pd G3 (82.9 mg, 0.11 mmol, 0.1 equiv.) and K₃PO₄ (0.91 g, 4.3 mmol, 4 equiv.). The mixture was degassed with N₂ and stirred at 100° C. for 16 hours. The mixture was diluted with water (10 mL), extracted with MeOH:DCM (1:9, 3×20 mL). The organic phase was dried over Na₂SO₄, filtered, and concentrated to give the residue, which was purified by flash column chromatography (FCC) (EtOAc:pet ether, 0:100 to 20:80) to give Intermediate 82-1 (100 mg, 0.21 mmol, 20% yield). LC-MS m/z 338 (M+H⁺).

7-amino-8-(3-methoxy-2,6-dimethylphenyl)quinoxaline-6-carboxamide (Intermediate 82-2): A solution of methyl 7-amino-8-(3-methoxy-2,6-dimethylphenyl)quinoxaline-6-carboxylate (140 mg, 0.41 mmol, 1 equiv.) in NH₃/MeOH (7 M, 2 mL) was stirred at 60° C. for 18 hours. The reaction mixture was concentrated directly to give 7-amino-8-(3-methoxy-2,6-dimethyl-phenyl)quinoxaline-6-carboxamide (Intermediate 82-2; 140 mg, crude) of sufficient purity for the next step. LC-MS m/z 323 (M+H⁺).

7-amino-8-(3-hydroxy-2,6-dimethylphenyl)quinoxaline-6-carboxamide (82): To a solution of 7-amino-8-(3-methoxy-2, 6-dimethylphenyl)quinoxaline-6-carboxamide (130 mg, 0.23 mmol, 58% purity, 1 equiv.) in DCM (6 mL) was added BBr₃ (0.1 mL) at 0° C. The reaction was stirred at 0° C. for 2 hours, quenched by the addition of water (20 mL) at 0° C., then concentrated to give the residue. The residue was purified by RP-HPLC to give 82 (50 mg, 0.16 mmol, 70% yield). ¹H NMR (400 MHz, DMSO-d₆) δ=9.10 (s, 1H), 8.58 (dd, 2H), 8.41 (s, 1H), 8.34 (s, 1H), 7.73 (s, 1H), 6.98 (d, 1H), 6.78 (d, 1H), 5.88 (s, 2H), 1.68 (s, 3H), 1.62 (s, 3H). LC-MS m/z 309 (M+H⁺).

### Example 75. (P)-7-Amino-8-(3-hydroxy-2,6-dimethylphenyl)quinoxaline-6-carboxamide (83) and (M)-7-amino-8-(3-hydroxy-2,6-dimethylphenyl)quinoxaline-6-carboxamide (85)

Chiral SFC separation of Compound 82 (40 mg, 0.13 mmol) (Instrument: Waters, SFC-80Q; Column: Daicel Chiralpak® AD, 30×250 mm, 10 um; Conditions: mobile phase: [0.1% NH₃H₂O EtOH]; B %: 35%-35%) provided Compounds 83 (10.7 mg, 0.034 mmol, 27% yield) and 85 (8.4 mg, 0.027 mmol, 21% yield).

Peak 1 (85): retention time 1.80 min. ¹H NMR (400 MHz, d6-DMSO) δ 9.10 (s, 1H), 8.58 (dd, 2H), 8.41 (s, 1H), 8.34 (s, 1H), 7.73 (s, 1H), 6.98 (d, 1H), 6.78 (d, 1H), 5.88 (s, 2H), 1.68 (s, 3H), 1.62 (s, 3H). LC-MS m/z 309.2 (M+H⁺).

Peak 2 (83): retention time 2.08 min. ¹H NMR (400 MHz, d6-DMSO) δ 9.10 (s, 1H), 8.58 (dd, 2H), 8.41-8.34 (m, 2H), 7.73 (s, 1H), 6.98 (d, 1H), 6.78 (d, 1H), 5.88 (s, 2H), 1.68 (s, 3H), 1.62 (s, 3H). LC-MS m/z 309.2 (M+H).

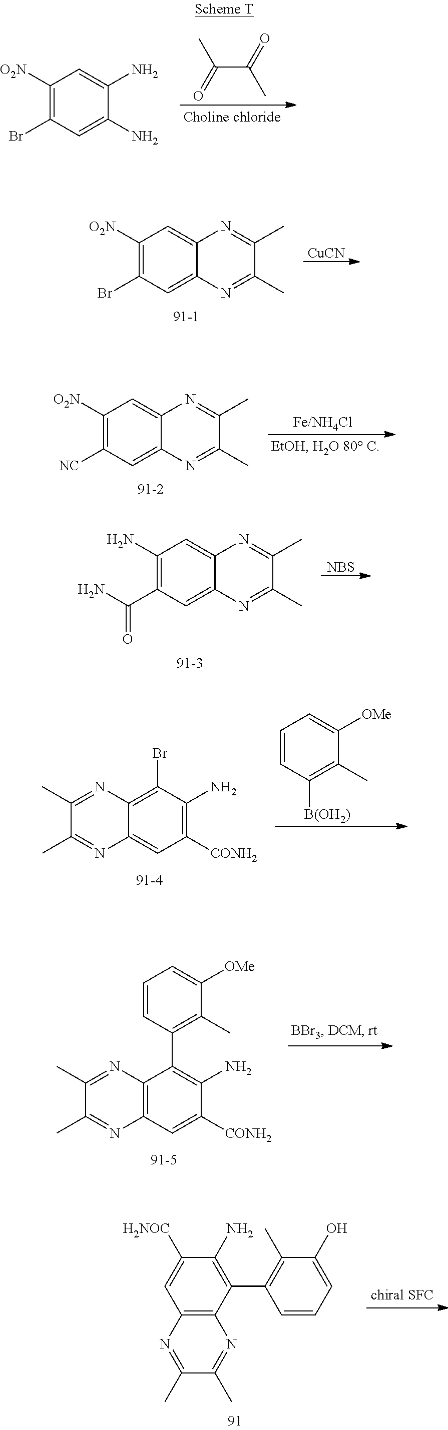

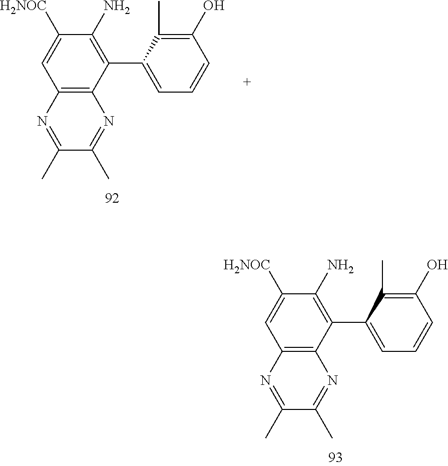

### Example 76. 7-Amino-8-(3-hydroxy-2-methylphenyl)-2,3-dimethylquinoxaline-6-carboxamide (91)

6-Bromo-2,3-dimethyl-7-nitroquinoxaline (Intermediate 91-1): A suspension of butane-2,3-dione (2.10 g, 24.4 mmol), 4-bromo-5-nitro-benzene-1,2-diamine (4.88 g, 21.0 mmol) in DCM/H₂O (1:3.33, 6 mL) was stirred at room temperature for 5 h. The reaction was quenched with water and filtered. The solid was washed with water followed by DCM/hexane (1/1) and dried to give the crude product 6-bromo-2,3-dimethyl-7-nitro-quinoxaline (Intermediate 91-1) (5.8 g, 98% Yield). ¹H NMR (400 MHz, DMSO) δ 8.68 (s, 1H), 8.52 (s, 1H), 2.74 (s, 3H), 2.72 (s, 3H); LC-MS m/z 282/284 (M+H⁺).

2,3-Dimethyl-7-nitroquinoxaline-6-carbonitrile (Intermediate 91-2): A mixture of 6-bromo-2,3-dimethyl-7-nitro-quinoxaline (2.82 g, 10.0 mmol) and CuCN (1.1 g, 12 mmol) in NMP (20 mL) was stirred at 130° C. for 2 h. The cooled reaction mixture was poured into ice-water (150 mL). The resulting suspension was filtered. The solid was washed with water and dried to give crude 2,3-dimethyl-7-nitro-quinoxaline-6-carbonitrile. This solid was suspended in DCM/MeOH (80/20, 150 ml), stirred at RT for 2 h, and filtered. The solid was washed with the same solvent. The filtrate and washes were combined and evaporated to give crude product 2,3-dimethyl-7-nitroquinoxaline-6-carbonitrile (Intermediate 91-2), LC-MS m/z 229 (M+H⁺).

7-Amino-2,3-dimethylquinoxaline-6-carboxamide (Intermediate 91-3): A mixture of 2,3-dimethyl-7-nitro-quinoxaline-6-carbonitrile (1.14 g, 5.00 mmol), Fe powder (1.4 g), and/NH₄Cl (2.7 g) in EtOH/water (30/10 mL) was stirred at 80° C. for 4 h. The mixture was filtered through Celite®, and the cake was extracted with DCM/MeOH (8/2) until no desired product remained in the cake. The washes were combined with the filtrate and concentrated to remove organic solvents. The residue was washed with water and dried to give crude 7-amino-2,3-dimethylquinoxaline-6-carboxamide (Intermediate 91-3) (0.77 g). LC-MS m/z 217 (M+H⁺).

7-Amino-8-bromo-2,3-dimethylquinoxaline-6-carboxamide(Intermediate 91-4): To a mixture of crude 7-amino-2,3-dimethylquinoxaline-6-carboxamide (0.54 g, 2.5 mmol) in DMF (5 mL) at 0° C., was added NBS (0.50 g, 2.8 mmol), and the mixture was stirred at 0-5° C. for 20 min. The reaction was quenched with water (30 mL). The resulting suspension was filtered. The solid was washed with water and dried to give crude 7-amino-8-bromo-2,3-dimethylquinoxaline-6-carboxamide (Intermediate 91-4). LC-MS m/z 295/297 (M+H⁺).

7-Amino-8-(3-methoxy-2-methylphenyl)-2,3-dimethylquinoxaline-6-carboxamide(Intermediate 91-5): A mixture of (3-methoxy-2-methyl-phenyl)boronic acid (67.0 mg, 0.404 mmol), 7-amino-8-bromo-2,3-dimethyl-quinoxaline-6-carboxamide (60.0 mg, 0.203 mmol), K3PO4 (130 mg, 0.612 mmol), dicyclohexyl-[2-(2,4,6-triisopropylphenyl)phenyl]phosphane (9.6 mg, 0.020 mmol) and [2-(2-aminophenyl)phenyl]-methylsulfonyloxy-palladium dicyclohexyl-[2-(2,6-dimethoxyphenyl)phenyl]phosphane (15.0 mg, 0.0192 mmol) in toluene (2 mL) and water (0.5 mL) was stirred under N2 at 110° C. overnight. The reaction mixture was diluted with water and extracted with EtOAc (2×), the EtOAc extract was washed with brine, dried over Na₂SO₄, filtered and concentrated to give crude 7-amino-8-(3-methoxy-2-methylphenyl)-2,3-dimethylquinoxaline-6-carboxamide (Intermediate 91-5), which was used without further purification. LC-MS m/z 337 (M+H⁺).

7-Amino-8-(3-hydroxy-2-methylphenyl)-2,3-dimethylquinoxaline-6-carboxamide (91): Crude 7-amino-8-(3-methoxy-2-methylphenyl)-2,3-dimethylquinoxaline-6-carboxamide was dissolved in DCM (5 mL). Then BBr₃ (0.7 mL, 1M in DCM) was added at RT. The resulting mixture was stirred at RT overnight, quenched with NaHCO₃, and extracted with EtOAc (2×). The extract was evaporated, and the residue was purified by prep-HPLC to give 7-amino-8-(3-hydroxy-2-methyl-phenyl)-2,3-dimethyl-quinoxaline-6-carboxamide (91) (33.0 mg, 50.4% Yield). ¹H NMR (400 MHz, DMSO) δ 9.30 (d, 1H), 8.22 (s, 1H), 8.16 (d, 1H), 7.55 (s, 1H), 7.03 (t, 1H), 6.79 (d, 1H), 6.46 (d, 1H), 5.70 (s, 2H), 2.49 (d, 3H), 2.36 (d, 3H), 1.63 (d, 3H); LC-MS m/z 323 (M+H⁺).

### Example 77. (P)-7-Amino-8-(3-hydroxy-2-methylphenyl)-2,3-dimethylquinoxaline-6-carboxamide (92) and (M)-7-Amino-8-(3-hydroxy-2-methylphenyl)-2,3-dimethylquinoxaline-6-carboxamide (93)

Chiral SFC separation of Compound 91 (25 mg) (Instrument: Waters SFC Prep 150 Mgm; Column: Chiralpak® AD, 30×150 mm, 5 um; Conditions: Isocratic at 35% methanol with 65% CO₂; Flow Rate: 100 mL/min) provided Compound 92 (10 mg) and Compound 93 (10 mg).

Peak 1: retention time 1.96 min. ¹H NMR (400 MHz, DMSO) δ 9.29 (d, 1H), 8.22 (s, 1H), 8.16 (t, 1H), 7.54 (s, 1H), 7.03 (t, 1H), 6.79 (d, J=8.0 Hz, 1H), 6.46 (d, 1H), 5.70 (s, 2H), 2.49 (d, 3H), 2.36 (d, 3H), 1.63 (d, 3H). LC-MS m/z 323 (M+H⁺).

Peak 2: retention time 2.78 min. ¹H NMR (400 MHz, DMSO) δ 9.30 (d, 1H), 8.22 (s, 1H), 8.16 (d, 1H), 7.55 (s, 1H), 7.03 (t, 1H), 6.79 (d, 1H), 6.46 (d, 1H), 5.70 (s, 2H), 2.49 (d, 3H), 2.36 (d, 3H), 1.63 (d, 3H). LC-MS m/z 323 (M+H⁺).

### Example 78. 7-Amino-8-(3-hydroxy-2,6-dimethylphenyl)-2,3-dimethylquinoxaline-6-carboxamide (94)

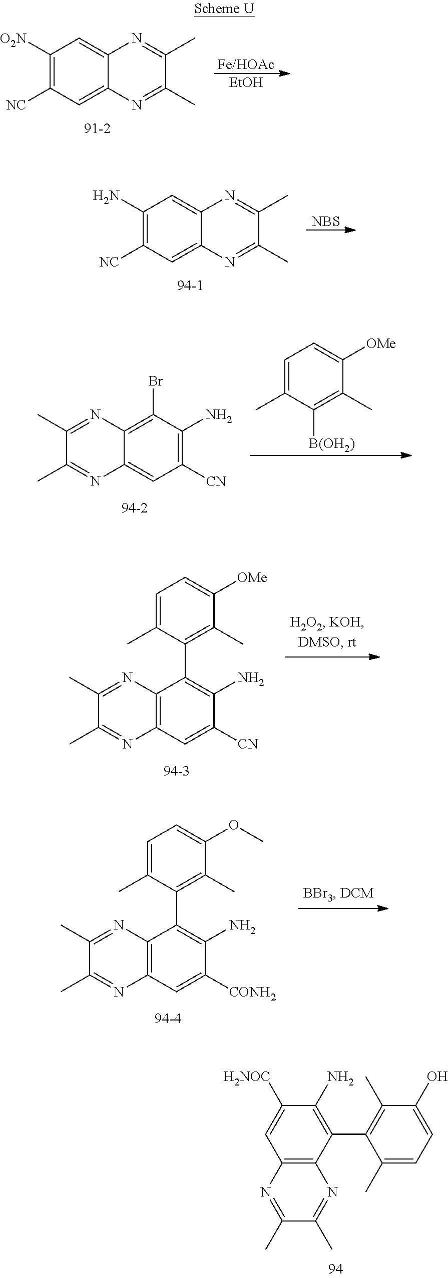

7-Amino-2,3-dimethylquinoxaline-6-carbonitrile (Intermediate 94-1): To a mixture of 2,3-dimethyl-7-nitro-quinoxaline-6-carbonitrile (1.85 g, 8.11 mmol) and HOAc (4 mL) in EtOH (30 mL) was added Fe powder (1.1 g). The resulting mixture was stirred at 70° C. for 1 h and filtered through Celite®. The cake was washed with MeOH/DCM (20/80) until no product remained in the cake. The filtrate and washes were combined and concentrated to give crude 7-amino-2,3-dimethyl-quinoxaline-6-carbonitrile (Intermediate 94-1) (1.23 g, 76.5% Yield). LC-MS m/z 199 (M+H⁺), containing approximately 10% hydrolyzed side product.

7-Amino-8-bromo-2,3-dimethylquinoxaline-6-carbonitrile (Intermediate 94-2): To a mixture of 7-amino-2,3-dimethyl-quinoxaline-6-carbonitrile (1.10 g, 5.55 mmol) in DMF (10 mL) at 0° C. was added NBS (1.01 g, 5.67 mmol). The mixture was stirred at 0-5° C. for 20 min and quenched with water (50 mL). The resulting suspension was filtered. The solids were washed with water and dried to give crude 7-amino-8-bromo-2,3-dimethyl-quinoxaline-6-carbonitrile (Intermediate 94-2) (1.30 g, 84.5% Yield). LC-MS m/z 277/279 (M+H⁺).

7-Amino-8-(3-methoxy-2,6-dimethylphenyl)-2,3-dimethylquinoxaline-6-carbonitrile (Intermediate 94-3): A mixture of (3-methoxy-2,6-dimethyl-phenyl)boronic acid (0.380 g, 2.11 mmol), 7-amino-8-bromo-2,3-dimethyl-quinoxaline-6-carbonitrile (0.250 g, 0.902 mmol), dicyclohexyl-[2-(2,6-dimethoxyphenyl)phenyl]phosphane (40.0 mg, 0.0974 mmol), [2-(2-aminophenyl)phenyl]-methylsulfonyloxy-palladium dicyclohexyl-[2-(2,6-dimethoxyphenyl)phenyl]phosphane (70.0 mg, 0.0897 mmol) and potassium phosphate (650 mg, 3.06 mmol) in toluene (6 mL) and water (1.5 mL) was stirred under N2 at 110° C. overnight. The reaction mixture was diluted with water and extracted with EtOAc (2×); the combined extracts were washed with brine, dried over Na₂SO₄, filtered and concentrated. The residue was purified through a silica gel plug to give 7-amino-8-(3-methoxy-2,6-dimethyl-phenyl)-2,3-dimethyl-quinoxaline-6-carbonitrile (Intermediate 94-3) (41.0 mg, 13.7% Yield). LC-MS n/z 333 (M+H⁺).

7-Amino-8-(3-methoxy-2,6-dimethylphenyl)-2,3-dimethylquinoxaline-6-carboxamide (Intermediate 94-4): Above obtained compound 7-amino-8-(3-methoxy-2,6-dimethylphenyl)-2,3-dimethyl-quinoxaline-6-carbonitrile (41.0 mg, 0.12 mmol) was dissolved in DMSO (5 mL), treated with NaOH (6M, 1 mL) and H₂O₂ (30%, 0.4 mL), and stirred at RT until completion. The mixture was neutralized to pH 7-8, extracted with EtOAc (2×). The extract was evaporated to dryness, and the residue was washed with DCM/hexane (1/1) to give crude 7-amino-8-(3-methoxy-2,6-dimethylphenyl)-2,3-dimethyl-quinoxaline-6-carboxamide (Intermediate 94-4). LC-MS m/z 351 (M+H⁺).

7-Amino-8-(3-hydroxy-2,6-dimethylphenyl)-2,3-dimethylquinoxaline-6-carboxamide (94): The material obtained from previous step was dissolved in DCM (5 mL), and then BBr3 (0.7 mL, 1M in DCM) was dropped in at RT. The resulting mixture was stirred at RT overnight, quenched with NaHCO₃, extracted with EtOAc (2×). The extract was concentrated and the residue was purified by prep HPLC to give 7-amino-8-(3-hydroxy-2,6-dimethylphenyl)-2,3-dimethyl-quinoxaline-6-carboxamide (94) (16.0 mg, 38.6% over 2 steps). H NMR (400 MHz, DMSO) δ 9.04 (s, 1H), 8.21 (d, 2H), 7.57-7.53 (m, 1H), 6.91 (d, 1H), 6.70 (d, 1H), 5.64 (s, 2H), 2.49 (s, 3H), 2.35 (s, 3H), 1.62 (s, 3H), 1.56 (s, 3H). LC-MS m/z 337 (M+H⁺).

### Example 79. Preparation of methyl 7-amino-8-bromo-3-(trifluoromethyl)quinoxaline-6-carboxylate (96-3A) and methyl 7-amino-8-bromo-2-(trifluoromethyl)quinoxaline-6-carboxylate (96-3B)

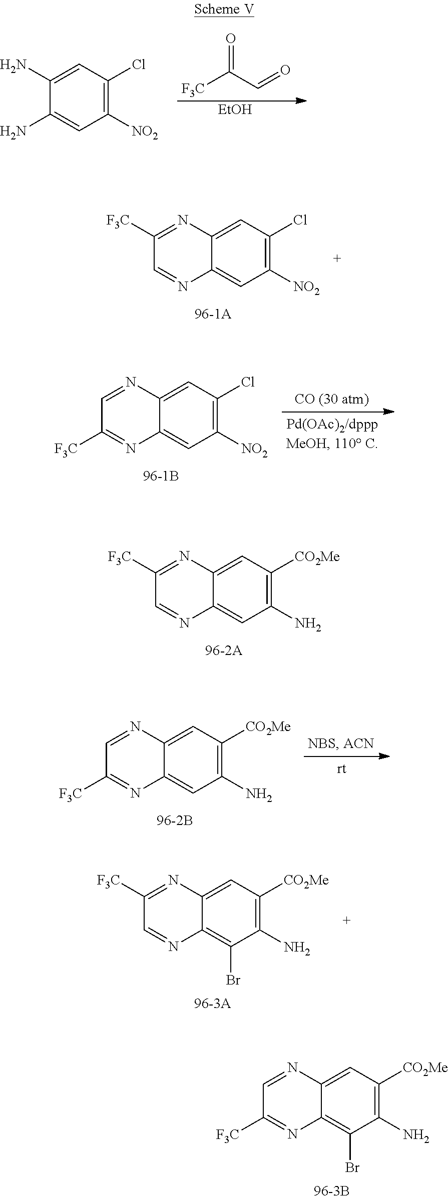

Step 1: 7-Chloro-6-nitro-2-(trifluoromethyl)quinoxaline and 6-chloro-7-nitro-2-(trifluoromethyl)quinoxaline (Intermediates 96-1A and 96-1B): A solution of 3,3-dibromo-1,1,1-trifluoropropan-2-one (5.9 g, 21.8 mmol) and sodium acetate (7.5 g, 91.3 mmol) in ethanol (50 mL) and water (40 mL) was heated at 98° C. for 30 min, followed by addition of 4-chloro-5-nitro-benzene-1,2-diamine (1.88 g, 10.0 mmol). The reaction mixture was stirred at RT for 17 h then concentrated to remove most volatiles and filtered. The solid was washed with water and dried to afford crude Intermediate 96-1A (1.6 g, 9:1 ratio of regioisomers). ¹H NMR (400 MHz, DMSO) δ 9.64 (t, 1H), 9.10 (d, 1H), 8.87-8.82 (m, 1H). LC-MS m/z 278 (M+H⁺). The filtrate was extracted with EtOAc (3×) to give 600 mg of a mixture isomers (about 1:1 ratio based on 1H NMR). ¹H NMR (400 MHz, DMSO) δ 9.66 (d, 1H), 9.64 (s, 1H), 9.14 (d, 1H), 9.10 (s, 1H), 8.85 (d, 1H), 8.80 (d, 1H). LC-MS m/z 278 (M+H⁺).

Step 2: Methyl 7-amino-3-(trifluoromethyl)quinoxaline-6-carboxylate and methyl 7-amino-2-(trifluoromethyl)quinoxaline-6-carboxylate (Intermediates 96-2A and 96-2B): The major isomer (crude 1.5 g) from Step 1 was mixed with Pd(OAc)2 (390 mg), dppp (650 mg), and TEA (4.3 g) in MeOH (80 mL). The mixture was stirred under CO (30-35 atm) at 110° C. for 2 days. The mixture was filtered through Celite® and evaporated. The crude residue was purified by silica gel (0-100% EtOAc in hexane), to give methyl 7-amino-3-(trifluoromethyl)quinoxaline-6-carboxylate (0.54 g, 37% Yield) and minor carboxylic acid side product (0.27 g). 1H NMR (400 MHz, DMSO) δ 9.14 (d, 1H), 8.56 (t, 1H), 7.34 (s, 2H), 7.28 (d, 1H), 3.93 (d, 3H). LC-MS m/z 272 (M+H⁺). The mixture of isomers (0.6 g) from Step 1 was treated in a similar manner, and the crude product was purified by silica gel (0-50% EtOAc in hexane) to give two isomers confirmed by small molecule x-ray crystallography.

Peak 1: methyl 7-amino-2-(trifluoromethyl)quinoxaline-6-carboxylate (121 mg, 21% yield), ¹H NMR (400 MHz, DMSO) δ 8.96 (d, 1H), 8.54 (d, 1H), 7.25 (d, 1H), 7.21 (s, 2H), 3.95 (s, 2H). LC-MS m/z 272 (M+H).

Peak 2: methyl 7-amino-3-(trifluoromethyl)quinoxaline-6-carboxylate (230 mg, 39%), ¹H NMR (400 MHz, DMSO) δ 9.12 (d, 1H), 8.56-8.51 (m, 1H), 7.33 (s, 2H), 7.26 (d, 1H), 3.94 (s, 1H). LC-MS m/z 272 (M+H⁺).

Step 3: Methyl 7-amino-8-bromo-3-(trifluoromethyl)quinoxaline-6-carboxylate and methyl 7-amino-8-bromo-2-(trifluoromethyl)quinoxaline-6-carboxylate (Intermediates 96-3A and 96-3B): To a solution of methyl 7-amino-3-(trifluoromethyl)quinoxaline-6-carboxylate (0.54 g, 2.0 mmol) in ACN (20 mL) was added NBS (1.1 eq) at RT and stirred at RT for 30 min. A suspension formed. The mixture was filtered to give 476 mg of yellow product; the mother liquor was concentrated to give an additional 126 mg of methyl 7-amino-8-bromo-3-(trifluoromethyl)quinoxaline-6-carboxylate (total 86% Yield). ¹H NMR (400 MHz, DMSO) δ 9.32 (t, 1H), 8.62 (t, 1H), 7.44 (s, 2H), 3.97 (s, 3H). LC-MS m/z 350/352 (M+H⁺).

The red isomer (121 mg) obtained in Step 2 was treated with NBS (1.1 eq) in ACN (5 mL) at rt for 30 min and a red solution formed, which was evaporated and residue purified by silica gel (0-40% EtOAc in hexane), to give 91 mg of methyl 7-amino-8-bromo-2-(trifluoromethyl)quinoxaline-6-carboxylate. ¹H NMR (400 MHz, DMSO) δ 9.11 (d, 1H), 8.60 (t, 1H), 7.31 (s, 2H), 3.97 (s, 3H). LC-MS m/z 350/352 (M+H⁺).

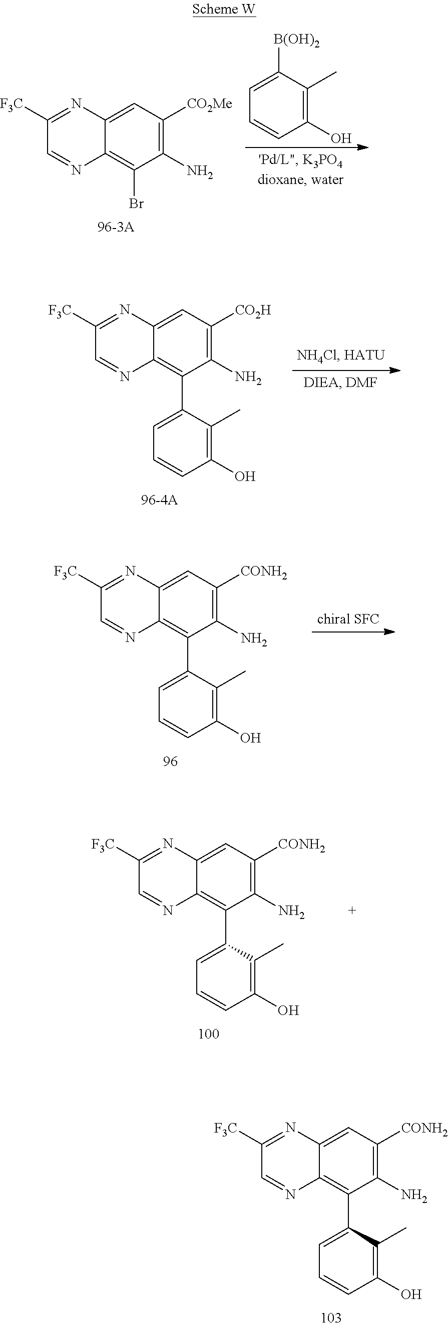

### Example 80. 7-Amino-8-(3-hydroxy-2-methylphenyl)-3-(trifluoromethyl)quinoxaline-6-carboxamide (96)

7-Amino-8-(3-hydroxy-2-methylphenyl)-3-(trifluoromethyl)quinoxaline-6-carboxylic acid (Intermediate 96-4A): A mixture of methyl 7-amino-8-bromo-3-(trifluoromethyl)quinoxaline-6-carboxylate (120 mg, 0.343 mmol), (3-hydroxy-2-methylphenyl)boronic acid (107.0 mg, 0.7042 mmol), potassium phosphate (0.400 g, 1.88 mmol), dicyclohexyl-[2-(2,6-dimethoxyphenyl)phenyl]phosphane (15.0 mg, 0.0365 mmol) and [2-(2-aminophenyl)phenyl]-methylsulfonyloxy-palladium dicyclohexyl-[2-(2,6-dimethoxyphenyl)phenyl]phosphane (26.0 mg, 0.0333 mmol) in 1,4-dioxane (4.0 mL) and water (0.8 mL) was stirred under N₂ at 110° C. overnight. The reaction mixture was purified by silica gel (0-100% EtOAc in Hexanes) to give 7-amino-8-(3-hydroxy-2-methylphenyl)-3-(trifluoromethyl)quinoxaline-6-carboxylic acid (81.0 mg, 65.0% Yield). LC-MS m/z 364 (M+H⁺).

7-Amino-8-(3-hydroxy-2-methylphenyl)-3-(trifluoromethyl)quinoxaline-6-carboxamide (96): A mixture of 7-amino-8-(3-hydroxy-2-methylphenyl)-3-(trifluoromethyl)quinoxaline-6-carboxylic acid (81 mg, 0.22 mmol), ammonium chloride (60 mg, 1.12 mmol), DIEA (140 mg, 1.08 mmol), and HATU (150 mg, 0.394 mmol) in DMF (3 mL) was stirred at RT for 30 min. The mixture was purified by prep HPLC, to give 7-amino-8-(3-hydroxy-2-methyl-phenyl)-3-(trifluoromethyl)quinoxaline-6-carboxamide (56 mg, 69%). ¹H NMR (400 MHz, DMSO) δ 9.44 (d, 1H), 9.04 (d, 1H), 8.49 (s, 2H), 7.90 (s, 1H), 7.11 (d, 1H), 6.89 (d, 1H), 6.56 (d, 1H), 6.42 (s, 2H), 1.71 (d, 3H). LC-MS m/z 363 (M+H⁺).

### Example 81. (P)-7-Amino-8-(3-hydroxy-2-methylphenyl)-3-(trifluoromethyl)quinoxaline-6-carboxamide (100) and (M)-7-Amino-8-(3-hydroxy-2-methylphenyl)-3-(trifluoromethyl)quinoxaline-6-carboxamide (103)

Chiral SFC separation of compound 96 (50 mg) (Instrument: Waters SFC Prep 150 Mgm; Column: Chiralcel® IC, 30×150 mm, 5 um; Conditions: Isocratic at 20% methanol with 80% CO2; Flow Rate: 100 mL/min) provided Compound 100 (22 mg) and Compound 103 (19 mg).

Peak 1: retention time 1.63 min. ¹H NMR (400 MHz, DMSO) δ 9.44 (d, 1H), 9.04 (d, 1H), 8.49 (s, 2H), 7.90 (s, 1H), 7.12 (t, 1H), 6.89 (d, 1H), 6.56 (d, 1H), 6.42 (s, 2H), 1.71 (s, 3H). LC-MS m/z 363 (M+H⁺).

Peak 2: retention time 1.93 min. ¹H NMR (400 MHz, DMSO) δ 9.44 (d, 1H), 9.04 (s, 1H), 8.49 (s, 2H), 7.90 (s, 1H), 7.12 (t, 1H), 6.89 (d, 1H), 6.56 (d, 1H), 6.42 (s, 2H), 1.71 (s, 3H). LC-MS m/z 363 (M+H⁺).

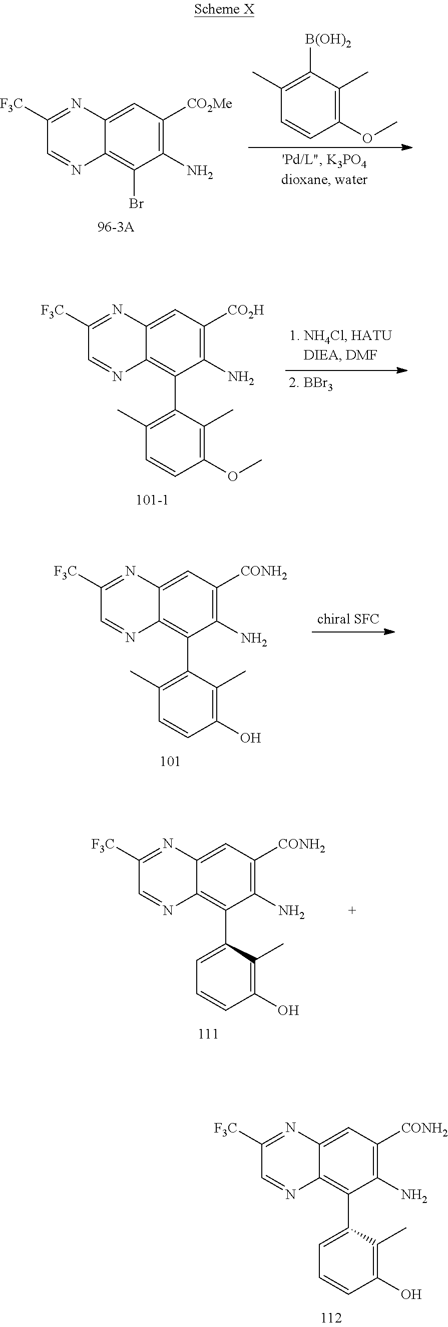

### Example 82. 7-Amino-8-(3-hydroxy-2,6-dimethylphenyl)-3-(trifluoromethyl)quinoxaline-6-carboxamide (101)

7-Amino-8-(3-methoxy-2,6-dimethylphenyl)-3-(trifluoromethyl) quinoxaline-6-carboxylic acid (Intermediate 101-1): A mixture of (3-methoxy-2,6-dimethylphenyl)boronic acid (250.0 mg, 1.389 mmol), methyl 7-amino-8-bromo-3-(trifluoromethyl)quinoxaline-6-carboxylate (163.0 mg, 0.4656 mmol), K₃PO₄ (526.0 mg, 2.5 mmol), dicyclohexyl-[2-(2,4,6-triisopropylphenyl)phenyl]phosphane (24 mg, 0.045 mmol), and [2-(2-aminophenyl)phenyl]-methylsulfonyloxy-palladium; dicyclohexyl-[2-(2,6-dimethoxyphenyl)phenyl]phosphane (36.0 mg, 0.046 mmol) in dioxane (4 mL) and water (1 mL) was stirred under N₂ at 110° C. overnight. The reaction mixture was diluted with water, acidified to pH 3-4, and extracted with EtOAc (2×). The EtOAc extract was washed with brine and concentrated. The residue was purified by silica gel column (0-100% EtOAc in hexane, to give 7-amino-8-(3-methoxy-2,6-dimethylphenyl)-3-(trifluoromethyl)quinoxaline-6-carboxylic acid (67.0 mg, 36.8% Yield). LC-MS m/z 392 (M+H⁺).

7-Amino-8-(3-hydroxy-2,6-dimethylphenyl)-3-(trifluoromethyl) quinoxaline-6-carboxamide (101): A mixture of above 7-amino-8-(3-methoxy-2,6-dimethylphenyl)-3-(trifluoromethyl)quinoxaline-6-carboxylic acid (67.0 mg, 0.171 mmol), NH4Cl (50 mg, 0.92 mmol), HATU (120 mg, 0.32 mmol), and DIEA (130 mg, 1.0 mmol) in DMF (2 mL) was stirred at rt for 30 min. The reaction mixture was treated with NaOH (3M) to a pH of greater than 14, extracted with EtOAc (2×); the EtOAc extract was washed with brine, and concentrated. The residue was purified by silica gel column (0-100% EtOAc in hexane) to give 7-amino-8-(3-methoxy-2,6-dimethylphenyl)-3-(trifluoromethyl)quinoxaline-6-carboxamide (56.0 mg, 83.8% Yield). LC-MS m/z 391 (M+H⁺).

The material was dissolved in DCM (3 mL) and treated with BBr₃ (1M in DCM, 0.6 mL). The mixture was stirred until reaction completion and quenched with NaHCO₃, extracted with EtOAc (3×). The extract was evaporated, and the residue was purified by prep-HPLC, to give 7-amino-8-(3-hydroxy-2,6-dimethylphenyl)-3-(trifluoromethyl)quinoxaline-6-carboxamide (45.0 mg, 69.8% Yield). ¹H NMR (400 MHz, DMSO) δ 9.18 (s, 1H), 9.04 (d, 1H), 8.51 (t, 2H), 7.91 (s, 1H), 7.00 (d, 1H), 6.84-6.77 (m, 1H), 6.38 (s, 2H), 1.70 (s, 3H), 1.64 (s, 3H). LC-MS m/z 377 (M+H⁺).

### Example 83. (M)-7-Amino-8-(3-hydroxy-2,6-dimethylphenyl)-3-(trifluoromethyl)quinoxaline-6-carboxamide (111) and (P)-7-Amino-8-(3-hydroxy-2,6-dimethylphenyl)-3-(trifluoromethyl)quinoxaline-6-carboxamide (112)

Chiral SFC separation of Compound 101 (40 mg) (Instrument: Waters SFC Prep 150 Mgm; Column: Chiralcel® OD, 30×150 mm, 5 um; Conditions: Isocratic at 25% methanol with 75% CO2; Flow Rate: 100 mL/min) provided Compound 111 (16 mg) and Compound 112 (16 mg).

Peak 1: retention time 1.85 min, ¹H NMR (400 MHz, DMSO) δ 9.10 (s, 1H), 8.97 (d, 1H), 8.43 (d, 2H), 7.83 (s, 1H), 6.93 (d, J=8.1 Hz, 1H), 6.73 (d, 1H), 6.31 (s, 2H), 1.62 (d, 3H), 1.57 (d, 3H). LC-MS m/z 377 (M+H⁺).

Peak 2: retention time 2.23 min, ¹H NMR (400 MHz, DMSO) δ 9.07 (s, 1H), 8.97 (d, 1H), 8.51-8.37 (m, 2H), 7.84 (s, 1H), 6.93 (d, 1H), 6.77-6.69 (m, 1H), 6.31 (s, 2H), 1.63 (d, 3H), 1.57 (d, 3H). LC-MS m/z 377 (M+H⁺).

### Example 84. 7-Amino-8-(3-hydroxy-2-methyl-phenyl)-2,3-dimethoxy-quinoxaline-6-carbonitrile (104)

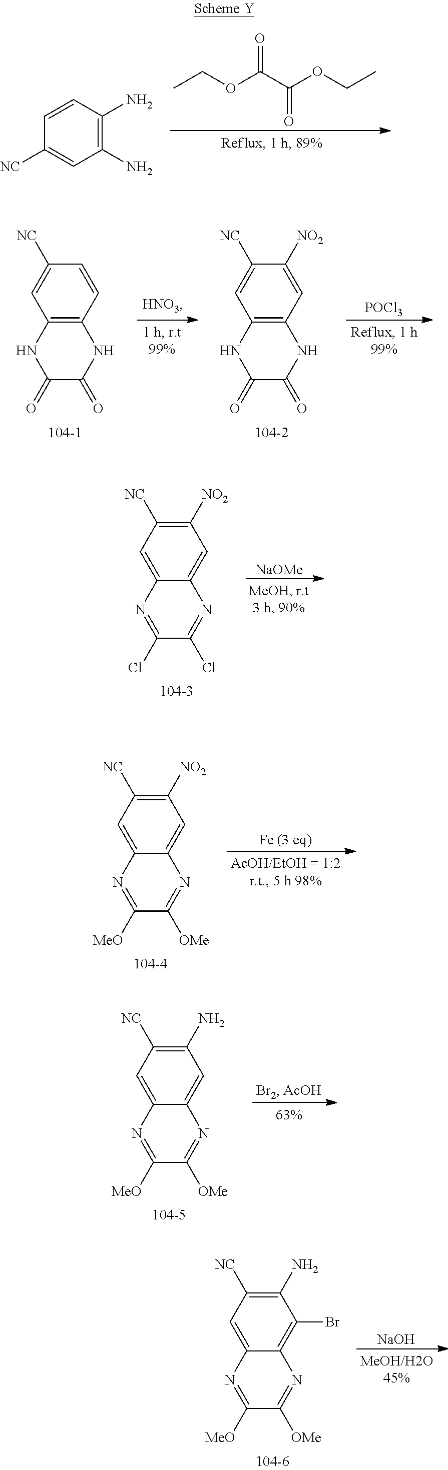

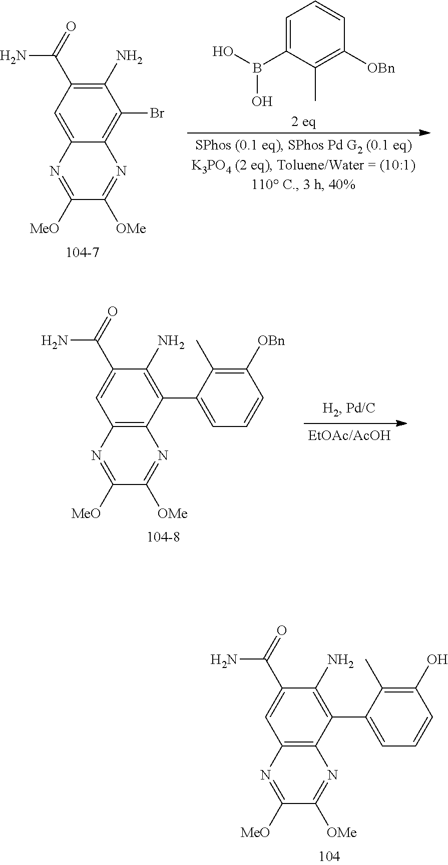

2,3-dioxo-1,2,3,4-tetrahydroquinoxaline-6-carbonitrile (Intermediate 104-1): A mixture of 3,4-diaminobenzonitrile (2.0 g, 15.0 mmol, 1.0 equiv.) and diethyl oxalate (20 mL), was heated to reflux for 1 h and then cooled. The precipitate of 2,3-dioxo-1,4-dihydroquinoxaline-6-carbonitrile was collected by vacuum filtration and washed with ethanol to give the title compound (2.5 g, 89% yield) which was used without further purification. ¹H NMR (400 MHz, DMSO) δ 12.24 (s, 1H), 12.11 (s, 1H), 7.57-7.44 (m, 1H), 7.40 (s, 1H), 7.22 (dd, 1H).

7-nitro-2,3-dioxo-1,4-dihydroquinoxaline-6-carbonitrile (Intermediate 104-2): 2,3-Dioxo-1,4-dihydroquinoxaline-6-carbonitrile (1.2 g, 6.4 mmol, 1.0 equiv.) was dissolved in nitric acid (10 mL) and stirred for 1 h. The reaction mixture was poured slowly into 200 ml of ice water. The resulting precipitate was collected by filtration to give the title compound 7-nitro-2,3-dioxo-1,4-dihydroquinoxaline-6-carbonitrile (1.48 g, 99% yield) which was used without further purification. LC-MS m/z [M+H]⁺=233.20.

2,3-dichloro-7-nitroquinoxaline-6-carbonitrile (Intermediate 104-3): 7-Nitro-2,3-dioxo-1,4-dihydroquinoxaline-6-carbonitrile (350 mg, 1.5 mmol, 1 equiv.) was added slowly to phosphoryl trichloride (5 mL) followed by several drops of DMF. The reaction mixture was stirred at 110° C. for one hour. The cooled reaction mixture was poured slowly into 100 ml of ice water and extracted with dichloromethane (3×). The combined organics were washed sequentially with Na₂SO₄ and brine, dried over anhydrous sodium sulfate, decanted and concentrated to give the title compound, 2,3-dichloro-7-nitroquinoxaline-6-carbonitrile (402 mg, 99% yield) which was used without further purification. ¹H NMR (400 MHz, DMSO) δ 9.08 (s, 1H), 9.05 (s, 1H).

2,3-dimethoxy-7-nitroquinoxaline-6-carbonitrile (Intermediate 104-4): 2,3-Dichloro-7-nitroquinoxaline-6-carbonitrile (400 mg, 1.48 mmol, 1 equiv.) was dissolved in methanol (10 mL) and treated with sodium methoxide (300 mg, 6 mmol, 4 equiv.). The reaction was stirred at room temperature for 3 h. Volatiles were removed under reduced pressure. Water (200 ml) was added to the mixture. The precipitate was filtered then washed with water to give the title product 2,3-dimethoxy-7-nitroquinoxaline-6-carbonitrile (350 mg, 90% yield) which was used without further purification. ¹H NMR (400 MHz, DMSO) δ 8.59 (d, 1H), 8.51 (d, 1H), 4.13 (s, 6H). LC-MS m/z [M+H]⁺=261.15.

7-amino-2,3-dimethoxyquinoxaline-6-carbonitrile (Intermediate 104-5): 2,3-Dimethoxy-7-nitroquinoxaline-6-carbonitrile (150 mg, 0.58 mmol, 1.0 equiv.) was dissolved in ethanol (3 mL) and treated with iron (97 mg, 1.7 mmol, 3.0 equiv.) and acetic acid (1.5 mL). The mixture was stirred at room temperature for 5 h. Volatiles were removed under reduced pressure. Water (100 ml) was added. The resulting precipitate was collected by vacuum filtration to give the title product 7-amino-2,3-dimethoxyquinoxaline-6-carbonitrile (130 mg, 98% yield) which was used without further purification. LC-MS m/z [M+H]⁺=231.15.

7-amino-8-bromo-2,3-dimethoxyquinoxaline-6-carbonitrile (Intermediate 104-6): 7-Amino-2,3-dimethoxyquinoxaline-6-carbonitrile (177 mg, 0.77 mmol, 1.0 equiv.) was dissolved in acetic acid (5 mL) then treated with bromine (0.06 ml, 1.15 mmol, 1.5 equiv.). The reaction was stirred 3 h at room temperature then quenched by the addition of 1N solution of NaOH. The reaction mixture was extracted with ethyl acetate (50 mL×3), dried over Na₂SO₄, decanted, and concentrated under reduced pressure. The crude material was purified by silica gel column chromatography (EtOAc 20%-40% in Hexane) to give 7-amino-8-bromo-2,3-dimethoxy-quinoxaline-6-carbonitrile (150 mg, 63% yield). LC-MS m/z [M+H]⁺=309.00

7-amino-8-bromo-2,3-dimethoxyquinoxaline-6-carboxamide (Intermediate 104-7): 7-Amino-8-bromo-2,3-dimethoxyquinoxaline-6-carbonitrile (50 mg, 0.16 mmol, 1.0 equiv.) dissolved in methanol (0.5 mL), hydrogen peroxide (0.5 mL), and water (0.5 mL). Sodium hydroxide (19 mg, 0.49 mmol, 3.0 equiv.) was added, and the reaction was stirred at 55° C. overnight. The cooled reaction mixture was extracted with ethyl acetate (20 mL×3), dried over Na₂SO₄, decanted and concentrated. The crude material was purified by silica gel column chromatography (EtOAc 20%-40% in Hexane) to give the title compound 7-amino-8-bromo-2,3-dimethoxyquinoxaline-6-carboxamide (24 mg, 45% yield). LC-MS m/z [M+H]⁺=329.10.

7-amino-8-(3-benzyloxy-2-methyl-phenyl)-2,3-dimethoxy-quinoxaline-6-carboxamide (Intermediate 104-8): 7-Amino-8-bromo-2,3-dimethoxyquinoxaline-6-carboxamide (24 mg, 0.07 mmol, 1.0 equiv.) SPhos (3 mg, 0.007 mol, 0.1 equiv.), SPhos Pd G2 (6.6 mg, 0.007 mol, 0.1 equiv.) and potassium phosphate (20 mg, 0.15 mol, 2.0 equiv.) were combined with toluene (1.0 mL) and water (0.1 mL) and then stirred under nitrogen environment for 3 h at 110° C. The cooled reaction mixture was then extracted with ethyl acetate (10 mL×3), dried over Na₂SO₄, decanted, and concentrated. The crude material was purified by silica gel column chromatography (EtOAc 40% in Hexane) to give the title compound 7-amino-8-(3-benzyloxy-2-methyl-phenyl)-2,3-dimethoxy-quinoxaline-6-carboxamide (13 mg, 40% yield). LC-MS m/z [M+H]⁺=445.20.

7-amino-8-(3-hydroxy-2-methyl-phenyl)-2,3-dimethoxy-quinoxaline-6-carbonitrile (104): To a solution of 7-amino-8-(3-benzyloxy-2-methyl-phenyl)-2,3-dimethoxy-quinoxaline-6-carbonitrile (10 mg, 0.02 mmol, 1.0 equiv.) in EtOAc (0.2 mL) and AcOH (0.2 mL) was added palladium on carbon (5 mg, 0.2 equiv.). The mixture was stirred under hydrogen gas for 1 h. The reaction mixture was filtered. The filtrate was concentrated, and the residue was purified by prep-HPLC give the title product 7-amino-8-(3-hydroxy-2-methyl-phenyl)-2,3-dimethoxy-quinoxaline-6-carbonitrile (3 mg, 38% yield). ¹H NMR (400 MHz, DMSO) δ 9.36 (s, 1H), 7.92 (d, 1H), 7.04 (t, 1H), 6.80 (d, 1H), 6.50 (d, 1H), 4.95 (s, 2H), 3.91 (s, 3H), 3.59 (s, 3H), 1.68 (s, 3H). LC-MS m/z [M+H]⁺=337.20.

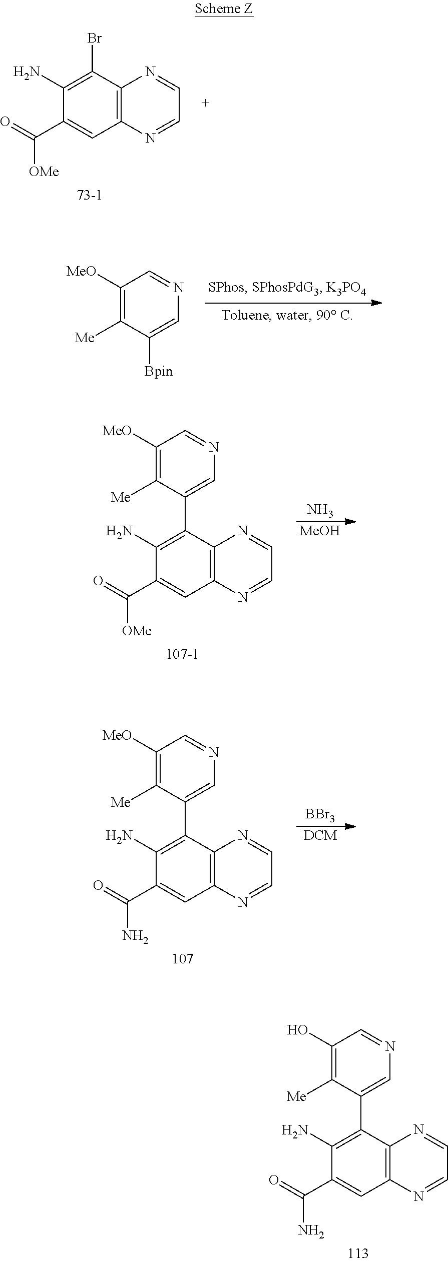

### Example 85. 7-amino-8-(5-methoxy-4-methyl pyridin-3-yl)quinoxaline-6-carboxamide (107)

Methyl 7-amino-8-(5-methoxy-4-methylpyridin-3-yl)quinoxaline-6-carboxylate (Intermediate 107-1): Prepared according to Example 74 using 3-methoxy-4-methyl-5-(4,4,5,5-tetramethyl-1,3,2-dioxaborolan-2-yl)pyri dine to give the title compound (166 mg, 88% yield) which was used without further purification in the next step. LC-MS m/z 325.2 (M+H⁺).

7-amino-8-(5-methoxy-4-methylpyridin-3-yl)quinoxaline-6-carboxamide (107): Methyl 7-amino-8-(5-methoxy-4-methylpyridin-3-yl)quinoxaline-6-carboxylate (158 mg, 0.490 mmol, 1 equiv.) was dissolved in a solution of NH₃ in MeOH (7 M, 4 mL, 28 mmol, 57 equiv.) in a microwave vial. The mixture was heated to 140° C. (approximately 14 bar) for 6 hr. The solvent was removed to yield the title compound (157 mg, 104% yield) which was used without further purification in the next step. Analytically pure material was obtained through RP-HPLC (0.1% FA, Water:MeCN, hold at 90:10, then to 0:100) to give Compound 107. ¹H NMR (400 MHz, DMSO) δ 8.53 (d, 2H), 8.39 (s, 1H), 8.31 (s, 1H), 8.25 (s, 1H), 7.85 (s, 1H), 7.72 (s, 1H), 6.22 (s, 2H), 3.90 (s, 3H), 1.73 (s, 3H). LC-MS m/z 310.2 (M+H⁺).

### Example 86. 7-amino-8-(5-hydroxy-4-methylpyridin-3-yl)quinoxaline-6-carboxamide (113)

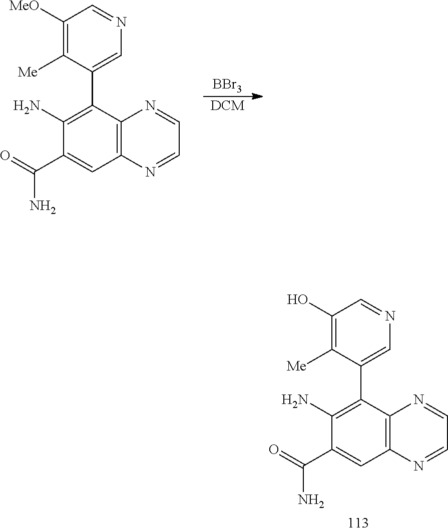

7-amino-8-(5-methoxy-4-methylpyridin-3-yl)quinoxaline-6-carboxamide (154 mg, 0.496 mmol, 1 equiv.) was suspended in DCM (10 mL) under an atmosphere of nitrogen gas in a microwave vessel. A solution of BBr₃ in DCM (1 M, 2.48 mL, 2.48 mmol, 5 equiv.) was added; and the reaction mixture was heated to 60° C. for 1 hr. A further 2.5 eq of BBr₃ was added and the mixture was heated to 80° C. for 2 hr. The solvent was removed. The residue was quenched with MeOH (10 mL) then the solvent was removed. The solid was suspended in water (3 mL) and neutralized with Et₃N (1 mL); then the solvent was removed. The residue was dissolved in MeOH, filtered, and subjected to RP-HPLC (0.1% FA, Water:MeCN, hold at 90:10, then to 0:100) to obtain Compound 113 (32.5 mg, 22% yield). ¹H NMR (400 MHz, DMSO) δ 12.77 (s, 1H), 10.00 (s, 1H), 8.60 (d, 2H), 8.46 (s, 1H), 8.38 (s, 1H), 8.20-8.09 (m, 1.47H), 7.80 (d, 2H), 6.29 (s, 2H), 1.79 (s, 3H). LC-MS m/z 296.2 (M+H⁺).

### Example 88. 7-Amino-8-(2-hydroxy-3-methylpyridin-4-yl)quinoxaline-6-carboxamide (110)

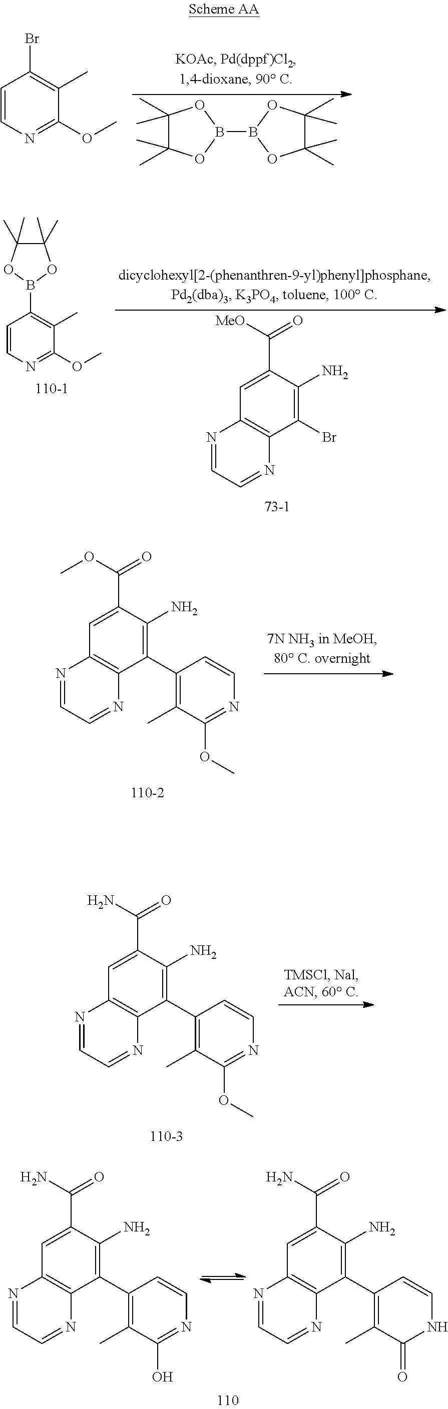

2-methoxy-3-methyl-4-(4,4,5,5-tetramethyl-1,3,2-dioxaborolan-2-yl)pyridine (Intermediate 110-1): A mixture of 4-bromo-2-methoxy-3-methyl-pyridine (390 mg, 1.93 mmol, 1.00 eq.), potassium acetate (580 mg, 5.91 mmol, 3.06 eq.), 4,4,5,5-tetramethyl-2-(4,4,5,5-tetramethyl-1,3,2-dioxaborolan-2-yl)-1,3,2-dioxaborolane (1.00 g, 3.94 mmol, 2.04 eq.) and Pd(dppf)Cl₂-DCM (80 mg, 0.10 mmol, 0.05 eq.) in anhydrous 1,4-dioxane (8 mL) was stirred at 90° C. overnight. The reaction mixture was filtered through Celite®, the cake was washed with EtOAc, the filtrate was concentrated and flash column chromatography (12 g ISCO) using 0-10% MeOH in DCM afforded the desired product (400 mg, 83% yield). LC-MS m/z 250 (M+H⁺)

Methyl 7-amino-8-(2-methoxy-3-methylpyridin-4-yl)quinoxaline-6-carboxylate (Intermediate 110-2): A mixture of 2-methoxy-3-methyl-4-(4,4,5,5-tetramethyl-1,3,2-dioxaborolan-2-yl)pyridine (190 mg, 0.76 mmol, 1.08 eq.), methyl 7-amino-8-bromo-quinoxaline-6-carboxylate (200 mg, 0.71 mmol, 1.00 eq.), potassium phosphate (460 mg, 2.17 mmol, 3.06 eq.), dicyclohexyl-[2-(9-phenanthryl)phenyl]phosphane (33 mg, 0.07 mmol, 0.10 eq.) and Pd₂(dba)₃ (71 mg, 0.08 mmol, 0.11 eq.) in toluene (6 mL) was degassed and stirred at 100° C. for 4 hours. The reaction mixture was concentrated onto Celite® and flash column chromatography (ISCO 12 g) using 0-10% MeOH in DCM afforded the desired product (50 mg, 21.7% yield). LC-MS m/z 325 (M+H⁺)

7-amino-8-(2-methoxy-3-methylpyridin-4-yl)quinoxaline-6-carboxamide (Intermediate 110-3): Methyl 7-amino-8-(2-methoxy-3-methylpyridin-4-yl)quinoxaline-6-carboxylate (50 mg, 0.15 mmol) was treated with 7N NH₃ in MeOH (10 mL) and stirred at 80° C. overnight. The reaction mixture was concentrated and flash column chromatography (ISCO 12 g) using 0-10% MeOH in DCM afforded the desired product (40 mg, 83.9% yield). ¹H NMR (400 MHz, DMSO) δ 8.60 (d, 2H), 8.45 (s, 1H), 8.37 (d, 1H), 8.08 (d, 1H), 7.78 (s, 1H), 6.75 (d, 1H), 6.26 (s, 2H), 5.76 (d, 1H), 3.94 (s, 3H), 1.77 (s, 3H), 1.06 (s, 1H) ppm. LC-MS m/z 310 (M+H⁺).

7-amino-8-(2-hydroxy-3-methylpyridin-4-yl)quinoxaline-6-carboxamide (110): Chloro(trimethyl)silane (0.10 mL, 0.79 mmol, 6.10 eq.) was added to a mixture of 7-amino-8-(2-methoxy-3-methyl-4-pyridyl)quinoxaline-6-carboxamide (40 mg, 0.13 mmol, 1.00 eq.) and sodium iodide (40 mg, 0.27 mmol, 2.06 eq.) in acetonitrile (4 mL) and the resulting dark red slurry was stirred at 60° C. overnight. The reaction mixture was concentrated, diluted in MeOH, water was added, reconcentrated, resuspended in MeOH, filtered through a frit and reverse prep-HPLC column chromatography using 0.1% formic acid in ACN and water (0-20%) afforded the desired product (2.50 mg, 6.55% yield). ¹H NMR (400 MHz, MeOD) δ 8.66-8.58 (m, 2H), 8.38 (t, 1H), 7.57-7.50 (m, 1H), 6.42-6.35 (m, 1H), 1.98-1.84 (m, 3H). ppm. LC-MS m/z 296 (M+H⁺).

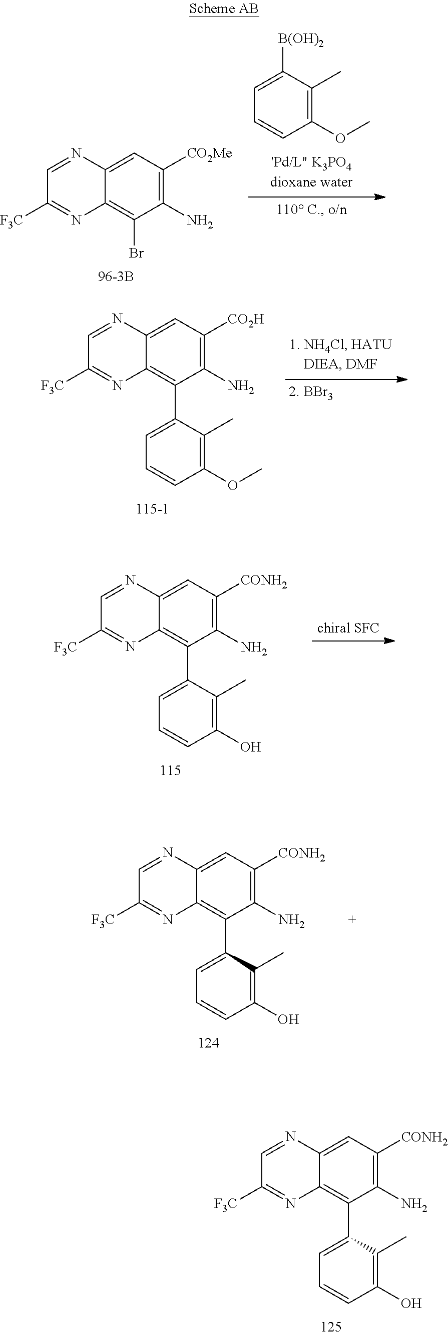

### Example 89. 7-Amino-8-(3-hydroxy-2-methylphenyl)-2-(trifluoromethyl)quinoxaline-6-carboxamide (115)

7-Amino-8-(3-methoxy-2-methylphenyl)-2-(trifluoromethyl)quinoxaline-6-carboxylic acid (Intermediate 115-1): A mixture of (3-methoxy-2-methylphenyl)boronic acid (66.0 mg, 0.398 mmol), methyl 7-amino-8-bromo-2-(trifluoromethyl)quinoxaline-6-carboxylate (67.0 mg, 0.191 mmol), potassium phosphate (0.336 g, 1.58 mmol), [2-(2-aminophenyl)phenyl]-methylsulfonyloxy-palladium; dicyclohexyl-[2-(2,6-dimethoxyphenyl)phenyl]phosphane (16.0 mg, 0.0205 mmol), and dicyclohexyl-[2-(2,6-dimethoxyphenyl)phenyl]phosphane (9.00 mg, 0.0219 mmol) in dioxane (2 mL) and water (0.5 mL) was stirred under N2 at 110° C. overnight. The reaction mixture was diluted with HCl (1M) to pH 3-4, extracted with EtOAc (2×). The extract was washed with brine, filtered and concentrated. The residue was used without purification (62 mg, crude). LC-MS m/z 378 (M+H⁺).

7-Amino-8-(3-hydroxy-2-methylphenyl)-2-(trifluoromethyl)quinoxaline-6-carboxamide(115): Above crude product was mixed with NH4Cl (50 mg), HATU (120 mg), DIEA (130 mg) in DMF (2 mL) and stirred at RT for 30 min. The reaction mixture was purified by prep-HPLC, to give crude target compound (46 mg). LC-MS m/z 377 (M+H⁺).

The material obtained above (46 mg) was dissolved in DCM (3 mL) and treated with BBr3 (1M in DCM, 0.5 mL). The mixture was stirred until reaction completion, quenched with NaHCO₃, and extracted with EtOAc (3×). The extract was evaporated, and the residue was purified by prep-HPLC, to give 7-amino-8-(3-hydroxy-2-methylphenyl)-2-(trifluoromethyl)quinoxaline-6-carboxamide (26 mg). ¹H NMR (400 MHz, DMSO) δ 9.45 (d, 1H), 8.97 (s, 1H), 8.54 (s, 1H), 8.42 (d, 1H), 7.90 (s, 1H), 7.12 (t, 1H), 6.88 (d, 1H), 6.57 (d, 1H), 6.19 (s, 2H), 1.70 (s, 3H). LC-MS m/z 363 (M+H⁺).

### Example 90. (P)-7-Amino-8-(3-hydroxy-2-methylphenyl)-2-(trifluoromethyl)quinoxaline-6-carboxamide (124) and (M)-7-Amino-8-(3-hydroxy-2-methylphenyl)-2-(trifluoromethyl)-quinoxaline-6-carboxamide (125)

Chiral SFC separation of Compound 115 (25 mg) (Instrument: Waters SFC Prep 150 Mgm; Column: Chiralpak® AD, 30×150 mm, 5 um; Conditions: Isocratic at 25% methanol with 75% CO₂; Flow Rate: 100 mL/min) provided Compound 124 (8 mg) and Compound 125 (9 mg).

Peak 1: retention time 2.56 min, 1H NMR (400 MHz, DMSO) δ 9.45 (s, 1H), 8.99 (d, 1H), 8.55 (s, 1H), 8.44 (d, 1H), 7.92 (s, 1H), 7.13 (t, 1H), 6.89 (d, 1H), 6.58 (d, 1H), 6.21 (s, 2H), 1.71 (s, 3H). LC-MS m/z 363 (M+H⁺).

Peak 2: retention time 3.09 min, 1H NMR (400 MHz, DMSO) δ 9.46 (d, 1H), 8.99 (d, 1H), 8.55 (s, 1H), 8.44 (d, 1H), 7.92 (s, 1H), 7.13 (t, 1H), 6.90 (d, 1H), 6.58 (d, 1H), 6.21 (s, 2H), 1.71 (s, 3H). LC-MS m/z 363 (M+H⁺).

### Example 91. 3-Amino-5-cyano-4-(3-hydroxy-2-methylphenyl)quinoline-2-carboxamide (129)

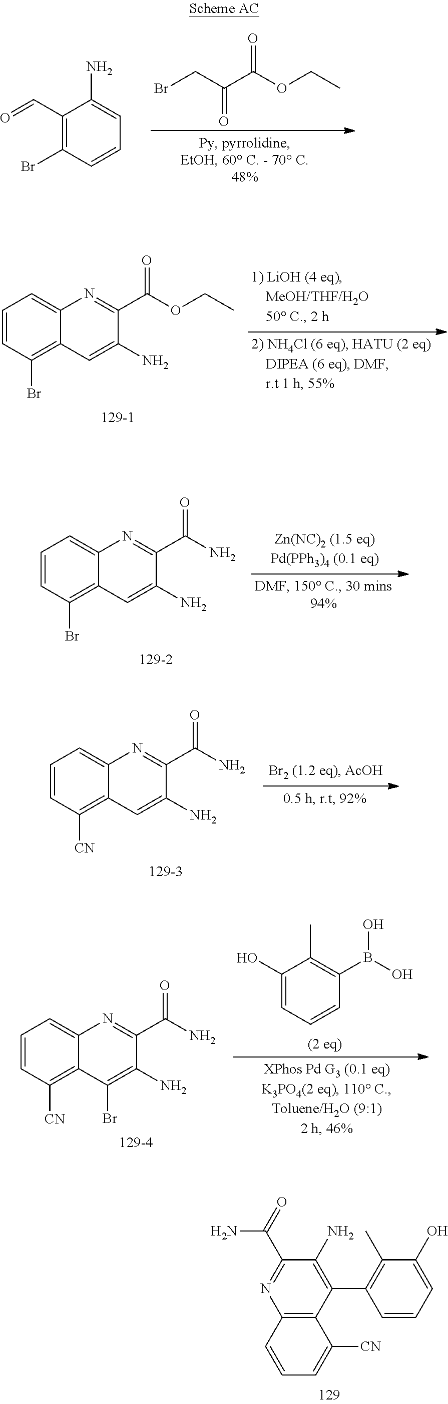

Ethyl 3-amino-5-bromoquinoline-2-carboxylate (Intermediate 129-1): To a mixture of pyridine (740 mg, 9.4 mol, 1.1 equiv.) and ethanol (24 mL) was added ethyl 2-bromopyruvate (1.8 g, 9.2 mmol, 1.1 equiv.) in ethanol (16 mL) dropwise over 20 min. The resulting mixture was heated at 60° C. for one hour and cooled to room temperature. 2-Amino-6-bromobenzaldehyde (1.7 g, 8.5 mmol) and pyridine (1.6 mL) were added. After heating at reflux for 5 h, pyrrolidine (1.2 g, 17 mmol, 2.0 equiv.) was added. The resulting mixture was heated to 70° C. for an additional 2 h and concentrated. The residue was purified by silica gel column chromatography (ethyl acetate/hexanes 1:3) to give the title compound ethyl 3-amino-5-bromoquinoline-2-carboxylate (1.2 g, 48%). LC-MS m/z [M+H]⁺=295.05.

3-amino-5-bromoquinoline-2-carboxamide (Intermediate 129-2): To a solution of ethyl 3-amino-5-bromoquinoline-2-carboxylate (1.2 g, 4.1 mmol) in MeOH/THF/H₂O (8 ml/8 ml/3.0 ml) added lithium hydroxide (490 mg, 5 equiv., 20 mmol). The reaction was stirred at 50° C. for 2 h. Volatiles were removed in vacuo. Water (20 mL) was added to the residue, and the pH was adjusted to 3-4 with 1 N hydrochloric acid. The resulting precipitate was collected by vacuum filtration. The filter cake was washed with water and dried to obtain the product 3-amino-5-bromo-quinoline-2-carboxylic acid (1.1 g, 84%).

A crude solution of 3-amino-5-bromo-quinoline-2-carboxylic acid (0.91 g, 3.41 mmol) in DMF (10 ml) was treated with ammonium chloride (1.21 g, 20.4 mmol, 6 equiv.) and DIPEA (3.6 ml, 20.4 mmol, 6 equiv.) followed by HATU (2.6 g, 6.8 mmol, 2 equiv.). The reaction mixture was stirred at room temperature for 1 h then quenched with saturated sodium bicarbonate solution. The mixture was extracted with dichloromethane (3×) The combined organic layers were dried over sodium sulfate, decanted and concentrated. The residue was purified by flash chromatography (EtOAc in heptane) to get the title product 3-amino-5-bromo-quinoline-2-carboxamide (501 mg, 55%). LC-MS m/z [M+H]+=266.10.

3-amino-5-cyanoquinoline-2-carboxamide (Intermediate 129-3): To the solution of 3-amino-5-bromo-quinoline-2-carboxamide (100 mg, 0.38 mmol) in DMF (1.5 mL), Pd(PPh₃)₄ (43 mg, 0.1 equiv., 0.038 mmol) and Zn(CN)₂ (66 mg, 1.5 equiv., 0.56 mmol) were added. The mixture was heated to 150° C. for 30 minutes using a microwave reactor. The mixture was cooled to room temperature, and a saturated aqueous sodium bicarbonate solution was added to the mixture. The mixture was filtered. The organic layer was extracted with ethyl acetate, dried over anhydrous sodium sulfate, and concentrated under reduced pressure. The residue was purified by silica gel column chromatography (heptane/ethyl acetate=90/10-50/50) to obtain the title compound 3-amino-5-cyano-quinoline-2-carboxamide (75 mg. 94% yield). LC-MS m/z [M+H]+=213.150.

3-amino-4-bromo-5-cyanoquinoline-2-carboxamide (Intermediate 129-4): To a solution of molecular bromine (67 mg, 1.2 equiv., 0.42 mmol) in DCM (0.2 mL) was added dropwise, under an argon atmosphere, a solution of 3-amino-5-cyano-quinoline-2-carboxamide (75 mg, 0.35 mmol) in acetic acid (2 mL). After 0.5 h of stirring at room temperature, the solvent was removed in vacuo. The residue was purified by silica gel column chromatography (ethyl acetate) to obtain the title compound 3-amino-4-bromo-5-cyano-quinoline-2-carboxamide (95 mg, 92% yield). LC-MS m/z [M+H]⁺=293.10.

3-amino-5-cyano-4-(3-hydroxy-2-methylphenyl)quinoline-2-carboxamide (129): To a solution of 3-amino-4-bromo-5-cyano-quinoline-2-carboxamide (50 mg, 0.17 mmol), potassium phosphate (46 mg, 2 equiv., 0.34 mmol), and (3-hydroxy-2-methyl-phenyl)boronic acid (52 mg, 2 equiv., 0.34 mmol) in toluene (1 mL) and water (0.1 mL) was added SPhos (7 mg, 0.1 equiv., 0.017 mmol) and SPhos Pd G3 (13.4 mg, 0.1 equiv., 0.017 mmol) under N₂ atmosphere. The reaction was stirred at 110° C. for 2 h. The cooled reaction mixture was purified by Prep-HPLC to give Compound 129 3-amino-5-cyano-4-(3-hydroxy-2-methyl-phenyl)quinoline-2-carboxamide (25 mg. 46% yield). ¹H NMR (400 MHz, DMSO) δ 9.43 (s, 1H), 8.48 (d, 1H), 8.15 (dd, 1H), 7.93-7.83 (m, 2H), 7.45 (dd, 1H), 7.07 (t, 1H), 6.89 (dd, 1H), 6.50 (dd, 1H), 6.27 (s, 2H), 1.70 (s, 3H). LC-MS m/z [M+H]⁺=319.20.

Compounds 98, 99, 102, and 130-339 were prepared according to the examples set forth above.

### BIOLOGICAL EXAMPLES

### Example A: PKMYT1 Inhibitory Activity

Biochemical IC₅₀ values were determined via ADP-Glo™ assay. Cellular IC₅₀ values were determined via AlphaLISA assay.

ADP-Glo™ PKMYT1 ATP-depletion assay. The activity of PKMYT1 is measured using a luciferase-luciferin based ATP detection reaction to quantify ATP depletion resulting from kinase-catalyzed phosphoryl transfer to CDK1 or autophosphorylation of PKMYT1. The ADP generated as a byproduct of the reaction is converted to ATP and used to drive a luciferase reaction. Test compounds were dissolved in DMSO and dispensed into Corning 3824 384-well plates prior to the experiment such that the final DMSO concentration was <1%. To the compounds was added 3 μL of PKMYT1 (93 kD, full length, N-terminal GST tag from Signal Chem). The reaction was started by the addition of 2 μL of CDK1 (58 kDa, full length, N-terminal GST tag from Signal Chem) and ATP in assay buffer (50 mM Hepes, 40 mM NaCl, 10 mM MgCl₂, 1 mM DTT, 0.1 mg/mL BSA, 2.5 mM MnCl2). Final assay concentrations were 50 nM PKMYT1, 13.3 nM CDK1, and 88 M ATP. Plates were centrifuged for 1 minute at 1000 RPM and incubated at RT for 120 minutes. After the incubation period, ADP-Glo™ reagent was added and the plates were incubated for another 40 minutes before finally adding the kinase detection reagent. 30 minutes after the addition, plates were placed on a PHERAstar® plate reader and read using the Luminescence Protocol, integration time at 0.5 seconds. Compounds exemplified above were tested by the PKMYT1 ATP depletion assay and exhibited IC₅₀ values as summarized in Table 2.

Cell lysate AlphaLISA® assay measuring inhibition of phosphorylation of CDK1 Thr14. Inhibition of phosphorylation of CDK1 at Thr14 was determined using AlphaLISA® SureFire® Ultra™ Kit from Perkin Elmer (ALSU-PCDK1-A10K). HCC1806 cells were seeded at 2K cells per well (50 μl/well) in 384-well plates (Corning 3570) and stored overnight at 37° C. and 5% CO₂. The following day, the compounds dissolved in DMSO were introduced to the cells and incubated for 4 hours at 37° C. and 5% CO₂. The cells were then washed with PBS using Blue®Washer and 10 μl of lysis buffer containing protease inhibitor was added. The plate was placed on an orbital shaker (at 4° C.) for 30 minutes and stored overnight at −80° C. for complete lysing. The next day, the plates were thawed at room temperature for at least 30 min. Reagents were prepared in a dimmed light setting (<100 lumens). The assay was run by first adding 5 μl of the acceptor mix (reaction buffer 1: reaction buffer 2: activation buffer: acceptor beads 47:47:4:2), covering the plate with foil, shaking for 2 minutes and incubating at RT for 60 minutes. The donor mix (dilution buffer: donor beads 47:3) was then added at 5 μl per well, placed on the shaker for 2 minutes and incubated at RT for 60 minutes before reading the plates on a PHERAstar® plate reader. The results were normalized to DMSO (0% Inhibition) and 3 μM of reference compound. Test compounds exhibited cellular IC₅₀ values as summarized in Table 2.

TABLE 2
Activity data for selected compounds of the invention

| Compound | Biochemical | Cellular |
|---|---|---|
| No. | IC₅₀ ^{a} | IC₅₀ ^{b} |
| 1 | + | † |
| 2 | +++++ | †††† |
| 3 | +++++ | †††† |
| 4 | +++++ | ††††† |
| 5 | +++ | †† |
| 6 | ++++ | †† |
| 7 | ++++ | †† |
| 8 | + | † |
| 9 | +++++ | ††††† |
| 10 | +++ | †† |
| 11 | +++++ | †††† |
| 12 | ++ | †††† |
| 13 | ++ | † |
| 14 | + | † |
| 15 | + | † |
| 17 | +++ | † |
| 18 | ++++ | †† |
| 19 | + | † |
| 20 | ++++ | †† |
| 22 | ++++ | †† |
| 23 | ++++ | †† |
| 24 | ++ | † |
| 25 | ++++ | †† |
| 27 | +++ | †† |
| 28 | ++++ | †††† |
| 30 | ++++ | ††††† |
| 31 | + | † |
| 32 | + | † |
| 33 | ++ | † |
| 34 | ++ | †† |
| 36 | ++++ | †† |
| 37 | ++ | †† |
| 38 | ++++ | †††† |
| 39 | + | † |
| 40 | +++ | †† |
| 41 | + | † |
| 42 | +++ | †† |
| 43 | ++++ | †† |
| 44 | ++++ | †† |
| 45 | ++ | † |
| 46 | ++ | † |
| 47 | ++ | †† |
| 48 | ++++ | †††† |
| 49 | +++ | †† |
| 50 | + | † |
| 52 | + | † |
| 53 | ++++ | †† |
| 54 | + | † |
| 55 | ++++ | †† |
| 56 | ++++ | ††† |
| 57 | +++++ | †††† |
| 58 | ++ | † |
| 59 | + | †† |
| 60 | +++ | † |
| 61 | +++++ | †††† |
| 62 | +++++ | †††† |
| 63 | ++ | † |
| 64 | ++++ | †††† |
| 65 | +++ | † |
| 66 | +++++ | †††† |
| 67 | ++++ | †† |
| 68 | ++++ | ††† |
| 69 | ++ | † |
| 70 | + | † |
| 72 | + | † |
| 73 | +++++ | †††† |
| 74 | +++ | † |
| 75 | + | † |
| 77 | ++++ | †† |
| 78 | ++++ | †† |
| 79 | + | † |
| 80 | ++ | † |
| 82 | +++++ | †††† |
| 83 | +++++ | †††† |
| 85 | ++ | † |
| 87 | +++++ | †††† |
| 89 | ++ | † |
| 91 | ++++ | †††† |
| 92 | +++++ | †††† |
| 93 | + | † |
| 94 | +++++ | †††† |
| 96 | ++++ | †† |
| 97 | +++++ | †††† |
| 100 | ++++ | †††† |
| 101 | ++++ | †††† |
| 103 | ++ | † |
| 104 | ++++ | †† |
| 105 | +++++ | †††† |
| 106 | + | † |
| 107 | ++++ | † |
| 108 | ++++ | †† |
| 109 | ++++ | † |
| 110 | + | † |
| 111 | ++ | † |
| 112 | +++++ | †††† |
| 113 | ++++ | †† |
| 114 | ++++ | †† |
| 115 | ++++ | ††† |
| 116 | ++++ | ††† |
| 117 | ++ | † |
| 118 | ++++ | †† |
| 119 | + | † |
| 120 | ++++ | †††† |
| 121 | ++++ | †† |
| 122 | ++ | † |
| 123 | ++++ | †† |
| 124 | +++++ | †††† |
| 125 | ++ | † |
| 126 | +++++ | †††† |
| 127 | +++++ | ††††† |
| 128 | ++ | † |
| 129 | +++++ | ††††† |
| 130 | + | † |
| 131 | + | † |
| 132 | + | † |
| 133 | ++++ | †† |
| 134 | + | † |
| 135 | ++++ | †† |
| 136 | + | † |
| 137 | ++ | † |
| 138 | ++++ | †† |
| 139 | +++++ | †††† |
| 140 | + | † |
| 141 | +++++ | †††† |
| 142 | ++++ | †† |
| 143 | ++++ | †† |
| 144 | ++++ | †† |
| 145 | ++++ | † |
| 146 | +++++ | †††† |
| 147 | ++ | †† |
| 148 | + | † |
| 149 | ++++ | †† |
| 150 | + | † |
| 151 | ++++ | †† |
| 152 | + | † |
| 153 | ++++ | ††† |
| 154 | +++++ | †††† |
| 155 | ++++ | ††† |
| 156 | +++++ | †††† |
| 157 | +++++ | †††† |
| 158 | ++ | † |
| 159 | + | † |
| 160 | + | ††† |
| 161 | +++++ | †††† |
| 162 | + | † |
| 163 | ++ | † |
| 164 | + | † |
| 165 | + | † |
| 166 | +++++ | †††† |
| 167 | ++ | † |
| 168 | +++++ | †††† |
| 169 | ++++ | ††† |
| 170 | + | † |
| 171 | ++++ | †† |
| 172 | ++++ | †††† |
| 173 | ++++ | †††† |
| 174 | +++++ | †††† |
| 175 | + | † |
| 176 | +++ | † |
| 177 | +++++ | †††† |
| 178 | + | † |
| 179 | +++++ | †††† |
| 180 | ++++ | †† |
| 181 | + | † |
| 182 | ++ | † |
| 183 | + | † |
| 184 | ++++ | † |
| 185 | + | † |
| 186 | ++++ | ††† |
| 187 | +++++ | †††† |
| 188 | +++++ | †††† |
| 189 | ++++ | ††† |
| 190 | ++++ | †† |
| 191 | ++ | † |
| 192 | + | † |
| 193 | + | † |
| 194 | +++++ | †††† |
| 195 | +++ | †† |
| 196 | ++++ | †† |
| 197 | + | † |
| 198 | + | † |
| 199 | ++++ | †† |
| 200 | ++++ | † |
| 201 | + | † |
| 202 | ++ | † |
| 203 | ++++ | †† |
| 204 | ++ | † |
| 205 | + | † |
| 206 | ++++ | †† |
| 207 | + | † |
| 208 | ++++ | ††† |
| 209 | +++++ | †††† |
| 210 | + | † |
| 211 | +++ | †† |
| 212 | ++++ | ††† |
| 213 | +++++ | †††† |
| 214 | +++ | † |
| 215 | + | † |
| 216 | ++ | † |
| 217 | +++++ | †††† |
| 218 | +++++ | ††† |
| 219 | ++ | † |
| 220 | ++++ | †† |
| 221 | + | † |
| 222 | +++++ | ††††† |
| 223 | + | † |
| 224 | ++ | † |
| 225 | + | † |
| 226 | +++++ | ††† |
| 227 | +++++ | †††† |
| 228 | + | † |
| 229 | ++ | † |
| 230 | ++++ | †† |
| 231 | +++ | † |
| 232 | ++ | † |
| 233 | ++ | † |
| 234 | + | † |
| 235 | ++ | † |
| 236 | ++ | † |
| 237 | ++++ | †† |
| 238 | ++ | † |
| 239 | + | † |
| 240 | +++++ | †††† |
| 241 | +++++ | ††††† |
| 242 | +++++ | ††††† |
| 243 | ++++ | †††† |
| 244 | ++++ | ††† |
| 245 | + | † |
| 246 | ++++ | ††† |
| 247 | ++ | † |
| 248 | ++++ | †† |
| 249 | + | † |
| 250 | + | † |
| 251 | +++++ | ††† |
| 252 | + | † |
| 253 | ++ | † |
| 254 | + | † |
| 256 | ++ | † |
| 257 | + | † |
| 258 | +++++ | †††† |
| 259 | +++ | † |
| 260 | + | † |
| 261 | + | † |
| 282 | ++++ | † |
| 283 | ++ | † |
| 284 | ++++ | † |
| 285 | + | † |
| 286 | +++ | † |
| 287 | + | † |
| 288 | +++++ | †††† |
| 289 | ++ | † |
| 290 | ++++ | †††† |
| 291 | ++ | † |
| 292 | ++++ | ††† |
| 293 | + | † |
| 294 | ++ | † |
| 295 | +++++ | †††† |
| 296 | + | † |
| 297 | + | † |
| 298 | ++ | † |
| 299 | + | † |
| 300 | ++++ | †† |
| 301 | + | † |
| 302 | ++ | † |
| 303 | + | † |
| 304 | + | † |
| 305 | ++++ | †† |
| 306 | ++++ | † |
| 307 | ++ | † |
| 308 | + | † |
| 309 | + | † |
| 310 | + | † |
| 311 | ++ | † |
| 312 | + | † |
| 313 | + | † |
| 314 | +++ | † |
| 315 | + | † |
| 316 | +++ | † |
| 317 | ++ | † |
| 318 | +++++ | †††† |
| 319 | ++++ | ††† |
| 320 | + | †† |
| 321 | +++++ | ††† |
| 322 | ++ | † |
| 323 | ++ | † |
| 324 | ++++ | ††† |
| 325 | ++ | †† |
| 326 | ++++ | †† |
| 327 | +++++ | ††††† |
| 328 | + | † |
| 329 | +++++ | †††† |
| 330 | +++++ | ††††† |
| 331 | ++++ | † |
| 332 | ++ | † |
| 333 | ++++ | ††††† |
| 334 | ++ | † |
| 335 | ++++ | †† |
| 336 | +++++ | †††† |
| 337 | +++ | †† |
| 338 | ++++ | †† |
| 339 | ++++ | †† |

^{a} +++++: IC₅₀ < 50 nM
++++: 50 nM ≤ IC₅₀ < 500 nM
+++: 500 nM ≤ IC₅₀ < 1 μM
++: 1 μM ≤ IC₅₀ < 5 μM
+: 5 μM ≤ IC₅₀
^{b} †††††: IC₅₀ < 50 nM
††††: 50 nM ≤ IC₅₀ < 500 nM
†††: 500 nM ≤ IC₅₀ < 1 μM
††: 1 μM ≤ IC₅₀ < 5 μM
†: 5 μM ≤ IC₅₀

### Example B: Heptatocyte Stability Assay

Metabolic stability of testing compounds can be evaluated using human, rat, mouse, or other animal hepatocytes to assess intrinsic clearance.

Cryopreserved hepatocytes were removed from the liquid nitrogen tank and thawed in a 37° C. water bath. As soon as the cells pulled away from the vial wall, they were decanted into 48 mL of warm HT medium. Cells were centrifuged for four minutes at 420 rpm (50 g). After removing the supernatant, pellet was re-suspended in warm DMEM medium. Cell density was counted by a hemacytometer.

The assay was carried out in 96-well microtiter plates. Compounds were incubated for 0, 60, 120, and 180 minutes at 37° C. with hepatocytes. The reaction mixtures (50 L) contained a final concentration of 1 μM test compound, 0.5 million cells/mL hepatocytes in the DMEM medium. At each of the time points (for example, 0, 1, 2, and 3 hours), 200 μL of quench solution (100% acetonitrile with 0.1% formic acid) with internal standard was transferred to each well. Midazolam was included as a positive control to verify assay performance. Plates were sealed and centrifuged at 4° C. for 15 minutes at 4000 rpm. The supernatant was transferred to fresh plates for LC/MS/MS analysis.

All samples were analyzed on LC/MS/MS using an AB Sciex API 4000 instrument, coupled to a Shimadzu LC-20AD LC Pump system. Analytical samples were separated using a Waters Atlantis T3 dC18 reverse phase HPLC column (20 mm×2.1 mm) at a flow rate of 0.5 mL/min. The mobile phase was 0.1% formic acid in water (solvent A) and 0.1% formic acid in 100% acetonitrile (solvent B).

The extent of metabolism was calculated as the disappearance of the test compound, compared to the 0-min control reaction incubations. Initial rates were calculated for the compound concentration and used to determine t_{1/2} values and subsequently, the intrinsic clearance, CLint=(0.693)(1/t_{1/2} (min))(mL incubation/million cells).

The testing results showed that certain compounds of the present disclosure have advantageously low rates of hepatic metabolic clearance.

### Example C: Other Kinase Inhibitory Activity

Kinome profiles for test compounds were determined using a panel of 378 assays (374 kinases) all run in a radiometric format using 33P-labeled ATP (full human WT kinase [Km ATP] KinaseProfiler LeadHunter Panel; Eurofins). Each assay was run using a Mg/ATP mixture such that the concentration of ATP was the Km for ATP in that kinase. Details for example assays are provided below.

c-Src Inhibition Assay. c-Src (h) was incubated with 8 mM MOPS pH 7.0, 0.2 mM EDTA, 250 μM KVEKIGEGTYGVVYK (Cdc2 peptide), 10 mM magnsium acetate and [γ-³³P]-ATP (specific activity and concentration as required). The reaction was initiated by the addition of the Mg/ATP mixture. After incubation for 40 minutes at room temperature, the reaction was stopped by the addition of phosphoric acid to a concentration of 0.5%. An aliquot of the reaction was then spotted onto a filter and washed four times for four minutes in 0.425% phosphoric acid and once in methanol prior to drying and scintillation counting.

Lek Inhibition Assay. Lck (h) was incubated with 50 mM Tris pH 7.5, 0.1 mM EGTA, 0.1 mM Na₃VO₄, 250 M KVEKIGEGTYGVVYK (Cdc2 peptide), 10 mM magnesium acetate and [γ-³³P]-ATP (specific activity and concentration as required). The reaction was initiated by the addition of the Mg/ATP mixture. After incubation for 40 minutes at room temperature, the reaction was stopped by the addition of phosphoric acid to a concentration of 0.5%. An aliquot of the reaction was then spotted onto a filter and washed four times for four minutes in 0.425% phosphoric acid and once in methanol prior to drying and scintillation counting.

A high selectivity for inhibition of PKMYT1 over other kinases was observed for certain compounds of the present disclosure.

### Example D: Assessment of CYP1A2, CYP2B6, and CYP3A4 Induction

To evaluate the induction of hepatocellular gene expression, the inducers omeprazole (50 μM), rifampicin (10 μM), and phenobarbital (1 mM) were used for CYP1A2, CYP3A4, and CYP2B6, respectively. Human hepatocytes were treated with each compound followed by incubation of the hepatocyte culture along with the addition of three marker substrates for specific CYP activity: phenacetin (CYP1A2; 100 PM), bupropion (CYP2B6; 500 μM), and testosterone (CYP3A4; 200 M). Induction of CYP1A2, CYP2B6, and CYP3A4 by inducers and test compounds were investigated using the cells.

The assessment of CYP activity was performed with a liquid chromatography-tandem mass spectrometry method which assessed activity of CYP1A2, CYP2B6, and CYP3A4 by examining the formation of the probe substrate metabolites, acetaminophen, hydroxybupropion, and 6β-hydroxytestosterone.

Certain compounds of the present disclosure exhibited an advantageously low propensity for induction of CYP3A4 expression.

### OTHER EMBODIMENTS

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A compound according to Formula I: 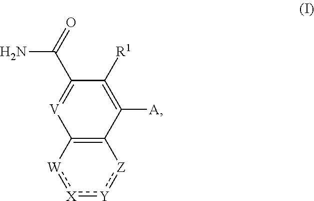
or a pharmaceutically acceptable salt thereof,
wherein:
each [symbol] represents a single bond, a double bond, or a delocalized π bond;
V is selected from the group consisting of CH and N;
W is selected from the group consisting of N, NR^{a}, CR², and C═O;
X is selected from the group consisting of CR³, C═O, N, and NR^{a};
Y is selected from the group consisting of CR⁴, C═O, N, and NR^{a};
Z is selected from the group consisting of N, NR^{a}, CR⁵, and C═O;
R¹ is selected from the group consisting of amino, hydroxy, C₁₋₆ alkyl, and hydrogen;
R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently substituted with 0, 1, 2, 3, or 4 R⁶;
each L is independently selected from the group consisting of a covalent bond, —O—, C₁₋₆ alkylene, and NR^{a};
any two adjacent R², R³, R⁴, and R⁵ are optionally taken together to form a 5- or 6-membered carbocyle or heterocycle;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂; and
each R^{a} is independently hydrogen or C₁₋₆ alkyl;
provided that when R¹ is hydroxy or hydrogen, ring A is selected from the group consisting of:
6- to 14-membered carbocyclyl substituted with (i) at least one R⁶ selected from the group consisting of hydroxy, C₁₋₆ hydroxyalkyl, C₁₋₆ alkoxy, and oxo, and (ii) at least one R⁶ selected from the group consisting of C₁₋₆ alkyl and halogen,
6- or 7-membered monocyclic heterocyclyl comprising at least one oxygen atom, nitrogen atom, or sulfur atom, which heterocycle is substituted with at least one R⁶ selected from the group consisting of hydroxy, C₁₋₆ hydroxyalkyl, oxo, C₁₋₆ alkoxy, C₁₋₆ alkyl, or halogen, and
9- to 14-membered bicyclic heterocyclyl comprising at least one oxygen atom, nitrogen atom, or sulfur atom, wherein the bicyclic heterocyclyl is substituted with 0, 1, 2, 3, or 4 R6.

2. The compound of claim 1, or a pharmaceutically acceptable salt thereof, having a structure according to Formula Ta:

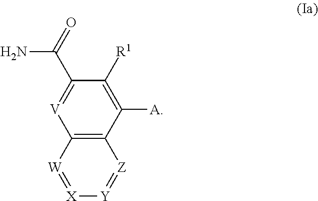

3. The compound of claim 2, or a pharmaceutically acceptable salt thereof, wherein at least one of V, W, X, Y, and Z is N.

4. The compound of claim 2, or a pharmaceutically acceptable salt thereof, wherein at least two of V, W, X, Y, and Z are N.

5. The compound of any one of claims 1-4, or a pharmaceutically acceptable salt thereof, wherein R¹ is selected from the group consisting of amino, hydroxy, and hydrogen.

6. The compound of any one of claims 1-5, or a pharmaceutically acceptable salt thereof, wherein R¹ is amino.

7. The compound of any one of claims 1-5, or a pharmaceutically acceptable salt thereof, wherein R¹ is hydrogen.

8. The compound of any one of claims 1-5, or a pharmaceutically acceptable salt thereof, wherein R¹ is hydroxy.

9. The compound of any one of claims 1-8, or a pharmaceutically acceptable salt thereof, wherein R², R³, R4, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently and optionally substituted with C₁₋₆ alkyl.

10. The compound of any one of claims 1-9, or a pharmaceutically acceptable salt thereof, wherein R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁₋₆ alkyl).

11. The compound of claim 10, or a pharmaceutically acceptable salt thereof, wherein W is CR², and R² is selected from the group consisting of hydrogen, halogen, cyano, C₁₋₆ alkyl, and C₁₋₆ haloalkyl.

12. The compound of any one of claims 9-11, or a pharmaceutically acceptable salt thereof, wherein:
X is CR³;
Y is CR⁴; or
X is CR³ and Y is CR⁴;
and wherein R³ and R⁴ are independently selected from the group consisting of hydrogen, hydroxy, halogen, cyano, C₁₋₆ alkyl, C₁₋₆ haloalkyl, and C₁₋₆ alkoxy.

13. The compound of any one of claims 9-12, or a pharmaceutically acceptable salt thereof, wherein Z is CR⁵, and R⁵ is selected from the group consisting of hydrogen, hydroxy, halogen, cyano, and C₁₋₆ alkyl.

14. The compound any one of claims 1-13, or a pharmaceutically acceptable salt thereof, wherein each R⁶ of ring A is independently selected from the group consisting of hydroxy, cyano, amino, halogen, —CHO, C₁₋₆ alkyl, C₁₋₆ haloalkyl, C₁₋₆ hydroxyalkyl, C₁₋₆ alkoxy, and oxo.

15. The compound of claim 14, or a pharmaceutically acceptable salt thereof, wherein each R⁶ of ring A is independently selected from the group consisting of hydroxy, C₁₋₆ hydroxyalkyl, C₁₋₆ alkoxy, and oxo.

16. The compound of any one of claims 9-15, or a pharmaceutically acceptable salt thereof, wherein R¹ is amino, and ring A is:
3- to 14-membered carbocyclyl substituted with 1, 2, 3, or 4 R⁶, or
5- to 14-membered nitrogen-containing heterocyclyl substituted with 0, 1, 2, 3, or 4 R⁶.

17. The compound of claim 16, or a pharmaceutically acceptable salt thereof, wherein ring A is 3- to 14-membered carbocyclyl substituted with 1, 2, or 3 R⁶, and each R⁶ is independently selected from the group consisting of hydroxy, cyano, amino, halogen, C₁₋₆ alkyl, C₁₋₆ haloalkyl, C₁₋₆ hydroxyalkyl, C₁₋₆ alkoxy, and oxo.

18. The compound of claim 17, or a pharmaceutically acceptable salt thereof, wherein ring A is phenyl substituted with 1, 2, or 3 R⁶.

19. The compound of claim 16, or a pharmaceutically acceptable salt thereof, wherein ring A is 5- to 14-membered nitrogen-containing heterocyclyl substituted with 0, 1, 2, or 3 R⁶, and each R⁶ is independently selected from the group consisting of hydroxy, amino, halogen, —CHO, C₁₋₆ alkyl, C₁₋₆ haloalkyl, C₁₋₆ hydroxyalkyl, C₁₋₆ alkoxy, and oxo.

20. The compound of claim 19, or a pharmaceutically acceptable salt thereof, wherein ring A is 5- to 7-membered monocyclic nitrogen-containing heterocyclyl substituted with 0, 1, 2, or 3 R⁶.

21. The compound of any one of claims 1-20, or a pharmaceutically acceptable salt thereof, wherein ring A is selected from the group consisting of: 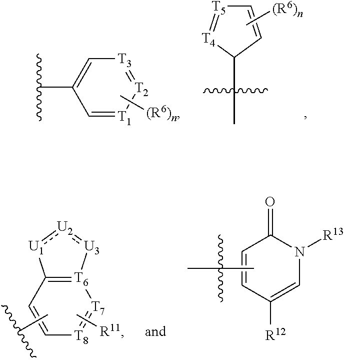
wherein:
each [symbol] represents a single bond, a double bond, or a delocalized π bond;
T₁, T₂, T₃, T₄, T₅, T₆, T₇, and T₈ are independently selected from the group consisting of N, C, and CH;
U₁, U₂, and U₃ are independently selected from the group consisting of N, NR¹⁰, O, S, and CR¹⁴;
each R⁶ is independently selected from the group consisting of amino, oxo, cyano, hydroxy, C₁₋₆ alkyl, C₁₋₆ alkoxy, C₁₋₆ haloalkyl, C₁₋₆ hydroxyalkyl, and halogen;
R¹¹, R¹², and R¹⁴ are independently selected from the group consisting of hydrogen, amino, oxo, cyano, hydroxy, C₁₋₆ alkyl, C₁₋₆ alkoxy, C₁₋₆ haloalkyl, C₁₋₆ hydroxyalkyl, and halogen;
each R¹⁰ and R¹³ are independently selected from the group consisting of hydrogen, C₁₋₆ alkyl, and CHO; and
n is 1, 2, 3, or 4.

22. The compound of claim 21, or a pharmaceutically acceptable salt thereof, wherein ring Ais 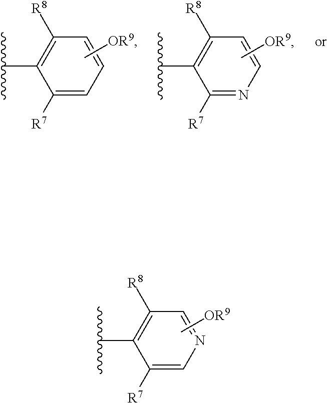
wherein R⁷ and R⁸ are independently selected from the group consisting of hydrogen, amino, cyano, C₁₋₆ alkyl, C₁₋₆ alkoxy, C₁₋₆ haloalkyl, C₁₋₆ hydroxyalkyl, and halogen; and
R⁹ is hydrogen or C₁₋₆ alkyl.

23. The compound of claim 21, or a pharmaceutically acceptable salt thereof, wherein ring A is selected from the group consisting of

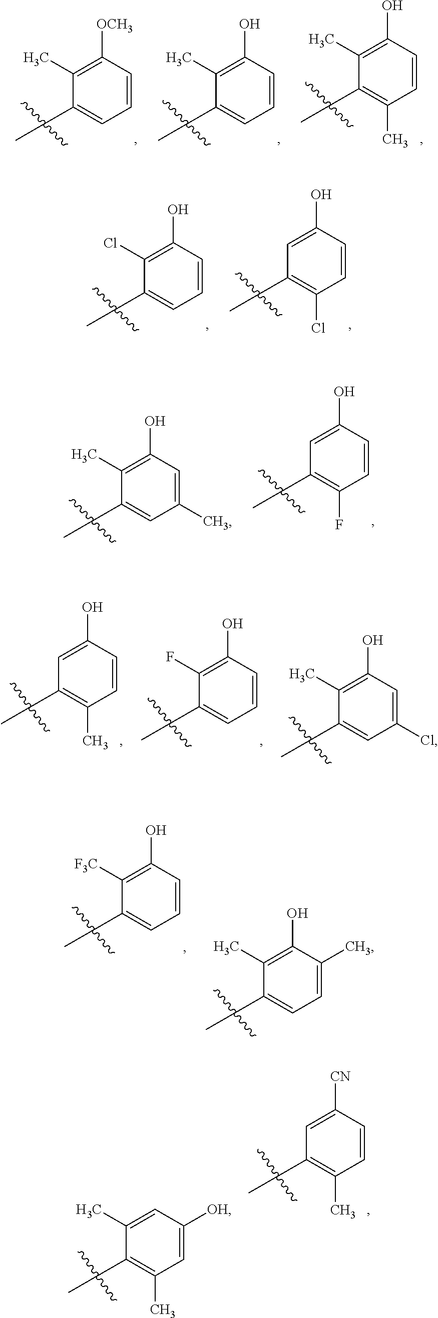

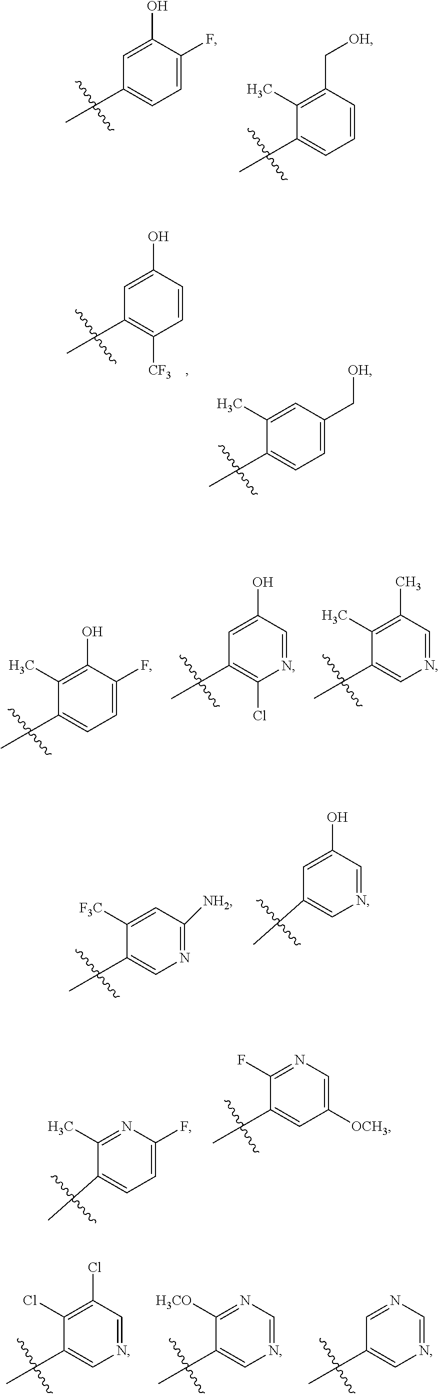

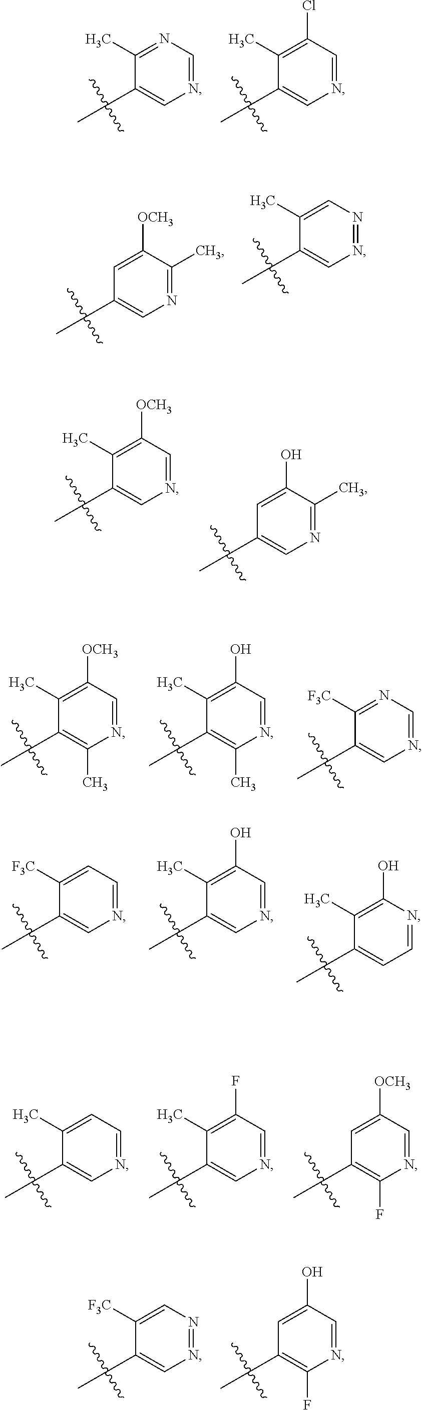

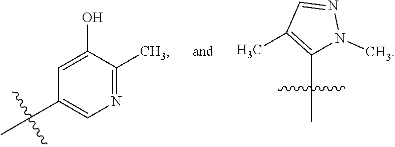

24. The compound of claim 21, or a pharmaceutically acceptable salt thereof, wherein ring A is 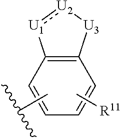
wherein [symbol]
represents a single bond or a double bond;
R¹¹ is selected from the group consisting of hydrogen, C₁₋₆ alkyl, and halogen; and
R¹⁴ is selected from the group consisting of hydrogen, amino, oxo, C₁₋₆ alkyl, and halogen.

25. The compound of claim 21, or a pharmaceutically acceptable salt thereof, wherein ring A is selected from the group consisting of

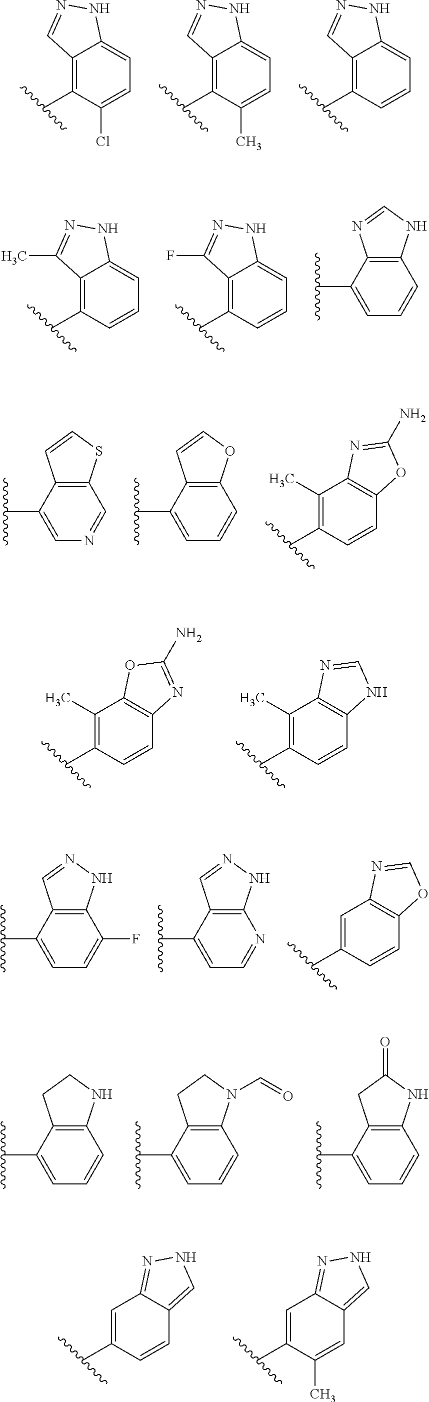

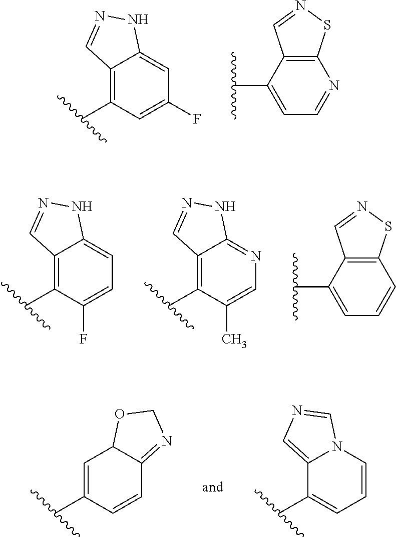

26. The compound of claim 21, or a pharmaceutically acceptable salt thereof, wherein ring A is

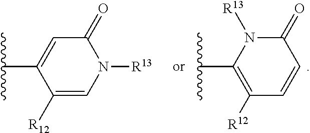

27. The compound of claim 26, or a pharmaceutically acceptable salt thereof, wherein R¹² and R¹³ are independently hydrogen or C₁₋₆ alkyl.

28. The compound of any one of claims 1-27, or a pharmaceutically acceptable salt thereof, having a structure according to Formula Ib:

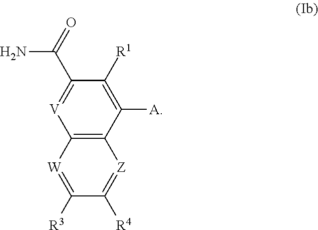

29. The compound of claim 28, or a pharmaceutically acceptable salt thereof, having a structure according to Formula Ic:

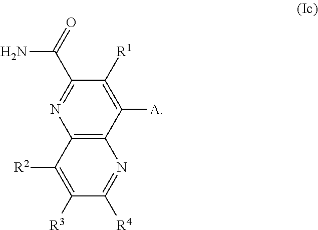

30. The compound of claim 29, or a pharmaceutically acceptable salt thereof, having a structure according to Formula Ic-1

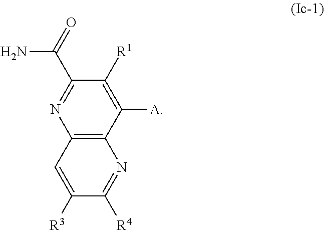

31. The compound of claim 28, or a pharmaceutically acceptable salt thereof, having a structure according to Formula Id:

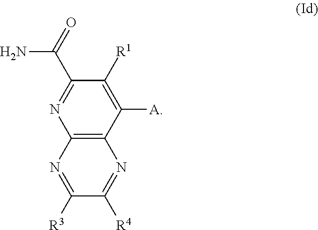

32. The compound of claim 28, or a pharmaceutically acceptable salt thereof, having a structure according to Formula Ie:

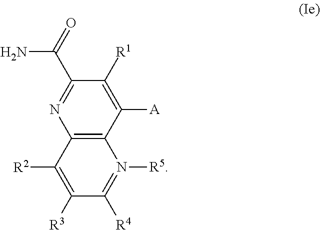

33. The compound of claim 28, or a pharmaceutically acceptable salt thereof, having a structure according to Formula If:

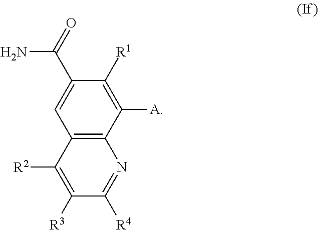

34. The compound of claim 33, or a pharmaceutically acceptable salt thereof, having a structure according to Formula If-1:

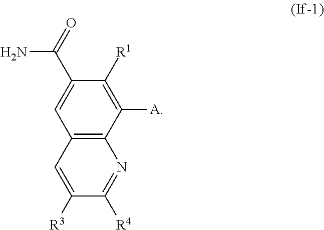

35. The compound of claim 28, or a pharmaceutically acceptable salt thereof, having a structure according to Formula Ig:

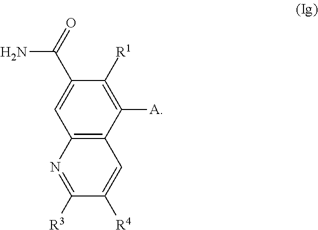

36. The compound of claim 28, or a pharmaceutically acceptable salt thereof, having a structure according to Formula Ih:

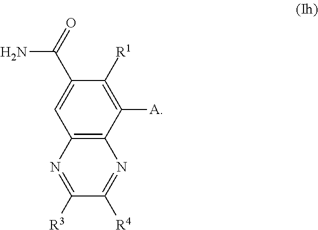

37. The compound of any one of claims 1-2 and 28-36, or a pharmaceutically acceptable salt thereof, wherein:
R², R³, R⁴, and R⁵, when present, are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, and —COO(C₁-6 alkyl); and
ring A is selected from the group consisting of 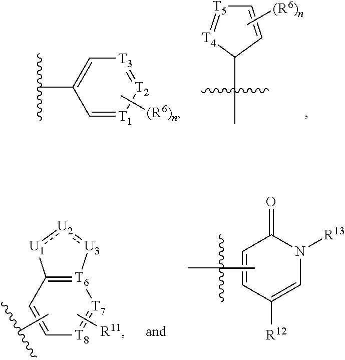
wherein:
each [symbol] represents a single bond, a double bond, or a delocalized π bond;
T₁, T₂, T₃, T₄, T₅, T₆, T₇, and T₈ are independently selected from the group consisting of N, C, and CH;
U₁, U₂, and U₃ are independently selected from the group consisting of N, NR¹⁰, O, S, and CR¹⁴;
each R⁶ is independently selected from the group consisting of amino, oxo, cyano, hydroxy, C₁₋₆ alkyl, C₁₋₆ alkoxy, C₁₋₆ haloalkyl, C₁₋₆ hydroxyalkyl, and halogen;
R¹¹, R¹², and R¹⁴ are independently selected from the group consisting of hydrogen, amino, oxo, cyano, hydroxyl, C₁₋₆ alkyl, C₁₋₆ alkoxy, C₁₋₆ haloalkyl, C₁₋₆ hydroxyalkyl, and halogen;
each R¹⁰ and R¹³ are independently selected from the group consisting of hydrogen, C₁₋₆ alkyl, and CHO; and
n is 1, 2, 3, or 4.

38. The compound of claim 37, or a pharmaceutically acceptable salt thereof, wherein:
R¹ is amino or hydrogen;
R², R³, R⁴, and R⁵ are independently selected from the group consisting of hydrogen, hydroxy, halogen, C₁₋₆ alkyl, C₁₋₆ haloalkyl, and C₁₋₆ alkoxy; and
ring A is selected from the group consisting of 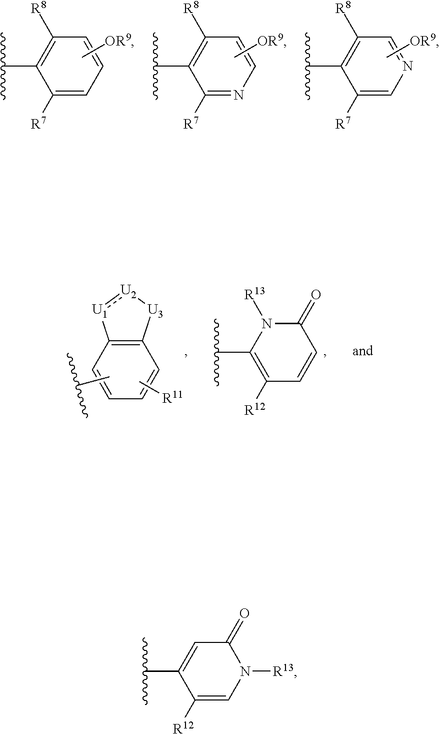
wherein R⁷ and R⁸ are independently selected from the group consisting of hydrogen, amino, cyano, C₁₋₆ alkyl, C₁₋₆ alkoxy, C₁₋₆ haloalkyl, C₁₋₆ hydroxyalkyl, and halogen;
R⁹ is hydrogen or C₁₋₆ alkyl;
R¹¹ is selected from the group consisting of hydrogen, C₁₋₆ alkyl, and halogen; and
R¹⁴ is selected from the group consisting of hydrogen, amino, oxo, C₁₋₆ alkyl, and halogen.

39. The compound of claim 38, or a pharmaceutically acceptable salt thereof, wherein:
R¹ is amino;
R², R³, R⁴, and R⁵ are independently selected from the group consisting of hydrogen, hydroxy, halogen, C₁₋₆ alkyl, C₁₋₆ haloalkyl, and C₁₋₆ alkoxy; and
ring A is selected from the group consisting of 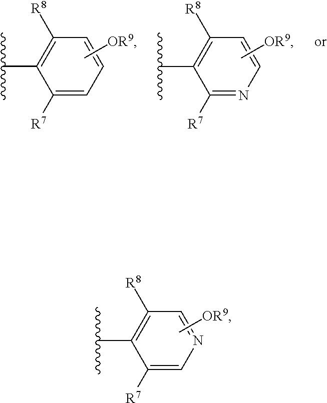
wherein R⁷ and R⁸ are independently selected from the group consisting of hydrogen, C₁₋₆ alkyl, and halogen.

40. The compound of claim 28, or a pharmaceutically acceptable salt thereof, having a structure according to Formula Ik: 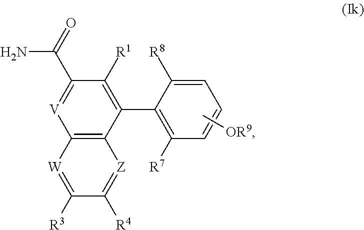
wherein:
at least one of V, W, and Z is N;
R⁷ and R⁸ are independently selected from the group consisting of hydrogen, C₁₋₆ alkyl, C₁₋₆ alkoxy, C₁₋₆ haloalkyl, and halogen; and
R⁹ is hydrogen or C₁₋₆ alkyl.

41. The compound of claim 40, or a pharmaceutically acceptable salt thereof, having a structure according to Formula Ik-3 or Ik-4:

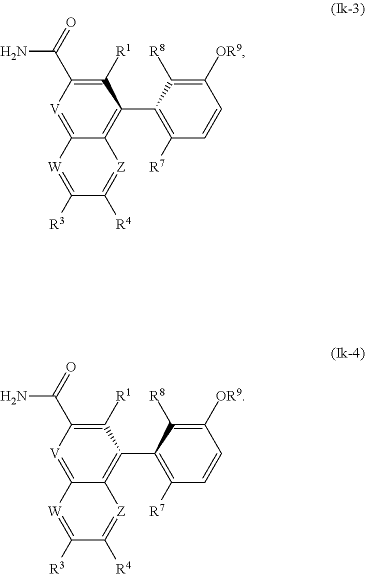

42. The compound of any one of claims 37-41, or a pharmaceutically acceptable salt thereof, wherein:
R³ and R⁴ are independently selected from the group consisting of hydrogen, hydroxy, halogen, C₁₋₆ alkyl, C₁₋₆ haloalkyl, and C₁₋₆ alkoxy; and
R⁷ and R⁸ are independently selected from the group consisting of hydrogen, C₁₋₆ alkyl, C₁₋₆ haloalkyl, and halogen.

43. The compound of claim 28, or a pharmaceutically acceptable salt thereof, having a structure according to Formula Im-1 or Im-2: 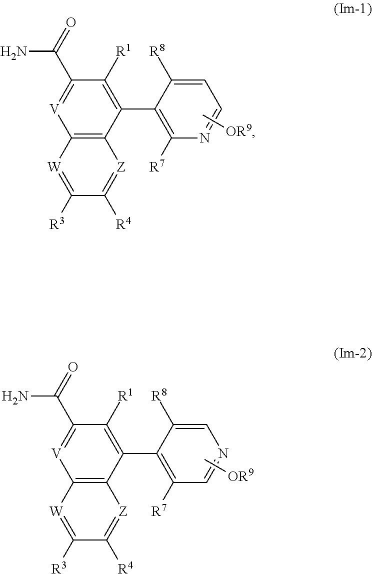
wherein:
at least one of V, W, and Z is N;
R⁷ and R⁸ are independently selected from the group consisting of hydrogen, C₁₋₆ alkyl, C₁₋₆ alkoxy, C₁₋₆ haloalkyl, and halogen; and
R⁹ is hydrogen or C₁₋₆ alkyl.

44. The compound of claim 28, or a pharmaceutically acceptable salt thereof, having a structure according to Formula In: 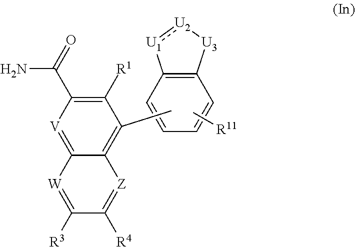
wherein at least one of V, W, and Z is N.

45. The compound of claim 43 or claim 44, or a pharmaceutically acceptable salt thereof, wherein:
R¹ is amino; and
R², R⁴, R⁴, and R⁵, when present, are independently selected from the group consisting of hydrogen, hydroxy, halogen, C₁₋₆ alkyl, C₁₋₆ haloalkyl, and C₁₋₆ alkoxy.

46. A compound, or a pharmaceutically acceptable salt thereof, selected from any of the compounds in Table 1A, Table 1B, and Table 1C.

47. A pharmaceutical composition comprising a compound according to any one of claims 1-46, or a pharmaceutically acceptable salt thereof, and one or more pharmaceutically acceptable excipients.

48. A method for treating cancer comprising administering a therapeutically effective amount of a compound according to any one of claims 1-46, or a pharmaceutically acceptable salt thereof, to a subject in need thereof.

49. A process for making a compound of Formula I, 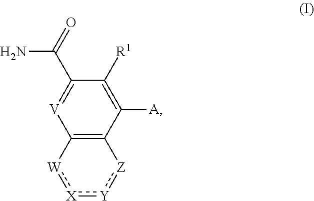
or a pharmaceutically acceptable salt thereof, comprising
(a) combining a compound of Formula (b) and a compound of Formula (c) to form a compound of Formula (d) 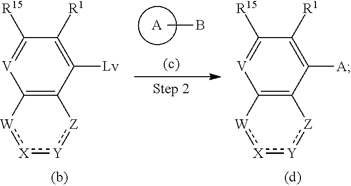
(b) converting the compound of Formula (d) to yield the compound of Formula I, 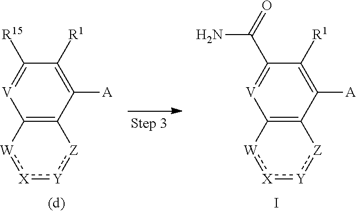
wherein
Lv is a leaving group; 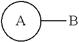
is a boronic acid or boronic ester of ring A;
each [symbol] represents a single bond, a double bond, or a delocalized π bond;
V is selected from the group consisting of N and CH;
W is selected from the group consisting of N, NR^{a}, CR², and C═O;
X is selected from the group consisting of N, NR^{a}, CR³, and C═O;
Y is selected from the group consisting of N, NR^{a}, CR⁴, and C═O;
Z is selected from the group consisting of N, NR^{a}, CR⁵, and C═O;
R¹ is selected from the group consisting of amino, hydroxy, C₁₋₆ alkyl, and hydrogen;
R², R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), 5- to 12-membered heterocyclyl, -L-(C₆₋₁₄ aryl), and -L-(5- to 14-membered heteroaryl), wherein the 5- to 12-membered heterocyclyl, C₆₋₁₄ aryl, and 5- to 14-membered heteroaryl are independently substituted with 0, 1, 2, 3, or 4 R⁶;
each L is independently selected from the group consisting of a covalent bond, —O—, C₁₋₆ alkylene, and NR^{a};
any two adjacent R², R³, R⁴, and R⁵ are optionally taken together to form a 5- or 6-membered carbocyle or heterocycle;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —CHO, —NO₂, —CO(C₁₋₆ alkyl), —COOR^{a}, —CON(R^{a})₂, —SO₂(C₁₋₆ alkyl), and —SO₂N(R^{a})₂;
R¹⁵ is —COOR^{a}, —COONH₂, or CN; and
each R^{a} is independently hydrogen or C₁₋₆ alkyl.

50. The process of claim 49, wherein the process further comprises converting a compound of formula (a) to the compound of formula (b)

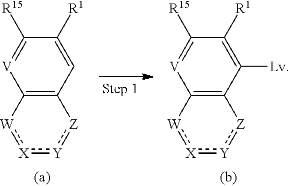

51. The process of claim 49 or claim 50, wherein the compound of Formula (c) is:

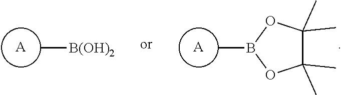

52. The process of any one of claims 49-51, wherein Lv is halogen.
